(19) United States
(12) Patent Application Publication
(10) Pub. No.: US 2003/0215899 A1
(43) Pub. Date: Nov. 20, 2003

(54) Reversible oxidation of protein tyrosine phosphatases

(75) Inventors: Tzu-Ching Meng, Oyster Bay, NY (US); Nicholas K. Tonks, Huntington, NY (US); Deborah E. Cool, Snohomish, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignees: CEPTYR, Inc., Bothell, WA; Cold Spring Harbor Laboratory, Cold Spring Harbor, NY

(21) Appl. No.: 10/366,547

(22) Filed: Feb. 12, 2003

Related U.S. Application Data
(60) Provisional application No. 60/356,810, filed on Feb. 13, 2002.

(51) Int. Cl. C12Q 1/42

(52) U.S. Cl. 435/21

(57) ABSTRACT

The invention relates to a method of identifying any protein tyrosine phosphatase (PTP) that undergoes reversible modification of PTP active site invariant cysteine within a cell, such that the phosphatase is transiently protected from irreversible active site invariant cysteine-directed PTP inactivating agents. Methods related to regulation of PTPs by reactive oxygen species (ROS) in a cellular environment are provided. Multiple PTPs are shown to be reversibly oxidized and inactivated following treatment of cells with H₂O₂ or with physiological stimuli that promote ROS formation, and inhibition of PTP function is shown to contribute to ROS-induced mitogenesis. Transient oxidation of the PTP catalytic site invariant cysteine is exploited in methods to identify which of multiple candidate PTPs are components of a given biological signal transduction pathway, without a requirement for first specifically purifying any particular candidate PTP.

### CROSS-REFERENCE TO RELATED APPLICATION

This application claims the benefit of U.S. Provisional Patent Application No. 60/356,810 filed Feb. 13, 2002, which is incorporated herein by reference in its entirety.

### STATEMENT OF GOVERNMENT INTEREST

The United States government may have certain rights in this invention under grant number R01-GM55989 from the National Institutes of Health.

### BACKGROUND OF THE INVENTION

The present invention relates generally to compositions and methods useful for treating conditions associated with defects in cell proliferation, cell differentiation and/or cell survival. The invention is more particularly related to identifying protein tyrosine phosphatases (PTPs) that are reversibly modified, including PTPs that are reversibly oxidized components of inducible biological signaling pathways.

Reversible protein tyrosine phosphorylation, coordinated by the action of protein tyrosine kinases (PTKs) that phosphorylate certain tyrosine residues in polypeptides, and protein tyrosine phosphatases (PTPs) that dephosphorylate certain phosphotyrosine residues, is a key mechanism in regulating many cellular activities. It is becoming apparent that the diversity and complexity of the PTPs and PTKs are comparable, and that PTPs are equally important in delivering both positive and negative signals for proper function of cellular machinery. Regulated tyrosine phosphorylation contributes to specific pathways for biological signal transduction, including those associated with cell division, cell survival, apoptosis, proliferation and differentiation. Defects and/or malfunctions in these pathways may underlie certain disease conditions for which effective means for intervention remain elusive, including for example, malignancy, autoimmune disorders, diabetes, obesity and infection.

The protein tyrosine phosphatase (PTP) family of enzymes consists of more than 500 structurally diverse proteins that have in common the highly conserved 250 amino acid PTP catalytic domain, but which display considerable variation in their non-catalytic segments (Charbonneau and Tonks, 1992 Annu. Rev. Cell Biol. 8:463-493; Tonks, 1993 Semin. Cell Biol. 4:373-453). This structural diversity presumably reflects the diversity of physiological roles of individual PTP family members, which in certain cases have been demonstrated to have specific functions in growth, development and differentiation (Desai et al., 1996 Cell 84:599-609; Kishihara et al., 1993 Cell 74:143-156; Perkins et al., 1992 Cell 70:225-236; Pingel and Thomas, 1989 Cell 58:1055-1065; Schultz et al., 1993 Cell 73:1445-1454; Fukada et al., 1999 Growth Factors 17:81-91; Gutch et al., 1998 Genes Dev. 12:571-85; Marengere et al., 1996 Science 272:1170-73). PTPs participate in a variety of physiologic functions, providing a number of opportunities for therapeutic intervention in physiologic processes through alteration (i.e., a statistically significant increase or decrease) or modulation (e.g., up-regulation or down-regulation) of PTP activity. For example, therapeutic inhibition of PTPs such as PTP1B in the insulin signaling pathway may serve to augment insulin action, thereby ameliorating the state of insulin resistance common in Type II diabetes patients.

Although recent studies have also generated considerable information regarding the structure, expression and regulation of PTPs, the nature of many tyrosine phosphorylated substrates through which the PTPs exert their effects remains to be determined. Studies with a limited number of synthetic phosphopeptide substrates have demonstrated some differences in the substrate selectivities of different PTPs (Cho et al., 1993 Protein Sci. 2: 977-984; Dechert et al., 1995 Eur. J Biochem. 231:673-681). Analyses of PTP-mediated dephosphorylation of PTP substrates suggest that catalytic activity may be favored by the presence of certain amino acid residues at specific positions in the substrate polypeptide relative to the phosphorylated tyrosine residue (Salmeen et al., 2000 Molecular Cell 6:1401; Myers et al., 2001 J. Biol. Chem. 276:47771; Myers et al., 1997 Proc. Natl. Acad. Sci. USA 94:9052; Ruzzene et al., 1993 Eur. J Biochem. 211:289-295; Zhang et al., 1994 Biochemistry 33:2285-2290). Thus, although the physiological relevance of the substrates used in these studies is unclear, PTPs display a certain level of substrate selectivity in vitro.

The PTP family of enzymes contains a common evolutionarily conserved segment of approximately 250 amino acids known as the PTP catalytic domain. Within this conserved domain is a unique signature sequence motif,

[I/V]HCXAGXXR[S/T)G SEQ ID NO:98,

that is invariant among all PTPs. The cysteine residue in this motif is invariant in members of the family and is known to be essential for catalysis of the phosphotyrosine dephosphorylation reaction. It functions as a nucleophile to attack the phosphate moiety present on a phosphotyrosine residue of the incoming substrate. If the cysteine residue is altered by site-directed mutagenesis to serine (e.g., in cysteine-to-serine or “CS” mutants) or alanine (e.g., cysteine-to-alanine or “CA” mutants), the resulting PTP is catalytically deficient but retains the ability to complex with, or bind, its substrate, at least in vitro.

CS mutants of certain PTP family members, for example, MKP-1 (Sun et al., 1993 Cell 75:487), may effectively bind phosphotyrosyl polypeptide substrates in vitro to form stable enzyme-substrate complexes, thereby functioning as “substrate trapping” mutant PTPs. Such complexes can be isolated from cells in which both the mutant PTP and the phosphotyrosyl polypeptide substrates are present. According to non-limiting theory, expression of such a CS mutant PTP can thus antagonize the normal function of the corresponding wildtype PTP (and potentially other PTPs and/or other components of a PTP signaling pathway) via a mechanism whereby the CS mutant binds to and sequesters the substrate, precluding substrate interaction with catalytically active, wildtype enzyme (e.g., Sun et al., 1993).

CS mutants of certain other PTP family members, however, may bind phosphotyrosyl polypeptide substrates and form complexes that exist transiently and are not stable when the CS mutant is expressed in cells, i.e., in vivo. The CS mutant of PTP1B is an example of such a PTP. Catalytically deficient mutants of such enzymes that are capable of forming stable complexes with phophotyrosyl polypeptide substrates may be derived by mutating a wildtype protein tyrosine phosphatase catalytic domain invariant aspartate residue and replacing it with an amino acid that does not cause significant alteration of the Km of the enzyme but that results in a reduction in Kcat, as disclosed, for example, in U.S. Pat. Nos. 5,912,138 and 5,951,979, in U.S. application Ser. No. 09/323,426 and in PCT/US97/13016. For instance, mutation of Asp 181 in PTP1B to alanine to create the aspartate-to-alanine (D to A or DA) mutant PTP1B-D181A results in a PTP1B “substrate trapping” mutant enzyme that forms a stable complex with its phosphotyrosyl polypeptide substrate (e.g., Flint et al., 1997 Proc. Nat. Acad. Sci. USA 94:1680). Substrates of other PTPs can be identified using a similar substrate trapping approach, for example substrates of the PTP family members PTP-PEST (Garton et al., 1996 J. Mol. Cell. Biol. 16:6408), TCPTP (Tiganis et al., 1998 Mol. Cell Biol. 18:1622), PTP-HSCF (Spencer et al., 1997 J. Cell Biol. 138:845) and PTP-H1 (Zhang et al., 1999 J. Biol. Chem. 274:17806).

Mitogen-activated protein kinases (MAP-kinases) are present as components of conserved cellular signal transduction pathways that have a variety of conserved members. MAP-kinases are activated by phosphorylation at a dual phosphorylation motif with the sequence Thr-X-Tyr (by MAP-kinase kinases), in which phosphorylation at the tyrosine and threonine residues is required for activity. Activated MAP-kinases phosphorylate several transduction targets, including transcription factors. Inactivation of MAP-kinases is mediated by dephosphorylation at this site by dual-specificity phosphatases referred to as MAP-kinase phosphatases. In higher eukaryotes, the physiological role of MAP-kinase signaling has been correlated with cellular events such as proliferation, oncogenesis, development and differentiation. Accordingly, the ability to regulate signal transduction via these pathways could lead to the development of treatments and preventive therapies for human diseases associated with MAP-kinase signaling, such as cancer.

Dual-specificity protein tyrosine phosphatases (dual-specificity phosphatases) are phosphatases that dephosphorylate both phosphotyrosine and phosphothreonine/serine residues (Walton et al., Ann. Rev. Biochem. 62:101-120, 1993). Several dual-specificity phosphatases that inactivate a MAP-kinase have been identified, including MKP-1 (WO 97/00315; Keyse and Emslie, Nature 59:644-647, 1992), MKP-2 (WO97/00315), MKP-4, MKP-5, MKP-7, Hb5 (WO 97/06245), PAC1 (Ward et al., Nature 367:651-654, 1994), HVH2 (Guan and Butch, J. Biol. Chem. 270:7197-7203, 1995) and PYST1 (Groom et al., EMBO J. 15:3621-3632, 1996). Expression of certain dual-specificity phosphatases is induced by stress or mitogens, but others appear to be expressed constitutively in specific cell types. The regulation of dual-specificity phosphatase expression and activity is critical for control of MAP-kinase mediated cellular functions, including cell proliferation, cell differentiation and cell survival. For example, dual-specificity phosphatases may function as negative regulators of cell proliferation. It is likely that there are many such dual-specificity phosphatases, with varying specificity with regard to cell type or activation. However, the regulation of dual specificity phosphatases remains poorly understood and only a relatively small number of dual-specificity phosphatases have been identified.

Currently, desirable goals for determining the molecular mechanisms that govern PTP-mediated cellular events include, inter alia, determination of PTP interacting molecules, substrates and binding partners, and identification of agents that regulate PTP activities. In some situations, however, current approaches may lead to an understanding of certain aspects of the regulation of tyrosine phosphorylation by PTPs, but still may not provide strategies to control specific tyrosine phosphorylation and/or dephosphorylation events within a cell. Accordingly, there is a need in the art for an improved ability to manipulate phosphotyrosine signaling, including intervention in the regulation of PTPs. An increased understanding of PTP regulation may facilitate the development of methods for modulating the activity of proteins involved in phosphotyrosine signaling pathways, and for treating conditions associated with such pathways.

Hence, and as also noted above, over the last fifteen years it has been established that the Protein Tyrosine Phosphatases (PTPs) are a large, structurally diverse family of receptor-like and non-transmembrane enzymes, which exhibit exquisite substrate specificity in vivo and are critical regulators of a wide array of cellular signaling pathways (Andersen et al., 2001 Mol. Cell. Biol. 21:7117; Tonks and Neel, 2001 Curr. Opin. Cell Biol. 13:182). An important area of investigation in the field remains the characterization of mechanisms by which the activity of the PTPs themselves may be regulated in vivo. Recently, the proposal that certain PTPs may be susceptible to oxidation and inactivation has introduced an additional tier of complexity to the regulation of this family of enzymes.

It is now apparent that reactive oxygen species (ROS) are not merely a harmful by-product of life in an aerobic environment. The importance of ROS in phagocytic cells, such as neutrophils, is well documented. Various stimuli lead to the assembly of a multi-component NADPH oxidase complex, which mediates a process known as the respiratory burst (DeLeo et al., 1996 J. Leukoc. Biol. 60:677). NADPH oxidase catalyses transfer of one electron from NADPH to molecular oxygen to generate superoxide anions, which in turn may yield hydrogen peroxide, either via protonation of superoxide or through the action of superoxide dismutase (Thelen et al., 1993 Physiol. Rev. 73:797). The large quantities of such ROS produced in phagocytic cells have been implicated as microbicidal agents and in certain pathological situations can result in host cell damage (Smith et al., 1991 Blood 77:673). However, many recent studies have revealed that the production of ROS is tightly regulated, engendering the concept that, at lower levels than those generated for a microbicidal function, ROS may also function in propagating a signaling response to extracellular stimuli (Finkel, 1998 Curr. Opin. Cell Biol. 10:248; Finkel, 2000 FEBS Lett. 476:52). Thus, in a manner analogous to reversible protein phosphorylation, the reversible oxidation of target proteins in a cell may regulate the function of those proteins in response to various agonists and thus elicit a cellular response to stimulation (Finkel, 1998).

Several lines of investigation have implicated ROS in the regulation of mitogenic signaling in mammalian cells (Adler et al., 1999 Oncogene 18:6104; Brummel et al., 1996 J. Biol. Chem. 271:1455-61; Chen et al., 1995 J. Biol. Chem. 270:28499; Sundaresan et al., 1995 Science 270:296). Mild oxidation can yield a stable sulfenic acid modification of cysteine residues (Cys-SOH) in selected proteins, including a variety of enzymes and transcription factors, which has the potential to regulate the function of those proteins (Claiborne et al., 1999 Biochemistry 38:15407). In order to understand the role of ROS and redox regulation in the control of signal transduction, it is particularly important to identify the targets of reversible oxidation in vivo. In this context, attention has been drawn to the PTPs, which together with the PTKs are responsible for maintaining a normal tyrosine phosphorylation status in vivo. As described above, the PTPs are characterized by a signature motif, I/V-H-C-X-X-G-X-X-R-S/T, which forms the base of the active site cleft and contains an invariant Cys residue (Barford et al., 1995 Nat. Struct. Biol. 2:1043). The catalytic mechanism involves a two-step process, commencing with nucleophilic attack by the Sγ atom of the catalytic Cys on the phosphorus atom of the phosphotyrosyl substrate, resulting in formation of a phospho-Cys intermediate. In the second step the transient phospho-enzyme intermediate is hydrolyzed by an activated water molecule (Barford et al., 1995). Due to the unusual environment of the PTP active site, the pKα of the sulfhydryl group of this Cys residue is extremely low (˜5.4 in PTP1B, (Lohse et al., 1997 Biochemistry 36:4568) and ˜4.7 in YOP, (Zhang et al., 1993 Biochemistry 32:9340)) compared to the typical pKα for Cys (˜8.5), which favors its function as a nucleophile but renders it susceptible to oxidation. It has now been shown in vitro that treatment with H₂O₂ of various PTPs (Lee et al., 1998 J. Biol. Chem. 273:15366), dual specificity phosphatses (Denu et al., 1998 Biochemistry 37:5633) and low molecular weight PTPs (Caselli et al., 1998 J. Biol. Chem. 273:32554) leads to oxidation of the active site Cys to sulfenic acid. Such oxidation results in inhibition of activity, because the modified Cys can no longer function as a phosphate acceptor in the first step of the PTP-catalyzed reaction.

Oxidation of Cys to sulfenic acid is reversible (Claiborne et al., 1999 Biochemistry 38:15407) and thus has the potential to form the basis of a mechanism for reversible regulation of PTP activity. In contrast, oxidation by the addition of 2 (sulfinic acid) or 3 (sulfonic acid) oxygens to the active site Cys is irreversible. Interestingly, glutathionylation of the sulfenic acid form of PTP1B has been reported (Barrett et al., 1999 Biochemistry 38:6699) and proposed as a mechanism to protect against further, irreversible oxidation and as an important step in the reverse, reduction mechanism. Stimulation of A431 cells with EGF was also shown to lead to the production of H₂O₂ and concomitant inhibition of PTP1B (Bae et al., 1997 J. Biol. Chem. 272:217). Increased production of intracellular oxidants may contribute to enhanced, tyrosine phosphorylation-dependent signaling, for example in response to growth factors (Bae et al., 1997; Bae et al., 2000 J. Biol. Chem. 275:10527; Sundaresan et al., 1995 Science 270:296), by transiently suppressing the enzymatic activity of members of the PTP family, thereby promoting a burst of PTK activity (Finkel, 1998; 2000).

However, it is unclear how broadly this phenomenon may apply across the PTP family, and methods have not previously been available for assessing potential reversible oxidation in a broad range of PTPs in a cellular context, i.e., within a living cell, or in vivo. In particular, there is a need for a method by which one or more oxidized/inactivated PTPs in a cell could be distinguished from reduced/activated PTPs in the cell, and in a manner which need not be specific for a particular PTP, or which need not require that each PTP being investigated be highly purified (e.g., specifically immunoprecipitated) or recombinantly cloned and expressed. An increased understanding of PTP regulation in biological signal transduction, including via inducible signaling pathways triggered by biological stimuli, may facilitate the development of methods for modulating the activity of proteins involved in PTK/PTP cascades, and for treating conditions associated with such cascades. The present invention fulfills these needs and further provides other related advantages.

### SUMMARY OF THE INVENTION

It is an aspect of the present invention to provide a method for identifying a protein tyrosine phosphatase that is reversibly oxidized in a cell, comprising contacting a biological sample comprising a cell that comprises at least one protein tyrosine phosphatase with a stimulus under conditions and for a time sufficient to induce reversible oxidation of at least one protein tyrosine phosphatase in the cell; isolating anaerobically the protein tyrosine phosphatase in the presence of a sulthydryl-reactive agent that is capable of irreversibly modifying a sulfhydryl group of a protein tyrosine phosphatase active site invariant cysteine; determining under reducing conditions a level of dephosphorylation of a detectably labeled protein tyrosine phosphatase substrate by the protein tyrosine phosphatase, wherein detectable substrate dephosphorylation indicates that an active protein tyrosine phosphatase is present, and therefrom identifying a protein tyrosine phosphatase that is reversibly oxidized in a cell. In one embodiment, the invention provides a method for identifying a SHP-2 protein tyrosine phosphatase (SHP-2) that is reversibly oxidized in a cell, comprising contacting a biological sample comprising a cell that comprises SHP-2 with a stimulus under conditions and for a time sufficient to induce reversible oxidation of SHP-2 in the cell; isolating anaerobically SHP-2 in the presence of a sulfhydryl-reactive agent that is capable of irreversibly modifying a sulfhydryl group of a SHP-2 active site invariant cysteine; determining under reducing conditions a level of dephosphorylation of a detectably labeled SHP-2 substrate by SHP-2, wherein SHP-2 comprises a polypeptide comprising an amino acid sequence set forth in any one of SEQ ID NOS: 14, 16, 26, 28, 30, and 32, and wherein detectable substrate dephosphorylation indicates that an active SHP-2 is present, and therefrom identifying a SHP-2 that is reversibly oxidized in a cell. In another embodiment, the invention provides a method for identifying a PTP1B protein tyrosine phosphatase (PTP1B) that is reversibly oxidized in a cell, comprising contacting a biological sample comprising a cell that comprises PTP1B with a stimulus under conditions and for a time sufficient to induce reversible oxidation of PTP1B in the cell; isolating anaerobically PTP1B in the presence of a sulfhydryl-reactive agent that is capable of irreversibly modifying a sulfhydryl group of a PTP1B active site invariant cysteine; and determining under reducing conditions a level of dephosphorylation of a detectably labeled PTP1B substrate by PTP1B, wherein PTP1B comprises a polypeptide comprising an amino acid sequence set forth in any one of SEQ ID NOS: 2, 4, 6, 8, 10, and wherein detectable substrate dephosphorylation indicates that an active PTP1B is present, and therefrom identifying a PTP1B that is reversibly oxidized in a cell. In certain other embodiments of the present invention, a method is provided for identifying a TC45 protein tyrosine phosphatase (TC45) that is reversibly oxidized in a cell, comprising contacting a biological sample comprising a cell that comprises TC45 with a stimulus under conditions and for a time sufficient to induce reversible oxidation of TC45 in the cell; isolating anaerobically TC45 in the presence of a sulfhydryl-reactive agent that is capable of irreversibly modifying a sulfhydryl group of a TC45 active site invariant cysteine; and determining under reducing conditions a level of dephosphorylation of a detectably labeled TC45 substrate by TC45, wherein TC45 comprises a polypeptide comprising an amino acid sequence set forth in NM_{—-}080422, and wherein detectable substrate dephosphorylation indicates that an active TC45 is present, and therefrom identifying a TC45 that is reversibly oxidized in a cell.

In certain embodiments the protein tyrosine phosphatase is PTP1B, PTP-PEST, PTPγ, LAR, MKP-1, CRYPα, PTPcryp2, DEP-1, SAP1, PCPTP1, PTPSL, STEP, HePTP, PTPIA2, PTPNP, PTPNE6, PTPμ, PTPX1, PTPX10, SHP-1, SHP-2, PTPBEM1, PTPBEM2, PTPBYP, PTPesp, PTPoc, PTP-PEZ, PTP-MEG1, MEG2, LC-PTP, TC-PTP, TC45, CD45, LAR, cdc14, RPTP-α, RPTP-ε, RKPTP, LyPTP, PEP, BDP1, PTP20, PTPK1, PTPS31, PTPGMC, GLEPP1, OSTPTP, PTPtep, PTPRL10, PTP2E, PTPD1, PTPD2, PTP36, PTPBAS, PTPBL, BTPBA14, PTPTyp, HDPTP, PTPTD14, PTPα, PTPβ, PTPδ, PTPε, PTPκ, PTPλ, PTPμ, PTPρ, PTPψ, PTPφ, PTPζ, PTPNU3 or PTPH1, or a PTP as presented in FIG. 8, or a dual specificity phosphatase including but not limited to PYST-1, MKP-1, MKP-2, MKP-4, MKP-5, MKP-7, hVH5, PAC1, VHR, or any dual specificity phosphatase disclosed in WO00/65069 (DSP-5), WO00/65068 (DSP-10), WO00/63393 (DSP-8), WO00/60100 (DSP-9), WO00/60099 (DSP-4), WO00/60098 (DSP-7), WO00/60092 (DSP-3), WO00/56899 (DSP-2), WO00/53636 (DSP-1), WO00/09656 (MKP), AU5475399 (MKP), AU8479498, WO99/02704, WO97/06245 (MKP), WO01/83723, WO01/57221, WO01/05983, WO01/02582, WO01/02581, U.S. application Ser. No. 09/955,732 (DSP-15), U.S. application Ser. No. 09/964,277 (DSP-16), U.S. A. No. 60/268,837 (DSP-17) or U.S. A. No. 60/291,476 (PTP). In certain embodiments the protein tyrosine phosphatase substrate comprises phosphorylated poly-(4:1)-Glu-Tyr, which in certain further embodiments comprises ³²P. In certain embodiments the detectably labeled protein tyrosine phosphatase substrate comprises a reporter molecule that is a fluorophore, a radionuclide, a chemiluminescent agent, an enzyme, an immunologically detectable epitope or a chromaphore. In certain further embodiments, the fluorophore is selected from fluorescein, rhodamine, Texas Red, AlexaFluor-594, AlexaFluor-488, Oregon Green, BODIPY-FL or Cy-5.

According to certain embodiments of the present invention, the protein tyrosine phosphatase substrate comprises a polypeptide sequence derived from a protein selected from a PDGF receptor, VCP, p130^{cas}, EGF receptor, p210 bcr:abl, MAP kinase, She, insulin receptor, lck, T cell receptor zeta chain, lysozyme, or reduced and carboxyamidomethylated and maleylated lysozyme (RCML). In certain embodiments the sulfhydryl-reactive agent that is capable of irreversibly modifying a sulfhydryl group of a protein tyrosine phosphatase active site invariant cysteine is an alkylating agent. In certain embodiments the sulfhydryl-reactive agent that is capable of irreversibly modifying a sulfhydryl group of a protein tyrosine phosphatase active site invariant cysteine is iodoacetamide, iodoacetic acid, arsenic oxide, maleimide analog, haloacetimido analog, 4-vinylpyrimidine analog or N-ethylmaleimide. In certain embodiments the cell is a mammalian cell, which in certain embodiments is derived from a cell line and in certain further embodiments is derived from Rat-1 fibroblasts, COS cells, CHO cells or HEK-293 cells. In certain embodiments the step of isolating the protein tyrosine phosphatase comprises cell lysis, and in certain further embodiments the step of isolating comprises gel electrophoresis of the protein tyrosine phosphatase, and in certain further embodiments this step comprises electrophoresis of the protein tyrosine phosphatase in a gel comprising the detectably labeled protein tyrosine phosphatase substrate. In certain embodiments the method further comprises detecting the protein tyrosine phosphatase with an antibody that specifically binds to the phosphatase.

In certain embodiments of the present invention the stimulus increases reactive oxygen species in the sample, and in certain further embodiments the stimulus is a cytokine, a growth factor, a hormone, a cell stressor or a peptide. In certain embodiments the cell stressor is ROS or ultraviolet light. In certain embodiments the stimulus is PDGF, EGF, bFGF, insulin, GM-CSF, TGF-β1, IL-1, IL-3, IFN-γ, TNF-α, PHA, AT-2, thrombin, thyrotropin, parathyroid hormone, LPA, sphingosine-1-phosphate, serotonin, endothelin, acetylcholine, platelet activating factor, bradykinin or G-CSF.

In certain embodiments of the present invention there is provided a method for identifying a protein tyrosine phosphatase that is reversibly modified by a PTP active site-binding agent in a cell, comprising contacting a PTP active site-binding agent that is capable of reversibly modifying a sulfhydryl group of a protein tyrosine phosphatase active site invariant cysteine with a biological sample comprising a cell that comprises at least one protein tyrosine phosphatase; isolating the protein tyrosine phosphatase in the presence of a sulfhydryl-reactive agent that is capable of irreversibly modifying a sulfhydryl group of a protein tyrosine phosphatase active site invariant cysteine; and determining, under conditions that are capable of reversing a reversible modification of a sulfhydryl group of a protein tyrosine phosphatase active site invariant cysteine, a level of dephosphorylation of a detectably labeled protein tyrosine phosphatase substrate by the protein tyrosine phosphatase, wherein detectable substrate dephosphorylation indicates that an active protein tyrosine phosphatase is present, and therefrom identifying a protein tyrosine phosphatase that is reversibly modified by a PTP active site-binding agent in a cell. In certain embodiments, the invention provides a method for identifying a SHP-2 protein tyrosine phosphatase (SHP-2) that is reversibly modified by a PTP active site-binding agent in a cell, comprising contacting a PTP active site-binding agent that is capable of reversibly modifying a sulfhydryl group of a SHP-2 active site invariant cysteine with a biological sample comprising a cell that comprises SHP-2; isolating SHP-2 in the presence of a sulfhydryl-reactive agent that is capable of irreversibly modifying a sulfhydryl group of a SHP-2 active site invariant cysteine; and determining, under conditions that are capable of reversing a reversible modification of a sulfhydryl group of a SHP-2 active site invariant cysteine, a level of dephosphorylation of a detectably labeled SHP-2 substrate by SHP-2, wherein SHP-2 comprises a polypeptide comprising an amino acid sequence set forth in any one of SEQ ID NOS: 14, 16, 26, 28, 30, and 32, and wherein detectable substrate dephosphorylation indicates that an active SHP-2 is present, and therefrom identifying a SHP-2 that is reversibly modified by a PTP active site-binding agent in a cell. In another embodiment, the invention provides a method for identifying a PTP1B protein tyrosine phosphatase (PTP1B) that is reversibly modified by a PTP active site-binding agent in a cell, comprising contacting a PTP active site-binding agent that is capable of reversibly modifying a sulfhydryl group of a PTP1B active site invariant cysteine with a biological sample comprising a cell that comprises PTP1B; isolating PTP1B in the presence of a sulfhydryl-reactive agent that is capable of irreversibly modifying a sulfhydryl group of a PTP1B active site invariant cysteine; and determining, under conditions that are capable of reversing a reversible modification of a sulfhydryl group of a PTP1B active site invariant cysteine, a level of dephosphorylation of a detectably labeled PTP1B substrate by PTP1B, wherein PTP1B comprises a polypeptide comprising an amino acid sequence set forth in any one of SEQ ID NOS: 2, 4, 6, 8, 10, and wherein detectable substrate dephosphorylation indicates that an active PTP1B is present, and therefrom identifying a PTP1B that is reversibly modified by a PTP active site-binding agent in a cell. In another embodiment, the invention provides a method for identifying a TC45 protein tyrosine phosphatase (TC45) that is reversibly modified by a PTP active site-binding agent in a cell, comprising contacting a PTP active site-binding agent that is capable of reversibly modifying a sulfhydryl group of a TC45 active site invariant cysteine with a biological sample comprising a cell that comprises TC45; isolating TC45 in the presence of a sulfhydryl-reactive agent that is capable of irreversibly modifying a sulfhydryl group of a TC45 active site invariant cysteine; and determining, under conditions that are capable of reversing a reversible modification of a sulfhydryl group of a TC45 active site invariant cysteine, a level of dephosphorylation of a detectably labeled TC45 substrate by TC45, wherein TC45 comprises a polypeptide comprising an amino acid sequence set forth in NM_{—}080422, and wherein detectable substrate dephosphorylation indicates that an active TC45 is present, and therefrom identifying a TC45 that is reversibly modified by a PTP active site-binding agent in a cell.

In certain further embodiments, the step of isolating is performed anaerobically. In certain embodiments the PTP active site-binding agent is an agent that covalently binds to the PTP active site or an agent that non-covalently binds to the PTP active site. In certain embodiments the PTP active site-binding agent is a sulfonated compound or a vanadate compound. In certain embodiments the PTP active site-binding agent covalently and reversibly modifies a sulfhydryl group of a PTP active site invariant cysteine. In certain further embodiments the step of determining comprises reversing a covalent modification of a sulfhydryl group of a PTP active site invariant cysteine. In certain still further embodiments the step of reversing comprises contacting the PTP with a reducing agent. In certain still further embodiments the reducing agent is dithiothreitol, dithioerythritol or 2-mercaptoethanol. In certain embodiments the sulfhydryl-reactive agent that is capable of irreversibly modifying a sulfhydryl group of a protein tyrosine phosphatase active site invariant cysteine is iodoacetamide, iodoacetic acid, arsenic oxide, maleimide analog, haloacetimido analog, 4-vinylpyrimidine analog or N-ethylmaleimide.

According to certain other embodiments of the present invention, there is provided a method for identifying a protein tyrosine phosphatase that is a reversibly modified component of an inducible biological signaling pathway in a cell, comprising contacting a biological sample comprising a cell that comprises at least one protein tyrosine phosphatase with a stimulus that induces a biological signaling pathway under conditions and for a time sufficient to induce the biological signaling pathway and thereby reversibly protect protein tyrosine phosphatase active site invariant cysteine from modification; isolating the protein tyrosine phosphatase in the presence of a sulfhydryl-reactive agent that is capable of irreversibly modifying a sulfhydryl group of a protein tyrosine phosphatase active site invariant cysteine; and determining, under conditions that reverse the reversible protection of the protein tyrosine phosphatase active site invariant cysteine from modification, a level of dephosphorylation of a detectably labeled protein tyrosine phosphatase substrate by the protein tyrosine phosphatase, wherein detectable substrate dephosphorylation indicates that an active protein tyrosine phosphatase is present, and therefrom identifying a protein tyrosine phosphatase that is a reversibly modified component of an inducible biological signaling pathway in a cell. In a certain embodiment, the invention provides a method for identifying a SHP-2 protein tyrosine phosphatase (SHP-2) that is a reversibly modified component of an inducible biological signaling pathway in a cell, comprising contacting a biological sample comprising a cell that comprises SHP-2 with a stimulus that induces a biological signaling pathway under conditions and for a time sufficient to induce the biological signaling pathway and thereby reversibly protect a SHP-2 active site invariant cysteine from modification; isolating the SHP-2 in the presence of a sulfhydryl-reactive agent that is capable of irreversibly modifying a sulfhydryl group of a SHP-2 active site invariant cysteine; and determining, under conditions that reverse the reversible protection of the SHP-2 active site invariant cysteine from modification, a level of dephosphorylation of a detectably labeled SHP-2 substrate by SHP-2, wherein SHP-2 comprises a polypeptide comprising an amino acid sequence set forth in any one of SEQ ID NOS: 14, 16, 26, 28, 30, and 32, and wherein detectable substrate dephosphorylation indicates that an active SHP-2 is present, and therefrom identifying a SHP-2 that is a reversibly modified component of an inducible biological signaling pathway in a cell. In another embodiment, that which is provided is a method for identifying a PTP1B protein tyrosine phosphatase (PTP1B) that is a reversibly modified component of an inducible biological signaling pathway in a cell, comprising contacting a biological sample comprising a cell that comprises PTP1B with a stimulus that induces a biological signaling pathway under conditions and for a time sufficient to induce the biological signaling pathway and thereby reversibly protect a PTP1B active site invariant cysteine from modification; isolating the PTP1B in the presence of a sulfhydryl-reactive agent that is capable of irreversibly modifying a sulfhydryl group of a PTP1B active site invariant cysteine; and determining, under conditions that reverse the reversible protection of the PTP1B active site invariant cysteine from modification, a level of dephosphorylation of a detectably labeled PTP1B substrate by PTP1B, wherein PTP1B comprises a polypeptide comprising an amino acid sequence set forth in any one of SEQ ID NOS: 2, 4, 6, 8, 10, and wherein detectable substrate dephosphorylation indicates that an active PTP1B is present, and therefrom identifying a PTP1B that is a reversibly modified component of an inducible biological signaling pathway in a cell. In a certain embodiment, the invention provides a method for identifying a TC45 protein tyrosine phosphatase (TC45) that is a reversibly modified component of an inducible biological signaling pathway in a cell, comprising contacting a biological sample comprising a cell that comprises TC45 with a stimulus that induces a biological signaling pathway under conditions and for a time sufficient to induce the biological signaling pathway and thereby reversibly protect a TC45 active site invariant cysteine from modification; isolating the TC45 in the presence of a sulfhydryl-reactive agent that is capable of irreversibly modifying a sulfhydryl group of a TC45 active site invariant cysteine; and determining, under conditions that reverse the reversible protection of the TC45 active site invariant cysteine from modification, a level of dephosphorylation of a detectably labeled TC45 substrate by TC45, wherein TC45 comprises a polypeptide comprising an amino acid sequence set forth in NM_{—}080422, and wherein detectable substrate dephosphorylation indicates that an active TC45 is present, and therefrom identifying a TC45 that is a reversibly modified component of an inducible biological signaling pathway in a cell.

In certain embodiments the step of isolating is performed anaerobically. In certain embodiments the sulfhydryl-reactive agent that is capable of irreversibly modifying a sulfhydryl group of a protein tyrosine phosphatase active site invariant cysteine is iodoacetamide, iodoacetic acid, arsenic oxide, maleimide analog, haloacetimido analog, 4-vinylpyrimidine analog or N-ethylmaleimide.

In certain other embodiments the invention provides a method for identifying an agent that alters an inducible biological signaling pathway, comprising (a) identifying a protein tyrosine phosphatase that is reversibly oxidized in a first biological sample comprising a cell that comprises at least one PTP according to the above described method steps of contacting, isolating and determining; (b) contacting, in the presence and absence of a candidate agent, a second biological sample comprising a cell that comprises the PTP that is reversibly oxidized as identified according to the method of (a) with the stimulus under conditions and for a time sufficient to induce reversible oxidation of the PTP; (c) isolating the protein tyrosine phosphatase in the presence of a sulfhydryl-reactive agent that is capable of covalently modifying a sulfhydryl group of a protein tyrosine phosphatase active site invariant cysteine; and (d) determining under reducing conditions a level of dephosphorylation of a detectably labeled protein tyrosine phosphatase substrate by the protein tyrosine phosphatase, wherein a level of substrate dephosphorylation that is decreased when the second sample is contacted with the stimulus in the presence of the candidate agent relative to the level of substrate dephosphorylation when the sample is contacted with the stimulus in the absence of the agent indicates that the agent is an inhibitor of an inducible biological signaling pathway, and wherein a level of substrate dephosphorylation that is increased when the sample is contacted with the stimulus in the presence of the candidate agent relative to the level of substrate dephosphorylation when the sample is contacted with the stimulus in the absence of the agent indicates that the agent is a potentiator of an inducible biological signaling pathway. In certain embodiments the step of isolating in the method recited in (a) is performed anaerobically, and in certain embodiments the step of isolating recited in (c) is performed anaerobically.

In a certain embodiment, the invention provides a method for identifying an agent that alters an inducible biological signaling pathway, comprising (a) identifying a SHP-2 protein tyrosine phosphatase (SHP-2) that is reversibly oxidized in a cell according to a method comprising (i) contacting a first biological sample comprising a cell that comprises SHP-2 with a stimulus under conditions and for a time sufficient to induce reversible oxidation of SHP-2 in the cell; (ii) isolating SHP-2 in the presence of a sulfhydryl-reactive agent that is capable of irreversibly modifying a sulfhydryl group of a SHP-2 active site invariant cysteine; (iii) determining under reducing conditions a level of dephosphorylation of a detectably labeled SHP-2 substrate by SHP-2, wherein detectable substrate dephosphorylation indicates that an active SHP-2 is present, and therefrom identifying a SHP-2 that is reversibly oxidized in a cell; (b) contacting, in the presence and absence of a candidate agent, a second biological sample comprising a cell that comprises SHP-2 that is reversibly oxidized as identified according to the method of (a) with the stimulus under conditions and for a time sufficient to induce reversible oxidation of SHP-2; (c) isolating SHP-2 in the presence of a sulfhydryl-reactive agent that is capable of covalently modifying a sulfhydryl group of a SHP-2 active site invariant cysteine; and (d) determining under reducing conditions a level of dephosphorylation of a detectably labeled SHP-2 substrate by SHP-2, wherein SHP-2 comprises a polypeptide comprising an amino acid sequence set forth in any one of SEQ ID NOS: 14, 16, 26, 28, 30, and 32, wherein a level of substrate dephosphorylation that is decreased when the second sample is contacted with the stimulus in the presence of the candidate agent relative to the level of substrate dephosphorylation when the sample is contacted with the stimulus in the absence of the agent indicates that the agent is an inhibitor of an inducible biological signaling pathway, wherein a level of substrate dephosphorylation that is increased when the sample is contacted with the stimulus in the presence of the candidate agent relative to the level of substrate dephosphorylation when the sample is contacted with the stimulus in the absence of the agent indicates that the agent is a potentiator of an inducible biological signaling pathway.

In another embodiment of the invention is provided a method for identifying an agent that alters an inducible biological signaling pathway, comprising (a) identifying a PTP1B protein tyrosine phosphatase (PTP1B) that is reversibly oxidized in a cell according to a method comprising (i) contacting a first biological sample comprising a cell that comprises PTP1B with a stimulus under conditions and for a time sufficient to induce reversible oxidation of PTP1B in the cell; (ii) isolating PTP1B in the presence of a sulfhydryl-reactive agent that is capable of irreversibly modifying a sulfhydryl group of a PTP1B active site invariant cysteine; (iii) determining under reducing conditions a level of dephosphorylation of a detectably labeled PTP1B substrate by PTP1B, wherein detectable substrate dephosphorylation indicates that an active PTP1B is present, and therefrom identifying a PTP1B that is reversibly oxidized in a cell; (b) contacting, in the presence and absence of a candidate agent, a second biological sample comprising a cell that comprises PTP1B that is reversibly oxidized as identified according to the method of (a) with the stimulus under conditions and for a time sufficient to induce reversible oxidation of PTP1B; (c) isolating PTP1B in the presence of a sulfhydryl-reactive agent that is capable of covalently modifying a sulfhydryl group of a PTP1B active site invariant cysteine; and (d) determining under reducing conditions a level of dephosphorylation of a detectably labeled PTP1B substrate by PTP1B, wherein PTP1B comprises a polypeptide comprising an amino acid sequence set forth in any one of SEQ ID NOS: 2, 4, 6, 8, 10, wherein a level of substrate dephosphorylation that is decreased when the second sample is contacted with the stimulus in the presence of the candidate agent relative to the level of substrate dephosphorylation when the sample is contacted with the stimulus in the absence of the agent indicates that the agent is an inhibitor of an inducible biological signaling pathway, and wherein a level of substrate dephosphorylation that is increased when the sample is contacted with the stimulus in the presence of the candidate agent relative to the level of substrate dephosphorylation when the sample is contacted with the stimulus in the absence of the agent indicates that the agent is a potentiator of an inducible biological signaling pathway.

The invention also provides a method for identifying an agent that alters an inducible biological signaling pathway, comprising (a) identifying a TC45 protein tyrosine phosphatase (TC45) that is reversibly oxidized in a cell according to a method comprising (i) contacting a first biological sample comprising a cell that comprises TC45 with a stimulus under conditions and for a time sufficient to induce reversible oxidation of TC45 in the cell; (ii) isolating TC45 in the presence of a sulfhydryl-reactive agent that is capable of irreversibly modifying a sulfhydryl group of a TC45 active site invariant cysteine; (iii) determining under reducing conditions a level of dephosphorylation of a detectably labeled TC45 substrate by TC45, wherein detectable substrate dephosphorylation indicates that an active TC45 is present, and therefrom identifying a TC45 that is reversibly oxidized in a cell; (b) contacting, in the presence and absence of a candidate agent, a second biological sample comprising a cell that comprises TC45 that is reversibly oxidized as identified according to the method of (a) with the stimulus under conditions and for a time sufficient to induce reversible oxidation of TC45; (c) isolating TC45 in the presence of a sulfhydryl-reactive agent that is capable of covalently modifying a sulfhydryl group of a TC45 active site invariant cysteine; and (d) determining under reducing conditions a level of dephosphorylation of a detectably labeled TC45 substrate by TC45, wherein TC45 comprises a polypeptide comprising an amino acid sequence set forth in NM_{—}080422, wherein a level of substrate dephosphorylation that is decreased when the second sample is contacted with the stimulus in the presence of the candidate agent relative to the level of substrate dephosphorylation when the sample is contacted with the stimulus in the absence of the agent indicates that the agent is an inhibitor of an inducible biological signaling pathway, and wherein a level of substrate dephosphorylation that is increased when the sample is contacted with the stimulus in the presence of the candidate agent relative to the level of substrate dephosphorylation when the sample is contacted with the stimulus in the absence of the agent indicates that the agent is a potentiator of an inducible biological signaling pathway.

These and other aspects of the present invention will become evident upon reference to the following detailed description and attached drawings. In addition, various references are set forth herein which describe in more detail certain aspects of this invention, and are therefore incorporated by reference in their entireties.

### BRIEF DESCRIPTION OF THE DRAWINGS

FIG. 1 shows a schematic for use of the “in-gel” phosphatase assay to identify PTPs that are susceptible to stimulus-induced oxidation.

FIG. 2 shows reversible oxidation of multiple PTPs concomitant with tyrosine phosphorylation in Rat-1 cells treated with H₂O₂. FIG. 2A illustrates an in-gel PTP assay. Serum-deprived Rat-1 cells were exposed to various concentrations of H₂O₂ for 1 min, harvested, and lysed in the absence (lane 1) or presence (lanes 2-7) of 10 mM iodoacetic acid (IAA). FIG. 2B presents an immunoblot of tyrosine phosphorylated proteins immunoprecipitated from lysates of H₂O₂-treated cells with Ab PT-66, then immunoblotted with anti-pTyr Ab (G104). FIG. 2C presents an in-gel PTP assay. After pre-incubation of Rat-1 cells in the absence or presence of 30 mM NAC, the cells were exposed to 200 μM H₂O₂ and lysed in the presence of 10 mM IAA at the indicated times. FIG. 2D shows an in-gel PTP assay of oxidized PTPs. Rat-1 cells were serum-starved in the absence or presence of 2.5 mM BSO for 16 h. H₂O₂ (200 μM) was added for 2 minutes, then removed by washing the cells with fresh culture media. Incubation was continued until the cells were harvested in lysis buffer containing 10 mM IAA at the times indicated. Arrows indicate PTPs for which reduction/reactivation displayed dependence on intracellular GSH.

FIG. 3 illustrates that H₂O₂-induced mitogenic signaling was associated with inactivation of PTPs. FIG. 3A presents an in-gel PTP assay. Purified SHP-2 (E76A mutant, 1 ng/lane) was incubated with PBS, H₂O₂, or t-BHP at 37° C. for 5 minutes. Aliquots were then incubated at room temperature for an additional 5 minutes, either in the absence (−IAA) or presence (+IAA) of 4 mM IAA. FIG. 3B shows images of ROS-induced DCF fluorescence in Rat-1 cells pre-loaded with 20 μM H₂DCFDA in the dark and then exposed to H₂O₂ or t-BHP (each at 200 EM). The cells are shown at magnification 400× (upper panels). Cells (1×10⁵) that underwent the same treatment as above were harvested and resuspended in Hanks' solution, then immediately subjected to flow cytometric analysis to measure ROS-induced DCF fluorescence (lower panels). The basal peak indicates background fluorescence, whereas the rightward shifted peak indicates ROS-induced DCF fluorescence. FIG. 3C depicts an in-gel PTP assay of oxidized PTPs. Cells were exposed to H₂O₂ and t-BHP (each at 200 μM) for the indicated times and lysed in the presence of 10 mM IAA. FIG. 3D presents an immunoblot of cell lysates prepared from cells exposed to H₂O₂ and t-BHP (each at 200 EM). Tyrosine phosphorylated proteins were immunoprecipitated with Ab PT-66, followed by immunoblotting with anti-pTyr Ab G104 (upper panel). An aliquot of lysate from each treatment was immunoblotted with anti-phospho-MAPK Ab and subsequently with anti-MAPK Ab (lower panel).

FIG. 4 shows PDGF induced oxidation of a 70 k PTP in Rat-1 cells. FIG. 4A represents an in-gel PTP assay. Serum-starved Rat-1 cells were exposed to 50 ng/ml PDGF-BB for the times indicated. Lysates were prepared in the presence of 10 mM IAA and subjected to in-gel PTP assay. The arrow indicates a 70 kDa PTP that was transiently oxidized following stimulation of Rat-1 cells with PDGF. The result shown is representative of four independent experiments. FIG. 4B: Cells were pre-incubated in the absence or presence of 30 mM NAC for 40 minutes. Excess NAC was removed prior to addition of PDGF (50 ng/ml). PDGF-induced oxidation of the 70 kDa PTP, which was impaired in the presence of NAC (arrow), was visualized by the modified in-gel PTP assay. FIG. 4C: Cells were treated with NAC and PDGF as described above. PDGFR was immunoprecipitated from lysates with Ab-X and immunoblotted with anti-pTyr Ab G104. The same filter was subsequently re-probed with Ab-X (upper panels). Aliquots of cell lysate from each treatment were immunoblotted with anti-phosho-MAPK Ab and re-probed with anti-MAPK Ab (lower panels).

FIG. 5 illustrates identification of the 70 kDa PTP that was susceptible to PDGF-induced oxidation as SHP-2. FIG. 5A: Serum-starved Rat-1 cells were exposed to PDGF (50 ng/ml) for the indicated times. The PDGFR and associated proteins were immunoprecipitated with antibody Ab-X, and pTyr proteins were visualized by immunoblotting with anti-pTyr Ab G104 (upper panel). The same filter was re-probed with anti-PDGFR, anti-SHP-2, anti-GAP, and anti-p85 PI3K Abs. The positions of PDGFR (solid arrow) and SHP-2 (open arrow) are indicated. FIG. 5B: Rat-1 cells, either untreated (−) or stimulated with 50 ng/ml PDGF (+), were harvested in lysis buffer containing 10 mM IAA. Lysates were incubated with antibody specific for either SHP-2 or SHP-1 and subjected to an in-gel PTP assay (upper panel). The arrow denotes the position of the 70 kDa PTP that was inactivated in response to PDGF and immunodepleted from cell lysates with antibodies to SHP-2. The lower panel illustrates an immunoblot to show the immunodepletion of SHP-2.

FIG. 6 demonstrates oxidation and inactivation of SHP-2 that was induced by PDGF but not by EGF or FGF. FIG. 6A: Rat-1 cells were incubated with 20 μM CM-H₂DCFDA in the dark for 20 minutes, then exposed to peptide growth factors (50 ng/ml) for an additional 10 mins. Images of ROS-induced DCF fluorescence are shown at 50× magnification. The data are representative of four independent experiments. FIG. 6B presents an in-gel PTP assay of oxidized PTPs. Cells were exposed to peptide growth factors for the indicated times and lysed in the presence of 10 mM IAA. FIG. 6C illustrates an immunoblot of cell lysates from each treatment group immunoblotted with anti-phosho-MAPK Ab (upper panel). The immunoblot was reprobed with anti-MAPK Ab (lower panel).

FIG. 7 shows that the pool of PDGFR-associated SHP-2, which was oxidized and inactivated in response to PDGF, was also involved in down-regulation of MAPK signaling. Rat-1 cells were transiently transfected with plasmids expressing WT or Y1009F mutant G-CSFR/PDGFR chimeric receptor, or with a plasmid encoding Green Fluorescence Protein (GFP) as a control for expression. FIG. 7A: After exposure to 100 ng/ml G-CSF for 5 min, the chimeric receptors were immunoprecipiated from lysates with antibody Ab-X and immunoblotted with anti-pTyr Ab G104. Immunoprecipitation of the receptors was verified by immunoblotting with Ab-X. The same filter was stripped and reprobed with anti-SHP-2 Ab. Expression of the chimeric receptors was verified by immunoblotting an aliquot of each lysate with Ab-X, which recognizes the intracellular segment of the PDGFR, and subsequently with anti-G-CSFR Ab, which recognizes the extracellular segment of chimeric receptors. FIG. 7B presents an in-gel PTP assay of Rat-1 cell lysates. Transfected Rat-1 cells were treated with G-CSF for the indicated times and then lysed in the presence of 10 mM IAA. The arrow denotes the position of SHP-2. FIG. 7C: The wild-type and mutant chimeric receptors were immunoprecipitated at the indicated times and immunoblotted with anti-pTyr Ab (G104) (top panel). The same filter was re-probed with anti-PDGFR Ab-X (bottom panel). FIG. 7D presents an immunoblot of cell lysates from each treatment blotted with anti-phosho-MAPK Ab (upper panel), and then re-probed with anti-MAPK Ab (lower panel). FIG. 7E presents a densitometric analysis of the gel image, which illustrates the ratio of phosphorylated MAPK (upper panel of 7D) over total MAPK (lower panel of 7D).

FIG. 8 presents a listing of PTPs.

FIG. 9 illustrates an in-gel PTP assay that shows protection from IAA-inactivation of PTP activity in PHA-stimulated peripheral blood mononuclear lymphocytes pre-treated with a PTP active site-binding agent.

FIG. 10 illustrates that hydrogen peroxide is a mediator of insulin signaling. FIG. 10A presents images of ROS-induced DCF fluorescence by fluorescence microscopy (50× magnification) of serum-starved Rat-1 cells exposed to 50 nM insulin. The data are representative of three independent experiments. FIG. 10B: Rat-1 cells were transiently transfected with different quantities of plasmid encoding human catalase. Two days after transfection, cells were serum-deprived and then stimulated with 50 nM insulin (INS) for 10 min. The cells were lysed, and catalase expression was verified by immunoblotting with anti-catalase antibody (top panel). The insulin receptor β (IR-β) subunit was immunoprecipitated from 400 μg of lysate with antibody 29B4. Immunoblotting was performed with anti-pYpY^{1162/1163} and subsequently with anti-IR-β antibody clone C-19 as a loading control (middle panel). An aliquot of lysate (30 μg) was subjected to immunoblotting with anti-phospho-PKB/AKT antibody. The same filter was then stripped and reprobed with anti-PKB/AKT antibody as a loading control (bottom panel).

FIG. 11 shows that insulin induced the transient oxidation of PTP1B and TC45. For each experiment, serum-starved Rat-1 cells were exposed to 50 nM insulin for the indicated times. Lysates were prepared under anaerobic conditions in the presence of 10 mM IAA and then subjected to in-gel PTP assays. FIG. 11A: The arrowheads indicate that 50 kDa and 45 kDa PTPs were transiently oxidized in response to insulin. Figure B and Figure C present in-gel PTP assays. Total lysate (400 μg) was immunoprecipitated with normal IgG (labeled C), anti-PTP1B antibody (FG6), or anti-TC45 antibody (191 OH) coupled to protein G-Sepharose beads. After immunoprecipitation, the immune complexes and supernatants were subjected to in-gel PTP assays. FIG. 11B shows immunodepletion of the 50 kDa PTP from the lysate with anti-PTP1B antibody. FIG. 11C illustrates immunodepletion of the 45 kDa PTP with antibody specific for TC45. The lane marked “Lys” represents cell lysate prior to immunodepletion. The lower panels illustrate immunoblots of total lysate and the supernatants following immunodepletion, using either anti-PTP1B antibody (FIG. 11B, lower panels) or anti-TC45 antibody (FIG. 11C, lower panels). The same blots were subsequently reprobed with anti-SHP-2 antibody to ensure loading of equal amounts of protein.

### DETAILED DESCRIPTION OF THE INVENTION

The present invention is directed to a method of identifying any PTP that has been reversibly modified (e.g., oxidized, or reversibly modified by a PTP active site-binding agent) in a cellular context (i.e., within a cell, or in vivo), and in particular to any modification of a PTP active site invariant cysteine residue that can be reversed with a reducing agent. As described herein, typically such modification/oxidation of a PTP is accompanied by transient inactivation of the enzyme. Described herein is the unexpected discovery that reversible oxidation of a PTP in a cellular context renders such a PTP resistant to irreversible inactivation of the enzyme by a sulfhydryl-reactive agent that is capable of covalently modifying a sulfhydryl group of a PTP active site invariant cysteine. This discovery is exploited to provide the invention method in a manner whereby one or more PTPs of interest may be non-specifically isolated from a cell—the invention method thus does not require any specific preparation and/or purification of a particular PTP that may be suspected of undergoing reversible modification/oxidation in vivo, such as recombinant cloning and expression of the PTP (which would require a polynucleotide encoding each PTP of interest) or immunoprecipitation of the PTP (which would require an antibody specific for each PTP of interest). Instead, the method may be practiced using a cell that comprises one or a plurality of PTPs, where the method permits determination of one or more reversibly modified/oxidized PTPs in a cell even where the identities of the particular PTPs that are expressed in the cell are not known a priori.

Accordingly, the one or more PTPs in a cell that are transiently modified/oxidized at the time the cell is contacted with the sulfhydryl-reactive agent that is capable of irreversibly (e.g., covalently) modifying a sulfhydryl group of a PTP active site invariant cysteine are not inactivated by the sulfhydryl-reactive agent, and such PTPs can subsequently be detected on the basis of their ability to catalytically dephosphorylate a PTP substrate after reversal (e.g., under reducing conditions) of the transient modification/oxidation event. Hence, and according to non-limiting theory, contact with a stimulus may induce a biological signaling pathway in a cell, which pathway comprises at least one PTP (and potentially a plurality of PTPs) that is reversibly modified at invariant cysteine (e.g., oxidized to form sulfenic acid) in response to the stimulus, and which is therefore reversibly protected from irreversible modification of its active site invariant cysteine during subsequent isolation of the PTP in the presence of a sulfhydryl-reactive agent (e.g., iodoacetamide) that is capable of so modifying the invariant cysteine. By way of contrast, any PTPs that are not reversibly and protectively modified in the course of the cellular response to the stimulus will be susceptible to permanent inactivation by the sulfhydryl agent during the PTP isolation procedure. Isolated PTPs are then exposed to conditions that reverse the reversible protection from modification of the PTP active site invariant cysteine (e.g., reducing conditions), such that PTP enzyme activity is restored only to those PTPs that have undergone the reversible protective modification. This activity can then be determined as a level of dephosphorylation of a detectably labeled PTP substrate as described herein. While this non-limiting theoretical model of the PTP modifications that may or may not occur in the course of practicing the subject invention method pertains to reversible oxidation of PTP active site invariant cysteine in response to a stimulus, as described herein the invention is not intended to be so limited, and also contemplates any other reversible modification to a PTP (e.g., by transient occupancy of the PTP active site by a PTP active site-binding agent that is capable of reversibly modifying a PTP active site invariant cysteine) that can be reversed, for example, a modification that is reversed by a reducing agent.

In certain embodiments the invention thus also provides a method for identifying a PTP that is a reversibly oxidized component of an inducible biological signaling pathway that is induced by a stimulus which may trigger reversible modification, for example, oxidation, of one or more PTPs. In such embodiments, any stimulus that is known to be, or suspected of being, capable of inducing a biological signaling pathway is contacted with a cell comprising one or a plurality of PTPs, and recoverable PTP catalytic activity is assessed following inactivation of unmodified (e.g., non-oxidized) PTPs with a sulfhydryl-reactive agent that is capable of irreversibly (e.g., covalently) modifying a sulfhydryl group of a PTP active site invariant cysteine. In certain related embodiments, prior to the step of contacting the cell with a stimulus, the cell may be contacted with a PTP active site-binding agent, to determine whether such a PTP active site-binding agent alters (i.e., increases or decreases in a statistically significant manner) the level of substrate dephosphorylation by one or more PTPs present in the cell, where PTPs that have retained the ability to dephosphorylate substrate have been reversibly and protectively modified (e.g., oxidized) as a result of the biological signaling pathway induced by the stimulus. Non-limiting examples of PTP active site-binding agents for use in such embodiments include PTP inhibitors as disclosed in Zhang et al. (2002 Ann. Rev. Pharmacol. Toxicol. 42:209-234), Iverson et al. (2001 Biochemistry 40:14812-20) and Jia et al. (2001 J. Med. Chem. 44:4584). Certain such agents may be sulfonated compounds or vanadate compounds (e.g., sodium orthovanadate); these and other PTP active site-binding agents are known to the art and/or may be identified according to established methodologies, including those described herein and in the cited references.

As described in greater detail below, in certain preferred embodiments determination of PTP substrate dephosphorylation, by one or more reversibly oxidized PTPs isolated anaerobically from a cell in the presence of a sulthydryl-reactive agent that is capable of covalently modifying a sulfhydryl group of a PTP active site invariant cysteine on any unmodified PTP, is accomplished using a modified “in-gel” PTP activity assay to allow visualization of a profile of PTPs that are reversibly oxidized following a particular stimulus. Anaerobic isolation conditions may be employed for one or more PTPs identified according to the present method, and whether and/or to what extent such conditions may be needed will vary with each PTP, as well as with the nature of the reversible modification (i.e., oxidative vs. non-oxidative) experienced by the PTP in a cell. Typically, anaerobic isolation of one or more PTPs relates to performing procedures for isolation of PTPs from a sample in an environment that is substantially reduced in its exposure to or content of oxygen gas, for instance, by conducting the isolation in an enclosure in which ambient air has been substantially replaced by an inert gas such as argon or nitrogen. Other procedures for creating an anaerobic atmosphere for PTP isolation may also be employed and will be familiar to those skilled in the art in view of the present disclosure, which describes examples of oxidative modification of PTPs that are detected following anaerobic isolation of the PTP.

Exemplary results using the modified “in-gel” PTP activity assay provided herein indicated that several PTPs could be identified that were oxidized and inactivated reversibly in Rat-1 cells following stimulation with H₂O₂, and that this event was important for peroxide-induced mitogenic signaling. Examples provided below show that platelet-derived growth factor (PDGF) stimulation of Rat-1 cells induced the oxidation and inhibition of the SH2 domain-containing PTP known as SHP-2 (see Hof et al., 1998 Cell 92:441-50), which facilitated mitogenic signaling in these cells in response to the growth factor. Additional examples provided show that insulin-induced signaling resulted in the oxidation and inhibition of two PTPs, PTP1B and the 45 kDa spliced variant of TC-PTP, TC45 (see Mosinger et al., 1992 Proc. Natl. Acad. Sci. USA 89:499-503; Tiganis et al., 1998 Mol. Cell Biol. 18:1622-34; Tiganis et al., 1999 J. Biol. Chem. 274:27768-75). The invention contemplates extending these analyses to identify and characterize other PTPs and their roles in the control of a broad array of biological signal transduction pathways.

Certain preferred embodiments of the invention therefore relate to a method wherein stimulus-induced oxidation within a cellular context (i.e., in vivo) provides a means of “tagging” (e.g., reversibly protecting from a sulfhydryl-reactive agent) those PTPs that are integral to the regulation of the cellular signal transduction pathways initiated by that stimulus. Alkylation with a sulfhydryl-reactive agent that is capable of covalently, and preferably irreversibly, modifying a sulfhydryl group of a PTP active site invariant cysteine, for example, iodoacetamide (IAA), can be used to inactivate and thereby functionally subtract out the bulk of the PTPs, which being unaffected by the stimulus and hence not transiently oxidized, are unprotected from the sulfhydryl reagent. Following reduction to reverse the transient oxidation and return the transiently inactivated PTP to an active state, the stimulus-responsive (i.e., oxidatively protected) PTPs can be isolated and identified on the basis of phosphatase activity, demonstrable as dephosphorylation of a PTP substrate using any of a variety of well established procedures as provided herein and as known to the art. (See, e.g., Flint et al., 1993 EMBO J. 12:1937-1946; Tonks et al., 1991 Meths. Enzymol. 201:427-42; Tonks et al., 1988 J. Biol. Chem. 263:6722). Reducing conditions that are suitable for determining PTP substrate dephosphorylation by a catalytically competent phosphatase (i.e., an “active” PTP) can be achieved using compositions and methods well known to the art in view of the present disclosure. The precise reducing conditions may vary as a function of the particular PTP for which activity following reversible inactivation is to be determined; common reducing agents for establishing such conditions include, by way of illustration and not limitation, dithiothreitol (Cleland's reagent), dithioerythritol and 2-mercaptoethanol (β-mercaptoethanol).

The “in-gel” phosphatase assay described herein comprises a modification of an existing technique (Burridge and Nelson, 1995 Anal. Biochem. 232, 56-64) and provides one such preferred procedure for demonstrating PTP activity toward (phosphorylated) PTP substrates as provided herein. The modified in-gel phosphatase assay features electrophoretic separation and renaturation, under reducing conditions, of a plurality of PTPs in a gel impregnated with a detectably labeled PTP substrate, but with regard to the step of determining dephosphorylation of a detectably labeled PTP substrate by a PTP according to the methods of disclosed herein, the invention is not intended to be so limited. For example some PTPs, in particular certain of the receptor-like forms, may not renature efficiently in the “in-gel” PTP activity assay (Burridge and Nelson, 1995). The invention therefore contemplates incorporation of any suitable method for determining a level of dephosphorylation of a detectably labeled PTP substrate by a PTP, which may vary according to the physicochemical properties (e.g., conformational stability in a variety of chemical environments) of particular PTPs, and which can be selected by a person having ordinary skill in the art readily and without undue experimentation based on the instant disclosure.

For example, suitable phosphatase assays may include in-gel assays using non-denaturing gel systems. Additional methodologies for assaying PTP-mediated substrate dephosphorylation may include proteomics-based strategies, for example, using solid-phase immobilized, broad specificity PTP active site-directed inhibitors (such as phenylarsine oxide coupled to agarose) as affinity matrices for the purification and identification of oxidation-sensitive PTPs. As also noted above, other embodiments contemplate exposure of cells comprising an inducible biological signaling pathway to one or more PTP active site-binding agents (e.g., Zhang et al. 2002 Ann. Rev. Pharmacol. Toxicol. 42:209-234; Iverson et al. 2001 Biochemistry 40:14812-20; Jia et al. 2001 J. Med. Chem. 44:4584) prior to contacting these cells with a stimulus that induces the signaling pathway. Recoverable activity may then be assayed in PTPs that are protectively modified, by reversible oxidation, when the PTPs are isolated in the presence of a sulfhydryl-reactive agent, wherein further the active site-binding agent may be employed to facilitate PTP isolation. By combining these approaches with the use of substrate-trapping mutant forms of the PTPs thus identified (e.g., Flint et al., 1997 Proc. Natl. Acad. Sci. USA 94:1680-1685), the physiological substrate specificities of these enzymes can be determined to further characterize the components of biological signaling pathways that comprise PTPs. Additional characterization of biological signaling pathway components identified using the methods of the present invention may be achieved using specific binding proteins to detect such components. Preferred examples of such binding proteins include antibodies, receptors, counterreceptors, ligands, and the like, for example, an antibody that, as provided herein, specifically binds to a phosphatase, or an antibody that specifically binds to a phosphopeptide such as phosphotyrosine, phosphoserine or phosphothreonine.

PTPs

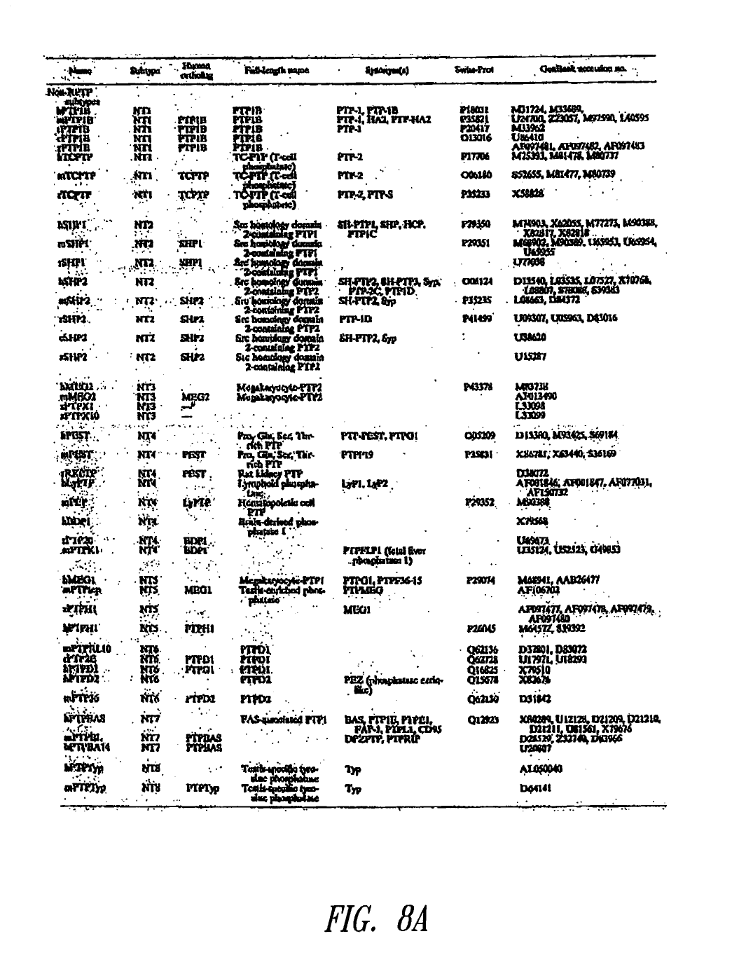

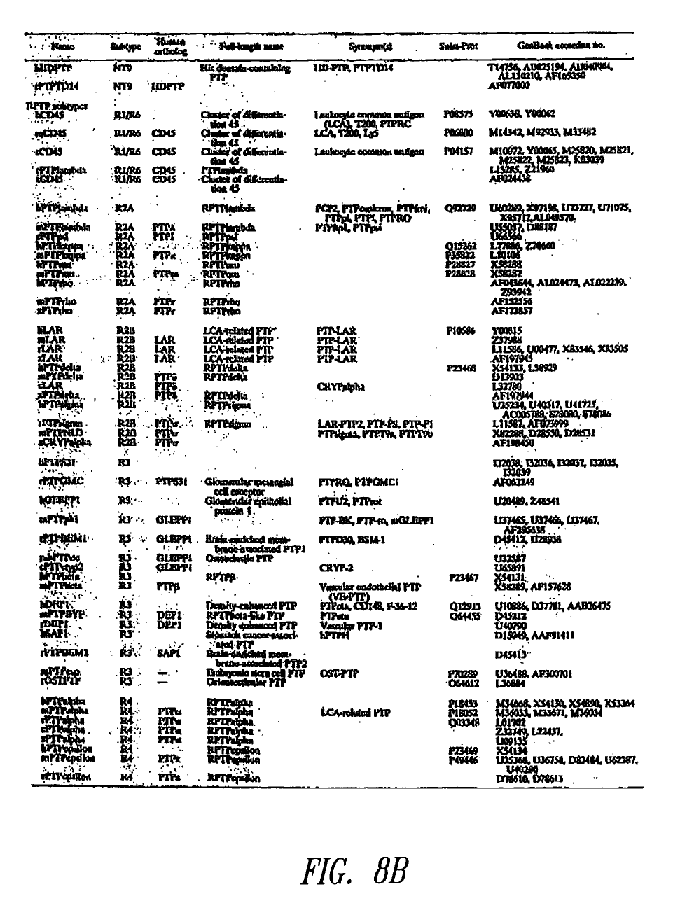

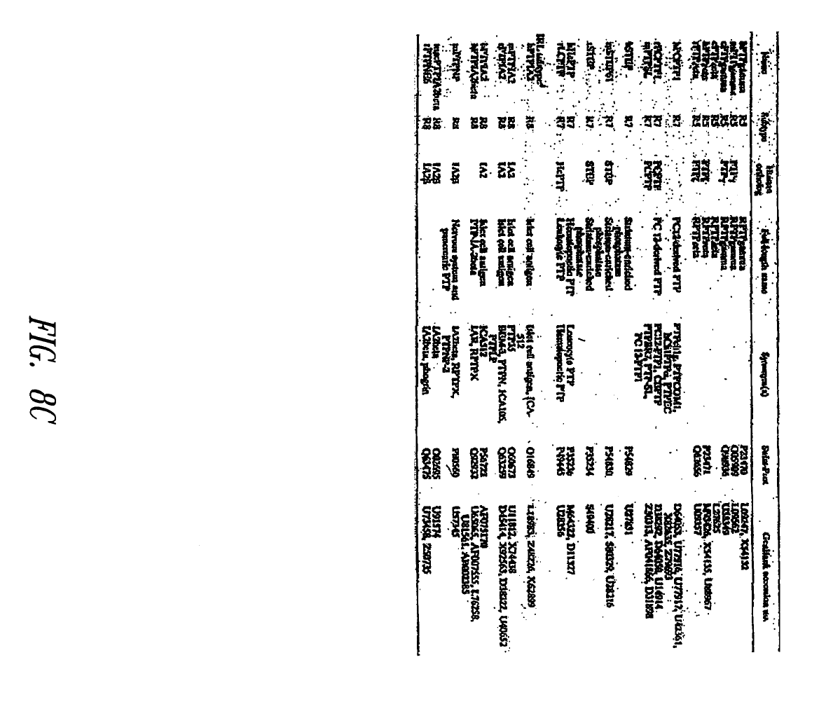

As used herein, a phosphatase is a member of the PTP family if it contains the signature motif [I/V]HCXAGXXR[S/T]G (SEQ ID NO:98). Dual specificity PTPs, i.e., PTPs which dephosphorylate both phosphorylated tyrosine and phosphorylated serine or threonine, are also suitable for use in the invention. Appropriate PTPs for use in the present invention include any PTP family member, for example, any PTP described in Andersen et al. (2001 Mol. Cell. Biol. 21:7117) or shown in FIG. 8, or any dual specificity phosphatase including but not limited to PYST-1, MKP-1, MKP-2, MKP-4, MKP-5, MKP-7, hVH5, PAC1, VHR, or any dual specificity phosphatase disclosed in WO0/65069 (DSP-5), WO00/65068 (DSP-10), WO00/63393 (DSP-8), WO00/60100 (DSP-9), WO00/60099 (DSP-4), WO00/60098 (DSP-7), WO00/60092 (DSP-3), WO00/56899 (DSP-2), WO00/53636 (DSP-1), WO00/09656 (MKP), AU5475399 (MKP), AU8479498, WO99/02704, WO97/06245 (MKP), WO01/83723, WO01/57221, WO01/05983, WO01/02582, WO01/02581, U.S. application Ser. No. 09/955,732 (DSP-15), U.S. application Ser. No. 09/964,277 (DSP-16), U.S. A. No. 60/268,837 (DSP-17) or U.S. A. No. 60/291,476 (PTP) and in certain preferred embodiments including, but not limited to, PTP1B (e.g., GenBank Accession Nos. M31724 (SEQ ID NOS: 1-2); NM_{—}002827 (SEQ ID NOS: 3-4); NM_{—}011201 (SEQ ID NOS: 5-6); M31724 (SEQ ID NOS: 7-8); M33689 (SEQ ID NOS: 9-10); M33962 (SEQ ID NOS: 11-12)), PTP-PEST (e.g., GenBank Accession Nos. D13380 (SEQ ID NOS: 68-69); M93425 (SEQ ID NOS: 70-71); S69184 (SEQ ID NOS: 72-73); X86781 (SEQ ID NOS: 74-75); D38072 (SEQ ID NOS: 76-77)), PTPγ, LAR, MKP-1, CRYPα, PTPcryp2, DEP-1 (e.g., GenBank Accession Nos. U10886 (SEQ ID NOS: 41-42); D37781 (SEQ ID NOS: 43-44); AAB26475 (SEQ ID NO: 45); D45212 (SEQ ID NOS: 46-47); U40790 (SEQ ID NOS: 48-49)), SAP1, PCPTP1, PTPSL, STEP, HePTP, PTPIA2, PTPNP, PTPNE6, PTPμ, PTPX1, PTPX10, SHP-1 (e.g., GenBank Accession Nos. M74903 (SEQ ID NOS: 86-87); X62055 (SEQ ID NOS: 88-89); M77273 (SEQ ID NOS: 90-91); X82817 (SEQ ID NO: 92); X82818 (SEQ ID NO: 93); M90388 (SEQ ID NOS: 94-95); U77038 (SEQ ID NOS: 96-97)), SHP-2 (e.g., GenBank Accession Nos. D13540 (SEQ ID NOS: 25-26); L03535 (SEQ ID NOS: 27-28); L07527 (SEQ ID NOS: 29-30); X70766 (SEQ ID NOS: 31-32); L08807 (SEQ ID NO: 33); S78088 (SEQ ID NOS: 34-35); S39383 (SEQ ID NO: 36); D84372 (SEQ ID NOS: 13-14); U09307 (SEQ ID NOS: 15-16)), PTPBEM1, PTPBEM2, PTPBYP, PTPesp, PTPoc, PTP-PEZ, PTP-MEG1, MEG2, LC-PTP, TC-PTP (e.g., GenBank Accession Nos. M25393 (SEQ ID NOS: 17-18); M81478 (SEQ ID NO: 19); M80737 (SEQ ID NO: 20); M81477 (SEQ ID NOS: 21-22); X58828 (SEQ ID NOS: 23-24); NM_{—}002828 (SEQ ID NOS: ______ and ______), TC45 (e.g., NM_{—}080422 (SEQ ID NOS: ______ and ______), CD45 (e.g., GenBank Accession Nos. Y00638 (SEQ ID NOS: 78-79); Y00062 (SEQ ID NOS: 80-81); M92933 (SEQ ID NOS: 82-83); M10072 (SEQ ID NOS: 84-85); LAR, cdc14 (which includes cdc14a (e.g., GenBank Accession Nos. AF122013 (SEQ ID NOS: 50-51); AF064102 (SEQ ID NOS: 52-53); AF064103 (SEQ ID NOS: 54-55); Li et al., 1997 J. Biol. Chem. 272:29403; U.S. Pat. No. 6,331,614) and cdc14b (e.g., GenBank Accession Nos. AF064104 (SEQ ID NOS: 56-57); AF064105 (SEQ ID NOS: 58-59); AF023158 (SEQ ID NOS: 60-61); Li et al., 1997 J Biol. Chem. 272:29403), RPTP-α, RPTP-ε, RKPTP, LyPTP, PEP, BDP1, PTP20, PTPK1, PTPS31, PTPGMC, GLEPP1, OSTPTP, PTPtep, PTPRL10, PTP2E, PTPD1, PTPD2, PTP36, PTPBAS, PTPBL, BTPBA14, PTPTyp, HDPTP, PTPTD14, PTPα, PTPβ, PTPδ, PTPε (e.g., GenBank Accession Nos. X54134 (SEQ ID NOS: 62-63); D83484 (SEQ ID NOS: 64-65); D78610 (SEQ ID NOS: 66-67)), PTPκ, PTPλ, PTPμ, PTPρ, PTPψ, PTPφ, PTPζ, PTPNU3 and PTPH1 (e.g., GenBank Accesion Nos. M64572 (SEQ ID NOS: 37-38) and S39392 (SEQ ID NOS: 39-40)), and mutated forms thereof.

As noted above, and particularly with regard to the identification and selection of suitable PTP substrates as provided herein, including peptide fragments having sequences derived from portions of polypeptides identified as physiological PTP substrates, the present invention relates in part to the use of substrate trapping mutant protein tyrosine phosphatases (PTPs) derived from a PTP that has been mutated in a manner that does not cause significant alteration of the Michaelis-Menten constant (Km) of the enzyme, but which results in a reduction of the catalytic rate constant (Kcat). In certain embodiments, the PTP catalytic domain invariant aspartate residue may be replaced with another amino acid. In certain other embodiments, the substrate trapping mutant PTP may be mutated by replacement of a catalytic domain cysteine residue. Under certain conditions in vivo, a PTP enzyme may itself undergo tyrosine phosphorylation in a manner that can alter interactions between the PTP and other molecules, including PTP substrates. Thus, in certain embodiments the substrate trapping mutant PTP may be further mutated by replacement of at least one tyrosine residue with an amino acid that is not capable of being phosphorylated. Substrate trapping mutant PTPs are disclosed, for example, in U.S. Pat. Nos. 5,912,138 and 5,951,979 and in U.S. application Ser. No. 09/334,575. Disclosure relating to the preparation and use of substrate trapping mutant PTPs, including PTPs having at least one tyrosine residue replaced with an amino acid that is not capable of being phosphorylated, and including identification of physiological PTP substrates, can be found in WO 00/75339.

According to particularly preferred embodiments of the methods of the present invention, PTPs in which the wildtype catalytic domain invariant cysteine residues are present, may be inactivated by sulfhydryl-reactive agents according to assay methods as disclosed herein. Preferably, such agents are sulfhydryl-reactive agents that are capable of covalently and irreversibly modifying a sulthydryl group of a PTP active site invariant cysteine, for example alkylating agents such as N-ethylmaleimide (NEM), iodoacetamide (IAA) or iodoacetic acid. Other sulfhydryl-reactive agents that are capable of covalently modifying a sulfhydryl group of a PTP active site invariant cysteine include arsenic oxide; 4-vinyl pyridine and analogs and derivatives thereof; maleimide analogs conforming to the following structural formula:

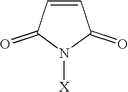

wherein X is the remainder of the molecule, including linkers;

or halo-acetamido analogs conforming to the following structural formula:

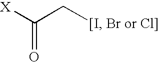

wherein X is the remainder of the molecule, including linkers.

Useful sulthydryl-reactive agents may also include other cysteine-reactive compounds, i.e., chemically reactive species that covalently modify cysteine and/or adjacent residues, further including such compounds which do so stoichiometrically and without selectivity for PTP proteins or polypeptides.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons).

Samples

According to the present invention, there is provided a method of identifying a protein tyrosine phosphatase that has been reversibly oxidized, typically in a biological sample. A “sample” as used herein refers to a biological sample containing at least one protein tyrosine phosphatase, and may be provided by obtaining a blood sample, biopsy specimen, tissue explant, organ culture or any other tissue or cell preparation from a subject or a biological source. A sample may further refer to a tissue or cell preparation in which the morphological integrity or physical state has been disrupted, for example, by dissection, dissociation, solubilization, fractionation, homogenization, biochemical or chemical extraction, pulverization, lyophilization, sonication or any other means for processing a sample derived from a subject or biological source. In certain preferred embodiments, the sample is a cell that comprises at least one PTP, and in certain particularly preferred embodiments the cell comprises an inducible biological signaling pathway, at least one component of which is a PTP. In particularly preferred embodiments the cell is a mammalian cell, for example, Rat-1 fibroblasts, COS cells, CHO cells, HEK-293 cells or other well known model cell lines, which are available from the American Type Culture Collection (ATCC, Manassas, Va.).

The subject or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines and the like. Optionally, in certain situations it may be desirable to treat cells in a biological sample with hydrogen peroxide and/or with another agent that directly or indirectly promotes reactive oxygen species (ROS) generation, including biological stimuli as described herein; in certain other situations it may be desirable to treat cells in a biological sample with a ROS scavenger, such as N-acetyl cysteine (NAC) or superoxide dismutase (SOD) or other ROS scavengers known in the art; in other situations cellular glutathione (GSH) may be depleted by treating cells with L-buthionine-SR-sulfoximine (Bso); and in other circumstances cells may be treated with pervanadate to enrich the sample in tyrosine phosphorylated proteins. Other means may also be employed to effect an increase in the population of tyrosine phosphorylated proteins present in the sample, including the use of a subject or biological source that is a cell line that has been transfected with at least one gene encoding a protein tyrosine kinase.

Additionally or alternatively, a biological signaling pathway may be induced in subject or biological source cells by contacting such cells with an appropriate stimulus, which may vary depending upon the signaling pathway under investigation, whether known or unknown. For example, a signaling pathway that, when induced, results in protein tyrosine phosphorylation and/or protein tyrosine dephosphorylation may be stimulated in subject or biological source cells using any one or more of a variety of well known methods and compositions known in the art to stimulate protein tyrosine kinase and/or PTP activity. These stimuli may include, without limitation, exposure of cells to cytokines, growth factors, hormones, peptides, small molecule mediators, cell stressors (e.g., ultraviolet light; temperature shifts; osmotic shock; ROS or a source thereof, such as hydrogen peroxide, superoxide, ozone, etc. or any agent that induces or promotes ROS production (see, e.g., Halliwell and Gutteridge, Free Radicals in Biology and Medicine (3^{rd} Ed.) 1999 Oxford University Press, Oxford, UK); heavy metals; alcohol) or other agents that induce PTK-mediated protein tyrosine phosphorylation and/or PTP-mediated phosphoprotein tyrosine dephosphorylation. Such agents may include, for example, interleukins (e.g., IL-1, IL-3), interferons (e.g., IFN-γ), human growth hormone, insulin, epidermal growth factor (EGF), platelet derived growth factor (PDGF), granulocyte colony stimulating factor (G-CSF), granulocyte-megakaryocyte colony stimulating factor (GM-CSF), transforming growth factor (e.g., TGF-β1), tumor necrosis factor (e.g., TNF-α) and fibroblast growth factor (FGF; e.g., basic FGF (bFGF)), any agent or combination of agents capable of triggering T lymphocyte activation via the T cell receptor for antigen (TCR; TCR-inducing agents may include superantigens, specifically recognized antigens and/or MHC-derived peptides, MHC peptide tetramers (e.g., Altman et al., 1996 Science 274:94-96) TCR-specific antibodies or fragments or derivatives thereof), lectins (e.g., PHA, PWM, ConA, etc.), mitogens, G-protein coupled receptor agonists such as angiotensin-2, thrombin, thyrotropin, parathyroid hormone, lysophosphatidic acid (LPA), sphingosine-1-phosphate, serotonin, endothelin, acetylcholine, platelet activating factor (PAF) or bradykinin, as well as other agents with which those having ordinary skill in the art will be familiar (see, e.g., Rhee et al., Oct. 10, 2000 Science's stke, <http:www.stke.org/cgl/content/full/OC_sigtrans;2000/53/pel, and references cited therein; see also Gross et al., 1999 J. Biol. Chem. 274:26378-86; Prenzel et al., 1999 Nature 402:884-88; Ushio-Fukai et al., 1999 J. Biol. Chem. 274:22699-704; Holland et al., 1998 Endothelium 6:113-21; Daub et al., 1997 EMBO J. 16:7032-44; Krypianou et al., 1997 Prostate 32:266-71; Marumo et al., 1997 Circulation 96:2361-67).

As noted above, regulated tyrosine phosphorylation contributes to specific pathways for biological signal transduction, including those associated with cell division, cell survival, apoptosis, proliferation and differentiation, and “inducible signaling pathways” in the context of the present invention include transient or stable associations or interactions among molecular components involved in the control of these and similar processes in cells. Depending on the particular pathway of interest, an appropriate parameter for determining induction of such pathway may be selected. For example, for signaling pathways associated with cell proliferation, there is available a variety of well known methodologies for quantifying proliferation, including, for example, incorporation of tritiated thymidine into cellular DNA, monitoring of detectable (e.g. fluorimetric or calorimetric) indicators of cellular respiratory activity, or cell counting, or the like. Similarly, in the cell biology arts there are known multiple techniques for assessing cell survival (e.g., vital dyes, metabolic indicators, etc.) and for determining apoptosis (e.g., annexin V binding, DNA fragmentation assays, caspase activation, etc.). Other signaling pathways will be associated with particular cellular phenotypes, for example specific induction of gene expression (e.g., detectable as transcription or translation products, or by bioassays of such products, or as nuclear localization of cytoplasmic factors), altered (e.g., statistically significant increases or decreases) levels of intracellular mediators (e.g., activated kinases or phosphatases, altered levels of cyclic nucleotides or of physiologically active ionic species, etc.), or altered cellular morphology, and the like, such that cellular responsiveness to a particular stimulus as provided herein can be readily identified to determine whether a particular cell comprises an inducible signaling pathway.

For example, a biological signaling pathway may be induced in a cell by a stimulus that induces or promotes ROS production. Cells may be stimulated with any one or more of a number of stimuli as provided herein, including those provided above, such as a cytokine, a growth factor (e.g., PDGF), a hormone such as a polypeptide hormone (e.g., insulin), a cell stressor, or a peptide. Intracellular production of ROS, including hydrogen peroxide, may be determined according to established methodologies using direct or indirect ROS indicators, for example, by using fluorescent ROS indicators such as 2′,7′-dichlorofluorescein diacetate (H₂DCFDA) or 5-(and-6)-chloromethyl-2′,7′-dichlorodihydrofluorescein diacetate (CM-H₂DCFDA). ROS-induced DCF fluorescence can then be measured, for instance, by fluorimetry, fluorescence microscopy or flow cytofluorimetry, or according to other methods known in the art. ROS may also be detected in biological systems by any of a variety of other techniques, including spin trapping, in which a reactive radical is allowed to react with a molecular trap to produce a long-lived radical, and also including molecular fingerprinting, which measures end-products of oxidative damage. Specific compositions and methods for such trapping, as well as other means for determining ROS, are known to the art and selection of a technique for identifying ROS may depend upon the particular reactive oxygen species that is to be detected (see, e.g., Halliwell and Gutteridge, supra).

The effect of ROS production on phosphorylation and/or dephosphorylation of one or more polypeptide components of a signaling pathway may be examined by determining the level of phosphorylation of components in the particular pathway. For example, treatment of Rat-1 cells with PDGF, which has been shown to induce ROS production in various cell types (Bae et al., 2000, supra; Sundaresan et al. supra), results in a rapid increase in the tyrosine phosphorylation of cellular proteins and enhanced phosphorylation of MAPKs (see also Bazenet et al., 1996 Mol. Cell Biol. 16:6926-36; Klinghoffer et al., 2001 Mol. Cell 78:343-54; Yu et al., 2000 J. Biol. Chem. 275:19076-82). As another example, the effect of ROS production in the signal transduction pathway induced by insulin may be evaluated by determining the level of tyrosine phosphorylation of insulin receptor beta (IR-β) and/or of the downstream signaling molecule PKB/Akt and/or of any other downstream polypeptide that may be a component of a particular signal transduction pathway as provided herein.

A number of methods are described herein and known in the art for detection of one or more particular signal transduction pathway component polypeptides, and for determination of whether such polypeptides may be tyrosine-phosphorylated in cells following stimulation as described herein. Also described herein are methods for detecting such polypeptides, including determination of altered (i.e., increased or decreased with statistical significance) tyrosine phosphorylation that may further include determination of the phosphorylation state of particular tyrosine residues at specified positions within a polypeptide sequence, which altered tyrosine phosphorylation may in certain embodiments be accompanied by the presence or absence of ROS production in the cells from which such polypeptides are obtained (e.g., as a result of exposure to a stimulus). Non-limiting examples of such detection methods include the use of reagents that specifically bind to signaling pathway components, for example, by immunological methods (e.g. immunoprecipitation, immunoblotting, ELISA, radioimmunoprecipitation, and the like) that employ antibodies as provided herein that are capable of specifically binding a particular signaling pathway component polypeptide or a particular tyrosine-phosphorylated polypeptide. Additionally and as described in greater detail herein, in certain embodiments cellular ROS production induced by a stimulus may be partially or completely impaired, abrogated, inhibited or otherwise counteracted by inclusion of a ROS-neutralizing agent, for instance, by the presence of enzymes such as catalase (H₂O₂:H₂O₂ oxidoreductase) or superoxide dismutase (SOD; superoxide:superoxide oxidoreductase), or of free-radical scavengers or other agents known to the art that are capable of neutralizing the effects of ROS (see, e.g., Halliwell and Gutteridge, supra).

Substrates

In preferred embodiments, a PTP substrate may be any naturally or non-naturally occurring phosphorylated peptide, polypeptide or protein that can specifically bind to and/or be dephosphorylated by a PTP (including dual specificity phosphatases) as provided herein, or any other phosphorylated molecule that can be a substrate of a PTP family member as provided herein. Non-limiting examples of known PTP substrates include the proteins VCP (see, e.g., Zhang et al., 1999 J. Biol. Chem. 274:17806, and references cited therein), p130^{cas}, EGF receptor, p210 bcr:abl, MAP kinase, Shc (Tiganis et al., 1998 Mol. Cell. Biol. 18:1622-1634), insulin receptor, lck (lymphocyte specific protein tyrosine kinase, Marth et al., 1985 Cell 43:393), T cell receptor zeta chain, and phosphatidylinositol 3,4,5-triphosphate (Maehama et al., 1998 J. Biol. Chem. 273:13375).

As another example, tyrosine phosphorylated peptides identified with mutant PTPs from peptide libraries by the methods of Songyang et al. (1995 Nature 373:536-539; 1993 Cell 72:767-778) can be used herein in place of the complete tyrosine phosphorylated protein in PTP binding and/or catalytic assays. Optionally, candidate peptide sequences may be selected and optimized for dephosphorylation or binding activity as described herein using other techniques such as affinity selection followed by mass spectrometric detection (e.g., Pellegrini et al., 1998 Biochemistry 37:15598; Huyer et al., 1998 Anal. Biochem. 258:19) or by “inverse alanine scanning” (e.g., Vetter et al., 2000 J. Biol. Chem. 275:2265). In certain particularly preferred embodiments, a PTP substrate is a tyrosine phosphorylated peptide, which may include a partial amino acid sequence, portion, region, fragment, variant, derivative or the like from a naturally or non-naturally tyrosine-phosphorylated peptide, polypeptide or protein that can specifically bind to and/or be dephosphorylated by a PTP. In preferred embodiments, the PTP substrate is detectably labeled as provided herein, such that it can be detectably dephosphorylated by a PTP family member, as also provided herein. A PTP substrate that is a tyrosine phosphorylated peptide typically comprises 2-700 amino acids. Preferred substrates as described herein include a random amino acid copolymer of poly-Glu-Tyr wherein the Glu:Tyr ratio is approximately 4:1; preparations of this copolymer may be polydisperse with respect to molecular mass and in certain preferred embodiments may have an average molecular mass of approximately 55-65 kDa. Other preferred substrates include reduced and carboxyamidomethylated and maleylated lysozyme (RCML, Flint et al., 1993 EMBO J. 12:1937-1946). In certain other embodiments, a PTP substrate may comprise a phosphotyrosine residue having an attached fluorescent label.

Identification and selection of PTP substrates as provided herein, for use in the present invention, may be performed according to procedures with which those having ordinary skill in the art will be familiar, or may, for example, be conducted according to the disclosures of WO 00/75339 or U.S. application Ser. No. 09/334,575 and references cited therein. The phosphorylated protein/PTP complex may be isolated, for example, by conventional isolation techniques as described in U.S. Pat. No. 5,352,660, including salting out, chromatography, electrophoresis, gel filtration, fractionation, absorption, polyacrylamide gel electrophoresis, agglutination, combinations thereof or other strategies. PTP substrates that are known may also be prepared according to well known procedures that employ principles of molecular biology and/or peptide synthesis (e.g., Ausubel et al., 1993 Current Protocols in Molecular Biology, Greene Publ. Assoc. Inc. & John Wiley & Sons, Inc., Boston, Mass.; Sambrook et al., 1989 Molecular Cloning, Second Ed., Cold Spring Harbor Laboratory, Plainview, N.Y.; Fox, 1995 Molec. Biotechnol. 3:249; Maeji et al., 1995 Pept. Res. 8:33).

The PTP substrate peptides of the present invention may therefore be derived from PTP substrate proteins, polypeptides and peptides as provided herein having amino acid sequences that are identical or similar to tyrosine phosphorylated PTP substrate sequences known in the art. For example by way of illustration and not limitation, peptide sequences derived from the known PTP substrate proteins referred to above are contemplated for use according to the instant invention, as are peptides having at least 70% similarity (preferably 70% identity), more preferably 90% similarity (more preferably 90% identity) and still more preferably 95% similarity (still more preferably 95% identity) to the polypeptides described in references cited herein and in the Examples and to portions of such polypeptides as disclosed herein. As known in the art “similarity” between two polypeptides is determined by comparing the amino acid sequence and conserved amino acid substitutes thereto of the polypeptide to the sequence of a second polypeptide (e.g., using GENEWORKS, Align or the BLAST algorithm, or another algorithm, as described above).

Thus, according to the present invention, substrates may include full length tyrosine phosphorylated proteins and polypeptides as well as fragments (e.g., portions), derivatives or analogs thereof that can be phosphorylated at a tyrosine residue. Such fragments, derivatives and analogs include any PTP substrate polypeptide that retains at least the biological function of interacting with a PTP as provided herein, for example by forming a complex with a PTP and/or, in certain embodiments, undergoing PTP-catalyzed dephosphorylation. A fragment, derivative or analog of a peptide, protein or polypeptide as provided herein, including a PTP substrate polypeptide, and further including PTP substrates that are fusion proteins, may be (i) one in which one or more of the amino acid residues are substituted with a conserved or non-conserved amino acid residue (preferably a conserved amino acid residue), and such substituted amino acid residue may or may not be one encoded by the genetic code, or (ii) one in which one or more of the amino acid residues includes a substituent group, or (iii) one in which the substrate polypeptide is fused with another compound, such as a compound to increase the half-life of the polypeptide (e.g., polyethylene glycol) or a detectable moiety such as a reporter molecule, or (iv) one in which additional amino acids are fused to the substrate polypeptide, including amino acids that are employed for purification of the substrate polypeptide or a proprotein sequence. Such fragments, derivatives and analogs are deemed to be within the scope of those skilled in the art.

Certain preferred substrates include phosphoproteins and phosphopeptide sequences that may be tyrosine phosphorylated and/or serine/threonine phosphorylated, for example, as may provide suitable phosphophorylated substrates for dual specificity phosphatases, which are described above. Examples of physiological substrates which may provide phosphoprotein or phosphopeptides sequences for use as PTP substrates, including fragments, variants and derivatives as provided herein, include PDGF receptor, VCP, p130^{cas}, EGF receptor, p210 bcr:abl, MAP kinase, Shc, insulin receptor, lck, and T cell receptor zeta chain. A number of non-physiological phosphoproteins and phosphopeptides are also known to be suitable PTP substrates, as described, for example, by Tonks et al. (1991 Meths. Enzymol. 201:427-42; 1988 J. Biol. Chem. 263:6722); these include, as non-limiting examples, poly-[Glu-Tyr], MBP and reduced and carboxyamidomethylated and maleylated lysozyme (RCML, Flint et al., 1993 EMBO J. 12:1937-1946).

In preferred embodiments the PTP substrate is detectably labeled, and in particularly preferred embodiments the PTP substrate is capable of generating a radioactive or a fluorescent signal. The PTP substrate can be detectably labeled by covalently or non-covalently attaching a suitable reporter molecule or moiety, for example a radionuclide such as ³²P (e.g., Pestka et al., 1999 Protein Expr. Purif. 17:203-14), a radiohalogen such as iodine [¹²⁵I or ¹³¹I] (e.g., Wilbur, 1992 Bioconjug. Chem. 3:433-70), or tritium [³H]; an enzyme; or any of various luminescent (e.g., chemiluminescent) or fluorescent materials (e.g., a fluorophore) selected according to the particular fluorescence detection technique to be employed, as known in the art and based upon the present disclosure. Fluorescent reporter moieties and methods for labeling PTP substrates as provided herein can be found, for example in Haugland (1996 Handbook of Fluorescent Probes and Research Chemicals—Sixth Ed., Molecular Probes, Eugene, Oreg.; 1999 Handbook of Fluorescent Probes and Research Chemicals—Seventh Ed., Molecular Probes, Eugene, Oreg., http://www.probes.com/lit/) and in references cited therein. Particularly preferred for use as such a fluorophore in the subject invention methods are fluorescein, rhodamine, Texas Red, AlexaFluor-594, AlexaFluor-488, Oregon Green, BODIPY-FL, umbelliferone, dichlorotriazinylamine fluorescein, dansyl chloride, phycoerythrin or Cy-5. Examples of suitable enzymes include, but are not limited to, horseradish peroxidase, biotin, alkaline phosphatase, β-galactosidase and acetylcholinesterase. Appropriate luminescent materials include luminol, and suitable radioactive materials include radioactive phosphorus [³²P].

Antibodies

Also contemplated by the present invention is the use according to certain embodiments of an antibody that specifically binds to a PTP, which may include peptides, polypeptides, and other non-peptide molecules that specifically bind to a PTP. As used herein, a molecule is said to “specifically bind” to a PTP if it reacts at a detectable level with the PTP, but does not react detectably with peptides containing an unrelated sequence, or a sequence of a different phosphatase. Preferred binding molecules include antibodies, which may be, for example, polyclonal, monoclonal, single chain, chimeric, anti-idiotypic, or CDR-grafted immunoglobulins, or fragments thereof, such as proteolytically generated or recombinantly produced immunoglobulin F(ab′)₂, Fab, Fv, and Fd fragments. Binding properties of an antibody to a PTP may generally be assessed using immunodetection methods including, for example, an enzyme-linked immunosorbent assay (ELISA), immunoprecipitation, immunoblotting and the like, which may be readily performed by those having ordinary skill in the art. In certain preferred embodiments, the invention method may comprise isolating one or more particular PTPs with an antibody that specifically binds to each phosphatase; such embodiments may include without limitation methodologies for immuno-isolation (e.g., immunoprecipitation, immunoaffinity chromatography) and/or immunodetection (e.g. western blot) of at least one PTP.

Methods well known in the art may be used to generate antibodies, polyclonal antisera or monoclonal antibodies that are specific for a PTP; a number of PTP-specific antibodies are also commercially available. Antibodies also may be produced as genetically engineered immunoglobulins (Ig) or Ig fragments designed to have desirable properties. For example, by way of illustration and not limitation, antibodies may include a recombinant IgG that is a chimeric fusion protein having at least one variable (V) region domain from a first mammalian species and at least one constant region domain from a second, distinct mammalian species. Most commonly, a chimeric antibody has murine variable region sequences and human constant region sequences. Such a murine/human chimeric immunoglobulin may be “humanized” by grafting the complementarity determining regions (CDRs) derived from a murine antibody, which confer binding specificity for an antigen, into human-derived V region framework regions and human-derived constant regions. Fragments of these molecules may be generated by proteolytic digestion, or optionally, by proteolytic digestion followed by mild reduction of disulfide bonds and alkylation. Alternatively, such fragments may also be generated by recombinant genetic engineering techniques.

As used herein, an antibody is said to be “immunospecific” or to “specifically bind” a PTP polypeptide if it reacts at a detectable level with PTP, preferably with an affinity constant, Kₐ, of greater than or equal to about 10⁴ M⁻¹, more preferably of greater than or equal to about 10⁵ M⁻¹, more preferably of greater than or equal to about 10⁶ M⁻¹, and still more preferably of greater than or equal to about 10⁷ M⁻¹. Affinities of binding partners or antibodies can be readily determined using conventional techniques, for example, those described by Scatchard et al. (Ann. N. Y Acad. Sci. USA 51:660 (1949)) and by surface plasmon resonance (SPR; BIAcore™, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the surface plasmon resonance signal can be analyzed to yield apparent rate constants for the association and dissociation phases of the binding reaction. The ratio of these values gives the apparent equilibrium constant (affinity). See, e.g., Wolff et al., Cancer Res. 53:2560-2565 (1993).

Antibodies may generally be prepared by any of a variety of techniques known to those having ordinary skill in the art. See, e.g., Harlow et al., Antibodies: A Laboratory Manual, Cold Spring Harbor Laboratory (1988). In one such technique, an animal is immunized with PTP as an antigen to generate polyclonal antisera. Suitable animals include, for example, rabbits, sheep, goats, pigs, cattle, and may also include smaller mammalian species, such as mice, rats, and hamsters, or other species.

An immunogen may be comprised of cells expressing PTP, purified or partially purified PTP polypeptides or variants or fragments (e.g., peptides) thereof, or PTP peptides. PTP peptides may be generated by proteolytic cleavage or may be chemically synthesized. For instance, nucleic acid sequences encoding PTP polypeptides are provided herein, such that those skilled in the art may routinely prepare these polypeptides for use as immunogens. Polypeptides or peptides useful for immunization may also be selected by analyzing the primary, secondary, and tertiary structure of PTP according to methods known to those skilled in the art, in order to determine amino acid sequences more likely to generate an antigenic response in a host animal. See, e.g., Novotny, 1991 Mol. Immunol. 28:201-207; Berzofsky, 1985 Science 229:932-40.

Preparation of the immunogen for injection into animals may include covalent coupling of the PTP polypeptide (or variant or fragment thereof), to another immunogenic protein, for example, a carrier protein such as keyhole limpet hemocyanin (KLH) or bovine serum albumin (BSA). In addition, the PTP peptide, polypeptide, or PTP-expressing cells to be used as immunogen may be emulsified in an adjuvant. See, e.g., Harlow et al., Antibodies: A Laboratory Manual, Cold Spring Harbor Laboratory (1988). In general, after the first injection, animals receive one or more booster immunizations according to a preferred schedule that may vary according to, inter alia, the antigen, the adjuvant (if any) and/or the particular animal species. The immune response may be monitored by periodically bleeding the animal, separating the sera out of the collected blood, and analyzing the sera in an immunoassay, such as an ELISA or Ouchterlony diffusion assay, or the like, to determine the specific antibody titer. Once an antibody titer is established, the animals may be bled periodically to accumulate the polyclonal antisera. Polyclonal antibodies that bind specifically to the PTP polypeptide or peptide may then be purified from such antisera, for example, by affinity chromatography using protein A, or the PTP polypeptide, immobilized on a suitable solid support.

Monoclonal antibodies that specifically bind to PTP polypeptides or fragments or variants thereof, and hybridomas, which are immortal eukaryotic cell lines, that produce monoclonal antibodies having the desired binding specificity, may also be prepared, for example, using the technique of Kohler and Milstein (Nature, 256:495-497; 1976, Eur. J Immunol. 6:511-519 (1975)) and improvements thereto. An animal—for example, a rat, hamster, or preferably mouse—is immunized with a PTP immunogen prepared as described above. Lymphoid cells that include antibody-forming cells, typically spleen cells, are obtained from an immunized animal and may be immortalized by fusion with a drug-sensitized myeloma (e.g., plasmacytoma) cell fusion partner, preferably one that is syngeneic with the immunized animal and that optionally has other desirable properties (e.g., inability to express endogenous Ig gene products). The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells, but not unfused myeloma cells. A preferred selection media is HAT (hypoxanthine, aminopterin, thymidine). After a sufficient time, usually about one to two weeks, colonies of cells are observed. Single colonies are isolated, and antibodies produced by the cells may be tested for binding activity to the PTP polypeptide, or variant or fragment thereof. Hybridomas producing monoclonal antibodies with high affinity and specificity for a PTP antigen are preferred. Hybridomas that produce monoclonal antibodies that specifically bind to a PTP polypeptide or variant or fragment thereof are therefore contemplated by the present invention.

Monoclonal antibodies may be isolated from the supernatants of hybridoma cultures. An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Contaminants may be removed from the subsequently (usually within 1-3 weeks) harvested ascites fluid by conventional techniques, such as chromatography, gel filtration, precipitation, extraction, or the like. For example, antibodies may be purified by affinity chromatography using an appropriate ligand selected based on particular properties of the monoclonal antibody (e.g., heavy or light chain isotype, binding specificity, etc.). Examples of a suitable ligand, immobilized on a solid support, include Protein A, Protein G, an anti-constant region (light chain or heavy chain) antibody, an anti-idiotype antibody and a PTP polypeptide or fragment or variant thereof.

Human monoclonal antibodies may be generated by any number of techniques with which those having ordinary skill in the art will be familiar. Such methods include but are not limited to, Epstein Barr Virus (EBV) transformation of human peripheral blood cells (e.g., containing B lymphocytes), in vitro immunization of human B cells, fusion of spleen cells from immunized transgenic mice carrying human immunoglobulin genes inserted by yeast artificial chromosomes (YAC), isolation from human immunoglobulin V region phage libraries, or other procedures as known in the art and based on the disclosure herein.

For example, one method for generating human monoclonal antibodies includes immortalizing human peripheral blood cells by EBV transformation. See, e.g., U.S. Pat. No. 4,464,456. An immortalized cell line producing a monoclonal antibody that specifically binds to a PTP polypeptide (or a variant or fragment thereof) can be identified by immunodetection methods as provided herein, for example, an ELISA, and then isolated by standard cloning techniques. Another method to generate human monoclonal antibodies, in vitro immunization, includes priming human splenic B cells with antigen, followed by fusion of primed B cells with a heterohybrid fusion partner. See, e.g., Boerner et al., 1991 J. Immunol. 147:86-95.

Still another method for the generation of human PTP-specific monoclonal antibodies and polyclonal antisera for use in the present invention relates to transgenic mice. See, e.g., U.S. Pat. No. 5,877,397; Bruggemann et al., 1997 Curr. Opin. Biotechnol. 8:455-58; Jakobovits et al., 1995 Ann. N. Y. Acad. Sci. 764:525-35. In these mice, human immunoglobulin heavy and light chain genes have been artificially introduced by genetic engineering in germline configuration, and the endogenous murine immunoglobulin genes have been inactivated. See, e.g., Bruggemann et al., 1997 Curr. Opin. Biotechnol. 8:455-58. For example, human immunoglobulin transgenes may be mini-gene constructs, or transloci on yeast artificial chromosomes, which undergo B cell-specific DNA rearrangement and hypermutation in the mouse lymphoid tissue. See, Bruggemann et al., 1997 Curr. Opin. Biotechnol. 8:455-58. Human monoclonal antibodies specifically binding to PTP may be obtained by immunizing the transgenic animals, fusing spleen cells with myeloma cells, selecting and then cloning cells producing antibody, as described above. Polyclonal sera containing human antibodies may also be obtained from the blood of the immunized animals.

Chimeric antibodies, specific for a PTP, including humanized antibodies, may also be generated according to the present invention. A chimeric antibody has at least one constant region domain derived from a first mammalian species and at least one variable region domain derived from a second, distinct mammalian species. See, e.g., Morrison et al., 1984, Proc. Natl. Acad. Sci. USA, 81:6851-55. In preferred embodiments, a chimeric antibody may be constructed by cloning the polynucleotide sequence that encodes at least one variable region domain derived from a non-human monoclonal antibody, such as the variable region derived from a murine, rat, or hamster monoclonal antibody, into a vector containing a nucleic acid sequence that encodes at least one human constant region. See, e.g., Shin et al., 1989 Methods Enzymol. 178:459-76; Walls et al., 1993 Nucleic Acids Res. 21:2921-29. By way of example, the polynucleotide sequence encoding the light chain variable region of a murine monoclonal antibody may be inserted into a vector containing a nucleic acid sequence encoding the human kappa light chain constant region sequence. In a separate vector, the polynucleotide sequence encoding the heavy chain variable region of the monoclonal antibody may be cloned in frame with sequences encoding the human IgG1 constant region. The particular human constant region selected may depend upon the effector functions desired for the particular antibody (e.g., complement fixing, binding to a particular Fc receptor, etc.). Another method known in the art for generating chimeric antibodies is homologous recombination (e.g., U.S. Pat. No. 5,482,856). Preferably, the vectors will be transfected into eukaryotic cells for stable expression of the chimeric antibody.

A non-human/human chimeric antibody may be further genetically engineered to create a “humanized” antibody. Such a humanized antibody may comprise a plurality of CDRs derived from an immunoglobulin of a non-human mammalian species, at least one human variable framework region, and at least one human immunoglobulin constant region. Humanization may in certain embodiments provide an antibody that has decreased binding affinity for a PTP when compared, for example, with either a non-human monoclonal antibody from which a PTP binding variable region is obtained, or a chimeric antibody having such a V region and at least one human C region, as described above. Useful strategies for designing humanized antibodies may therefore include, for example by way of illustration and not limitation, identification of human variable framework regions that are most homologous to the non-human framework regions of the chimeric antibody. Without wishing to be bound by theory, such a strategy may increase the likelihood that the humanized antibody will retain specific binding affinity for a PTP, which in some preferred embodiments may be substantially the same affinity for a PTP polypeptide or variant or fragment thereof, and in certain other preferred embodiments may be a greater affinity for PTP. See, e.g., Jones et al., 1986 Nature 321:522-25; Riechmann et al., 1988 Nature 332:323-27. Designing such a humanized antibody may therefore include determining CDR loop conformations and structural determinants of the non-human variable regions, for example, by computer modeling, and then comparing the CDR loops and determinants to known human CDR loop structures and determinants. See, e.g., Padlan et al., 1995 FASEB 9:133-39; Chothia et al., 1989 Nature, 342:377-383. Computer modeling may also be used to compare human structural templates selected by sequence homology with the non-human variable regions. See, e.g., Bajorath et al., 1995 Ther. Immunol. 2:95-103; EP-0578515-A3. If humanization of the non-human CDRs results in a decrease in binding affinity, computer modeling may aid in identifying specific amino acid residues that could be changed by site-directed or other mutagenesis techniques to partially, completely or supra-optimally (i.e., increase to a level greater than that of the non-humanized antibody) restore affinity. Those having ordinary skill in the art are familiar with these techniques, and will readily appreciate numerous variations and modifications to such design strategies.

Within certain embodiments, the use of antigen-binding fragments of antibodies may be preferred. Such fragments include Fab fragments or F(ab′)₂ fragments, which may be prepared by proteolytic digestion with papain or pepsin, respectively. The antigen binding fragments may be separated from the Fc fragments by affinity chromatography, for example, using immobilized protein A or protein G, or immobilized PTP polypeptide, or a suitable variant or fragment thereof. Those having ordinary skill in the art can routinely and without undue experimentation determine what is a suitable variant or fragment based on characterization of affinity purified antibodies obtained, for example, using immunodetection methods as provided herein. An alternative method to generate Fab fragments includes mild reduction of F(ab′)₂ fragments followed by alkylation. See, e.g., Weir, Handbook of Experimental Immunology, 1986, Blackwell Scientific, Boston.

According to certain embodiments, non-human, human, or humanized heavy chain and light chain variable regions of any of the above described Ig molecules may be constructed as single chain Fv (sFv) polypeptide fragments (single chain antibodies). See, e.g., Bird et al., 1988 Science 242:423-426; Huston et al., 1988 Proc. Natl. Acad. Sci. USA 85:5879-5883. Multi-functional sFv fusion proteins may be generated by linking a polynucleotide sequence encoding an sFv polypeptide in-frame with at least one polynucleotide sequence encoding any of a variety of known effector proteins. These methods are known in the art, and are disclosed, for example, in EP-B1-0318554, U.S. Pat. No. 5,132,405, U.S. Pat. No. 5,091,513, and U.S. Pat. No. 5,476,786. By way of example, effector proteins may include immunoglobulin constant region sequences. See, e.g., Hollenbaugh et al., 1995 J. Immunol. Methods 188:1-7. Other examples of effector proteins are enzymes. As a non-limiting example, such an enzyme may provide a biological activity for therapeutic purposes (see, e.g., Siemers et al., 1997 Bioconjug. Chem. 8:510-19), or may provide a detectable activity, such as horseradish peroxidase-catalyzed conversion of any of a number of well-known substrates into a detectable product, for diagnostic uses. Still other examples of sFv fusion proteins include Ig-toxin fusions, or immunotoxins, wherein the sFv polypeptide is linked to a toxin. Those having ordinary skill in the art will appreciate that a wide variety of polypeptide sequences have been identified that, under appropriate conditions, are toxic to cells. As used herein, a toxin polypeptide for inclusion in an immunoglobulin-toxin fusion protein may be any polypeptide capable of being introduced to a cell in a manner that compromises cell survival, for example, by directly interfering with a vital function or by inducing apoptosis. Toxins thus may include, for example, ribosome-inactivating proteins, such as Pseudomonas aeruginosa exotoxin A, plant gelonin, bryodin from Bryonia dioica, or the like. See, e.g., Thrush et al., 1996 Annu. Rev. Immunol. 14:49-71; Frankel et al., 1996 Cancer Res. 56:926-32. Numerous other toxins, including chemotherapeutic agents, anti-mitotic agents, antibiotics, inducers of apoptosis (or “apoptogens”, see, e.g., Green and Reed, 1998, Science 281:1309-1312), or the like, are known to those familiar with the art, and the examples provided herein are intended to be illustrative without limiting the scope and spirit of the invention.

The sFv may, in certain embodiments, be fused to peptide or polypeptide domains that permit detection of specific binding between the fusion protein and antigen (e.g., a PTP). For example, the fusion polypeptide domain may be an affinity tag polypeptide. Binding of the sFv fusion protein to a binding partner (e.g., a PTP) may therefore be detected using an affinity polypeptide or peptide tag, such as an avidin, streptavidin or a His (e.g., polyhistidine) tag, by any of a variety of techniques with which those skilled in the art will be familiar. Detection techniques may also include, for example, binding of an avidin or streptavidin fusion protein to biotin or to a biotin mimetic sequence (see, e.g., Luo et al., 1998 J. Biotechnol. 65:225 and references cited therein), direct covalent modification of a fusion protein with a detectable moiety (e.g., a labeling moiety), non-covalent binding of the fusion protein to a specific labeled reporter molecule, enzymatic modification of a detectable substrate by a fusion protein that includes a portion having enzyme activity, or immobilization (covalent or non-covalent) of the fusion protein on a solid-phase support.

The sFv fusion protein of the present invention, comprising a PTP-specific immunoglobulin-derived polypeptide fused to another polypeptide such as an effector peptide having desirable affinity properties, may therefore include, for example, a fusion protein wherein the effector peptide is an enzyme such as glutathione-S-transferase. As another example, sFv fusion proteins may also comprise a PTP-specific Ig polypeptide fused to a Staphylococcus aureus protein A polypeptide; protein A encoding nucleic acids and their use in constructing fusion proteins having affinity for immunoglobulin constant regions are disclosed generally, for example, in U.S. Pat. No. 5,100,788. Other useful affinity polypeptides for construction of sFv fusion proteins may include streptavidin fusion proteins, as disclosed, for example, in WO 89/03422; U.S. Pat. Nos. 5,489,528; 5,672,691; WO 93/24631; U.S. Pat. Nos. 5,168,049; 5,272,254 and elsewhere, and avidin fusion proteins (see, e.g., EP 511,747). As provided herein, sFv polypeptide sequences may be fused to fusion polypeptide sequences, including effector protein sequences, that may include full length fusion polypeptides and that may alternatively contain variants or fragments thereof.

An additional method for selecting antibodies that specifically bind to a PTP polypeptide or variant or fragment thereof is by phage display. See, e.g., Winter et al., 1994 Annul. Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to a PTP polypeptide or variant or fragment thereof. See, e.g., U.S. Pat. No. 5,223,409; Huse et al., 1989 Science 246:1275-81; Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein, for instance, gene III or gene VIII of M13, to create an M13 fusion protein. A fusion protein may be a fusion of the coat protein with the light chain variable region domain and/or with the heavy chain variable region domain.

According to certain embodiments, immunoglobulin Fab fragments may also be displayed on the phage particle, as follows. Polynucleotide sequences encoding Ig constant region domains may be inserted into the phage genome in frame with a coat protein. The phage coat fusion protein may thus be fused to an Ig light chain or heavy chain fragment (Fd). For example, from a human Ig library, the polynucleotide sequence encoding the human kappa constant region may be inserted into a vector in frame with the sequence encoding at least one of the phage coat proteins. Additionally or alternatively, the polynucleotide sequence encoding the human IgG1 CH1 domain may be inserted in frame with the sequence encoding at least one other of the phage coat proteins. A plurality of polynucleotide sequences encoding variable region domains (e.g., derived from a DNA library) may then be inserted into the vector in frame with the constant region-coat protein fusions, for expression of Fab fragments fused to a bacteriophage coat protein.

Phage that display an Ig fragment (e.g., an Ig V-region or Fab) that binds to a PTP polypeptide may be selected by mixing the phage library with PTP or a variant or a fragment thereof, or by contacting the phage library with a PTP polypeptide immobilized on a solid matrix under conditions and for a time sufficient to allow binding. Unbound phage are removed by a wash, which typically may be a buffer containing salt (e.g., NaCl) at a low concentration, preferably with less than 100 mM NaCl, more preferably with less than 50 mM NaCl, most preferably with less than 10 mM NaCl, or, alternatively, a buffer containing no salt. Specifically bound phage are then eluted with an NaCl-containing buffer, for example, by increasing the salt concentration in a step-wise manner. Typically, phage that bind the PTP with higher affinity will require higher salt concentrations to be released. Eluted phage may be propagated in an appropriate bacterial host, and generally, successive rounds of PTP binding and elution can be repeated to increase the yield of phage expressing PTP-specific immunoglobulin. Combinatorial phage libraries may also be used for humanization of non-human variable regions. See, e.g., Rosok et al., 1996 J. Biol. Chem. 271:22611-18; Rader et al., 1998 Proc. Natl. Acad. Sci. USA 95:8910-15. The DNA sequence of the inserted immunoglobulin gene in the phage so selected may be determined by standard techniques. See, Sambrook et al., 1989 Molecular Cloning: A Laboratory Manual, Cold Spring Harbor Press. The affinity selected Ig-encoding sequence may then be cloned into another suitable vector for expression of the Ig fragment or, optionally, may be cloned into a vector containing Ig constant regions, for expression of whole immunoglobulin chains.

Phage display techniques may also be used to select polypeptides, peptides or single chain antibodies that bind to PTP. For examples of suitable vectors having multicloning sites into which candidate nucleic acid molecules (e.g., DNA) encoding such peptides or antibodies may be inserted, see, e.g., McLafferty et al., Gene 128:29-36, 1993; Scott et al., 1990 Science 249:386-390; Smith et al., 1993 Methods Enzymol. 217:228-257; Fisch et al., 1996, Proc. Natl. Acad. Sci. USA 93:7761-66. The inserted DNA molecules may comprise randomly generated sequences, or may encode variants of a known peptide or polypeptide domain that specifically binds to a PTP polypeptide, or variant or fragment thereof, as provided herein. Generally, the nucleic acid insert encodes a peptide of up to 60 amino acids, more preferably a peptide of 3 to 35 amino acids, and still more preferably a peptide of 6 to 20 amino acids. The peptide encoded by the inserted sequence is displayed on the surface of the bacteriophage. Phage expressing a binding domain for a PTP polypeptide may be selected on the basis of specific binding to an immobilized PTP polypeptide as described above. As provided herein, well-known recombinant genetic techniques may be used to construct fusion proteins containing the fragment thereof. For example, a polypeptide may be generated that comprises a tandem array of two or more similar or dissimilar affinity selected PTP binding peptide domains, in order to maximize binding affinity for PTP of the resulting product.

In certain other embodiments, the invention contemplates PTP-specific antibodies that are multimeric antibody fragments. Useful methodologies are described generally, for example in Hayden et al. 1997, Curr Opin. Immunol. 9:201-12; Coloma et al., 1997 Nat. Biotechnol. 15:159-63). For example, multimeric antibody fragments may be created by phage techniques to form miniantibodies (U.S. Pat. No. 5,910,573) or diabodies (Holliger et al., 1997, Cancer Immunol. Immunother. 45:128-130). Multimeric fragments may be generated that are multimers of a PTP-specific Fv, or that are bispecific antibodies comprising a PTP-specific Fv noncovalently associated with a second Fv having a different antigen specificity. See, e.g., Koelemij et al., 1999 J. Immunother. 22:514-24. As another example, a multimeric antibody may comprise a bispecific antibody having two single chain antibodies or Fab fragments. According to certain related embodiments, a first Ig fragment may be specific for a first antigenic determinant on a PTP polypeptide (or variant or fragment thereof), while a second Ig fragment may be specific for a second antigenic determinant of the PTP polypeptide. Alternatively, in certain other related embodiments, a first immunoglobulin fragment may be specific for an antigenic determinant on a PTP polypeptide or variant or fragment thereof, and a second immunoglobulin fragment may be specific for an antigenic determinant on a second, distinct (i.e., non-PTP) molecule. Also contemplated are bispecific antibodies that specifically bind PTP, wherein at least one antigen-binding domain is present as a fusion protein.

Introducing amino acid mutations into PTP-binding immunoglobulin molecules may be useful to increase the specificity or affinity for PTP, or to alter an effector function. Immunoglobulins with higher affinity for PTP may be generated by site-directed mutagenesis of particular residues. Computer assisted three-dimensional molecular modeling may be employed to identify the amino acid residues to be changed, in order to improve affinity for the PTP polypeptide. See, e.g., Mountain et al., 1992, Biotechnol. Genet. Eng. Rev. 10: 1-142. Alternatively, combinatorial libraries of CDRs may be generated in M13 phage and screened for immunoglobulin fragments with improved affinity. See, e.g., Glaser et al., 1992, J. Immunol. 149:3903-3913; Barbas et al., 1994 Proc. Natl. Acad. Sci. USA 91:3809-13; U.S. Pat. No. 5,792,456).

Effector functions may also be altered by site-directed mutagenesis. See, e.g., Duncan et al., 1988 Nature 332:563-64; Morgan et al., 1995 Immunology 86:319-24; Eghtedarzedeh-Kondri et al., 1997 Biotechniques 23:830-34. For example, mutation of the glycosylation site on the Fe portion of the immunoglobulin may alter the ability of the immunoglobulin to fix complement. See, e.g., Wright et al., 1997 Trends Biotechnol. 15:26-32. Other mutations in the constant region domains may alter the ability of the immunoglobulin to fix complement, or to effect antibody-dependent cellular cytotoxicity. See, e.g., Duncan et al., 1988 Nature 332:563-64; Morgan et al., 1995 Immunology 86:319-24; Sensel et al., 1997 Mol. Immunol. 34:1019-29.

The nucleic acid molecules encoding an antibody or fragment thereof that specifically binds PTP, as described herein, may be propagated and expressed according to any of a variety of well-known procedures for nucleic acid excision, ligation, transformation and transfection. Thus, in certain embodiments expression of an antibody fragment may be preferred in a prokaryotic host, such as Escherichia coli (see, e.g., Pluckthun et al., 1989 Methods Enzymol. 178:497-515). In certain other embodiments, expression of the antibody or a fragment thereof may be preferred in a eukaryotic host cell, including yeast (e.g., Saccharomyces cerevisiae, Schizosaccharomyces pombe, and Pichia pastoris), animal cells (including mammalian cells) or plant cells. Examples of suitable animal cells include, but are not limited to, myeloma, COS, CHO, or hybridoma cells. Examples of plant cells include tobacco, corn, soybean, and rice cells. By methods known to those having ordinary skill in the art and based on the present disclosure, a nucleic acid vector may be designed for expressing foreign sequences in a particular host system, and then polynucleotide sequences encoding the PTP binding antibody (or fragment thereof) may be inserted. The regulatory elements will vary according to the particular host.

A PTP-binding immunoglobulin (or fragment thereof) as described herein may contain a detectable moiety or label such as an enzyme, cytotoxic agent or other reporter molecule, including a dye, radionuclide, luminescent group, fluorescent group, or biotin, or the like. The PTP-specific immunoglobulin or fragment thereof may be radiolabeled for diagnostic or therapeutic applications. Techniques for radiolabeling of antibodies are known in the art. See, e.g., Adams 1998 In Vivo 12:11-21; Hiltunen 1993 Acta Oncol. 32:831-9. Therapeutic applications are described in greater detail below and may include use of the PTP-binding antibody (or fragment thereof) in conjunction with other therapeutic agents. The antibody or fragment may also be conjugated to a cytotoxic agent as known in the art and provided herein, for example, a toxin, such as a ribosome-inactivating protein, a chemotherapeutic agent, an anti-mitotic agent, an antibiotic or the like.

As provided herein and according to methodologies well known in the art, polyclonal and monoclonal antibodies may be used for the affinity isolation of PTP polypeptides. See, e.g., Hermanson et al., Immobilized Affinity Ligand Techniques, Academic Press, Inc. New York, 1992. Briefly, an antibody (or antigen-binding fragment thereof) may be immobilized on a solid support material, which is then contacted with a sample comprising the polypeptide of interest (e.g., a PTP). Following separation from the remainder of the sample, the polypeptide is then released from the immobilized antibody.

Methods For Detecting PTP Expression

Certain embodiments of the present invention provide methods that employ antibodies raised against PTP for assay purposes. Certain assays involve using an antibody or other agent to detect the presence or absence of PTP, or proteolytic fragments thereof. Assays may generally be performed using any of a variety of samples obtained from a biological source, as provided herein.

To detect a PTP protein, the reagent is typically an antibody, as provided herein. There are a variety of assay formats known to those having ordinary skill in the art for using an antibody to detect a polypeptide in a sample. See, e.g., Harlow and Lane, Antibodies: A Laboratory Manual, Cold Spring Harbor Laboratory, 1988. For example, the assay may be performed in a Western blot format, wherein a protein preparation from the biological sample is resolved by gel electrophoresis, transferred to a suitable membrane and allowed to react with the antibody. The presence of the antibody on the membrane may then be detected using a suitable detection reagent, as described below. In certain embodiments of the present invention, this format may be preferred to determine, establish or confirm the specific identity of a PTP that is identified as being reversibly modified or reversibly oxidized in a cell.

In another embodiment, isolation of a PTP may involve the use of antibody immobilized on a solid support to bind to the target PTP and remove it from the remainder of the sample. The bound PTP may then be detected using a second antibody or reagent that contains a reporter group. Alternatively, a competitive assay may be utilized, in which a PTP polypeptide is labeled with a reporter group and allowed to bind to the immobilized antibody after incubation of the antibody with the sample. The extent to which components of the sample inhibit the binding of the labeled polypeptide to the antibody is indicative of the reactivity of the sample with the immobilized antibody, and as a result, indicative of the level of PTP in the sample.

The solid support may be any material known to those having ordinary skill in the art to which the antibody may be attached, such as a test well in a microtiter plate, a nitrocellulose filter or another suitable membrane. Alternatively, the support may be a bead or disc, such as glass, fiberglass, latex or a plastic such as polystyrene or polyvinylchloride. The antibody may be immobilized on the solid support using a variety of techniques known to those in the art, which are amply described in the patent and scientific literature.

In certain embodiments, the assay for detection of PTP in a sample is a two-antibody sandwich assay. This assay may be performed by first contacting an antibody that has been immobilized on a solid support, commonly the well of a microtiter plate, with the biological sample, such that PTP within the sample is allowed to bind to the immobilized antibody (a 30 minute incubation time at room temperature is generally sufficient). Unbound sample is then removed from the immobilized PTP/antibody complexes and a second antibody (containing a reporter group such as an enzyme, dye, radionuclide, luminescent group, fluorescent group or biotin) capable of binding to a different site on the PTP is added. The amount of second antibody that remains bound to the solid support is then determined using a method appropriate for the specific reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products. Standards and standard additions may be used to determine the level of PTP in a sample, using well known techniques.

In a related aspect of the present invention, kits for detecting a reversibly modified PTP, and for determining PTP phosphatase activity, are provided. Such kits may be designed for detecting the level of PTP, or may detect phosphatase activity of PTP in a direct phosphatase assay or a coupled phosphatase assay. In general, the kits of the present invention comprise one or more containers enclosing elements, such as reagents or buffers, to be used in the assay. A kit for detecting the level of a PTP typically contains a reagent that specifically binds to the PTP protein; the reagent is typically an antibody. Such kits also contain a reporter group suitable for direct or indirect detection of the reagent (i.e., the reporter group may be covalently bound to the reagent or may be bound to a second molecule, such as Protein A, Protein G, immunoglobulin or lectin, which is itself capable of binding to the reagent). Suitable reporter groups include, but are not limited to, enzymes (e.g., horseradish peroxidase), substrates, cofactors, inhibitors, dyes, radionuclides, luminescent groups, fluorescent groups and biotin. Such reporter groups may be used to directly or indirectly detect binding of the reagent to a sample component using standard methods known to those having ordinary skill in the art.

Kits for detecting PTP activity typically comprise a PTP substrate in combination with a suitable buffer. PTP activity may be specifically detected by performing an immunoprecipitation step with a PTP-specific antibody prior to performing a phosphatase assay as described above. Other reagents for use in detecting dephosphorylation of substrate may also be provided.

Screening Assays for Agents

Where a PTP is identified that is a reversibly modified/oxidized component of a biological signaling pathway as provided herein, by using the methods of the present invention, it is further contemplated that in certain further embodiments the invention provides a screening assay for an agent that alters an inducible biological signaling pathway. According to such assays, a cell comprising the PTP (and hence the inducible pathway wherein the PTP is reversibly modified) is contacted with a stimulus that induces the pathway in the absence and presence of a candidate agent, under conditions permissive for induction of the pathway by the stimulus. PTPs are then isolated from the cell in the presence of a sulfhydryl-reactive agent that is capable of covalently (e.g., irreversibly) modifying a sulfhydryl group of the PTP active site invariant cysteine where, as described herein, the signaling pathway component PTP that is reversibly modified (e.g., oxidized) is protected from inactivation by such sulfhydryl agent, and PTP catalytic activity is determined by any of a variety of established methods, as also provided herein, after the reversibly modified PTP is reactivated by reversal of the modification (e.g., under reducing conditions). Decreased substrate dephosphorylation when the pathway is induced in the presence of the candidate agent, relative to the level of dephosphorylation when induction transpires in the absence of the candidate agent, indicates that the agent is an inhibitor or antagonist (e.g., results in PTP catalytic activity in the cell that is decreased in a statistically significant manner) of the reversibly modified PTP. Conversely, increased substrate dephosphorylation when the pathway is induced in the presence of the candidate agent, relative to the level of dephosphorylation when induction transpires in the absence of the candidate agent, indicates that the agent is a potentiator or agonist (i.e., an activity enhancer) of the reversibly modified PTP (e.g., results in PTP catalytic activity in the cell that is increased in a statistically significant manner). The assays of this embodiment of the invention therefore provide a method for identifying an agent that alters an inducible biological signaling pathway, which agent will be useful where specific manipulation of or intervention in a particular stimulus-inducible pathway may be desirable.

Candidate agents for use in a method for identifying an agent that alters (e.g., increases or decreases in a statistically significant manner at least one phenotype associated with pathway induction) an inducible biological signaling pathway according to the present invention may be provided as “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. For example, members of a library of test compounds can be administered to a plurality of samples, each containing at least one biological sample comprising a cell that comprises a PTP which has been identified as a reversibly modified (e.g., oxidized) component of an inducible biological signaling pathway as provided herein, and then assayed for their ability to enhance or inhibit dephosphorylation of a PTP substrate by the PTP. Compounds so identified as capable of influencing PTP function (e.g., phosphotyrosine and/or phosphoserine/threonine dephosphorylation) are valuable for therapeutic and/or diagnostic purposes, since they permit treatment and/or detection of diseases associated with PTP activity. Such compounds are also valuable in research directed to molecular signaling mechanisms that involve PTP, and to refinements in the discovery and development of future PTP compounds exhibiting greater specificity.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666, which are hereby incorporated by reference in their entireties) or other compositions that may include small molecules as provided herein (see e.g., PCT/US94/08542, EP 0774464, U.S. Pat. Nos. 5,798,035, 5,789,172, 5,751,629, which are hereby incorporated by reference in their entireties). Those having ordinary skill in the art will appreciate that a diverse assortment of such libraries may be prepared according to established procedures, and tested using PTP according to the present disclosure.

Therapeutic Methods

One or more agents capable of altering an inducible biological signaling pathway and identified according to the above described methods may also be used to modulate (e.g., inhibit or potentiate) PTP activity in a patient. As used herein, a “patient” may be any mammal, including a human, and may be afflicted with a condition associated with PTP activity or may be free of detectable disease. Accordingly, the treatment may be of an existing disease or may be prophylactic. Conditions associated with PTP activity include any disorder associated with cell proliferation, including cancer, graft-versus-host disease (GVHD), autoimmune diseases, allergy or other conditions in which immunosuppression may be involved, metabolic diseases, abnormal cell growth or proliferation and cell cycle abnormalities.

For administration to a patient, one or more modulating agents are generally formulated as a pharmaceutical composition. A pharmaceutical composition may be a sterile aqueous or non-aqueous solution, suspension or emulsion, which additionally comprises a physiologically acceptable carrier (i.e., a non-toxic material that does not interfere with the activity of the active ingredient). Such compositions may be in the form of a solid, liquid or gas (aerosol). Alternatively, compositions of the present invention may be formulated as a lyophilizate or compounds may be encapsulated within liposomes using well known technology. Pharmaceutical compositions within the scope of the present invention may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable carrier known to those of ordinary skill in the art may be employed in the pharmaceutical compositions of the present invention. Carriers for therapeutic use are well known, and are described, for example, in Remingtons Pharmaceutical Sciences, Mack Publishing Co. (A. R. Gennaro ed. 1985). In general, the type of carrier is selected based on the mode of administration. Pharmaceutical compositions may be formulated for any appropriate manner of administration, including, for example, topical, oral, nasal, intrathecal, rectal, vaginal, sublingual or parenteral administration, including subcutaneous, intravenous, intramuscular, intrastemal, intracavernous, intrameatal or intraurethral injection or infusion. For parenteral administration, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid (e.g., an elixir, syrup, solution, emulsion or suspension). A liquid pharmaceutical composition may include, for example, one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

The compositions described herein may be formulated for sustained release (i.e., a formulation such as a capsule or sponge that effects a slow release of compound following administration). Such compositions may generally be prepared using well known technology and administered by, for example, oral, rectal or subcutaneous implantation, or by implantation at the desired target site. Sustained-release formulations may contain an agent dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Within a pharmaceutical composition, a PTP modulating agent may be linked to any of a variety of compounds. For example, such an agent may be linked to a targeting moiety (e.g., a monoclonal or polyclonal antibody, a protein or a liposome) that facilitates the delivery of the agent to the target site. As used herein, a “targeting moiety” may be any substance (such as a compound or cell) that, when linked to an agent enhances the transport of the agent to a target cell or tissue, thereby increasing the local concentration of the agent. Targeting moieties include antibodies or fragments thereof, receptors, ligands and other molecules that bind to cells of, or in the vicinity of, the target tissue. An antibody targeting agent may be an intact (whole) molecule, a fragment thereof, or a functional equivalent thereof. Examples of antibody fragments are F(ab′)₂, −Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Targeting moieties may be selected based on the cell(s) or tissue(s) toward which the agent is expected to exert a therapeutic benefit.

Pharmaceutical compositions may be administered in a manner appropriate to the disease to be treated (or prevented). An appropriate dosage and a suitable duration and frequency of administration will be determined by such factors as the condition of the patient, the type and severity of the patient's disease, the particular form of the active ingredient and the method of administration. In general, an appropriate dosage and treatment regimen provides the agent(s) in an amount sufficient to provide therapeutic and/or prophylactic benefit (e.g., an improved clinical outcome, such as more frequent complete or partial remissions, or longer disease-free and/or overall survival). For prophylactic use, a dose should be sufficient to prevent, delay the onset of or diminish the severity of a disease associated with cell proliferation.

Optimal dosages may generally be determined using experimental models and/or clinical trials. In general, the amount of polypeptide present in a dose, or produced in situ by DNA present in a dose, ranges from about 0.01 μg to about 100 μg per kg of host, typically from about 0.1 μg to about 10 μg. The use of the minimum dosage that is sufficient to provide effective therapy is usually preferred. Patients may generally be monitored for therapeutic or prophylactic effectiveness using assays suitable for the condition being treated or prevented, which will be familiar to those having ordinary skill in the art. Suitable dose sizes will vary with the size of the patient, but will typically range from about 10 mL to about 500 mL for 10-60 kg animal.

The following Examples are offered for the purpose of illustrating the present invention and are not to be construed to limit the scope of this invention.

### EXAMPLES

### Example 1

### Reversible Inactivation of PTPs in Rat-1 Cells by Hydrogen Peroxide

Cell culture, transient transfection, immunoprecipitation and immunoblotting: Rat-1 fibroblasts (American Type Culture Collection, Manassas, Va.) were routinely maintained in DMEM supplemented with 10% FBS, 1% glutamine, 100 U/ml penicillin and 100 μg/ml streptomycin (all reagents Sigma, St. Louis, Mo., unless otherwise noted). For stimulation with H₂O₂ and peptide growth factors, cells were plated in media containing 10% FBS for 48 hours, then serum-starved for 16 hours before treatment. For transient transfection, Rat-1 cells were plated in DMEM medium supplemented with 10% FBS, for 16 hours. The culture medium was replaced by OptiMEM™ (Invitrogen Life Technologies, Inc. Gaithersburg, Md.) without serum, then plasmid (5 μg/dish) was introduced into cells by LipofectAMINE™ and PLUS™ reagents (Life Technologies), according to the manufacture's recommendations. The transfection efficiency was routinely 40%.

For immunoprecipitation, cells were rinsed with ice-cold PBS, then lysed in ice-cold 20 mM Hepes (pH 7.5), 1% NP-40, 150 mM NaCl, 10% glycerol, 200 μM Na₃VO₅ and protease inhibitors (25 μg/ml of aprotinin and leupeptin). Antibodies having the indicated specificities were purchased from the following suppliers: SHP-1 (C-19), SHP-2 (C-18) and P13K (Z-8), Santa Cruz Biotecnology, Santa Cruz, Calif.; phospho-MAPK and MAPK, Cell Signaling, Inc. (Beverly, Mass.); GAP, BD Transduction Laboratories (Lexington, Ky.); and pTyr mAb PT66, Sigma, St. Louis, Mo. The anti-pTyr antibody G104 was described previously (Garton et al., 1997 Oncogene 15, 877-885). Anti-PDGFRββ antibody (Ab-X) was a gift from Dr. Daniel DiMaio at Yale University (Irusta and DiMaio, 1998 EMBO J. 17, 6912-6923). Anti-human G-CSF receptor (G-CSFR) antibody was provided by Dr. Toshio Hirano at Osaka University, Japan (Fukada et al., 1996 Immunity 5, 449-460). Lysate (400 μg) was incubated with 5 μg of antibody conjugated to protein A/G-Sepharose (Amersham Pharmacia, Arlington Heights, Ill.) for 2 hours at 4° C. For immunoblotting, aliquots of total lysates (30 μg per sample) or immunoprecipitates were subjected to SDS-PAGE and transferred to nitrocellulose filters, which were incubated with appropriate primary and secondary antibodies and the specific signals were visualized by the ECL detection system (Amersham Pharmacia).

To determine whether ROS stimulated intracellular tyrosine phosphorylation through the oxidation and inhibition of cellular PTPs, a modified in-gel PTP activity assay was devised, as follows: As substrate, poly (4:1) Glu-Tyr (Sigma) was labeled with [γ-³²P]-ATP using the GST-FER fusion PTK, as described previously (Shen et al., 1998 J. Biol. Chem. 273:6474-81). The labeled substrates were used within three weeks to limit the variation of its specific activity from experiment to experiment. The lysis buffer (25 mM CH₃COONa, 1% NP-40, 150 mM NaCl, 10% glycerol, pH 5.5) was degassed at 4° C. for overnight, before catalase and superoxide dismutase (both 100 μg/ml), protease inhibitors and 10 mM iodoacetic acid (IAA) were added. Following stimulation, cells were lysed under anaerobic conditions in an argon chamber. Lysates (25 μg) were processed as described herein and an “in-gel” phosphatase assay (Burridge and Nelson, 1995) was conducted using SDS-PAGE gels containing a radioactively-labeled substrate (1.5×10⁶ cpm/20 ml gel solution, approximately 2 μM p-Tyr).

Cells were triggered with the appropriate stimulus and harvested under anaerobic conditions in lysis buffer containing IAA. Those PTPs that had not encountered ROS in the cell became irreversibly inactivated by alkylation of their active site Cys with IAA. However, in contrast, any PTPs in which the active site Cys had been oxidized in response to the stimulus were resistant to alkylation. For the “in-gel” phosphatase assay, a 10% SDS-PAGE gel was cast containing a radioactively-labeled substrate. An aliquot of cell lysate was subjected to SDS-PAGE and proteins in the gel were sequentially denatured, then renatured in the presence of reducing reagents. Under these conditions, the activity of the PTPs in which the active site Cys had been subjected to stimulus-dependent oxidation to sulfenic acid was recovered, whereas those that were not oxidized in response to the initial stimulus, and were irreversibly alkylated in the lysis step, remained inactive. The reaction was then terminated by fixing, staining and destaining the gel. Finally the gel was dried and exposed to film. The presence of a PTP was visualized by substrate dephosphorylation, as the appearance of a clear, white area on the black background of labeled substrate. As shown in FIG. 1, the PTPs that exhibited catalytic phosphatase activity in this assay would be those originally protected from post-lysis alkylation by a stimulus-dependent modification at the active site Cys, which was reversed by DTT, consistent with oxidation of the Cys to sulfenic acid.

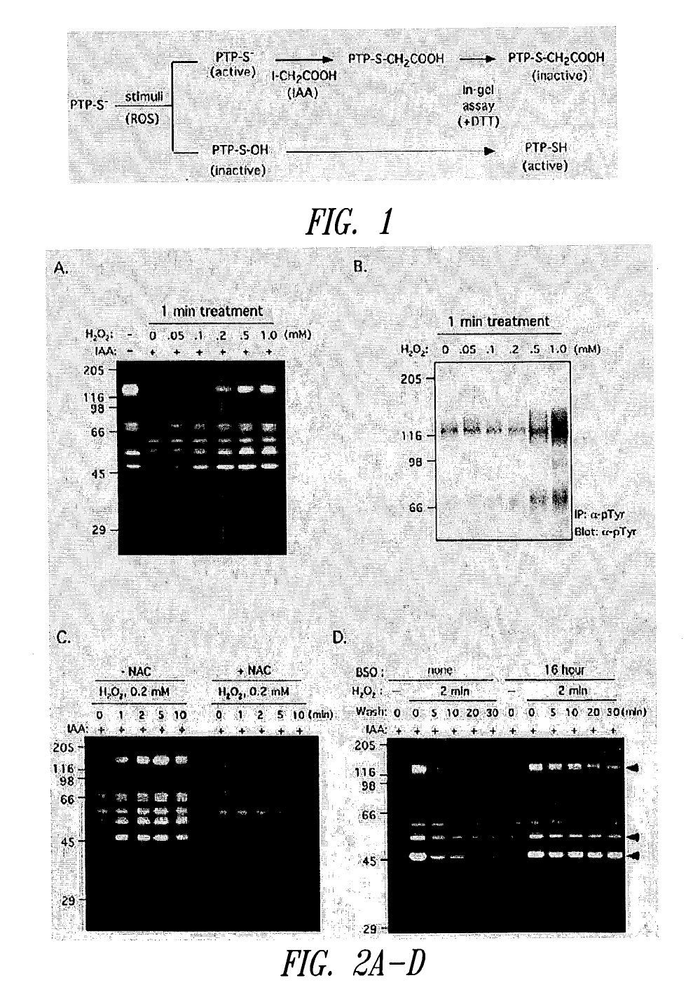

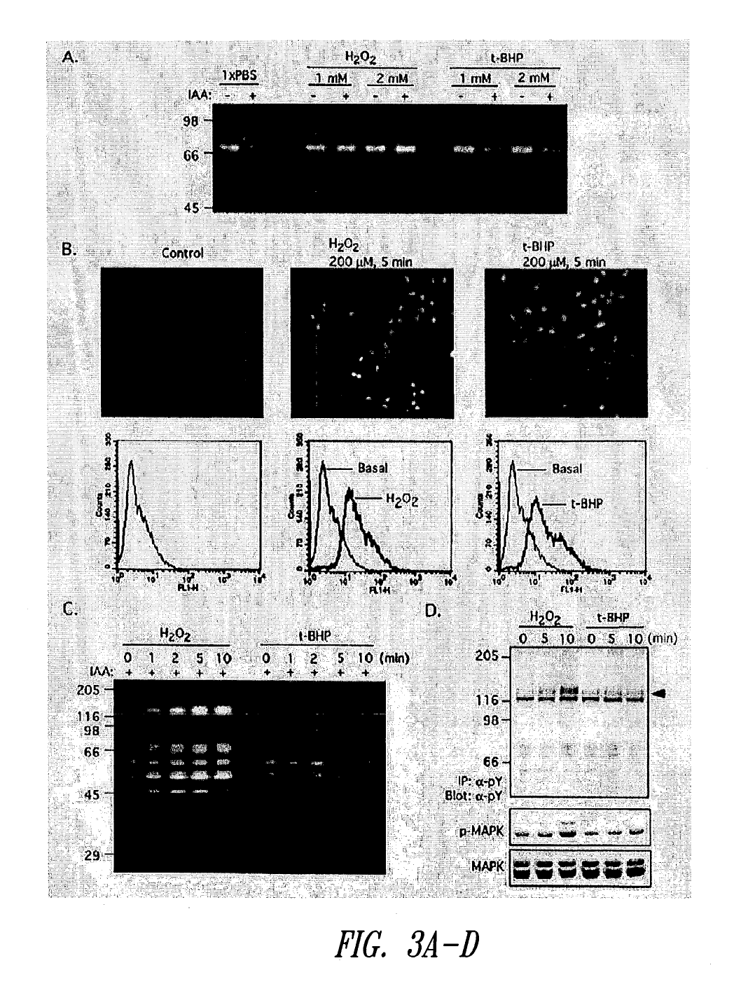

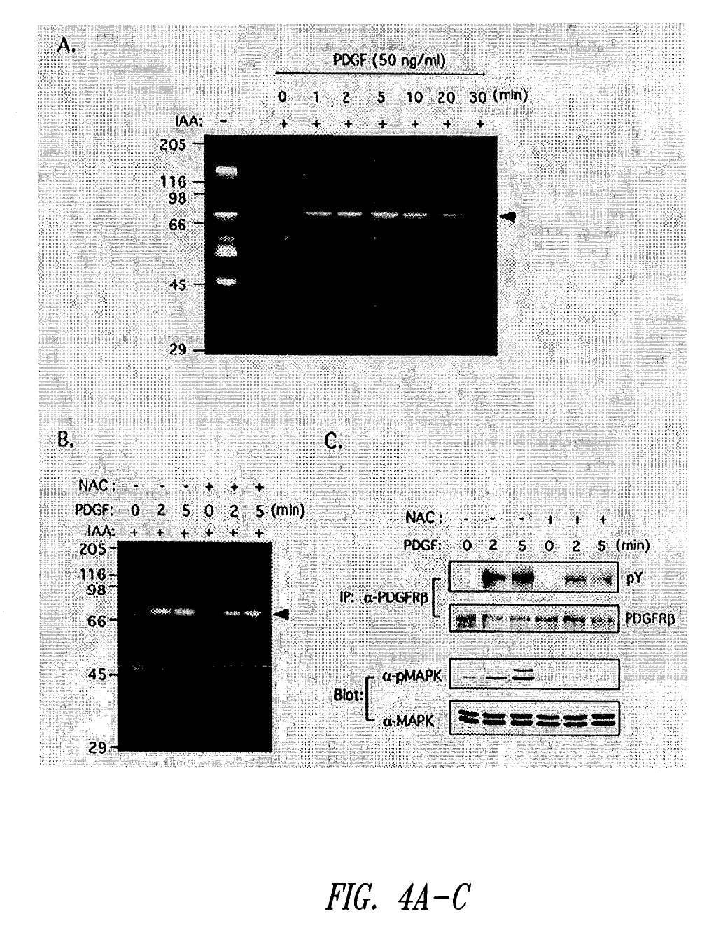

The data shown in FIG. 2A illustrate that iodoacetic acid (IAA) in the lysis buffer effectively inactivated PTPs in a lysate of Rat-1 cells (lane 2, compared to lane 1), via irreversible alkylation of the invariant, active site Cys residue of these enzymes (Zhang and Dixon, 1993 Biochemistry 32:9340-45). FIG. 2A shows the results when serum-deprived Rat-1 cells were exposed to various concentrations of H₂O₂ for 1 min, harvested and lysed in the absence (lane 1) or presence (lanes 2-7) of 10 mM IAA. Aliquots of lysate were subjected to the in-gel PTP assay. When H₂O₂ was added to the culture media, it gained rapid access to the intracellular environment and within 1 minute the active site Cys residue of various PTPs was oxidized, thereby protecting them from alkylation by IAA (lanes 3-7, FIG. 2A). Furthermore, 200 μM H₂O₂ was sufficient to oxidize all of the PTPs detectable in this assay format, but more extensive oxidation occurred at higher concentrations of H₂O₂ (FIG. 2A).

FIG. 2B shows results obtained when tyrosine phosphorylated proteins were immunoprecipitated from lysates of H₂O₂-treated cells with Ab PT-66, then immunoblotted with anti-pTyr Ab (G104). The tyrosine phosphorylation of proteins of 120 kDa and 70 kDa was induced in a dose-dependent fashion coincident with exposure of cells to H₂O₂ (FIG. 2B), suggesting a link between oxidation/inhibition of PTPs and enhanced tyrosine phosphorylation in Rat-1 cells. This stimulation also triggered the phosphorylation of ERK MAP kinases (MAPKs). N-acetyl cysteine (NAC), a widely used ROS scavenger, blocked PTP oxidation and inactivation induced by 200 μM H₂O₂, thus confirming that the effects on PTP activity shown in the in-gel assay were due to H₂O₂-induced intracellular oxidation (FIG. 2C). FIG. 2C depicts the results obtained when cells were preincubated in the absence or presence of 30 mM NAC for 40 minutes and excess NAC removed by two washes with fresh culture medium, after which the Rat-1 cells were exposed to 200 μM H₂O₂ and lysed in the presence of 10 mM IAA at the indicated times. Lysates were subjected to the in-gel PTP assay.

In addition, depletion of the cellular pool of glutathione (GSH) by exposure of the cells to L-buthionine-SR-sulfoximine (BSO), a specific inhibitor of γ-glutamylcysteine synthetase, markedly attenuated the recovery of PTP activity following removal of an H₂O₂ stimulus (FIG. 2D). To obtain the data presented in FIG. 2D, Rat-1 cells were serum-starved in the absence or presence of 2.5 mM BSO for 16 h. H₂O₂ (200 μM) was added for 2 minutes, then removed by washing the cells with fresh culture media. Incubation was then continued until harvesting in lysis buffer containing 10 mM IAA at the times indicated. Oxidized PTPs were visualized by the in-gel phosphatase activity assay. Arrows indicate PTPs for which reduction/reactivation exhibited dependence on intracellular GSH. Stimulation with H₂O₂ led to oxidation of several PTPs (lane 2), which were quickly reduced once H₂O₂ was removed (FIG. 2D, lanes 3-6). Recovery was essentially complete within 10-20 minutes of removal of H₂O₂. However, when the same analysis was performed on Rat-1 cells that had been subjected to pretreatment with BSO, oxidation persisted even 30 minutes after removal of H₂O₂ (FIG. 2D lanes 8-12). Surprisingly, these observations provide the first demonstration that multiple PTPs may be oxidized and inactivated by ROS in a cellular environment.

### Example 2

### H₂O₂-Induced Mitogenic Signaling Associated with PTP Inactivation

In order to explore the importance of oxidation and inhibition of PTP function for ROS-induced mitogenesis, the effects of H₂O₂ and the synthetic ROS t-butyl hydroperoxide (t-BHP) were tested. Initially, the susceptibility of an activated mutant form of SHP-2 (E76A) to alkylation by IAA was compared following treatment with either H₂O₂ or t-BHP. Using the modified in-gel PTP assay described in Example 1, SHP-2, which had been pre-treated with PBS, was inactivated by IAA (lane 2, compared to lane 1, FIG. 3A), whereas oxidation with H₂O₂ protected SHP-2 from alkylation (FIG. 3A). Briefly, purified SHP-2 (E76A mutant) was incubated with PBS, H₂O₂ or t-BHP at 37° C. for 5 mins. Aliquots were then incubated at room temp for a further 5 minutes, either in the absence (−IAA) or presence (+IAA) of 4 mM IAA, and subjected to the in-gel PTP activity assay (1 ng SHP-2/lane). Even at 2 mM H₂O₂, SHP-2 was not irreversibly oxidized since its activity was recovered in the in-gel assay (FIG. 3A). In contrast, t-BHP was unable to oxidize and inactivate SHP-2 in vitro and thus did not protect the invariant Cys residue of SHP-2 from alkylation (FIG. 3A).

The effects of H₂O₂ and t-BHP on inactivation of PTPs and activation of MAPK signaling pathways were next compared in a cellular context. Intracellular ROS were measured using 2′,7′-dichlorofluorescein diacetate (H₂DCFDA) and 5-(and-6)-chloromethyl-2′,7′-dichlorodihydrofluorescein diacetate (CM-H₂DCFDA) (all fluorescent ROS indicators from Molecular Probes, Eugene, Oreg.) either by fluorescence microscopy, using a Zeiss Axiovert 405M inverted microscope equipped with a fluorescence attachment and digital camera, or by cell sorting, using a FACSCalibur System (Coulter Instruments, Hialeah, Fla.), according to the manufacturer's recommendations. Rat-1 cells were pre-loaded with 20 μM H₂DCFDA in the dark for 20 mins, then exposed to H₂O₂ and t-BHP (both 200 μM) for 5 mins. Images of ROS-induced DCF fluorescence are shown at magnification 400× (FIG. 3B upper panel). Cells (1×10⁵) that underwent the same treatment as above were harvested and resuspended in Hanks' solution, then immediately subjected to flow cytometric analysis to measure ROS-induced DCF fluorescence. The basal peak indicates background fluorescence, whereas the rightward shifted peak indicates ROS-induced DCF fluorescence (FIG. 3B, lower panels). Initially, fluorescence microscopy of Rat-1 cells, preloaded with H₂DCFDA, showed that treatment with either H₂O₂ or t-BHP led to rapid oxidation and the appearance of the fluorescent derivative, DCF (upper panels, FIG. 3B). Furthermore, upon flow cytometric analysis no quantitative difference was observed between the H₂O₂- and t-BHP-induced shift of fluorescence (FIG. 3B, lower panels). However, when the ability to oxidize PTPs in the cells was examined, reproducible inactivation of PTPs was detected in response to H₂O₂ but not in response to t-BHP (FIG. 3C). Cells were exposed to H₂O₂ and t-BHP (each at 200 μM) for the indicated times, lysed in the presence of 10 mM IAA and oxidized PTPs were visualized in the in-gel PTP activity assay.

H₂O₂ and t-BHP were next compared for their effects on tyrosine phosphorylation of cellular proteins, and on activation of MAPKs. As shown in FIG. 3D, after exposure to H₂O₂ and t-BHP (each at 200 μM), lysates were prepared and pTyr proteins were immunoprecipitated with Ab PT-66, then immunoblotted with anti-pTyr Ab G104 (FIG. 3D, upper panel). An aliquot of lysate from each treatment group was immunoblotted with anti-phospho-MAPK Ab and subsequently with anti-MAPK Ab (FIG. 3D, lower panel). As shown in FIG. 3D, the inactivation of PTPs by H₂O₂ was associated with enhanced tyrosine phosphorylation and mitogenic signaling. In contrast, t-BHP elicited less pronounced effects on tyrosine phosphorylation and was unable to activate MAPKs (FIG. 3D), presumably due to its inability to oxidize and inactivate the PTPs. Without wishing to be bound by theory, these results are consistent with a role of PTP inactivation in the mitogenic effects of ROS.

### Example 3

### Oxidation of a 70 kDa PTP Associated with PDGF-Induced Mitogenic Signaling in Rat-1 Cells and Identification of the 70 kDa PTP as SHP-2

As described above, treatment of Rat-1 cells with H₂O₂ led to inactivation of multiple PTPs (FIGS. 2-3). This Example describes studies to determine whether the production of ROS in response to physiological stimuli also resulted in oxidation and inactivation of members of the PTP family, and whether there was specificity in the response. Initially examined were the effects of PDGF, a peptide growth factor, which has been shown to produce ROS in various cell types (Bae et al., 2000; Sundaresan et al., 1995). Preliminary experiments showed that treatment of Rat-1 cells with PDGF resulted in a rapid increase in the tyrosine phosphorylation of cellular proteins and the enhanced phosphorylation of MAPKs. Lysates of PDGF-stimulated Rat-1 cells were then analyzed using the modified in-gel PTP activity assay described above. The results, as shown in FIG. 4, demonstrated that PDGF stimulation induced a rapid and transient oxidation of a PTP having an apparent molecular mass of ˜70 kDa. Serum-starved Rat-1 cells were exposed to 50 ng/ml PDGF-BB for the times indicated (FIG. 4A). Lysates were prepared in the presence of 10 mM IAA and subjected to the in-gel PTP assay. The arrow indicates a 70 k PTP that was transiently oxidized following stimulation of Rat-1 cells with PDGF. The result shown is representative of four independent experiments. Oxidation of this 70 kDa PTP was reversible, reaching a maximum at 5 minutes, followed by marked reduction, almost to basal levels, within 20 minutes of PDGF treatment (FIG. 4A).

A possible role of oxidation/inactivation of the 70 k PTP in regulating PDGFR-mediated signaling was next investigated by testing the effects of the antioxidant NAC. Cells were incubated for 40 minutes in the presence or absence of 30 mM NAC prior to PDGF stimulation. Excess NAC was removed prior to addition of PDGF (50 ng/ml). PDGF-induced oxidation of the 70 k PTP, which was impaired in the presence of NAC (FIG. 4B, arrow), was visualized by the modified in-gel PTP assay. Then the modified in-gel PTP assay was used to examine the effects of the growth factor on the activity of the 70 k PTP. When the levels of PDGF-induced ROS were reduced by pretreatment with NAC, oxidation of the 70 k PTP was markedly attenuated (FIG. 4B). Furthermore, the ligand-induced tyrosine phosphorylation of the PDGFR was greatly diminished, and the activation of MAPKs was completely eliminated, in NAC-treated cells (FIG. 4C). Cells were treated with NAC and PDGF as described above. PDGFR was immunoprecipitated from lysates with Ab-X and immunoblotted with anti-pTyr Ab G104. The same filter was subsequently re-probed with Ab-X (FIG. 4C, upper panels). Aliquots of cell lysate from each treatment were immunoblotted with anti-phosho-MAPK Ab and re-probed with anti-MAPK Ab (FIG. 4C, lower panels). These data suggest that the rapid, transient inactivation of 70 k PTP may be important for concomitant PDGFR-mediated phosphorylation and mitogenic signaling.

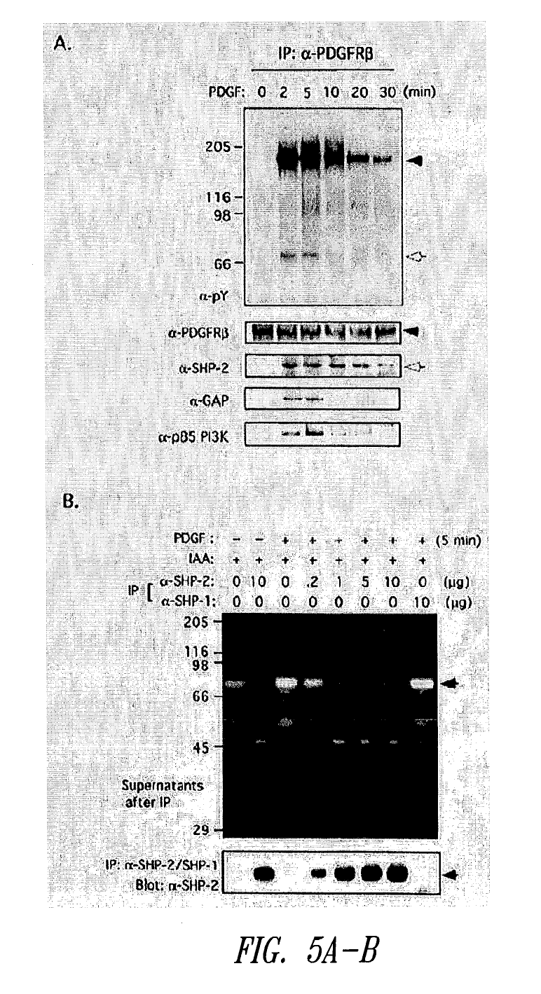

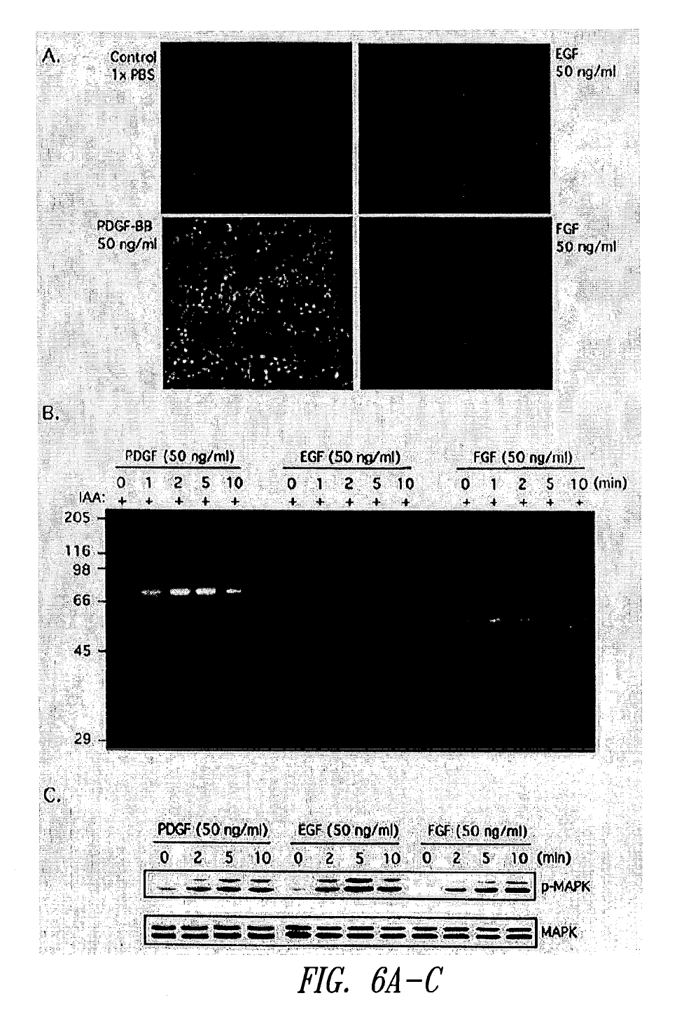

In attempting to identify the 70 k PTP that was oxidized following PDGF stimulation, attention was drawn to the SH2 domain-containing PTP, SHP-2. This PTP has been shown to be associated with tyrosine phosphorylated PDGFR (Lechleider et al., 1993 J Biol. Chem. 268:21478-81). In addition, the apparent molecular weight of SHP-2 on SDS-PAGE is similar to that of the PDGF-responsive 70 k PTP detected in FIG. 4. Initially, it was confirmed that SHP-2 could be recruited by the ligand-activated PDGFR in Rat-1 cells. Serum-starved Rat-1 cells were exposed to PDGF (50 ng/ml) for the indicated times (FIG. 5A). The PDGFR and associated proteins were immunoprecipitated with antibody Ab-X, and pTyr proteins visualized by immunoblotting with anti-pTyr Ab G104 (FIG. 5A, upper panel). The same filter was re-probed with anti-PDGFR, anti-SHP-2, anti-GAP and anti-p85 P13K Abs. The positions of PDGFR (FIG. 5A, solid arrow) and SHP-2 (FIG. 5A, open arrow) are indicated. As shown in FIG. 5A (upper panel), a tyrosine phosphorylated protein of 70 kDa by SDS-PAGE associated rapidly with the PDGFR in response to ligand activation. Furthermore, immunoblotting was used to show that SHP-2 comigrated with this 70 k phosphoprotein (FIG. 5A, lower panels). The complex between PDGFR and SHP-2 persisted for up to 20 minutes after stimulation, then the level of association decreased (FIG. 5A, lower panels).

To test whether SHP-2 was the 70 k PTP that was oxidized following PDGF stimulation, SHP-2 protein was immunodepleted from cell lysates with increasing amounts of anti-SHP-2 antibody, and the supernatants were subjected to the modified in-gel PTP assay. Rat-1 cells, either untreated (−) or stimulated with 50 ng/ml PDGF (+), were harvested in lysis buffer containing 10 mM IAA. Lysates were incubated with antibody to either SHP-2 or SHP-1 and subjected to an in-gel PTP assay (FIG. 5B, upper panel). The arrow denotes the position of the 70 k PTP that was inactivated in response to PDGF and immunodepleted from cell lysates with antibodies to SHP-2. The lower panel of FIG. 5B illustrates an immunoblot to show the immunodepletion of SHP-2. As shown in FIG. 5B, anti-SHP-2 antibody depleted the 70 k PTP from Rat-1 cell lysates, whereas an anti-SHP-1 antibody control did not. These data identify SHP-2 as a PTP that was rapidly oxidized and inactivated following PDGF stimulation.

Association of other SH2 domain-containing proteins with activated PDGFR was also examined. It has been shown that SHP-2 dephosphorylates the PDGFR on the autophosphorylation sites that function as binding sites for GTPase-activating protein (GAP) and phosphatidylinositol 3 kinase (P13K) (Klinghoffer and Kazlauskas, 1995 J. Biol. Chem. 270:22208-17) (see also Kazlauskas et al., 1992 Mol. Cell Biol. 12:2534-44). However, both GAP and the p85 subunit of P13K were recruited by PDGFR rapidly after ligand stimulation, even though SHP-2 was associated with the receptor at this time (FIG. 5A). These results suggest that oxidation and inactivation of SHP-2 in response to PDGF may be important for permitting recruitment of GAP and P13K by the activated PDGFR. Interestingly, GAP and P13K dissociated from the receptor by 10 minutes after PDGF stimulation (FIG. 5A), coincident with dephosphorylation of PDGFRβ (FIG. 5A) and reactivation of SHP-2 (FIG. 4A).

### Example 4

### Specificity of ROS Production and SHP-2 Oxidation and Inactivation in Response to Growth Factor Stimulation

SHP-2 was one of the first PTPs to be recognized as capable of both negative signaling (by antagonizing PTK function) and positive signaling following a PTP-mediated dephosphorylation event, playing such a role, for example, in the context of EGF and FGF receptor signaling (Bennett et al., 1996 Mol. Biol. Cell 16:1189-1202; Saxton et al., 1997 EMBO J. 16:2352-64). The data described above, showing oxidation and inhibition of SHP-2 in response to PDGF, appear to be indicative of a negative role in signaling. This example describes additional characterization of a PTP response to a stimulus that induces a biological signaling pathway.

Treatment of Rat-1 cells with PDGF triggered production of intracellular ROS (FIG. 6A), concomitant with oxidation and inactivation of SHP-2 (FIG. 6B). In contrast, ROS production was not detected in response to either EGF or FGF (FIG. 6A). Rat-1 cells were incubated with 20 μM CM-H₂DCFDA in the dark for 20 mins, then exposed to peptide growth factors (50 ng/ml) for an additional 10 mins. Images of ROS-induced DCF fluorescence are shown at 50× magnification. (FIG. 6A) The data are representative of 4 independent experiments. In FIG. 6B, Rat-1 cells were exposed to peptide growth factors for the indicated times, lysed in the presence of 10 mM IAA, and oxidized PTPs were visualized by the in-gel PTP assay. In this assay, too, oxidation and inhibition of SHP-2 was observed following PDGF stimulation of the cells but not following exposure of these cells to EGF or FGF. EGF, FGF and PDGF all activated MAPK to a similar extent in Rat-1 cells (FIG. 6C). Aliquots of cell lysate from each treatment group were immunoblotted with anti-phospho-MAPK Ab and re-probed with anti-MAPK Ab. These results indicate that, of the stimuli examined in Rat-1 cells, transient oxidation and inactivation of SHP-2 is a specific response to PDGF, consistent with differences in the function of SHP-2 in these distinct growth factor signaling pathways.

The next set of experiments demonstrated that the PDGFR-associated pool of SHP-2 was susceptible to oxidation and inactivation. Recent studies have suggested that a Rac1-associated, plasma membrane-bound NADPH oxidase is responsible for PDGF-induced generation of ROS in non-phagocytic cells (Bae et al., 2000). In light of the short half-life of such ROS, it is possible that their influence on PTPs may be spatially restricted to the subcellular regions proximal to their production.

In preliminary studies only ˜10% of the total population of SHP-2 was recruited into a complex with the PDGFR following ligand stimulation in Rat-1 cells. To examine whether this recruitment was required for oxidation and inactivation of SHP-2 in response to PDGF, mutant forms of the PDGFR were constructed that were deficient in their association with SHP-2. Chimeric cell surface signal transduction receptors were also constructed which consisted of the extracellular segment of human granulocyte colony stimulating factor (G-CSF) receptor and the transmembrane and cytoplasmic segments of human PDGFR. The ability of these mutant PDGFRs to induce oxidation of the PTP in response to ligand was then tested.

Full length cDNA encoding wild type (WT) and Y1009F mutant forms of human PDGFRβ was provided by Dr. Jonathan Cooper (Fred Hutchinson Cancer Center, Seattle, Wash.; (Kashishian and Cooper, 1993 Mol. Biol. Cell 4:49-57)). The cDNA encoding the extracellular segment of human G-CSFR was a gift from Dr. Shigekazu Nagata (Osaka University, Japan; (Fukada et al., 1996)). Chimeric receptors comprising the extracellular segment of G-CSFR fused to the transmembrane and intracellular (WT and Y1009F) segments of PDGFRβ were constructed in the pcDNA3.1A vector (Invitrogen) by standard PCR protocols then inserted into a pRK5 expression vector for transient transfection experiments. The integrity of the constructs was confirmed by sequencing. These chimeric receptors permitted examination of G-CSF-induced recruitment of SHP-2 to the chimeric receptors and signaling in Rat-1 cells, which do not express endogenous G-CSF receptor (G-CSFR), while avoiding activation of endogenous PDGFR. The autophosphorylation site at Y 1009 of human PDGFR has been shown to be the major docking site for the N-terminal SH2 domain of SHP-2 (Lechleider et al., 1993).

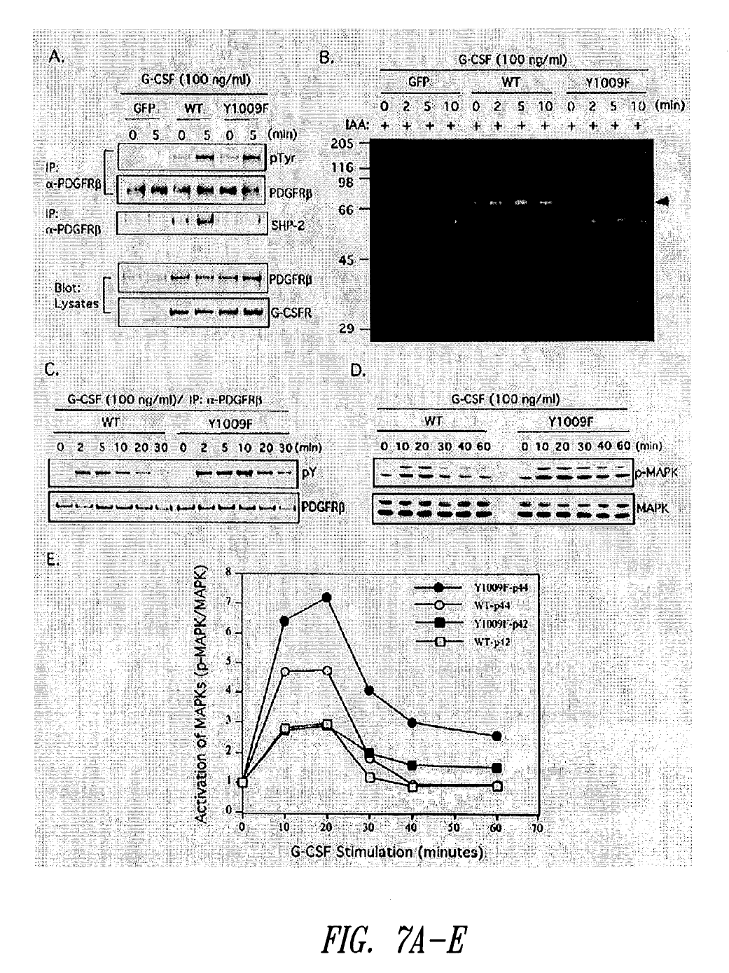

Expression constructs encoding chimeric receptors comprising either wild type (WT) or Y1009F forms of the PDGFR intracellular segment were transiently transfected into Rat-1 cells. Upon stimulation with G-CSF, both WT and Y1009F chimeric receptors were tyrosine phosphorylated (FIG. 7A). Although both receptors were activated following treatment with G-CSF, only the WT recruited SHP-2, which was recovered in immune-complexes precipitated with antibodies to the intracellular segment of the PDGFR (FIG. 7A). Using the modified in-gel PTP assay, WT chimeric receptors triggered rapid oxidation and inactivation of SHP-2 in response to G-CSF stimulation. Rat-1 cells were transiently transfected with plasmids expressing WT or Y1009F mutant G-CSFR/PDGFR chimeric receptor, or with a plasmid encoding Green Fluorescence Protein (GFP) as a control for expression. After exposure to 100 ng/ml G-CSF for 5 min, the chimeric receptors were immunoprecipiated from lysates with antibody Ab-X and immunoblotted with anti-pTyr Ab G104. (FIG. 7A) Immunoprecipitation of the receptors was verified by immunoblotting with Ab-X. The same filter was stripped and reprobed with anti-SHP-2 Ab. Expression of the chimeric receptors was verified by immunoblotting an aliquot of each lysate with Ab-X, which recognizes the intracellular segment of the PDGFR, and subsequently with anti-G-CSFR Ab, which recognizes the extracellular segment of chimeric receptors, as also shown in FIG. 7A.

Next, transfected Rat-1 cells were treated with G-CSF for the indicated times, lysed in the presence of 10 mM IAA and the lysates subjected to an in-gel PTP assay. (FIG. 7B) Activation of Y1009F mutant receptors did not induce oxidation of SHP-2 (FIG. 7B), suggesting according to non-limiting theory that recruitment of SHP-2 by activated, chimeric PDGFR was required for oxidation of the PTP by ROS generated in response to ligand. The arrow denotes the position of SHP-2.

Using the G-CSF:PDGF receptor chimeras, a time course of exposure to G-CSF illustrated that both WT and Y1009F, SHP-2 docking site mutant receptors were rapidly tyrosine phosphorylated following ligand stimulation. However, whereas tyrosine phosphorylation of the WT receptor was transient, the mutant receptor was maintained at a higher level of phosphorylation throughout the time course (FIG. 7C). The differences were particularly striking at the later time points, following 20 and 30 minutes of ligand stimulation. As shown in FIG. 7C, the WT and mutant chimeric receptors were immunoprecipitated at the indicated times and immunoblotted with anti-pTyr Ab (G104). The same filter was re-probed with anti-PDGFR Ab-X.

The phosphorylation status of MAPKs in the cell lysates was also investigated by immunoblotting analysis with antibodies specific for the phosphorylated and dephosphorylated forms of MAPK. Maximal phosphorylation of p42 and p44 ERKs following 20 minutes of stimulation (FIG. 7D). FIG. 7D shows the results obtained when aliquots of lysate from each treatment group were also subjected to immunoblotting with anti-phosho-MAPK Ab, and then re-probed with anti-MAPK Ab. However, both the extent and duration of ERK phosphorylation was higher in cells expressing the mutant receptor, which was deficient in binding of SHP-2, compared to those expressing the wild type receptor (FIGS. 7D & E). As shown in FIG. 7E, densitometric analysis of the gel image of FIG. 7D illustrates the ratio of phosphorylated (upper panel of 7D) over total (lower panel of 7D) MAPK. Without wishing to be bound by theory, these results suggest that recruitment of SHP-2 into the PDGF receptor-containing signaling complex is important for down-regulation of both receptor tyrosine phosphorylation and activation of MAPK, and that oxidation and inhibition of SHP-2 in the early phase of the response to PDGF is important for establishment of the signaling response.

### Example 5

### Prior Treatment of Cells with a PTP Active Site-Binding Agent Protects Against IAA-Mediated PTP Inactivation

An in-gel protection assay was developed to show that a small molecule PTP inhibitor could bind to the active site of the PTP and protect the active site cysteine from alkylation or from other irreversible modifications. An independently developed PTP inhibitor was shown to inhibit PTP catalytic activity and characterized by X-ray crystallography as a PTP active site-binding agent. This PTP inhibitor, referred to here as ASBA-1, was used to demonstrate that the PTP inhibitor could specifically bind to a PTP in an activated blood cell.

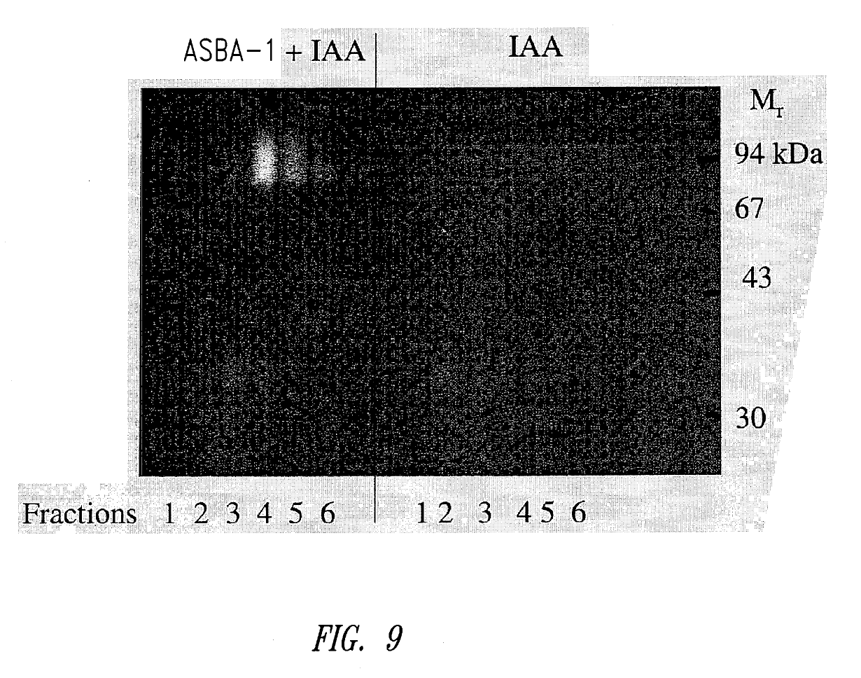

Peripheral blood mononuclear lymphocytes were purified from human blood. In 5 ml media (RPMI), 2×10⁷ cells were incubated in 50 μM ASBA-1 (PTP specific inhibitor) for 90 minutes and stimulated with phytohemagglutinin (PHA, 0.5 μL of 5.0 mg/ml stock) for 2, 10 or 30 min. Cells were pelleted, washed and lysed in buffer in the presence or absence of 50 mM iodoacetic acid (IAA) in extraction buffer (50 mM Tris, pH 7.5; 1 mM EDTA; 1 mM EGTA; 0.25% Triton X-100; 1 ug/mL pepstatin, aprotinin, and leupeptin; 1 mM benzamidine). Desalted proteins were separated on a 2 ml Source Q anion exchange column (Amersham Pharmacia Biotech) using a 0-1M NaCl gradient in 20 mM Tris, pH 7.5; 1 mM EDTA; 0.05% Triton X-100. Samples of each fraction were analyzed by the in-gel PTP assay (described above) and the results are shown in FIG. 9. At least two IAA-insensitive, PTP activity bands were observed from ASBA-1-treated cells, following autoradiography of the dried gel (FIG. 9, left panel). In samples from cells in which these proteins were not protected by ASBA-1 pretreatment, the PTPs were inactivated by IAA and PTP activity was not observed in the corresponding gel region using the in-gel PTP activity assay (FIG. 9, right panel). Therefore, and according to non-limiting theory, specific binding of ASBA-1 to the active sites of at least two PTPs in these cells prevented complete inactivation of the PTPs by IAA.

### Example 6

### Insulin Signaling Mediated by ROS Production

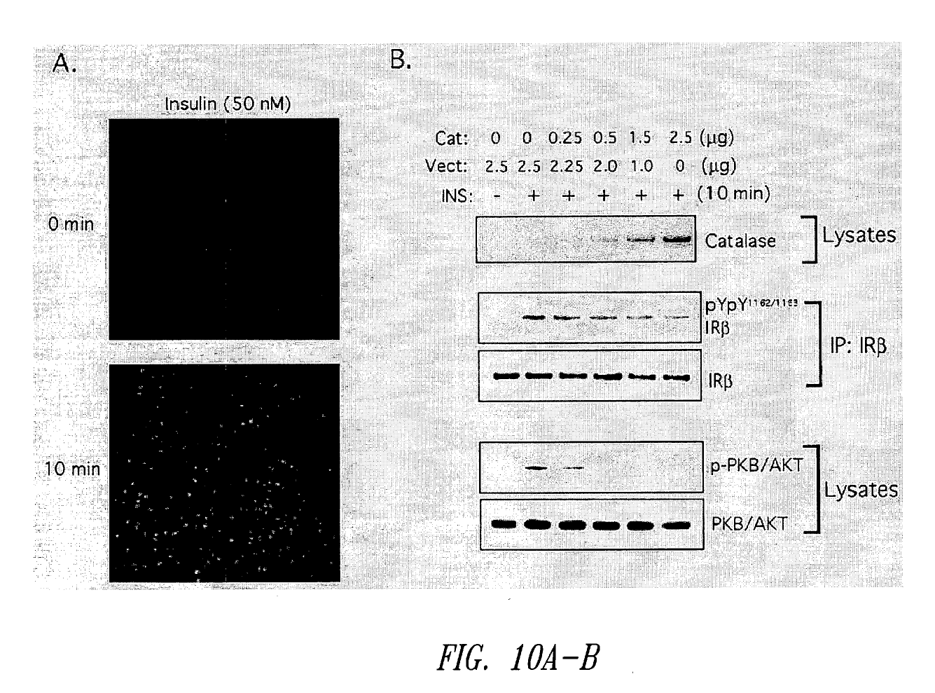

The role of intracellular production of ROS (e.g., H₂O₂) in insulin-mediated signal transduction was examined. Rat-1 fibroblasts were cultured and then serum starved for 16 hours as described in Example 1. The cells were preloaded with 5 μM CM-H₂DCFDA (Molecular Probes, Eugene, Oreg., Cat. No. D-399) in the dark for 15 min and then exposed to 50 nM insulin for 10 minutes. Images of ROS-induced DCF fluorescence were captured by fluorescence microscopy using a Zeiss Axiovert 405M inverted microscope equipped with a fluorescence attachment and digital camera (see Example 2), and are shown at 50× magnification in FIG. 10A.

Ectopic expression of catalase, which suppresses intracellular H₂O₂ production, impaired both tyrosine phosphorylation of the β-subunit of the insulin receptor (IR-β) and the phosphorylation of the downstream signaling molecule PKB/Akt in response to insulin stimulation. Rat-1 cells were transiently transfected as described in Example 1 with different quantities of plasmid encoding human catalase (a gift from Dr. Toren Finkle, NIH, Bethesda Md.) or with empty vector. Two days after transfection, cells were serum-deprived, then stimulated with 50 nM insulin (INS) for 10 min. The cells were lysed in 20 mM Hepes (pH 7.5), 1% NP-40, 150 mM NaCl, 10% glycerol, and 200 μM Na₃VO₄ containing 25 μg/ml each of aprotinin and leupeptin. Immunoblotting and immunoprecipitation were then performed essentially as described in Example 1. Catalase expression was verified by immunoblotting with an anti-catalase antibody (Calbiochem®, San Diego, Calif.) as shown in FIG. 10B (top panel). The IR-β subunit was immunoprecipitated from 400 μg of the cell lysate with antibody 29B4 (Santa Cruz). The lysate was separated by SDS-PAGE and then immunoblotted with anti-pYpY^{1162/1163} (Biosource International, Camarillo, Calif.) to examine the phosphorylation status of the receptor. The immunoblot was subsequently probed with anti-IR-β antibody clone C-19 (Santa Cruz) as a loading control (FIG. 10B, middle panel). An aliquot of lysate (30 μg) was subjected to immunoblotting with anti-phospho-PKB/AKT antibody (Cell Signaling). The same filter was then stripped and re-probed with anti-PKB/AKT antibody (Cell Signaling) as a loading control (FIG. 10B, bottom panel).

### Example 7

### Insulin Induces Transient Oxidation of PTP1B and TC45

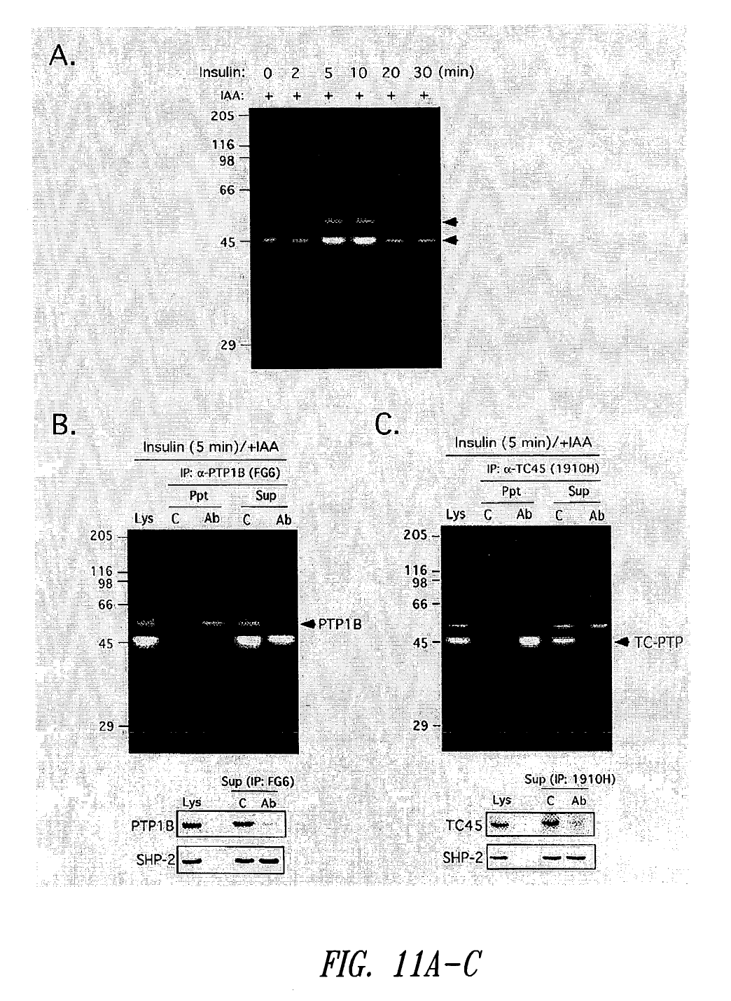

The effect of insulin-induced H₂O₂ production on PTP oxidation was examined using the modified in-gel PTP assay essentially as described in Example 1. Serum-starved Rat-1 cells were exposed to 50 nM insulin for 2, 5, 10, 20, and 30 minutes. Lysates were prepared under anaerobic conditions in the presence of 10 mM IAA and then subjected to in-gel PTP assays. The substrate incorporated into the SDS-PAGE gels for these assays was ³²P-labeled reduced, carboxamidomethylated and maleylated lysozyme (RCML) (1.5×10⁶ cpm/20 ml gel solution, ˜2 μM p-Tyr). FIG. 11A shows that a PTP having an approximate molecular weight of 50 kDa and a PTP with an approximate molecular weight of 45 kDa were transiently oxidized in response to insulin.

The oxidized 45 kDa and 50 kDa PTPs were identified as TC-45 and PTP1B, respectively, by immunodepletion and immunoblotting. Total cell lysates were prepared as described in Example 1. Lysate (400 μg) was incubated with normal IgG, anti-PTP1B antibody (FG6, LaMontagne et al., Mol. Cell. Biol. 18:2965-75 (1998)), or anti-TC45 antibody (1910H, Lorenzen et al., J. Cell. Biol. 131:631-43 (1995)) coupled to protein G-Sepharose ^{T} beads (Amersham Biosciences). After the immunoprecipitation step, the immune complexes and supernatants were collected and subjected to in-gel PTP assays. Immunodepletion of the 50 kDa PTP from the lysate with anti-PTP1B antibody is shown in FIG. 11B, and immunodepletion of the 45 kDa PTP with antibody specific for TC45 is shown in FIG. 11C. Cell lysate prior to immunodepletion is represented in the lane marked “Lys” in FIGS. 11B and 11C. Total cell lysate and supernatants were separated by SDS-PAGE, transferred to nitrocellulose membranes, and immunoblotted with either anti-PTP1B antibody (FIG. 11B) or anti-TC45 antibody (FIG. 11C). The immunoblots show that each PTP protein is depleted after immunoprecipitation with the specific antibody. The same immunoblots were subsequently reprobed with anti-SHP-2 antibody to illustrate that comparable amounts of polypeptide were loaded onto each gel.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for identifying a protein tyrosine phosphatase that is reversibly oxidized in a cell, comprising:
contacting a biological sample comprising a cell that comprises at least one protein tyrosine phosphatase with a stimulus under conditions and for a time sufficient to induce reversible oxidation of at least one protein tyrosine phosphatase in the cell;
isolating anaerobically the protein tyrosine phosphatase in the presence of a sulfhydryl-reactive agent that is capable of irreversibly modifying a sulfhydryl group of a protein tyrosine phosphatase active site invariant cysteine; and
determining under reducing conditions a level of dephosphorylation of a detectably labeled protein tyrosine phosphatase substrate by the protein tyrosine phosphatase, wherein detectable substrate dephosphorylation indicates that an active protein tyrosine phosphatase is present, and therefrom identifying a protein tyrosine phosphatase that is reversibly oxidized in a cell.

2. The method of claim 1 wherein the protein tyrosine phosphatase is selected from the group consisting of SHP-2, PTP1B, and TC45.

3. The method of claim 1 wherein the protein tyrosine phosphatase is selected from the group consisting of PTP1B, PTP-PEST, PTPγ, LAR, MKP-1, CRYPα, PTPcryp2, DEP-1, SAP1, PCPTP1, PTPSL, STEP, HePTP, PTPIA2, PTPNP, PTPNE6, PTPμ, PTPX1, PTPX10, SHP-1, SHP-2, PTPBEM1, PTPBEM2, PTPBYP, PTPesp, PTPoc, PTP-PEZ, PTP-MEG1, MEG2, LC-PTP, TC-PTP, TC45, CD45, LAR, cdc14, RPTP-α, RPTP-ε, RKPTP, LyPTP, PEP, BDP1, PTP20, PTPK1, PTPS31, PTPGMC, GLEPP1, OSTPTP, PTPtep, PTPRL10, PTP2E, PTPD1, PTPD2, PTP36, PTPBAS, PTPBL, BTPBA14, PTPTyp, HDPTP, PTPTD14, PTPα, PTPβ, PTPδ, PTPε, PTPκ, PTPλ, PTPμ, PTPρ, PTPψ, PTPφ, PTPζ, PTPNU3 and PTPH1.

4. The method of claim 1 wherein the protein tyrosine phosphatase is a protein tyrosine phosphatase as presented in FIG. 8.

5. The method of claim 1 wherein the protein tyrosine phosphatase is a dual specificity phosphatase.

6. The method of claim 1 wherein the protein tyrosine phosphatase substrate comprises phosphorylated poly-(4:1)-Glu-Tyr.

7. The method of claim 6 wherein the phosphorylated poly-(4:1)-Glu-Tyr comprises ³²P.

8. The method of claim 1 wherein the detectably labeled protein tyrosine phosphatase substrate comprises a reporter molecule selected from the group consisting of a fluorophore, a radionuclide, a chemiluminescent agent, an enzyme, an immunologically detectable epitope and a chromaphore.

9. The method of claim 8 wherein the fluorophore is selected from the group consisting of fluorescein, rhodamine, Texas Red, AlexaFluor-594, AlexaFluor-488, Oregon Green, BODIPY-FL and Cy-5.

10. The method of claim 1 wherein the protein tyrosine phosphatase substrate comprises a polypeptide sequence derived from a protein selected from the group consisting of PDGF receptor, VCP, p130^{cas}, EGF receptor, p210 bcr:abl, MAP kinase, Shc, insulin receptor, lck, T cell receptor zeta chain, and reduced and carboxyamidomethylated and maleylated lysozyme (RCML).

11. The method of claim 1 wherein the sulfhydryl-reactive agent that is capable of irreversibly modifying a sulfhydryl group of a protein tyrosine phosphatase active site invariant cysteine is an alkylating agent.

12. The method of claim 1 wherein the sulfhydryl-reactive agent that is capable of covalently modifying a sulfhydryl group of a protein tyrosine phosphatase active site invariant cysteine is selected from the group consisting of iodoacetamide, iodoacetic acid, arsenic oxide, maleimide analog, haloacetimido analog, 4-vinylpyrimidine analog and N-ethylmaleimide.

13. The method of claim 1 wherein the cell is a mammalian cell.

14. The method of claim 13 wherein the mammalian cell is derived from a cell line.

15. The method of claim 14 wherein the cell line is selected from the group consisting of Rat-1 fibroblasts, COS cells, CHO cells and HEK-293 cells.

16. The method of claim 1 wherein the step of isolating the protein tyrosine phosphatase comprises cell lysis.

17. The method of claim 16 wherein the step of isolating further comprises gel electrophoresis of the protein tyrosine phosphatase.

18. The method of claim 17 wherein the step of isolating further comprises electrophoresis of the protein tyrosine phosphatase in a gel comprising the detectably labeled protein tyrosine phosphatase substrate.

19. The method of claim 16 wherein the step of isolating further comprises detecting the protein tyrosine phosphatase with an antibody that specifically binds to the phosphatase.

20. The method of claim 1 wherein the stimulus increases reactive oxygen species in the sample.

21. The method of claim 1 wherein the stimulus is selected from the group consisting of a cytokine, a growth factor, a hormone, a cell stressor and a peptide.

22. The method of claim 21 wherein the cell stressor is selected from the group consisting of a source of ROS and ultraviolet light.

23. The method of claim 1 wherein the stimulus is selected from the group consisting of PDGF, EGF, bFGF, insulin, GM-CSF, TGF-β1, IL-1, IL-3, IFN-γ, TNF-α, PHA, AT-2, thrombin, thyrotropin, parathyroid hormone, LPA, sphingosine-1-phosphate, serotonin, endothelin, acetylcholine, platelet activating factor, bradykinin and G-CSF.

24. A method for identifying a protein tyrosine phosphatase that is reversibly modified by a PTP active site-binding agent in a cell, comprising:
contacting a PTP active site-binding agent that is capable of reversibly modifying a sulfhydryl group of a protein tyrosine phosphatase active site invariant cysteine with a biological sample comprising a cell that comprises at least one protein tyrosine phosphatase;
isolating the protein tyrosine phosphatase in the presence of a sulfhydryl-reactive agent that is capable of irreversibly modifying a sulfhydryl group of a protein tyrosine phosphatase active site invariant cysteine; and
determining, under conditions that are capable of reversing a reversible modification of a sulfhydryl group of a protein tyrosine phosphatase active site invariant cysteine, a level of dephosphorylation of a detectably labeled protein tyrosine phosphatase substrate by the protein tyrosine phosphatase, wherein detectable substrate dephosphorylation indicates that an active protein tyrosine phosphatase is present, and therefrom identifying a protein tyrosine phosphatase that is reversibly modified by a PTP active site-binding agent in a cell.

25. The method of claim 24 wherein the step of isolating is performed anaerobically.

26. The method of claim 24 wherein the PTP active site-binding agent is selected from the group consisting of an agent that covalently binds to the PTP active site and an agent that non-covalently binds to the PTP active site.

27. The method of claim 24 wherein the PTP active site-binding agent is selected from the group consisting of a sulfonated compound and a vanadate compound.

28. The method of claim 24 wherein the PTP active site-binding agent covalently and reversibly modifies a sulfhydryl group of a PTP active site invariant cysteine.

29. The method of claim 28 wherein the step of determining comprises reversing a covalent modification of a sulfhydryl group of a PTP active site invariant cysteine.

30. The method of claim 29 wherein the step of reversing comprises contacting the PTP with a reducing agent.

31. The method of claim 30 wherein the reducing agent is selected from the group consisting of dithiothreitol, dithioerythritol, and 2-mercaptoethanol.

32. The method of claim 24 wherein the sulfhydryl-reactive agent that is capable of irreversibly modifying a sulfhydryl group of a protein tyrosine phosphatase active site invariant cysteine is selected from the group consisting of iodoacetamide, iodoacetic acid, arsenic oxide, maleimide analog, haloacetimido analog, 4-vinylpyrimidine analog, and N-ethylmaleimide.

33. A method for identifying a protein tyrosine phosphatase that is a reversibly modified component of an inducible biological signaling pathway in a cell, comprising:
contacting a biological sample comprising a cell that comprises at least one protein tyrosine phosphatase with a stimulus that induces a biological signaling pathway under conditions and for a time sufficient to induce the biological signaling pathway and thereby reversibly protect a protein tyrosine phosphatase active site invariant cysteine from modification;
isolating the protein tyrosine phosphatase in the presence of a sulfhydryl-reactive agent that is capable of irreversibly modifying a sulfhydryl group of a protein tyrosine phosphatase active site invariant cysteine; and
determining, under conditions that reverse the reversible protection of the protein tyrosine phosphatase active site invariant cysteine from modification, a level of dephosphorylation of a detectably labeled protein tyrosine phosphatase substrate by the protein tyrosine phosphatase, wherein detectable substrate dephosphorylation indicates that an active protein tyrosine phosphatase is present, and therefrom identifying a protein tyrosine phosphatase that is a reversibly modified component of an inducible biological signaling pathway in a cell.

34. The method of claim 33 wherein the step of isolating is performed anaerobically.

35. The method of claim 33 wherein the sulfhydryl-reactive agent that is capable of irreversibly modifying a sulfhydryl group of a protein tyrosine phosphatase active site invariant cysteine is selected from the group consisting of iodoacetamide, iodoacetic acid, arsenic oxide, maleimide analog, haloacetimido analog, 4-vinylpyrimidine analog, and N-ethylmaleimide.

36. A method for identifying an agent that alters an inducible biological signaling pathway, comprising:
(a) identifying a protein tyrosine phosphatase that is reversibly oxidized in a cell according to a method comprising:
(i) contacting a first biological sample comprising a cell that comprises at least one protein tyrosine phosphatase with a stimulus under conditions and for a time sufficient to induce reversible oxidation of at least one protein tyrosine phosphatase in the cell;
(ii) isolating the protein tyrosine phosphatase in the presence of a sulfhydryl-reactive agent that is capable of irreversibly modifying a sulfhydryl group of a protein tyrosine phosphatase active site invariant cysteine;
(iii) determining under reducing conditions a level of dephosphorylation of a detectably labeled protein tyrosine phosphatase substrate by the protein tyrosine phosphatase, wherein detectable substrate dephosphorylation indicates that an active protein tyrosine phosphatase is present, and therefrom identifying a protein tyrosine phosphatase that is reversibly oxidized in a cell;
(b) contacting, in the presence and absence of a candidate agent, a second biological sample comprising a cell that comprises the PTP that is reversibly oxidized as identified according to the method of (a) with the stimulus under conditions and for a time sufficient to induce reversible oxidation of the PTP;
(c) isolating the protein tyrosine phosphatase in the presence of a sulfhydryl-reactive agent that is capable of covalently modifying a sulfhydryl group of a protein tyrosine phosphatase active site invariant cysteine; and
(d) determining under reducing conditions a level of dephosphorylation of a detectably labeled protein tyrosine phosphatase substrate by the protein tyrosine phosphatase,
wherein a level of substrate dephosphorylation that is decreased when the second sample is contacted with the stimulus in the presence of the candidate agent relative to the level of substrate dephosphorylation when the sample is contacted with the stimulus in the absence of the agent indicates that the agent is an inhibitor of an inducible biological signaling pathway,
and wherein a level of substrate dephosphorylation that is increased when the sample is contacted with the stimulus in the presence of the candidate agent relative to the level of substrate dephosphorylation when the sample is contacted with the stimulus in the absence of the agent indicates that the agent is a potentiator of an inducible biological signaling pathway.

37. The method of claim 36 wherein the step of isolating in the method recited in (a) is performed anaerobically.

38. The method of claim 36 wherein the step of isolating recited in (c) is performed anaerobically.

39. A method for identifying a SHP-2 protein tyrosine phosphatase (SHP-2) that is reversibly oxidized in a cell, comprising:
contacting a biological sample comprising a cell that comprises SHP-2 with a stimulus under conditions and for a time sufficient to induce reversible oxidation of SHP-2 in the cell;
isolating anaerobically SHP-2 in the presence of a sulfhydryl-reactive agent that is capable of irreversibly modifying a sulfhydryl group of a SHP-2 active site invariant cysteine; and
determining under reducing conditions a level of dephosphorylation of a detectably labeled SHP-2 substrate by SHP-2, wherein SHP-2 comprises a polypeptide comprising an amino acid sequence set forth in any one of SEQ ID NOS: 14, 16, 26, 28, 30, and 32, wherein detectable substrate dephosphorylation indicates that an active SHP-2 is present, and therefrom identifying a SHP-2 that is reversibly oxidized in a cell.

40. A method for identifying a PTP1B protein tyrosine phosphatase (PTP1B) that is reversibly oxidized in a cell, comprising:
contacting a biological sample comprising a cell that comprises PTP1B with a stimulus under conditions and for a time sufficient to induce reversible oxidation of PTP1B in the cell;
isolating anaerobically PTP1B in the presence of a sulfhydryl-reactive agent that is capable of irreversibly modifying a sulfhydryl group of a PTP1B active site invariant cysteine; and
determining under reducing conditions a level of dephosphorylation of a detectably labeled PTP1B substrate by PTP1B, wherein PTP1B comprises a polypeptide comprising an amino acid sequence set forth in any one of SEQ ID NOS: 2, 4, 6, 8, 10, and 12, and wherein detectable substrate dephosphorylation indicates that an active PTP1B is present, and therefrom identifying a PTP1B that is reversibly oxidized in a cell.

41. A method for identifying a TC45 protein tyrosine phosphatase (TC45) that is reversibly oxidized in a cell, comprising:
contacting a biological sample comprising a cell that comprises TC45 with a stimulus under conditions and for a time sufficient to induce reversible oxidation of TC45 in the cell;
isolating anaerobically TC45 in the presence of a sulfhydryl-reactive agent that is capable of irreversibly modifying a sulfhydryl group of a TC45 active site invariant cysteine; and
determining under reducing conditions a level of dephosphorylation of a detectably labeled TC45 substrate by TC45, wherein TC45 comprises a polypeptide comprising an amino acid sequence set forth in NM_{—}080422, and wherein detectable substrate dephosphorylation indicates that an active TC45 is present, and therefrom identifying a TC45 that is reversibly oxidized in a cell.

42. A method for identifying a SHP-2 protein tyrosine phosphatase (SHP-2) that is reversibly modified by a PTP active site-binding agent in a cell, comprising:
contacting a PTP active site-binding agent that is capable of reversibly modifying a sulfhydryl group of a SHP-2 active site invariant cysteine with a biological sample comprising a cell that comprises SHP-2;
isolating SHP-2 in the presence of a sulfhydryl-reactive agent that is capable of irreversibly modifying a sulfhydryl group of a SHP-2 active site invariant cysteine; and
determining, under conditions that are capable of reversing a reversible modification of a sulfhydryl group of a SHP-2 active site invariant cysteine, a level of dephosphorylation of a detectably labeled SHP-2 substrate by SHP-2, wherein SHP-2 comprises a polypeptide comprising an amino acid sequence set forth in any one of SEQ ID NOS: 14, 16, 26, 28, 30, and 32, wherein detectable substrate dephosphorylation indicates that an active SHP-2 is present, and therefrom identifying a SHP-2 that is reversibly modified by a PTP active site-binding agent in a cell.

43. A method for identifying a PTP1B protein tyrosine phosphatase (PTP1B) that is reversibly modified by a PTP active site-binding agent in a cell, comprising:
contacting a PTP active site-binding agent that is capable of reversibly modifying a sulfhydryl group of a PTP1B active site invariant cysteine with a biological sample comprising a cell that comprises PTP1B;
isolating PTP1B in the presence of a sulfhydryl-reactive agent that is capable of irreversibly modifying a sulfhydryl group of a PTP1B active site invariant cysteine; and
determining, under conditions that are capable of reversing a reversible modification of a sulfhydryl group of a PTP1B active site invariant cysteine, a level of dephosphorylation of a detectably labeled PTP1B substrate by PTP1B, wherein PTP1B comprises a polypeptide comprising an amino acid sequence set forth in any one of SEQ ID NOS: 2, 4, 6, 8, 10, and 12, and wherein detectable substrate dephosphorylation indicates that an active PTP1B is present, and therefrom identifying a PTP1B that is reversibly modified by a PTP active site-binding agent in a cell.

44. A method for identifying a TC45 protein tyrosine phosphatase (TC45) that is reversibly modified by a PTP active site-binding agent in a cell, comprising:
contacting a PTP active site-binding agent that is capable of reversibly modifying a sulfhydryl group of a TC45 active site invariant cysteine with a biological sample comprising a cell that comprises TC45;
isolating TC45 in the presence of a sulfhydryl-reactive agent that is capable of irreversibly modifying a sulfhydryl group of a TC45 active site invariant cysteine; and
determining, under conditions that are capable of reversing a reversible modification of a sulfhydryl group of a TC45 active site invariant cysteine, a level of dephosphorylation of a detectably labeled TC45 substrate by TC45, wherein TC45 comprises a polypeptide comprising an amino acid sequence set forth in NM_{—}080422, and wherein detectable substrate dephosphorylation indicates that an active TC45 is present, and therefrom identifying a TC45 that is reversibly modified by a PTP active site-binding agent in a cell.

45. A method for identifying a SHP-2 protein tyrosine phosphatase (SHP-2) that is a reversibly modified component of an inducible biological signaling pathway in a cell, comprising:
contacting a biological sample comprising a cell that comprises SHP-2 with a stimulus that induces a biological signaling pathway under conditions and for a time sufficient to induce the biological signaling pathway and thereby reversibly protect a SHP-2 active site invariant cysteine from modification;
isolating the SHP-2 in the presence of a sulfhydryl-reactive agent that is capable of irreversibly modifying a sulfhydryl group of a SHP-2 active site invariant cysteine; and
determining, under conditions that reverse the reversible protection of the SHP-2 active site invariant cysteine from modification, a level of dephosphorylation of a detectably labeled SHP-2 substrate by SHP-2, wherein SHP-2 comprises a polypeptide comprising an amino acid sequence set forth in any one of SEQ ID NOS: 14, 16, 26, 28, 30, and 32, and wherein detectable substrate dephosphorylation indicates that an active SHP-2 is present, and therefrom identifying a SHP-2 that is a reversibly modified component of an inducible biological signaling pathway in a cell.

46. A method for identifying a PTP1B protein tyrosine phosphatase (PTP1B) that is a reversibly modified component of an inducible biological signaling pathway in a cell, comprising:
contacting a biological sample comprising a cell that comprises PTP1B with a stimulus that induces a biological signaling pathway under conditions and for a time sufficient to induce the biological signaling pathway and thereby reversibly protect a PTP1B active site invariant cysteine from modification;
isolating the PTP1B in the presence of a sulfhydryl-reactive agent that is capable of irreversibly modifying a sulfhydryl group of a PTP1B active site invariant cysteine; and
determining, under conditions that reverse the reversible protection of the PTP1B active site invariant cysteine from modification, a level of dephosphorylation of a detectably labeled PTP1B substrate by PTP1B, wherein PTP1B comprises a polypeptide comprising an amino acid sequence set forth in any one of SEQ ID NOS: 2, 4, 6, 8, 10, and 12, and wherein detectable substrate dephosphorylation indicates that an active PTP1B is present, and therefrom identifying a PTP1B that is a reversibly modified component of an inducible biological signaling pathway in a cell.

47. A method for identifying a TC45 protein tyrosine phosphatase (TC45) that is a reversibly modified component of an inducible biological signaling pathway in a cell, comprising:
contacting a biological sample comprising a cell that comprises TC45 with a stimulus that induces a biological signaling pathway under conditions and for a time sufficient to induce the biological signaling pathway and thereby reversibly protect a TC45 active site invariant cysteine from modification;
isolating the TC45 in the presence of a sulfhydryl-reactive agent that is capable of irreversibly modifying a sulfhydryl group of a TC45 active site invariant cysteine; and
determining, under conditions that reverse the reversible protection of the TC45 active site invariant cysteine from modification, a level of dephosphorylation of a detectably labeled TC45 substrate by TC45, wherein TC45 comprises a polypeptide comprising an amino acid sequence set forth in NM_{—}080422, and wherein detectable substrate dephosphorylation indicates that an active TC45 is present, and therefrom identifying a TC45 that is a reversibly modified component of an inducible biological signaling pathway in a cell.

48. A method for identifying an agent that alters an inducible biological signaling pathway, comprising:
(a) identifying a SHP-2 protein tyrosine phosphatase (SHP-2) that is reversibly oxidized in a cell according to a method comprising:
(i) contacting a first biological sample comprising a cell that comprises SHP-2 with a stimulus under conditions and for a time sufficient to induce reversible oxidation of SHP-2 in the cell;
(ii) isolating SHP-2 in the presence of a sulfhydryl-reactive agent that is capable of irreversibly modifying a sulfhydryl group of a SHP-2 active site invariant cysteine;
(iii) determining under reducing conditions a level of dephosphorylation of a detectably labeled SHP-2 substrate by SHP-2, wherein detectable substrate dephosphorylation indicates that an active SHP-2 is present, and therefrom identifying a SHP-2 that is reversibly oxidized in a cell;
(b) contacting, in the presence and absence of a candidate agent, a second biological sample comprising a cell that comprises SHP-2 that is reversibly oxidized as identified according to the method of (a) with the stimulus under conditions and for a time sufficient to induce reversible oxidation of SHP-2;
(c) isolating SHP-2 in the presence of a sulfhydryl-reactive agent that is capable of covalently modifying a sulfhydryl group of a SHP-2 active site invariant cysteine; and
(d) determining under reducing conditions a level of dephosphorylation of a detectably labeled SHP-2 substrate by SHP-2, wherein SHP-2 comprises a polypeptide comprising an amino acid sequence set forth in any one of SEQ ID NOS: 14, 16, 26, 28, 30, and 32,
wherein a level of substrate dephosphorylation that is decreased when the second sample is contacted with the stimulus in the presence of the candidate agent relative to the level of substrate dephosphorylation when the sample is contacted with the stimulus in the absence of the agent indicates that the agent is an inhibitor of an inducible biological signaling pathway,
wherein a level of substrate dephosphorylation that is increased when the sample is contacted with the stimulus in the presence of the candidate agent relative to the level of substrate dephosphorylation when the sample is contacted with the stimulus in the absence of the agent indicates that the agent is a potentiator of an inducible biological signaling pathway.

49. A method for identifying an agent that alters an inducible biological signaling pathway, comprising:
(a) identifying a PTP1B protein tyrosine phosphatase (PTP1B) that is reversibly oxidized in a cell according to a method comprising:
(i) contacting a first biological sample comprising a cell that comprises PTP1B with a stimulus under conditions and for a time sufficient to induce reversible oxidation of PTP1B in the cell;
(ii) isolating PTP1B in the presence of a sulfhydryl-reactive agent that is capable of irreversibly modifying a sulfhydryl group of a PTP1B active site invariant cysteine;
(iii) determining under reducing conditions a level of dephosphorylation of a detectably labeled PTP1B substrate by PTP1B, wherein detectable substrate dephosphorylation indicates that an active PTP1B is present, and therefrom identifying a PTP1B that is reversibly oxidized in a cell;
(b) contacting, in the presence and absence of a candidate agent, a second biological sample comprising a cell that comprises PTP1B that is reversibly oxidized as identified according to the method of (a) with the stimulus under conditions and for a time sufficient to induce reversible oxidation of PTP1B;
(c) isolating PTP1B in the presence of a sulfhydryl-reactive agent that is capable of covalently modifying a sulfhydryl group of a PTP1B active site invariant cysteine; and
(d) determining under reducing conditions a level of dephosphorylation of a detectably labeled PTP1B substrate by PTP1B, wherein PTP1B comprises a polypeptide comprising an amino acid sequence set forth in any one of SEQ ID NOS: 2, 4, 6, 8, 10, and 12,
wherein a level of substrate dephosphorylation that is decreased when the second sample is contacted with the stimulus in the presence of the candidate agent relative to the level of substrate dephosphorylation when the sample is contacted with the stimulus in the absence of the agent indicates that the agent is an inhibitor of an inducible biological signaling pathway,
and wherein a level of substrate dephosphorylation that is increased when the sample is contacted with the stimulus in the presence of the candidate agent relative to the level of substrate dephosphorylation when the sample is contacted with the stimulus in the absence of the agent indicates that the agent is a potentiator of an inducible biological signaling pathway.

50. A method for identifying an agent that alters an inducible biological signaling pathway, comprising:
(a) identifying a TC45 protein tyrosine phosphatase (TC45) that is reversibly oxidized in a cell according to a method comprising:
(i) contacting a first biological sample comprising a cell that comprises TC45 with a stimulus under conditions and for a time sufficient to induce reversible oxidation of TC45 in the cell;
(ii) isolating TC45 in the presence of a sulfhydryl-reactive agent that is capable of irreversibly modifying a sulfhydryl group of a TC45 active site invariant cysteine;
(iii) determining under reducing conditions a level of dephosphorylation of a detectably labeled TC45 substrate by TC45, wherein detectable substrate dephosphorylation indicates that an active TC45 is present, and therefrom identifying a TC45 that is reversibly oxidized in a cell;
(b) contacting, in the presence and absence of a candidate agent, a second biological sample comprising a cell that comprises TC45 that is reversibly oxidized as identified according to the method of (a) with the stimulus under conditions and for a time sufficient to induce reversible oxidation of TC45;
(c) isolating TC45 in the presence of a sulfhydryl-reactive agent that is capable of covalently modifying a sulfhydryl group of a TC45 active site invariant cysteine; and
(d) determining under reducing conditions a level of dephosphorylation of a detectably labeled TC45 substrate by TC45, wherein TC45 comprises a polypeptide comprising an amino acid sequence set forth in NM_{—}080422,
wherein a level of substrate dephosphorylation that is decreased when the second sample is contacted with the stimulus in the presence of the candidate agent relative to the level of substrate dephosphorylation when the sample is contacted with the stimulus in the absence of the agent indicates that the agent is an inhibitor of an inducible biological signaling pathway,
and wherein a level of substrate dephosphorylation that is increased when the sample is contacted with the stimulus in the presence of the candidate agent relative to the level of substrate dephosphorylation when the sample is contacted with the stimulus in the absence of the agent indicates that the agent is a potentiator of an inducible biological signaling pathway.


SEQUENCE LISTING (98 sequences)

SEQ ID NO: 1 - DNA, 3247 bp, Homo sapiens
gggcgggcctcggggctaagagcgcgacgcctagagcggcagacggcgcagtgggccgagaaggaggcgcagcagccgccctggcccgtcatggagatggaaaaggagttcgagcagatcgacaagtccgggagctgggcggccatttaccaggatatccgacatgaagccagtgacttcccatgtagagtggccaagcttcctaagaacaaaaaccgaaataggtacagagacgtcagtccctttgaccatagtcggattaaactacatcaagaagataatgactatatcaacgctagtttgataaaaatggaagaagcccaaaggagttacattcttacccagggccctttgcctaacacatgcggtcacttttgggagatggtgtgggagcagaaaagcaggggtgtcgtcatgctcaacagagtgatggagaaaggttcgttaaaatgcgcacaatactggccacaaaaagaagaaaaagagatgatctttgaagacacaaatttgaaattaacattgatctctgaagatatcaagtcatattatacagtgcgacagctagaattggaaaaccttacaacccaagaaactcgagagatcttacatttccactataccacatggcctgactttggagtccctgaatcaccagcctcattcttgaactttcttttcaaagtccgagagtcagggtcactcagcccggagcacgggcccgttgtggtgcactgcagtgcaggcatcggcaggtctggaaccttctgtctggctgatacctgcctcctgctgatggacaagaggaaagacccttcttccgttgatatcaagaaagtgctgttagaaatgaggaagtttcggatggggttgatccagacagccgaccagctgcgcttctcctacctggctgtgatcgaaggtgccaaattcatcatgggggactcttccgtgcaggatcagtggaaggagctttcccacgaggacctggagcccccacccgagcatatccccccacctccccggccacccaaacgaatcctggagccacacaatgggaaatgcagggagttcttcccaaatcaccagtgggtgaaggaagagacccaggaggataaagactgccccatcaaggaagaaaaaggaagccccttaaatgccgcaccctacggcatcgaaagcatgagtcaagacactgaagttagaagtcgggtcgtggggggaagtcttcgaggtgcccaggctgcctccccagccaaaggggagccgtcactgcccgagaaggacgaggaccatgcactgagttactggaagcccttcctggtcaacatgtgcgtggctacggtcctcacggccggcgcttacctctgctacaggttcctgttcaacagcaacacatagcctgaccctcctccactccacctccacccactgtccgcctctgcccgcagagcccacgcccgactagcaggcatgccgcggtaggtaagggccgccggaccgcgtagagagccgggccccggacggacgttggttctgcactaaaacccatcttccccggatgtgtgtctcacccctcatccttttactttttgccccttccactttgagtaccaaatccacaagccattttttgaggagagtgaaagagagtaccatgctggcggcgcagagggaaggggcctacacccgtcttggggctcgccccacccagggctccctcctggagcatcccaggcggcgcacgccaacagccccccccttgaatctgcagggagcaactctccactccatatttatttaaacaattttttccccaaaggcatccatagtgcactagcattttcttgaaccaataatgtattaaaattttttgatgtcagccttgcatcaagggctttatcaaaaagtacaataataaatcctcaggtagtactgggaatggaaggctttgccatgggcctgctgcgtcagaccagtactgggaaggaggacggttgtaagcagttgttatttagtgatattgtgggtaacgtgagaagatagaacaatgctataatatataatgaacacgtgggtatttaataagaaacatgatgtgagattactttgtcccgcttattctcctccctgttatctgctagatctagttctcaatcactgctcccccgtgtgtattagaatgcatgtaaggtcttcttgtgtcctgatgaaaaatatgtgcttgaaatgagaaactttgatctctgcttactaatgtgccccatgtccaagtccaacctgcctgtgcatgacctgatcattacatggctgtggttcctaagcctgttgctgaagtcattgtcgctcagcaatagggtgcagttttccaggaataggcatttgctaattcctggcatgacactctagtgacttcctggtgaggcccagcctgtcctggtacagcagggtcttgctgtaactcagacattccaagggtatgggaagccatattcacacctcacgctctggacatgatttagggaagcagggacaccccccgccccccacctttgggatcagcctccgccattccaagtcaacactcttcttgagcagaccgtgatttggaagagaggcacctgctggaaaccacacttcttgaaacagcctgggtgacggtcctttaggcagcctgccgccgtctctgtcccggttcaccttgccgagagaggcgcgtctgccccaccctcaaaccctgtggggcctgatggtgctcacgactcttcctgcaaagggaactgaagacctccacattaagtggctttttaacatgaaaaacacggcagctgtagctcccgagctactctcttgccagcattttcacattttgcctttctcgtggtagaagccagtacagagaaattctgtggtgggaacattcgaggtgtcaccctgcagagctatggtgaggtgtggataaggcttaggtgccaggctgtaagcattctgagctggcttgttgtttttaagtcctgtatatgtatgtagtagtttgggtgtgtatatatagtagcatttcaaaatggacgtactggtttaacctcctatccttggagagcagctggctctccaccttgttacacattatgttagagaggtagcgagctgctctgctatatgccttaagccaatatttactcatcaggtcattattttttacaatggccatggaataaaccatttttacaaaaataaaaacaaaaaaagc

SEQ ID NO: 2 - protein, 435 aa, Homo sapiens
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNGKCREFFPNHQWVKEETQEDKDCPIKEEKGSPLNAAPYGIESMSQDTEVRSRVVGGSLRGAQAASPAKGEPSLPEKDEDHALSYWKPFLVNMCVATVLTAGAYLCYRFLFNSNT

SEQ ID NO: 3 - DNA, 3318 bp, Homo sapiens
gtgatgcgtagttccggctgccggttgacatgaagaagcagcagcggctagggcggcggtagctgcaggggtcggggattgcagcgggcctcggggctaagagcgcgacgcggcctagagcggcagacggcgcagtgggccgagaaggaggcgcagcagccgccctggcccgtcatggagatggaaaaggagttcgagcagatcgacaagtccgggagctgggcggccatttaccaggatatccgacatgaagccagtgacttcccatgtagagtggccaagcttcctaagaacaaaaaccgaaataggtacagagacgtcagtccctttgaccatagtcggattaaactacatcaagaagataatgactatatcaacgctagtttgataaaaatggaagaagcccaaaggagttacattcttacccagggccctttgcctaacacatgcggtcacttttgggagatggtgtgggagcagaaaagcaggggtgtcgtcatgctcaacagagtgatggagaaaggttcgttaaaatgcgcacaatactggccacaaaaagaagaaaaagagatgatctttgaagacacaaatttgaaattaacattgatctctgaagatatcaagtcatattatacagtgcgacagctagaattggaaaaccttacaacccaagaaactcgagagatcttacatttccactataccacatggcctgactttggagtccctgaatcaccagcctcattcttgaactttcttttcaaagtccgagagtcagggtcactcagcccggagcacgggcccgttgtggtgcactgcagtgcaggcatcggcaggtctggaaccttctgtctggctgatacctgcctcttgctgatggacaagaggaaagacccttcttccgttgatatcaagaaagtgctgttagaaatgaggaagtttcggatggggctgatccagacagccgaccagctgcgcttctcctacctggctgtgatcgaaggtgccaaattcatcatgggggactcttccgtgcaggatcagtggaaggagctttcccacgaggacctggagcccccacccgagcatatccccccacctccccggccacccaaacgaatcctggagccacacaatgggaaatgcagggagttcttcccaaatcaccagtgggtgaaggaagagacccaggaggataaagactgccccatcaaggaagaaaaaggaagccccttaaatgccgcaccctacggcatcgaaagcatgagtcaagacactgaagttagaagtcgggtcgtggggggaagtcttcgaggtgcccaggctgcctccccagccaaaggggagccgtcactgcccgagaaggacgaggaccatgcactgagttactggaagcccttcctggtcaacatgtgcgtggctacggtcctcacggccggcgcttacctctgctacaggttcctgttcaacagcaacacatagcctgaccctcctccactccacctccacccactgtccgcctctgcccgcagagcccacgcccgactagcaggcatgccgcggtaggtaagggccgccggaccgcgtagagagccgggccccggacggacgttggttctgcactaaaacccatcttccccggatgtgtgtctcacccctcatccttttactttttgccccttccactttgagtaccaaatccacaagccattttttgaggagagtgaaagagagtaccatgctggcggcgcagagggaaggggcctacacccgtcttggggctcgccccacccagggctccctcctggagcatcccaggcgggcggcacgccaacagccccccccttgaatctgcagggagcaactctccactccatatttatttaaacaattttttccccaaaggcatccatagtgcactagcattttcttgaaccaataatgtattaaaattttttgatgtcagccttgcatcaagggctttatcaaaaagtacaataataaatcctcaggtagtactgggaatggaaggctttgccatgggcctgctgcgtcagaccagtactgggaaggaggacggttgtaagcagttgttatttagtgatattgtgggtaacgtgagaagatagaacaatgctataatatataatgaacacgtgggtatttaataagaaacatgatgtgagattactttgtcccgcttattctcctccctgttatctgctagatctagttctcaatcactgctcccccgtgtgtattagaatgcatgtaaggtcttcttgtgtcctgatgaaaaatatgtgcttgaaatgagaaactttgatctctgcttactaatgtgccccatgtccaagtccaacctgcctgtgcatgacctgatcattacatggctgtggttcctaagcctgttgctgaagtcattgtcgctcagcaatagggtgcagttttccaggaataggcatttgcctaattcctggcatgacactctagtgacttcctggtgaggcccagcctgtcctggtacagcagggtcttgctgtaactcagacattccaagggtatgggaagccatattcacacctcacgctctggacatgatttagggaagcagggacaccccccgccccccacctttgggatcagcctccgccattccaagtcaacactcttcttgagcagaccgtgatttggaagagaggcacctgctggaaaccacacttcttgaaacagcctgggtgacggtcctttaggcagcctgccgccgtctctgtcccggttcaccttgccgagagaggcgcgtctgccccaccctcaaaccctgtggggcctgatggtgctcacgactcttcctgcaaagggaactgaagacctccacattaagtggctttttaacatgaaaaacacggcagctgtagctcccgagctactctcttgccagcattttcacattttgcctttctcgtggtagaagccagtacagagaaattctgtggtgggaacattcgaggtgtcaccctgcagagctatggtgaggtgtggataaggcttaggtgccaggctgtaagcattctgagctgggcttgttgtttttaagtcctgtatatgtatgtagtagtttgggtgtgtatatatagtagcatttcaaaatggacgtactggtttaacctcctatccttggagagcagctggctctccaccttgttacacattatgttagagaggtagcgagctgctctgctatatgccttaagccaatatttactcatcaggtcattattttttacaatggccatggaataaaccatttttacaaaa

SEQ ID NO: 4 - protein, 435 aa, Homo sapiens; identical to SEQ ID NO: 2

SEQ ID NO: 5 - DNA, 2346 bp, Mus musculus
gaattcgggatccttttgcacattcctagttagcagtgcatactcatcagactggagatgtttaatgacatcagggaaccaaacggacaacccatagtacccgaagacagggtgaaccagacaatcgtaagcttgatggtgttttccctgactgggtagttgaagcatctcatgaatgtcagccaaattccgtacagttcggtgcggatccgaacgaaacacctcctgtaccaggttcccgtgtcgctctcaatttcaatcagctcatctatttgtttgggagtcttgattttatttaccgtgaagaccttctctggctggccccgggctctcatgttggtgtcatgaattaacttcagaatcatccaggcttcatcatgttttcccacctccagcaagaaccgagggctttctggcatgaaggtgagagccaccacagaggagacgcatgggagcgcacagacgatgacgaagacgcgccacgtgtggaactggtaggctgaacccatgctgaagctccacccgtagtggggaatgatggcccaggcatggcggaggctagatgccgccaatcatccagaacatgcagaagccgctgctggggagcttggggctgcggtggtggcgggtgacgggcttcgggacgcggagcgacgcggcctagcgcggcggacggccgtgggaactcgggcagccgacccgtcccgccatggagatggagaaggagttcgaggagatcgacaaggctgggaactgggcggctatttaccaggacattcgacatgaagccagcgacttcccatgcaaagtcgcgaagcttcctaagaacaaaaaccggaacaggtaccgagatgtcagcccttttgaccacagtcggattaaattgcaccaggaagataatgactatatcaatgccagcttgataaaaatggaagaagcccagaggagctatattctcacccagggccctttaccaaacacatgtgggcacttctgggagatggtgtgggagcagaagagcaggggcgtggtcatgctcaaccgcatcatggagaaaggctcgttaaaatgtgcccagtattggccacagcaagaagaaaaggagatggtctttgatgacacaggtttgaagttgacactaatctctgaagatgtcaagtcatattacacagtacgacagttggagttggaaaacctgactaccaaggagactcgagagatcctgcatttccactacaccacatggcctgactttggagtccccgagtcaccggcttctttcctcaatttccttttcaaagtccgagagtcaggctcactcagcctggagcatggccccattgtggtccactgcagcgccggcatcgggaggtcagggaccttctgtctggctgacacctgcctcttactgatggacaagaggaaagacccatcttccgtggacatcaagaaagtactgctggagatgcgcaggttccgcatggggctcatccagactgccgaccagctgcgcttctcctacctggctgtcatcgagggcgccaagttcatcatgggcgactcgtcagtgcaggatcagtggaaggagctctcccgggaggatctagaccttccacccgagcacgtgcccccacctccccggccacccaaacgcacactggagcctcacaacgggaagtgcaaggagctcttctccagccaccagtgggtgagcgaggagacctgtggggatgaagacagcctggccagagaggaaggcagagcccagtcaagtgccatgcacagcgtgagcagcatgagtccagacactgaagttaggagacggatggtgggtggaggtcttcaaagtgctcaggcgtctgtccccaccgaggaagagctgtcctccactgaggaggaacacaaggcacattggccaagtcactggaagcccttcctggtcaatgtgtgcatggccacgctcctggccaccggcgcgtacttgtgctaccgggtgtgttttcactgacagactgggaggcactgccactgcccagcttaggatgcggtctgcggcgtctgacctggtgtagagggaacaacaactcgcaagcctgctctggaactggaagggcctgccccaggagggtattagtgcactgggctttgaaggagcccctggtcccacgaacagagtctaatctcagggccttaacctgttcaggagaagtagaggaaatgccaaatactcttcttgctctcacctcactcctcccctttctctgattcatttgtttttggaaaaaaaaaaaaaaagaattacaacacattgttgtttttaacatttataaaggcaggcccgaattc

SEQ ID NO: 6 - protein, 432 aa, Mus musculus
MEMEKEFEEIDKAGNWAAIYQDIRHEASDFPCKVAKLPKNKNRNRYRDVSPFDHSRIKLHQEDNDYINASLIKMEEAQRSYILTQGPLPNTCGHFWEMVWEQKSRGVVMLNRIMEKGSLKCAQYWPQQEEKEMVFDDTGLKLTLISEDVKSYYTVRQLELENLTTKETREILHFHYTTWPDFGVPESPASFLNFLFKVRESGSLSLEHGPIVVHCSAGIGRSGTFCLADTCLLLMDKRKDPSSVDIKKVLLEMRRFRMGLIQTADQLRFSYLAVIEGAKFIMGDSSVQDQWKELSREDLDLPPEHVPPPPRPPKRTLEPHNGKCKELFSSHQWVSEETCGDEDSLAREEGRAQSSAMHSVSSMSPDTEVRRRMVGGGLQSAQASVPTEEELSSTEEEHKAHWPSHWKPFLVNVCMATLLATGAYLCYRVCFH

SEQ ID NO: 7 - DNA, 3247 bp, Homo sapiens; identical to SEQ ID NO: 1

SEQ ID NO: 8 - protein, 435 aa, Homo sapiens; identical to SEQ ID NO: 2

SEQ ID NO: 9 - DNA, 3215 bp, Homo sapiens
gcgcgacgcggcctagagcggcagacggcgcagtgggccgagaaggaggcgcagcagccgccctggcccgtcatggagatggaaaaggagttcgagcagatcgacaagtccgggagctgggcggccatttaccaggatatccgacatgaagccagtgacttcccatgtagagtggccaagcttcctaagaacaaaaaccgaaataggtacagagacgtcagtccctttgaccatagtcggattaaactacatcaagaagataatgactatatcaacgctagtttgataaaaatggaagaagcccaaaggagttacattcttacccagggccctttgcctaacacatgcggtcacttttgggagatggtgtgggagcagaaaagcaggggtgtcgtcatgctcaacagagtgatggagaaaggttcgttaaaatgcgcacaatactggccacaaaaagaagaaaaagagatgatctttgaagacacaaatttgaaattaacattgatctctgaagatatcaagtcatattatacagtgcgacagctagaattggaaaaccttacaacccaagaaactcgagagatcttacatttccactataccacatggcctgactttggagtccctgaatcaccagcctcattcttgaactttcttttcaaagtccgagagtcagggtcactcagcccggagcacgggcccgttgtggtgcactgcagtgcaggcatcggcaggtctggaaccttctgtctggctgatacctgcctcttgctgatggacaagaggaaagacccttcttccgttgatatcaagaaagtgctgttagaaatgaggaagtttcggatggggctgatccagacagccgaccagctgcgcttctcctacctggctgtgatcgaaggtgccaaattcatcatgggggactcttccgtgcaggatcagtggaaggagctttcccacgaggacctggagcccccacccgagcatatccccccacctccccggccacccaaacgaatcctggagccacacaatgggaaatgcagggagttcttcccaaatcaccagtgggtgaaggaagagacccaggaggataaagactgccccatcaaggaagaaaaaggaagccccttaaatgccgcaccctacggcatcgaaagcatgagtcaagacactgaagttagaagtcgggtcgtggggggaagtcttcgaggtgcccaggctgcctccccagccaaaggggagccgtcactgcccgagaaggacgaggaccatgcactgagttactggaagcccttcctggtcaacatgtgcgtggctacggtcctcacggccggcgcttacctctgctacaggttcctgttcaacagcaacacatagcctgaccctcctccactccacctccacccactgtccgcctctgcccgcagagcccacgcccgactagcaggcatgccgcggtaggtaagggccgccggaccgcgtagagagccgggccccggacggacgttggttctgcactaaaacccatcttccccggatgtgtgtctcacccctcatccttttactttttgccccttccactttgagtaccaaatccacaagccattttttgaggagagtgaaagagagtaccatgctggcggcgcagagggaaggggcctacacccgtcttggggctcgccccacccagggctccctcctggagcatcccaggcgggcggcacgccagacagccccccccttgaatctgcagggagcaactctccactccatatttatttaaacaattttttccccaaaggcatccatagtgcactagcattttcttgaaccaataatgtattaaaattttttgatgtcagccttgcatcaagggctttatcaaaaagtacaataataaatcctcaggtagtactgggaatggaaggctttgccatgggcctgctgcgtcagaccagtactgggaaggaggacggttgtaagcagttgttatttagtgatattgtgggtaacgtgagaagatagaacaatgctataatatataatgaacacgtgggtatttaataagaaacatgatgtgagattactttgtcccgcttattctgctccctgttatctgctagatctagttctcaatcactgctcccccgtgtgtattagaatgcatgtaaggtcttcttgtgtcctgatgaaaaatatgtgcttgaaatgagaaactttgatctctgcttactaatgtgccccatgtccaagtccaacctgcctgtgcatgacctgatcattacatggctgtggttcctaagcctgttgctgaagtcattgtcgctcagcaatagggtgcagttttccaggaataggcatttgcctaattcctggcatgacactctagtgacttcctggtgaggcccagcctgtcctggtacagcagggtcttgctgtaactcagacattccaagggtatgggaagccatattcacacctcacgctctggacatgatttagggaagcagggacaccccccgccccccacctttgggatcagcctccgccattccaagtcgacactcttcttgagcagaccgtgatttggaagagaggcacctgctggaaaccacacttcttgaaacagcctgggtgacggtcctttaggcagcctgccgccgtctctgtcccggttcaccttgccgagagaggcgcgtctgccccaccctcaaaccctgtggggcctgatggtgctcacgactcttcctgcaaagggaactgaagacctccacattaagtggctttttaacatgaaaaacacggcagctgtagctcccgagctactctcttgccagcattttcacattttgcctttctcgtggtagaagccagtacagagaaattctgtggtgggaacattcgaggtgtcaccctgcagagctatggtgaggtgtggataaggcttaggtgccaggctgtaagcattctgagctggcttgttgtttttaagtcctgtatatgtatgtagtagtttgggtgtgtatatatagtagcatttcaaaatggacgtactggtttaacctcctatccttggagagcagctggctctccaccttgttacacattatgttagagaggtagcgagctgctctgctatgtccttaagccaatatttactcatcaggtcattattttttacaatggccatggaataaaccatttttacaaaa

SEQ ID NO: 10 - protein, 435 aa, Homo sapiens; identical to SEQ ID NO: 2

SEQ ID NO: 11 - DNA, 4127 bp, Rattus norvegicus
agccgctgctggggaggttggggctgaggtggtggcgggcgacgggcctcgagacgcggagcgacgcggcctagcgcggcggacggccgagggaactcgggcagtcgtcccgtcccgccatggaaatggagaaggaattcgagcagatcgataaggctgggaactgggcggctatttaccaggatattcgacatgaagccagtgacttcccatgcagaatagcgaaacttcctaagaacaaaaaccggaacaggtaccgagatgtcagcccttttgaccacagtcggattaaattgcatcaggaagataatgactatatcaatgccagcttgataaaaatggaggaagcccagaggagctatatcctcacccagggccctttaccaaacacgtgcgggcacttctgggagatggtgtgggagcagaagagcaggggcgtggtcatgctcaaccgcatcatggagaaaggctcgttaaaatgtgcccagtattggccacagaaagaagaaaaagagatggtcttcgatgacaccaatttgaagctgacactgatctctgaagatgtcaagtcatattacacagtacggcagttggagttggagaacctggctacccaggaggctcgagagatcctgcatttccactacaccacctggcctgactttggagtccctgagtcacctgcctctttcctcaatttcctattcaaagtccgagagtcaggctcactcagcccagagcacggccccattgtggtccactgcagtgctggcattggcaggtcagggaccttctgcctggctgacacctgcctcttactgatggacaagaggaaagacccgtcctctgtggacatcaagaaagtgctgttggagatgcgcaggttccgcatggggctcatccagacggccgaccaactgcgcttctcctacctggctgtgatcgagggtgcaaagttcatcatgggcgactcgtcagtgcaggatcagtggaaggagctttcccatgaagacctggagcctccccctgagcacgtgcccccacctccccggccacccaaacgcacattggagcctcacaatggcaagtgcaaggagctcttctccaaccaccagtgggtgagcgaggagagctgtgaggatgaggacatcctggccagagaggaaagcagagccccctcaattgctgtgcacagcatgagcagtatgagtcaagacactgaagttaggaaacggatggtgggtggaggtcttcaaagtgctcaggcatctgtccccactgaggaagagctgtccccaaccgaggaggaacaaaaggcacacaggccagttcactggaagcccttcctggtcaacgtgtgcatggccacggccctggcgactggcgcgtacctctgttaccgggtatgttttcactgacagactgctgtgaggcatgagcgtggtgggcgctgccactgcccaggttaggatttggtctgcggcgtctaacctggtgtagaagaaacaacagcttacaagcctgtggtggaactggaagggccagccccaggaggggcatctgtgcactgggctttgaaggagcccctggtcccaagaacagagtctaatctcagggccttaacctgttcaggagaagtagaggaaatgccaaatactcttcttgctctcacctcactcctcccctttctctggttcgtttgtttttggaaaaaaaaaaaaaagaattacaacacattgttgtttttaacatttataaaggcaggtttttgttatttttagagaaaacaaaagatgctaggcactggtgagattctcttgtgccctttggcatgtgatcagattcacgatttacgtttatttccgggggagggtcccacctgtcaggactgtaaagttcctgctggcttggtcagcccccccacccccccaccccgagcttgcaggtgccctgctgtgaggagagcagcagcagaggctgcccctggacagaagcccagctctgcttccctcaggtgtccctgcgtttccatcctccttctttgtgaccgccatcttgcagatgacccagtcctcagcaccccacccctgcagatgggtttctccgagggcctgcctcagggtcatcagaggttggctgccagcttagagctggggcttccatttgattggaaagtcattactattctatgtagaagccactccactgaggtgtaaagcaagactcataaaggaggagccttggtgtcatggaagtcactccgcgcgcaggacctgtaacaacctctgaaacactcagtcctgctgcagtgacgtccttgaaggcatcagacagatgatttgcagactgccaagacttgtcctgagccgtgatttttagagtctggactcatgaaacaccgccgagcgcttactgtgcagcctctgatgctggttggctgaggctgcggggaggtggacactgtgggtgcatccagtgcagttgcttttgtgcagttgggtccagcagcacagcccgcactccagcctcagctgcaggccacagtggccatggaggccgccagagcgagctggggtggatgcttgttcacttggagcagccttcccaggacgtgcagctcccttcctgctttgtccttctgcttccttccctggagtagcaagcccacgagcaatcgtgaggggtgtgagggagctgcagaggcatcagagtggcctgcagcggcgtgaggccccttcccctccgacacccccctccagaggagccgctccactgttatttattcactttgcccacagacacccctgagtgagcacaccctgaaactgaccgtgtaaggtgtcagcctgcacccaggaccgtcaggtgcagcaccgggtcagtcctagggttgaggtaggactgacacagccactgtgtggctggtgctggggcaggggcaggagctgagggtcttagaagcaatcttcaggaacagacaacagtggtgacatgtaaagtccctgtggctactgatgacatgtgtaggatgaaggctggcctttctcccatgactttctagatcccgttccccgtctgctttccctgtgagttagaaaacacacaggctcctgtcctggtggtgccgtgtgcttgacatgggaaacttagatgcctgctcactggcgggcacctcggcatcgccaccactcagagtgagagcagtgctgtccagtgccgaggccgcctgactcccggcaggactcttcaggctctggcctgccccagcacaccccgctggatctcagacattccacacccacacctcattccctggacacttgggcaagcaggcccgcccttccacctctggggtcagcccctccattccgagttcacactgctctggagcaggccaggaccggaagcaaggcagctggtgaggagcaccctcctgggaacagtgtaggtgacagtcctgagagtcagcttgctagcgctgctggcaccagtcaccttgctcagaagtgtgtggctcttgaggctgaagagactgatgatggtgctcatgactcttctgtgaggggaacttgaccttcacattgggtggctttttttaaaataagcgaaggcagctggaactccagtctgcctcttgccagcacttcacattttgcctttcacccagagaagccagcacagagccactggggaaggcgatggccttgcctgcacaggctgaggagatggctcagccggcgtccaggctgtgtctggagcagggggtgcacagcagcctcacaggtgggggcctcagagcaggcgctgccctgtcccctgccccgctggaggcagcaaagctgctgcatgccttaagtcaatacttactcagcagggcgctctcgttctctctctctctctctctctctctctctctctctctctctctctctctaaatggccatagaataaaccattttacaaaaataaaagccaacaacaaagtgctctggaatagcacctttgcaggagcggggggtgtctcagggtcttctgtgacctcaccgaactgtccgactgcaccgtttccaacttgtgtctcactaatgggtctgcattagttgcaacaataaatgtttttaaagaac

SEQ ID NO: 12 - protein, 432 aa, Rattus norvegicus
MEMEKEFEQIDKAGNWAAIYQDIRHEASDFPCRIAKLPKNKNRNRYRDVSPFDHSRIKLHQEDNDYINASLIKMEEAQRSYILTQGPLPNTCGHFWEMVWEQKSRGVVMLNRIMEKGSLKCAQYWPQKEEKEMVFDDTNLKLTLISEDVKSYYTVRQLELENLATQEAREILHFHYTTWPDFGVPESPASFLNFLFKVRESGSLSPEHGPIVVHCSAGIGRSGTFCLADTCLLLMDKRKDPSSVDIKKVLLEMRRFRMGLIQTADQLRFSYLAVIEGAKFIMGDSSVQDQWKELSHEDLEPPPEHVPPPPRPPKRTLEPHNGKCKELFSNHQWVSEESCEDEDILAREESRAPSIAVHSMSSMSQDTEVRKRMVGGGLQSAQASVPTEEELSPTEEEQKAHRPVHWKPFLVNVCMATALATGAYLCYRVCFH

SEQ ID NO: 13 - DNA, 2229 bp, Mus musculus
ggtaccccgcggagcctgagcgagcaggcgtccgtgcggagccgaagacgggaggaacatgacatcgcggagatggtttcaccccaacatcactggtgtggaggcagagaatctcctgctgaccagaggagtcgatggcagttttttagcaaggcccagtaagagtaaccctggagacttcactctgtctgttagaagaaatggagctgttacccacatcaagattcagaacactggggactactatgacctctatggtggggagaagtttgccactttggctgaactggttcagtattacatggaacaccatgggcagctgaaagagaagaatggagatgttatcgagctcaagtacccgctgaactgtgcagaccctacctctgaaaggtggttccatggtcacttgtctggaaaagaagcagagaagctgctgacggagaagggcaagcatggcagcttcctcgttcgagagagccagagccaccccggagacttcgttctctccgtgcgcactggtgacgacaaaggggagagcaacgacggcaagtccaaagtgacccacgtcatgatccgctgtcaggagctgaaatacgacgttggtgggggagagcgctttgactctctgacagacctggtggagcattacaagaagaaccccatggtggagacgctgggcacagtcctgcagctcaagcagcccctcaacacaactcgtatcaatgctgctgaaattgaaagccgggttcgagagttaagcaagctggctgagaccacagataaagtcaagcagggcttttgggaagagtttgagacgctccagcaacaggaatgcaaacttctctatagccgaaaagaaggacagagacaagaaaataaaaacaaaaacagatacaaaaacatcctgccctttgatcataccagggtcgttctgcatgatggggatcccaatgagcctgtttctgattacattaatgcaaacatcatcatgcctgagtttgagaccaagtgcaacaattccaaacccaaaaagagttacattgccactcaaggctgcctgcagaacacggtgaatgacttctggcggatggtgttccaggagaactctcgagtcattgtcatgaccacaaaggaagtggagagagggaagagcaaatgtgtcaagtactggcctgatgagtatgcgctcaaagaatacggggtcatgcgtgttaggaacgtcaaagaaagtgccgcccatgactacactttacgagagctcaaactctctaaggtcggacaagctctactccagggaaacacagagagaaccgtctggcagtaccactttcggacctggccagaccatggcgtgcctagtgaccctggaggtgtgctggacttcctggaggaggtccaccacaagcaggagagcatcgtggatgcaggccctgtcgtggttcactgcagcgctgggattggccggacaggaaccttcattgtgattgacatccttattgacatcattcgagagaaaggtgtggactgtgacatcgacgttcctaaaaccattcagatggtgcggtcccagaggtcggggatggtccagacagaagcacagtaccggtttatctacatggctgtccagcactacatagagacgctgcagcgccggatcgaggaggagcagaaaagcaaaagaaaaggacatgaatataccaatattaagtattccttggtggaccagacaagtggtgatcagagtcccctgccaccctgcaccccaacgccaccctgtgcagaaatgagggaggacagcgcccgagtctatgagaacgtgggcctcatgcagcagcagaggagtttcagatgagcccacggcgacagaacgcagatgtgaactttcacccctttcctaaaatgtcaagactagacgagcgttcccaggacccgtgcgtgcgttcagaagccggcactggctggactgcctcttgagaagcgaagtttggaaccatttgaacagcacgtgcctaattggcacctcctttcctcagctaaggagagactgctctgcgttcttgacaatgctattttcatagaattggttttgaattgtggaagcagctaaattgtgctctgtattttctacattatgggactcaaattctagttatgggcaggattttgtttctttttatgaccttaacagatctgattttttttttctttctctctctttggggaatcatg

SEQ ID NO: 14 - protein, 597 aa, Mus musculus
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIVDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLVDQTSGDQSPLPPCTPTPPCAEMREDSARVYENVGLMQQQRSFR

SEQ ID NO: 15 - DNA, 2236 bp, Rattus norvegicus
cgggctcccgagcggggcctcactcggccccctccatgtgacggcgcctcgtggagcctgagtgagcagcgggtccgtgcggagccggaggcgggaggaacatgacatcccggagatggtttcaccccaatatcactggtgtggaggcagagaatctcctgctgacccgaggagtcgatggcagtttcttagcgaggcccagcaagagtaaccctggagacttcactctgtctgttagaagaaatggagccgttacccacatcaagattcagaacactggggactactatgacctctatggcggggaaaagtttgccaccttgcctgaactggtccagtattacatggagcatcacgggcagctgaaagagaagaatggagatgttattgagctcaagtacccactgaactgtgcagaccctacctctgaaaggtggttccacggtcacttgtctggaaaagaagcagagaagctgctgacagagaaggggaagcatggcagtttcctcgtccgggagagccagagccaccctggggacttcgtcctctccgtccgcactggtgatgacaaaggggagagcaatgacagcaagtccaaagtgacccatgtcatgatccgctgtcaggagctgaaatatgatgttggtggaggagagcgctttgactctttgacagacctggtggagcattacaagaagaaccccatggtggagacactgggcacagtcctgcagctcaaacagcccctcaacacaactcgtattaatgccgctgaaatcgaaagccgggttcgggagttaagcaagctagccgagaccacagataaagtcaaacagggcttttgggaagaatttgagactctacagcaacaggaatgcaaacttctctacagccgaaaagaaggacagagacaagaaaataaaaacaaaaatagatacaaaaacatcctgccctttgatcataccagggttgtcctgcacgatggggatcccaacgagccagtttctgattacatcaatgccaacatcatcatgcctgaatttgaaaccaagtgcaacaattcaaaacccaaaaagagttacattgccactcaaggctgcctgcagaacacggtgaatgacttctggcggatggtgttccaggagaactctcgagtcattgtcatgaccacaaaggaagtggagagagggaagagcaagtgtgtcaagtactggcctgatgagtgtgcactcaaagagtatggcgtcatgcgtgtgaggaacgtcagagaaagtgctgcgcatgactacaccttacgagaactcaaactctctaaggtcggacaaggaaacacagagagaaccgtctggcagtaccactttcggacctggccagaccacggtgtgcctagtgaccctggaggtgtgctggacttcctggaggaggtccaccacaagcaggagagcatcgtggatgcaggccctgtcgtggttcactgcagtgctgggattggccggacaggaacgttcattgtgattgatatccttattgacatcatccgagagaaaggtgtggactgtgacatcgacgttcctaaaaccattcagatggtacggtcccagaggtcagggatggtccagacagaagcacagtaccggttcatctacatggccgtccagcactacatagagacgctgcaacgcaggatcgaggaggagcagaaaagcaaaaggaaaggacatgaatataccaatattaagtattccctggtggaccagacaagtggcgatcagagtcccctgccaccttgcaccccaacgccaccctgtgcggaaatgcgggaggacagcgctcgagtctatgagaacgtgggcctcatgcagcagcagaggagtttcagatgagcttcgggtggcagcgcgcagatgtgaactttcaccccttccctaaaatgtcaagaatagacgagaaagtttccaggacccgtgttcagaagctcgccggggttgactgactgccttttgagaagcgaagtttggaaccatttgaaagagcacgtgcctaattggcacctcctttcctcagctaaggagaaactgctctgcgttgtcgacagtgctattgtcatggaattggttttgaattgtgaagcagctaaattgtgctctgtattttctacattgtgggactcaaattctagtcacgggcaggattttgtttgtttctttttatgaccttaacagacctgttaagaccg

SEQ ID NO: 16 - protein, 593 aa, Rattus norvegicus
MTSRRWFHPNITGVEAENLLLTRGVDGSFLARPSKSNPGDFTLSVRRNGAVTHIKIQNTGDYYDLYGGEKFATLPELVQYYMEHHGQLKEKNGDVIELKYPLNCADPTSERWFHGHLSGKEAEKLLTEKGKHGSFLVRESQSHPGDFVLSVRTGDDKGESNDSKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDECALKEYGVMRVRNVRESAAHDYTLRELKLSKVGQGNTERTVWQYHFRTWPDHGVPSDPGGVLDFLEEVHHKQESIVDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLVDQTSGDQSPLPPCTPTPPCAEMREDSARVYENVGLMQQQRSFR

SEQ ID NO: 17 - DNA, 2287 bp, Homo sapiens
ggggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggtggttatattggcaacctattctcactaagatggggtttatgtcagtcattttggttggcgcttttgttggctggagactgttttttcagcaaaatgccctataaacaattaattttgcccagcaagcttctgcactagtaactgacagtgctacattaatcataggggtttgtctgcagcaaacgcctcatatcccaaaaacggtgcagtagaatagacatcaaccagataagtgatatttacagtcacaagcccaacatctcaggactcttgactgcaggttcctctgaaccccaaactgtaaatggctgtctaaaataaagacattcatgtttgttaaaaactggtaaattttgcaactgtattcatacatgtcaaacacagtatttcacctgaccaacattgagatatcctttatcacaggatttgtttttggaggctatctggattttaacctgcacttgatataagcaataaatattgtggttttatctacgttattggaaagaaaatgacatttaaataatgtgtgtaatgtataatgtactattgacatgggcatcaacacttttattcttaagcatttcagggtaaatatattttataagtatctatttaatcttttgtagttaactgtactttttaagagctcaatttgaaaaatctgttactaaaaaaaaaaattgtatgtcgattgaattgtactggatacattttccatttttctaaaaagaagtttgatatgagcagttagaagttggaataagcaatttctactatatattgcatttcttttatgttttacagttttccccattttaaaaagaaaagcaaacaaagaaacaaaagtttttcctaaaaatatctttgaaggaaaattctccttactgggatagtcaggtaaacagttggtcaagactttgtaaagaaattggtttctgtaaatcccattattgatatgtttatttttcatgaaaatttcaatgtagttggggtagattatgatttaggaagcaaaagtaagaagcagcattttatgattcataatttcagtttactagactgaagttttgaagtaaaccc

SEQ ID NO: 18 - protein, 415 aa, Homo sapiens
MPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRWLYWQPILTKMGFMSVILVGAFVGWRLFFQQNAL

SEQ ID NO: 19 - DNA, 462 bp, Homo sapiens
cggaaacgtattcgagaggacagaaaggccaccacagctcagaaggtgcagcagatgaaacagaggctaaatgagaatgaacgaaaaagaaaaaggccaagattgacagacacctaatattcatgacttaagaatattctgcagctataaattttgaaccattgatgtgcaaagcaagacctgaagcccactccggaaactaaagtgaggctcgctaaccctctagattgcctcacagttgtttgtttacaaagtaaactttacatccaggggatgaagagcacccaccagcagaagactttgcagaacctttaattggatgtgttaagtgtttttaatgagtgtatgaaatgtagaaagatgtacaagaaataaattaggagagattactttgtattgtactgccattcctactgtatttttatactttttggcagcattaaatatttttgttaaatagtc

SEQ ID NO: 20 - DNA, 462 bp, Homo sapiens; identical to SEQ ID NO: 19

SEQ ID NO: 21 - DNA, 1555 bp, Mus musculus
tctccccggatagagcggggcccgagcctgtccgctgtggtagttccgctcggctgccccgccgccatgtcggcaaccatcgagcgggagttcgaggaactggatgctcagtgtcgctggcagccgttatacttggaaattcgaaatgaatcccatgactatcctcatagagtggccaagtttccagaaaacagaaaccgaaacagatacagagatgtaagcccatatgatcacagtcgtgttaaactgcaaagtactgaaaatgattatattaatgccagcttagttgacatagaagaggcacaaagaagttacatcttaacacagggcccacttccgaacacatgctgccatttctggctcatggtgtggcagcaaaagaccaaagcagttgtcatgctaaaccgaactgtagaaaaagaatcggttaaatgtgcacagtactggccaacggatgacagagaaatggtgtttaaggaaacgggattcagtgtgaagctcttatctgaagatgtaaaatcatattatacagtacatctactacagttagaaaatatcaatactggtgaaacgagaaccatatctcacttccattataccacctggccagattttggggttccagagtcaccagcttcatttctaaacttcttgtttaaagttagagaatctggttgtttgacccctgaccatggacctgcagtgatccattgcagtgcgggcatcgggcgctctggcaccttctctcttgtagatacctgtcttgttctgatggaaaaaggagaggatgttaatgtgaaacaattattactgaatatgagaaagtatcgaatgggacttattcagacaccggaccaactcagattctcctacatggccataatagaaggagcaaagtacacaaaaggagattcaaatatacagaaacggtggaaagaactttctaaagaagatttatctcctatttgtgatcattcacagaacagagtgatggttgagaagtacaatgggaagagaataggttcagaagatgaaaagttaacagggcttccttctaaggtgcaggatactgtggaggagagcagtgagagcattctacggaaacgtattcgagaggatagaaaggctacgacggctcagaaggtgcagcagatgaaacagaggctaaatgaaactgaacgaaaaagaaaaaggccaagattgacagacacctaaatgttcatgacttgagactattctgcagctataaaatttgaacctttgatgtgcaaagcaagacctgaagcccactccggaaactaaagtgaggcttgctaaccctgtagattgcctcacaagttgtctgtttacaaagtaagctttccatccaggggatgaagaacgccaccagcagaagacttgcaaaccctttaatttgatgtattgttttttaacatgtgtatgaaatgtagaaagatgtaaaggaaataaattaggagcgactactttgtattgtactgccattcctaatgtatttttatactttttggcagcattaaatatttttattaaatag

SEQ ID NO: 22 - protein, 382 aa, Mus musculus
MSATIEREFEELDAQCRWQPLYLEIRNESHDYPHRVAKFPENRNRNRYRDVSPYDHSRVKLQSTENDYINASLVDIEEAQRSYILTQGPLPNTCCHFWLMVWQQKTKAVVMLNRTVEKESVKCAQYWPTDDREMVFKETGFSVKLLSEDVKSYYTVHLLQLENINTGETRTISHFHYTTWPDFGVPESPASFLNFLFKVRESGCLTPDHGPAVIHCSAGIGRSGTFSLVDTCLVLMEKGEDVNVKQLLLNMRKYRMGLIQTPDQLRFSYMAIIEGAKYTKGDSNIQKRWKELSKEDLSPICDHSQNRVMVEKYNGKRIGSEDEKLTGLPSKVQDTVEESSESILRKRIREDRKATTAQKVQQMKQRLNETERKRKRPRLTDT

SEQ ID NO: 23 - DNA, 1494 bp, Rattus novegicus
ttccgctcgcgctcccccgccgccatgtcggctaccatcgagcgggagttcgaggaactggatgctcagtgtcgctggcagccgttatacttggaaattcgaaatgaatcccatgactatcctcatagagtggccaagtttccagaaaacagaaatcgaaacagatacagagatgtaagcccatatgatcacagtcgtgttaaactgcagagtgctgaaaatgattatattaatgccagcttagttgacatagaagaggcacaaagaagttacatcttaacacagggcccacttcctaacacgtgctgccatttctggctcatggtgtggcagcaaaagaccagagcagttgtcatgctaaaccgaactgtagagaaagaatcggttaaatgtgcacagtactggccaacggatgaccgagagatggtgtttaaggaaacaggattcagcgtgaagctcttatctgaagatgtgaaatcatattatacagtacatctactacagttagaaaatatcaatagtggtgaaaccagaaccatatctcactttcattataccacctggccagattttggcgttccggagtcaccagcttcattcctaaatttcttgtttaaagttagagaatctggttctttgaaccctgaccatgggcctgcagtgatccattgcagtgcaggcatcgggcgttctggcaccttctctcttgtagatacctgtctcgttctgatggagaaaggagaggatgttaatgtgaaacaaatattactgagtatgagaaagtatcgaatgggactcattcagactccggaccagctcagattctcctacatggccataatagaaggagcaaagtatacaaaaggagattcaaatatacagaacagaacaatgactgagaagtacaacgggaagagaatagggtcagaagatgaaaagttaacaggactttcttctaaggttccagatactgtggaagagagcagtgagagtattctccggaaacgcattcgagaggatagaaaggctacaaccgctcagaaggtgcagcagatgagacagaggctaaatgaaactgaacggaaaaggaaaaggccaagattgacagacacctaaatgttcatgacttgagactattctgcagctataaattttgaacctttgatgtgcaaagcaagacctgaagcccactccggaaactaaagtgaggcttgctaaccctgtagattgcctcacaagttgtctgtttacaaagtaagctttacatccaggggatgaagaacgccaccagcagaagacttgcaaaccctttaatttgacgtattgttttttaacatgtgtatgaattgtagaaagatgtaaagaaaataaaattaggagagactactttgtattgtactgccattcctaatgtatttttatactttttggcagcattaaatatttttattaaatagacaaaaaaaaaaaaaaaaaaaaaaaaaaaaaaaaaaa

SEQ ID NO: 24 - protein, 363 aa, Rattus norvegicus
MSATIEREFEELDAQCRWQPLYLEIRNESHDYPHRVAKFPENRNRNRYRDVSPYDHSRVKLQSAENDYINASLVDIEEAQRSYILTQGPLPNTCCHFWLMVWQQKTRAVVMLNRTVEKESVKCAQYWPTDDREMVFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGEDVNVKQILLSMRKYRMGLIQTPDQLRFSYMAIIEGAKYTKGDSNIQNRTMTEKYNGKRIGSEDEKLTGLSSKVPDTVEESSESILRKRIREDRKATTAQKVQQMRQRLNETERKRKRPRLTDT

SEQ ID NO: 25 - DNA, 2543 bp, Homo sapiens
cggccccaggcctggaggggggtctgtgcgcggccggctggctctgccccgcgtccggtcccgagcgggcctccctcgggccagcccgatgtgaccgagcccagcggagcctgagcaaggagcgggtccgtcgcggagccggagggcgggaggaacatgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaaggaagaggaaagggcacgaatatacaaatattaagtatcctctagcggaccagacgagtggagatcagagccctctcccgccttgtactccaacgccaccctgtgcagaaatgagagaagacagtgctagagtctatgaaaacgtgggcctgatgcaacagcagaaaagtttcagatgagaaaacctgccaaaacttcagcacagaaatagatgtggactttcaccctctccctaaaaagatcaagaacagacgcaagaaagtttatgtgaagacagaatttggatttggaaggcttgcaatgtggttgactaccttttgataagcaaaatttgaaaccatttaaagaccactgtattttaactcaacaatacctgcttcccaattactcatttcctcagataagaagaaatcatctctacaatgtagacaacattatattttatagaatttgtttgaaattgaggaagcagttaaattgtgcgctgtattttgcagattatggggattcaaattctagtaataggcttttttatttttatttttatacccttaaccagtttaattttttttttcctcattgttggggatgatgagaagaaatgatttgggaaaattaagtaacaacgacctagaaaagtgagaacaatctcatttaccatcatgtatccagtagtggataattcattttgatggcttctatttttggccaaatgagaataagccagtgcctgagactgtcagaagttgacctttgcactggcattaaagagtcatagaaaaaa

SEQ ID NO: 26 - protein, 593 aa, Homo sapiens [sequence omitted]

SEQ ID NO: 27 - DNA, 2276 bp, Homo sapiens
ctgccccgcgtccggtcccgagcgggcctccctcgggccagcccgatgtgaccgagcccagcggagcctgagcaaggagcgggtccgtcgcggagccggagggcgggaggaacatgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatgagaaaacctgccaaaacttcagcacagaaatagatgtggactttcaccctctccctaaaaagatcaagaacagacgcaagaaagtttatgtgaagacagaatttggatttggaaggcttgcaatgtggttgactaccttttgataagcaaaatttgaaaccatttaaagaccactgtattttaactcaacaatacctgcttcccaattactcatttcctcagataagaagaaatcatctctacaatgtagacaacattatattttatagaatttgtttgaaattgaggaagcagttaaattgtgcgctgtattttgcagattatggggattcaaattctagtaataggcttttttatttttatttttatacccttaaccagg

SEQ ID NO: 28 - protein, 593 aa, Homo sapiens [sequence omitted]

SEQ ID NO: 29 - DNA, 2121 bp, Homo sapiens
cgccaggcctggaggggggtctgtgcgcggccggctggctctgccccgcgtccggtcccgagcgggcctccctcgggccagcccgatgtgaccgagcccagcggagcctgagcaaggagcgggtccgtcgcggagccggagggcgggaggaacatgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatgagaaaacctgccaaaacttcagcacagaaatagatgtggactttcaccctctccctaaaaagatcaagaacagacgcaagaaagtttatgtgaagacagaatttggatttggaaggcttgcaatgtggttgactaccttttgataagcaaaatttgaaaccatttaaagaccactgtattttaactc

SEQ ID NO: 30 - protein, 593 aa, Homo sapiens [sequence omitted]

SEQ ID NO: 31 - DNA, 1980 bp, Homo sapiens
ggcacgagcggctggctctgcccgcgtccggtcccgagcgggcctccctcgggccagcccgatgtgaccgagcccagcggagcctgagcaaggagcgggtccgtcgcggagccggagggcgggaggaacatgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatgagaaaacctgccaaaacttcagcacagaaatagatgtggactttcacctctccctaaaaagatcaggacc

SEQ ID NO: 32 - protein, 593 aa, Homo sapiens [sequence omitted]

SEQ ID NO: 33 - DNA, 2338 bp, Homo sapiens
ggaggagcgagccgggccggggggcagctgcacagtctccgggatccccaggcctggaggggggtctgtgcgcggccggctggctctgccccgcgtccggtcccgagcgggcctccctcgggccagcccgatgtgaccgagcccagcggagcctgagcaaggagcgggtccgtcgcggagccggagggcgggaggaacatgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatgagaaaacctgccaaaacttcagcacagaaatagatgtggactttcaccctctccctaaaaagatcaagaacagacgcaagaaagtttatgtgaagacagaatttggatttgggaaggcttgcaatgtggttgactaccttttgataagcaaaatttgaaaccatttaaagaccactgtattttaactcaacaatacctgcttcccaattactcatttcctcagataagaagaaatcatctctacaatgtagacaacattatattttatagaatttgtttgaaattgaggaagcagttaaattgtgcgctgtattttgcagaggattatggggattcaaattctagtaataggccttttt

SEQ ID NO: 34 - DNA, 274 bp, Homo sapiens
cagtactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgca

SEQ ID NO: 35 - protein, 91 aa, Homo sapiens
QYWPDEYALKEYGVMRVRNVKESAAHDYTLRELKLSKVGQGNTERTVWQYHFRTWPDHGVPSDPGGVLDFLEEVHHKQESIMDAGPVVVHC

SEQ ID NO: 36 - DNA, 90 bp, Homo sapiens
acggagagaacggtctggcaataccactttcggacctggccggaccacggcgtgcccagcgaccctgggggcgtgctggacttcctggag

SEQ ID NO: 37 - DNA, 3984 bp, Homo sapiens
ctgcaggttattcagcgatagttatgacctcccggttacgtgcgttgggtggaagaattaataatatacgcacctcggagttacccaaagagaaaactcgatcagaagtcatttgcagcatccactttttagatggcgtggtacagacctttaaagttactaaacaagacactggccaggttcttctggatatggtgcacaaccacctgggtgtgactgaaaaggaatattttggtttacagcatgatgacgactccgtggactctcctagatggctggaagcaagcaaacccatcaggaagcagttaaaaggaggtttcccctgtaccctgcattttcgagtaagattttttatacctgatcccaacacactgcagcaagaacaaaccaggcacttgtatttcttacaactgaagatggatatttgcgaaggaaggttaacctgccctcttaactcagcagtggttctagcgtcctatgccgtacaatctcattttggagactataattcttccatacatcatccaggctatctttccgatagtcactttatacccgatcaaaatgaggactttttaacaaaagtcgaatctctgcatgagcagcacagtgggctaaaacaatcagaagcagaatcctgctatatcaacatagcgcggaccctcgacttctatggagtagaactgcacagtggtagggatctgcacaatttagacctaatgattggaattgcttccgcgggtgttgctgtgtaccgaaaatacatttgcacaagtttctatccttgggtgaacattctcaaaatttctttcaaaaggaaaaagttcttcatacatcagcgacagaaacaggctgaatccagggaacatattgtggccttcaacatgctgaattaccgatcttgcaaaaacttgtggaaatcctgtgttgagcaccatacgttctttcaggcaaagaagctactacctcaggaaaagaatgttctgtctcagtactggactatgggctctcggaacaccaaaaagtcggtaaataaccaatattgcaaaaaggtgattggcgggatggtgtggaacccagccatgcggagatccttatcagtggagcacttagaaaccaagagtctgccttctcgttcccctcccattactcccaactggcgaagtcctcggctccggcacgaaatccgaaagccacgccactcttctgcagataaccttgcaaatgaaatgacctacatcacggaaacggaagatgtattttacacgtacaagggctctctggcccctcaagacagcgattctgaagtttctcagaaccgaagcccgcaccaagagagtttatccgagaacaatccggcacaaagctacctgacccagaagtcatccagttctgtgtctccatcttcaaatgctccaggctcctgctcacctgacggcgttgatcagcagctcttagatgacttccacagggtgaccaaagggggctccaccgaggacgccagccagtactactgtgacaagaatgataatggtgacagctacttagtcttgatccgtatcacaccagatgaagatggaaaatttggatttaatcttaagggaggagtggatcaaaagatgcctcttgtggtatcaaggataaacccagagtcacctgcggacacctgcattcctaagctgaacgaaggggatcaaatcgtgttaatcaatggccgggacatctcagaacacacgcatgaccaagtggtgatgttcatcaaagccagccgggagtcccactcacgggagctggccctggtgatcaggaggagagctgtccgctcatttgctgacttcaagtctgaagatgaactgaaccagcttttccccgaagccattttccccatgtgtccggagggtggggacactttggagggatccatggcacagctaaagaagggcctcgaaagcgggacggtgctgatccagtttgagcaactctacagaaaaaagccaggtttggccatcacgtttgcaaagctgcctcaaaatttggacaaaaaccgatataaagatgtgctgccttatgacaccacccgggtattattgcagggaaatgaagattatattaatgcaagttacgtgaacatggaaattcctgctgctaaccttgtgaacaagtacatcgccactcaggggcccctgccgcatacctgtgcacagttttggcaggttgtctgggatcagaagttgtcactcattgtcatgttgacgactctcacagaacgagggcggaccaaatgtcaccagtactggccagatccccccgacgtcatgaaccacggcggctttcacatccagtgtcagtcagaggactgcaccatcgcctatgtgtcccgagaaatgctggtcacaaacacccagaccggggaagaacacacagtgacacatctccagtacgtcgcatggcctgaccacggtatacccgatgactcctccgactttctggaatttgtaaactatgtgaggtctctgagagtggacagcgagcctgtcctagttcactgcagtgctggaataggtcgaaccggtgtgttggtcactatggaaacagccatgtgcctaactgagaggaacctgcccatttacccactggatattgtccgaaaaatgcgagaccagcgcgccatgatggtgcagacatcaagccagtacaagtttgtgtgtgaagcgattcttcgtgtgtatgaagaaggtttagtccaaatgctggatcctagttaagacaactgtgaaaaagttcattcctctttcccaagggcatcctccttgaaagaggaggacagacctctctggaagcagcaagaggaaccagtagctgtgggaaaggaatgggcacctctgaacccaggcactttaaacttctatagaaaagatatcgtgtacataggaactggtgtagataagcatgcaattatggcatcatttaggcctgtatttctatggaaagatacaaaaaggatctcagtttggggcctgtcctaatgccttcttccctaacatcaccacacacacccctgtcggcatcctggagcaattgagaccggacacccacagagctgttgtcctcccagcaacaagatggtgtggttatcttgggtcatttggatgttttgtttgtttctgtgtgtcagactgtaagggctgagctttctgtgcttctaggtggagctggaacaattcagattcacccgccctgatgctaaggaaaccctgacgtatgtactagatggcagggcactgggggtcaggctgaaggctgagcaacacctctctgccctccctccctttgtcccatctcccagcgacttccaatattcatgtttctgagaattgtgtccctcttcagttccctcttggtgcctaacctggattagtaatgtgcattcaggtgaattttcagctgaggctctgagaactggtactctcagtgtgttctggtcatcttgtggcttagttgtagaagcaggtgtgtctcttgcctctgcttgcctcctactgcacactcagcacccaggactggaatcaccgactactgaatctcctacatgtattgctgctacttcaagctcctccacttgaaaccttatgattttccaaggggagatgggacagtgtcatctaaatattccgaatgtttggccttctgagaaaagagcttctagtaattgaaccatgggtttcccagcttctggagggttggccgtgggctgtgtacatgtgtgtgcccaggggtgagtgtttctcaggattcctaacgattcaaattaccgttgagtatatataaagaatcgagtctctgtatggaagaacaaatgtgtgcattcacccccagtcacaatggtctccattgcatttcaaaggagaggatcagactatctgaatataaacacaatctgatgttaatttattctaagaacaccatcattttgattgtcctaaa

SEQ ID NO: 38 - protein, 913 aa, Homo sapiens [sequence omitted]

SEQ ID NO: 39 - DNA, 2111 bp, Homo sapiens
agcagcacagtgggctaaaacaatcagaagcagaatcctgctatatcaacatagcgcggaccctcgacttctatggagtagaactgcacagtggtagggatctgcacaatttagacctaatgattggaattgcttccgcgggtgttgctgtgtaccgaaaatacatttgcacaagtttctatccttgggtgaacattctcaaaatttctttcaaaaggaaaaagttcttcatacatcagcgacagaaacaggctgaatccagggaacatattgtggccttcaacatgctgaattaccgatcttgcaaaaacttgtggaaatcctgtgttgagcaccatacgttctttcaggcaaagaagctactacctcaggaaaagaatgttctgtctcagtactggactatgggctctcggaacaccaaaaagtcggtaaataaccaatattgcaaaaaggtgattggcgggatggtgtggaacccagccatgcggagatccttatcagtggagcacttagaaaccaagagtctgccttctcgttcccctcccattactcccaactggcgaagtcctcggctccggcacgaaatccgaaagccacgccactcttctgcagataaccttgcaaatgaaatgacctacatcacggaaacggaagatgtattttacacgtacaagggctctctggcccctcaagacagcgattctgaagtttctcagaaccgaagcccgcaccaagagagtttatccgagaacaatccggcacaaagctacctgacccagaagtcatccagttctgtgtctccatcttcaaatgctccaggctcctgctcacctgacggcgttgatcagcagctcttagatgacttccacagggtgaccaaagggggctccaccgaggacgccagccagtactactgtgacaagaatgataatggtgacagctacttagtcttgatccgtatcacaccagatgaagatggaaaatttggatttaatcttaagggaggagtggatcaaaagatgcctcttgtggtatcaaggataaacccagagtcacctgcggacacctgcattcctaagctgaacgaaggggatcaaatcgtgttaatcaatggccgggacatctcagaacacacgcatgaccaagtggtgatgttcatcaaagccagccgggagtcccactcacgggagctggccctggtgatcaggaggagagctgtccgctcatttgctgacttcaagtctgaagatgaactgaaccagcttttccccgaagccattttccccatgtgtccggagggtggggacactttggagggatccatggcacagctaaagaagggcctcgaaagcgggacggtgctgatccagtttgagcaactctacagaaaaaagccaggtttggccatcacgtttgcaaagctgcctcaaaatttggacaaaaaccgatataaagatgtgctgccttatgacaccacccgggtattattgcagggaaatgaagattatattaatgcaagttacgtgaacatggaaattcctgctgctaaccttgtgaacaagtacatcgccactcaggggcccctgccgcatacctgtgcacagttttggcaggttgtctgggatcagaagttgtcactcattgtcatgttgacgactctcacagaacgagggcggaccaaatgtcaccagtactggccagatccccccgacgtcatgaaccacggcggctttcacatccagtgtcagtcagaggactgcaccatcgcctatgtgtcccgagaaatgctggtcacaaacacccagaccggggaagaacacacagtgacacatctccagtacgtcgcatggcctgaccacggtatacccgatgactcctccgactttctggaatttgtaaactatgtgaggtctctgagagtggacagcgagcctgtcctagttcactgcagtgctggaataggtcgaaccggtgtgttggtcactatggaaacagccatgtgcctaactgagaggaacctgcccatttacccactggatattgtccgaaaaatgcgagaccagcgcgccatgatggtgcagacatcaagccagtacaagtttgtgtgtgaa

SEQ ID NO: 40 - protein, 703 aa, Homo sapiens
QHSGLKQSEAESCYINIARTLDFYGVELHSGRDLHNLDLMIGIASAGVAVYRKYICTSFYPWVNILKISFKRKKFFIHQRQKQAESREHIVAFNMLNYRSCKNLWKSCVEHHTFFQAKKLLPQEKNVLSQYWTMGSRNTKKSVNNQYCKKVIGGMVWNPAMRRSLSVEHLETKSLPSRSPPITPNWRSPRLRHEIRKPRHSSADNLANEMTYITETEDVFYTYKGSLAPQDSDSEVSQNRSPHQESLSENNPAQSYLTQKSSSSVSPSSNAPGSCSPDGVDQQLLDDFHRVTKGGSTEDASQYYCDKNDNGDSYLVLIRITPDEDGKFGFNLKGGVDQKMPLVVSRINPESPADTCIPKLNEGDQIVLINGRDISEHTHDQVVMFIKASRESHSRELALVIRRRAVRSFADFKSEDELNQLFPEAIFPMCPEGGDTLEGSMAQLKKGLESGTVLIQFEQLYRKKPGLAITFAKLPQNLDKNRYKDVLPYDTTRVLLQGNEDYINASYVNMEIPAANLVNKYIATQGPLPHTCAQFWQVVWDQKLSLIVMLTTLTERGRTKCHQYWPDPPDVMNHGGFHIQCQSEDCTIAYVSREMLVTNTQTGEEHTVTHLQYVAWPDHGIPDDSSDFLEFVNYVRSLRVDSEPVLVHCSAGIGRTGVLVTMETAMCLTERNLPIYPLDIVRKMRDQRAMMVQTSSQYKFVCE

SEQ ID NO: 41 - DNA, 5117 bp, Homo sapiens
ccccagccgcatgacgcgcggaggaggcagcgggacgagcgcgggagccgggaccgggtagccgcgcgctgggggtgggcgccgctcgctccgccccgcgaagcccctgcgcgctcagggacgcggcccccccgcggcagccgcgctaggctccggcgtgtggccgcggccgccgccgcgctgccatgtctccgggcaagccggggcgggcggagcggggacgaggcggaccggctggcggaggaggaggcgaaggagacggcaggaggcggcgacgacggtgcccgggctcgggcgcacggcggggcccgattcgcgcgtccggggcacgttccagggcgcgcggggcatgaagccggcggcgcgggaggcgcggctgcctccgcgctcgcccgggctgcgctgggcgctgccgctgctgctgctgctgctgcgcctgggccagatcctgtgcgcaggtggcacccctagtccaattcctgacccttcagtagcaactgttgccacaggggaaaatggcataacgcagatcagcagtacagcagaatcctttcataaacagaatggaactggaacacctcaggtggaaacaaacaccagtgaggatggtgaaagctctggagccaacgatagtttaagaacacctgaacaaggatctaatgggactgatggggcatctcaaaaaactcccagtagcactgggcccagtcctgtgtttgacattaaagctgtttccatcagtccaaccaatgtgatcttaacttggaaaagtaatgacacagctgcttctgagtacaagtatgtagtaaagcataagatggaaaatgagaagacaattactgttgtgcatcaaccatggtgtaacatcacaggcttacgtccagcgacttcatatgtattctccatcactccaggaataggcaatgagacttggggagatcccagagtcataaaagtcatcacagagccgatcccagtttctgatctccgtgttgccctcacgggtgtgaggaaggctgctctctcctggagcaatggcaatggcaccgcctcctgccgggttcttcttgaaagcattggaagccatgaggagttgactcaagactcaagacttcaggtcaatatctcggacctgaagccaggggttcaatacaacatcaacccgtatcttctacaatcaaataagacaaagggagaccccttgggcacagaaggtggcttggatgccagcaatacagagagaagccgggcagggagccccaccgcccctgtgcatgatgagtccctcgtgggacctgtggacccatcctccggccagcagtcccgagacacggaagtcctgcttgtcgggttagagcctggcacccgatacaatgccaccgtttattcccaagcagcgaatggcacagaaggacagccccaggccatagagttcaggacaaatgctattcaggtttttgacgtcaccgctgtgaacatcagtgccacaagcctgaccctgatctggaaagtcagcgataacgagtcgtcatctaactatacctacaagatacatgtggcgggggagacagattcttccaatctcaacgtcagtgagcctcgcgctgtcatccccggactccgctccagcaccttctacaacatcacagtgtgtcctgtcctaggtgacatcgagggcacgccgggcttcctccaagtgcacaccccccctgttccagtttctgacttccgagtgacagtggtcagcacgacggagatcggcttagcatggagcagccatgatgcagaatcatttcagatgcatatcacacaggagggagctggcaattctcgggtagaaataaccaccaaccaaagtattatcattggtggcttgttccctggaaccaagtattgctttgaaatagttccaaaaggaccaaatgggactgaaggggcatctcggacagtttgcaatagaactgttcccagtgcagtgtttgacatccacgtggtctacgtcaccaccacggagatgtggctggactggaagagccctgacggtgcttccgagtatgtctaccatttagtcatagagtccaagcatggctctaaccacacaagcacgtatgacaaagcgattactctccagggcctgattccgggcaccttatataacatcaccatctctccagaagtggaccacgtctggggggaccccaactccactgcacagtacacacggcccagcaatgtgtccaacattgatgtaagtaccaacaccacagcagcaactttaagttggcagaactttgatgacgcctctcccacgtactcctactgccttcttattgagaaggctggaaattccagcaacgcaacacaagtagtcacggacattggaattactgacgctacagtcactgaattaatacctggctcatcatacacagtggagatctttgcacaagtaggggatgggatcaagtcactggaacctggccggaagtcattctgtacagatcctgcgtccatggcctccttcgactgcgaagtggtccccaaagagccagccctggttctcaaatggacctgccctcctggcgccaatgcaggctttgagctggaggtcagcagtggagcctggaacaatgcgacccacctggagagctgctcctctgagaatggcactgagtatagaacggaagtcacgtatttgaatttttctacctcgtacaacatcagcatcaccactgtgtcctgtggaaagatggcagcccccacccggaacacctgcactactggcatcacagatccccctcctccagatggatcccctaatattacatctgtcagtcacaattcagtaaaggtcaagttcagtggatttgaagccagccacggacccatcaaagcctatgctgtcattctcaccaccggggaagctggtcacccttctgcagatgtcctgaaatacacgtatgacgatttcaaaaagggagcctcagatacttatgtgacatacctcataagaacagaagaaaagggacgttctcagagcttgtctgaagttttgaaatatgaaattgacgttgggaatgagtcaaccacacttggttattacaatgggaagctggaacctctgggctcctaccgggcttgtgtggctggcttcaccaacattaccttccaccctcaaaacaaggggctcattgatggggctgagagctatgtgtccttcagtcgctactcagatgctgtttccttgccccaggatccaggtgtcatctgtggagcggtttttggctgtatctttggtgccctggttattgtgactgtgggaggcttcatcttctggagaaagaagaggaaagatgcaaagaataatgaagtgtccttttctcaaattaaacctaaaaaatctaagttaatcagagtggagaattttgaggcctacttcaagaagcagcaagctgactccaactgtgggttcgcagaggaatacgaagatctgaagcttgttggaattagtcaacctaaatatgcagcagaactggctgagaatagaggaaagaatcgctataataatgttctgccctatgatatttcccgtgtcaaactttcggtccagacccattcaacggatgactacatcaatgccaactacatgcctggctaccactccaagaaagattttattgccacacaaggacctttaccgaacactttgaaagatttttggcgtatggtttgggagaaaaatgtatatgccatcattatgttgactaaatgtgttgaacagggaagaaccaaatgtgaggagtattggccctccaagcaggctcaggactatggagacataactgtggcaatgacatcagaaattgttcttccggaatggaccatcagagatttcacagtgaaaaatatccagacaagtgagagtcaccctctgagacagttccatttcacctcctggccagaccacggtgttcccgacaccactgacctgctcatcaacttccggtacctcgttcgtgactacatgaagcagagtcctcccgaatcgccgattctggtgcattgcagtgctggggtcggaaggacgggcactttcattgccattgatcgtctcatctaccagatagagaatgagaacaccgtggatgtgtatgggattgtgtatgaccttcgaatgcataggcctttaatggtgcagacagaggaccagtatgttttcctcaatcagtgtgttttggatattgtcagatcccagaaagactcaaaagtagatcttatctaccagaacacaactgcaatgacaatctatgaaaaccttgcgcccgtgaccacatttggaaagaccaatggttacatcgcctaattccaaaggaataacctttctggagtgaaccagaccgtcgcacccacagcgaaggcacatgccccgatgtcgacatgtttttatatgtctaatatcttaattctttgttctgttttgtgagaactaattttgagggcatgaagctgcatatgatagatgacaaattggggctgtcgggggctgtggatgggtggggagcaaatcatctgcattcctgatgaccaatgggatgaggtcacttttttttttttcccccttgaggattgcggaaaaccaggaaaagggatctatgatttttttttccaaaacaatttcttttttaaaaagactattttatatgattcacatgctaaagccaggattgtgttgggttgaatatattttaagtatcagaggtctatttttacctactgtgtcttggaatctagccgatggaaaatacctaattgtggatgatgattgcgcagggaggggtacgtggcacctcttccgaatgggttttctatttgaacatgtgccttttctgaattatgcttccacaggcaaaactcagtagagatctatatttttgtactgaatctcataattggaatatacggaatatttaaacagtagcttagcatcagaggtttgcttcctcagtaacatttctgttctcatttgatcaggggaggcctctttgccccggccccgcttcccctgcccccgtgtgatttgtgctccattttttcttcccttttccctcccagttttc

SEQ ID NO: 42 - protein, 1337 aa, Homo sapiens [sequence omitted]

SEQ ID NO: 43 - DNA, 4756 bp, Homo sapiens
gcatgacgcgcggaggaggcagcgggagcagccgcgggagccgggaccgggtagccgcgcgctgggggtgggcgccgctcgctccgccccgcgaagcccctgcgcgctcagggacgcggcccccccgcggcagccgcgctaggctccggcgtgtggccgcggccgccgccgccgctgccatgtctccggggaagcccggggcgggcggagcggggacgaggcggaccggctggcggaggaggaggcgaaggagacggcaggaggcggcgacgacggtgcccgggctcgggcgcacggcggggcccgattcgcgcgtccggggcacgttccagggcgcgcggggcatgaagccggcggcgcgggaggcgcggctgcctccgcgctcgcccgggctgcgctgggcgctgccgctgctgctgctgctgctgcgcctgggccagatcctgtgcgcaggtggcacccctagtccaattcctgacccttcagtagcaactgttgccacaggggaaaatggcataacgcagatcagcagtacagcagaatcctttcataaacagaatggaactggaacacctcaggtggaaacaaacaccagtgaggatggtgaaagctctggagccaacgatagtttaagaacacctgaacaaggatctaatgggactgatggggcatctcaaaaaactcccagtagcactgggcccagtcctgtgtttgacattaaagctgtttccatcagtccaaccaatgtgatcttaacttggaaaagtaatgacacagctgcttctgagtacaagtatgtagtaaagcataagatggaaaatgagaagacaattactgttgtgcatcaaccatggtgtaacatcacaggcttacgtccagcgacttcatatgtattctccatcactccaggaataggcaatgagacttggggagatcccagagtcataaaagtcatcacagaaccgatcccagtttctgatctccgtgttgccctcacgggtgtgaggaaggctgctctctcctggagcaatggcaatggcactgcctcctgccgggttcttcttgaaagcattggaagccatgaggagttgactcaagactcaagacttcaggtcaatatctcgggcctgaagccaggggttcaatacaacatcaacccgtatcttctacaatcaaataagacaaagggagaccccttgggcacagaaggtggcttggatgccagcaatacagagagaagccgggcagggagccccaccgcccctgtgcatgatgagtccctcgtgggacctgtggacccatcctccggccagcagtcccgagacacggaagtcctgcttgtcgggttagagcctggcacccgatacaatgccaccgtttattcccaagcagcgaatggcacagaaggacagccccaggccatagagttcaggacaaatgctattcaggtttttgacgtcaccgctgtgaacatcagtgccacaagcctgaccctgatctggaaagtcagcgataacgagtcgtcatctaactatacctacaagatacatgtggcgggggagacagattcttccaatctcaacgtcagtgagcctcgcgctgtcatccccggactccgctccagcaccttctacaacatcacagtgtgtcctgtcctaggtgacatcgagggcacgccgggcttcctccaagtgcacaccccccctgttccagtttctgacttccgagtgacagtggtcagcacgacggagatcggcttagcatggagcagccatgatgcagaatcatttcagatgcatatcacacaggagggagctggcaattctcgggtagaaataaccaccaaccaaagtattatcattggtggcttgttccctggaaccaagtattgctttgaaatagttccaaaaggaccaaatgggactgaaggggcatctcggacagtttgcaatagaactgttcccagtgcagtgtttgacatccacgtggtctacgtcaccaccacggagatgtggctggactggaagagccctgacggtgcttccgagtatgtctaccatttagtcatagagtccaagcatggctctaaccacacaagcacgtatgacaaagcgattactctccagggcctgattccgggcaccttatataacatcaccatctctccagaagtggaccacgtctggggggaccccaactccactgcacagtacacacggcccagcaatgtgtccaacattgatgtaagtaccaacaccacagcagcaactttaagttggcagaactttgatgacgcctctcccacgtactcctactgccttcttattgagaaggctggaaattccagcaacgcaacacaagtagtcacggacattggaattactgacgctacagtcactgaattaatacctggctcatcatacacagtggagatctttgcacaagtaggggatgggatcaagtcactggaacctggccggaagtcattctgtacagatcctgcgtccatggcctccttcgactgcgaagtggtccccaaagagccagccctggttctcaaatggacctgccctcctggcgccaatgcaggctttgagctggaggtcagcagtggagcctggaacaatgcgacccacctggagagctgctcctctgagaatggcactgagtatagaacggaagtcacgtatttgaatttttctacctcgtacaacatcagcatcaccactgtgtcctgtggaaagatggcagcccccacccggaacacctgcactactggcatcacagatccccctcctccagatggatcccctaatattacatctgtcagtcacaattcagtaaaggtcaagttcagtggatttgaagccagccacggacccatcaaagcctatgctgtcattctcaccaccggggaagctggtcacccttctgcagatgtcctgaaatacacgtatgacgatttcaaaaagggagcctcagatacttatgtgacatacctcataagaacagaagaaaagggacgttctcagagcttgtctgaagttttgaaatatgaaattgacgttgggaatgagtcaaccacacttggttatttacaatgggaagctggaacctctgggctcctaccggcttgtgtggctggcttcaccaacattaccttccaccctcaaaacaaggggctcattgatggggctgagagctatgtgtccttcagtcgctactcagatgctgtttccttgccccaggatccaggtgtcatctgtggagcggtttttggctgtatctttggtgccctggttattgtgactgtgggaggcttcatcttctggagaaagaagaggaaagatgcaaagaataatgaagtgtccttttctcaaattaaacctaaaaaatctaagttaatcagagtggagaattttgaggcctacttcaagaagcagcaagctgactccaactgtgggttcgcagaggaatacgaagatctgaagcttgttggaattagtcaacctaaatatgcagcagaactggctgagaatagaggaaagaatcgctataataatgttctgccctatgatatttcccgtgtcaaactttcggtccagacccattcaacggatgactacatcaatgccaactacatgcctggctaccactccaagaaagattttattgccacacaaggacctttaccgaacactttgaaagatttttggcgtatggtttgggagaaaaatgtatatgccatcattatgttgactaaatgtgttgaacagggaagaaccaaatgtgaggagtattggccctccaagcaggctcaggactatggagacataactgtggcaatgacatcagaaattgttcttccggaatggaccatcagagatttcacagtgaaaaatatccagacaagtgagagtcaccctctgagacagttccatttcacctcctggccagaccacggtgttcccgacaccactgacctgctcatcaacttccggtacctcgttcgtgactacatgaagcagagtcctcccgaatcgccgattctggtgcattgcagtgctggggtcggaaggacgggcactttcattgccattgatcgtctcatctaccagatagagaatgagaacaccgtggatgtgtatgggattgtgtatgaccttcgaatgcataggcctttaatggtgcagacagaggaccagtatgttttcctcaatcagtgtgttttggatattgtcagatcccagaaagactcaaaagtagatcttatctaccagaacacaactgcaatgacaatctatgaaaaccttgcgcccgtgaccacatttggaaagaccaatggttacatcgcctaattccaaaggaataacctttctggagtgaaccagaccgtcgcacccacagcgaaggcacatgcccgatgtcgacatgttttatatgctaatatcttaattctttgttctgttttgtgagaactaattttgagggcatgaagctgcatatcatagatgacaaattggggctgtcgggggctgtggatgggtggggagcaaatcatctgcattcctgatgaccaatgggatgaggtcactttttttttttttcccccttcgaaggattgtggaaaaccaggaaaagggagctatgatttttttttccaaaacaatttcttttttaaaaaagactatttatatgattcacatgctaaagccaggattgtgttgggttgaatatattttaagtatcagaggtc

SEQ ID NO: 44 - protein, 1337 aa, Homo sapiens
MKPAAREARLPPRSPGLRWALPLLLLLLRLGQILCAGGTPSPIPDPSVATVATGENGITQISSTAESFHKQNGTGTPQVETNTSEDGESSGANDSLRTPEQGSNGTDGASQKTPSSTGPSPVFDIKAVSISPTNVILTWKSNDTAASEYKYVVKHKMENEKTITVVHQPWCNITGLRPATSYVFSITPGIGNETWGDPRVIKVITEPIPVSDLRVALTGVRKAALSWSNGNGTASCRVLLESIGSHEELTQDSRLQVNISGLKPGVQYNINPYLLQSNKTKGDPLGTEGGLDASNTERSRAGSPTAPVHDESLVGPVDPSSGQQSRDTEVLLVGLEPGTRYNATVYSQAANGTEGQPQAIEFRTNAIQVFDVTAVNISATSLTLIWKVSDNESSSNYTYKIHVAGETDSSNLNVSEPRAVIPGLRSSTFYNITVCPVLGDIEGTPGFLQVHTPPVPVSDFRVTVVSTTEIGLAWSSHDAESFQMHITQEGAGNSRVEITTNQSIIIGGLFPGTKYCFEIVPKGPNGTEGASRTVCNRTVPSAVFDIHVVYVTTTEMWLDWKSPDGASEYVYHLVIESKHGSNHTSTYDKAITLQGLIPGTLYNITISPEVDHVWGDPNSTAQYTRPSNVSNIDVSTNTTAATLSWQNFDDASPTYSYCLLIEKAGNSSNATQVVTDIGITDATVTELIPGSSYTVEIFAQVGDGIKSLEPGRKSFCTDPASMASFDCEVVPKEPALVLKWTCPPGANAGFELEVSSGAWNNATHLESCSSENGTEYRTEVTYLNFSTSYNISITTVSCGKMAAPTRNTCTTGITDPPPPDGSPNITSVSHNSVKVKFSGFEASHGPIKAYAVILTTGEAGHPSADVLKYTYDDFKKGASDTYVTYLIRTEEKGRSQSLSEVLKYEIDVGNESTTLGYLQWEAGTSGLLPACVAGFTNITFHPQNKGLIDGAESYVSFSRYSDAVSLPQDPGVICGAVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 45 - protein, 122 aa, Homo sapiens [sequence omitted]

SEQ ID NO: 46 - DNA, 4426 bp, Mus musculus
ccgttgctgtcgccgttgctgtcggacgcgcggaggaggcagagggagcagccggggccgcgggagccgggagctgggagccacgcgcggagggtgggcgccgctcgctccgccccgcgaagccctgcgagtctaaggccgcggccgctccgcgccaggcgcgctaggctccgcgtgtggccgccgccgccgctgccatgtccccggggaagcccggggcgggcgacgggggactaggcggaccggctggcggagaaggaggcggaggcgtcggctggagacggagacgagggcgcccggcttcgggcacacggcggggcgcgtcccgggcacgttccagggcgcgcagggcatgaagcccgcggcgcgggagacgcggacacccccgcgctcgcccgggctccgctgggcgctgctgccgctgctgctgttgctacgccagggccaggtcctgtgcgcaggtgcggcaccgaatcctatatttgacattgaagctgtcgtcagcccaactagtgtgttattaacatggaagcacaatgactcaggcgcttcagaatgtagaatagagaataagatggagagcaatctgacgtttcctgttaaaaaccagacatcatgtaacattacaggcttaagcccaggcacttcgtatacattctccatcatctctgtaacaaccaatgagaccttgaacaaaactatcacaacagagccctggccagtgtctgatctccatgtcacctctgtgggtgtgacacaggctcgtctcacctggagcaatgcaaatggcactgcctcctaccggatgctgattgaagagttgaccacacattcctcagtcaatatttcaggtctgaagccggggaccaataatacgttcgctttcccagaatcaaatgagacacaggctgactttgcagttgcagaggaggtcccggatgccaatggtaccaagagaatcccagtgaccaacctatcccaactacacaagaattctcttgtctctgtggacccaccctctggccaggatccctccctcacagagatcttgcttactgacctaaagcctgatactcagtacaatgccaccatctattctcaagcagcaaatggcactgaaggacagcccaggaacaaagtgtttaaaacaaattccacccaggtttctgacgtccgagctatgaacatcagtgcctcaagcatgaccctgacctggaaaagcaattacgatgggtcccgtacttcaattgtctacaaaatacacgtggctggggggacccactccgtcaaccaaactgtcaataagactgaggccatcatcctcggactcagctccagcaccttgtacaacatcacagttcatcctttcctgggtcagacggagggcacaccaggcttcctccaagtgtacacttcccccgatcaggtctctgacttccgagtgacaaatgtcagcacaagggcaattggtttggcttggaggagcaatgactccaagtccttcgagattttcatcaagcaggacggaggtgagaagcatcgaaatgcttcgacgggaaaccagagctatatggttgaagatttaaagcctggaaccagttaccattttgagataattccacgaggaccagacgggacagaagggctgtccagtacagtgaatgggagcactgaccccagtgctgtgactgacatccgggtggtcaacattagcaccactgaaatgcagttggagtggcagaatacggacgatgcctctggatacacttaccatttagttctagagtctaaaagtggctccatcatcaggaccaacagttctcagaagtggatcacagtagggagcctcaccccaggcaccttatacaatgtcacaatctttccagaagtggaccagatccagggaatctccaactccattacccagtacacacggcccagcagtgtgtcccacattgaagtaaacaccaccaccaccacggcagccatccgatggaagaacgaggacgcagcctctgcttcctatgcctactccgtccttatcttgaagactggagatggcagcaatgtaaccagcaacttcacaaaagacccttctattctaatccctgagttaatccctggtgtctcttacacagtgaagatccttacacaagttggggatggtacaacatcactggtacctggttggaatctgttctgtacggaacctgaaccagtgacctccttccactgtgaagtggtccctaaggagccagcattggttctcaagtgggcctgcccctttggcatgtacacaggcttcgagctgggggtcaggagtgattcctgggacaatatgacacgcctagagaactgcacttcggatgatgacacagagtgcaggacggaagtcgcctatttgaatttttctacctcgtacaacatcagcatcgccaccttgtcatgtgggaagatggcgcttcccgcccagaacatctgcaccactggcatcacagacccacctactccggatggatcccctaatattacatcggtcagtcacaattcagtaaaggttaagttcagcgggtttgaagccagccacggacctatcaaagcctatgctgtcatcctcaccaccggggaagctgcccaaccttctgcagatgttttgaagtacacgtatgaggatttcaaaaggggagcctcggatacttatgtcacatacctcataagaatagaagagaagggacagtctcagggcttgtctgaagtcttgaactatgaaattgatgtggggaaccaatccactaccctcggctactacaacgggaggctggagcctctgggctcctaccgggcttgtgttgctggctttaccaatattacctacaaccttcagaatgacggcctcatcaatggggatgagagctatgtgtctttcagtccatattcagaggccgtgttcttgccccaggacccaggtgtcatctgcggagcagtgtttggatgtatctttggtgccctggccatcacagctgtgggaggcttcatcttctggagaaagaaaaggacagatgccaagaataatgaagtgtccttttctcaaattaaacctaaaaaatccaagttaatccgagtggagaattttgaggcctactttaagaagcagcaagctgactctaactgtgggtttgcagaggaatatgaggacctgaagctgattgggataagtttacctaaatacacagctgagatagccgagaacagagggaagaaccgctacaacaatgttctgccctatgatatttctcgagtcaaactttcagtccagacccattcgacagatgactacatcaatgccaactatatgcctggctaccattccaagaaagatttcattgccacacaaggacctttacccaacactttgaaagatttctggcgtatggtttgggagaaaaacgtatatgccattgttatgttgaccaaatgcgtggagcagggaaggaccaaatgtgaggagtactggccttccaagcaggctcaggactacggggacataactgtggcgatgacatcagaagtcgttcttccagaatggaccatcagagattttgtggtgaaaaatatgcagaatagcgagagccatcctctgcggcagttccatttcacctcctggcctgaccacggtgttcctgacaccactgacctgctcatcaactttcggtacctggtccgggattacatgaagcagataccccccgagtcaccaattctggtgcattgcagtgctggggttggaaggacgggcactttcatcgccatcgatcgcctgatctatcagatagagaatgagaacaccgtggacgtgtatgggattgtctatgaccttcggatgcacaggcctctgatggtgcagacagaggaccagtatgttttcctcaatcagtgtgttttggatattatcagagcccagaaagactcaaaagttgatctcatctatcagaacacaacggcaatgacaatctatgaaaacctcgagccagtgagcatgtttggaaagactaatggttacatcgcctagttccaaagcaaacccttttctgaagctaaccggaccgtcacacccacagtgaaggagagcgccctgacgggacatgttttatatgctcctttctcagttctttgttctgttctgttagaactacttggagggcatggggctgtgcgtgcaaaagcatggcagatagccagtggggtgtcaggggctgtggacgggtggtgtggaaatcagctgccttcctgatcactaatgggatgggtcactttttgtttttttccttgagaattgtggaaaaccaggaaaagtgagctatgttttgttttccaaactggtttgttctcctcttttctccccttgtctcccccctctccctccccc

SEQ ID NO: 47 - protein, 1238 aa, Mus musculus
MKPAARETRTPPRSPGLRWALLPLLLLLRQGQVLCAGAAPNPIFDIEAVVSPTSVLLTWKHNDSGASECRIENKMESNLTFPVKNQTSCNITGLSPGTSYTFSIISVTTNETLNKTITTEPWPVSDLHVTSVGVTQARLTWSNANGTASYRMLIEELTTHSSVNISGLKPGTNNTFAFPESNETQADFAVAEEVPDANGTKRIPVTNLSQLHKNSLVSVDPPSGQDPSLTEILLTDLKPDTQYNATIYSQAANGTEGQPRNKVFKTNSTQVSDVRAMNISASSMTLTWKSNYDGSRTSIVYKIHVAGGTHSVNQTVNKTEAIILGLSSSTLYNITVHPFLGQTEGTPGFLQVYTSPDQVSDFRVTNVSTRAIGLAWRSNDSKSFEIFIKQDGGEKHRNASTGNQSYMVEDLKPGTSYHFEIIPRGPDGTEGLSSTVNGSTDPSAVTDIRVVNISTTEMQLEWQNTDDASGYTYHLVLESKSGSIIRTNSSQKWITVGSLTPGTLYNVTIFPEVDQIQGISNSITQYTRPSSVSHIEVNTTTTTAAIRWKNEDAASASYAYSVLILKTGDGSNVTSNFTKDPSILIPELIPGVSYTVKILTQVGDGTTSLVPGWNLFCTEPEPVTSFHCEVVPKEPALVLKWACPFGMYTGFELGVRSDSWDNMTRLENCTSDDDTECRTEVAYLNFSTSYNISIATLSCGKMALPAQNICTTGITDPPTPDGSPNITSVSHNSVKVKFSGFEASHGPIKAYAVILTTGEAAQPSADVLKYTYEDFKRGASDTYVTYLIRIEEKGQSQGLSEVLNYEIDVGNQSTTLGYYNGRLEPLGSYRACVAGFTNITYNLQNDGLINGDESYVSFSPYSEAVFLPQDPGVICGAVFGCIFGALAITAVGGFIFWRKKRTDAKNNEVSFSQIKPKKSKLIRVENFEAYFKKQQADSNCGFAEEYEDLKLIGISLPKYTAEIAENRGKNRYNNVLPYDISRVKLSVQTHSTDDYINANYMPGYHSKKDFIATQGPLPNTLKDFWRMVWEKNVYAIVMLTKCVEQGRTKCEEYWPSKQAQDYGDITVAMTSEVVLPEWTIRDFVVKNMQNSESHPLRQFHFTSWPDHGVPDTTDLLINFRYLVRDYMKQIPPESPILVHCSAGVGRTGTFIAIDRLIYQIENENTVDVYGIVYDLRMHRPLMVQTEDQYVFLNQCVLDIIRAQKDSKVDLIYQNTTAMTIYENLEPVSMFGKTNGYIA

SEQ ID NO: 48 - DNA, 6314 bp, Rattus norvegicus`
ggaaacccgggacggacggagcggggactaggcggaccgactggcggagaaggaggcggaggcgtcgactggagacggagaccagggcgcccggcttcgggcacacggcggggcgcgtcccgggcacgttccagggcgcgcagggcatgaagcccgcggcgcgggagacgcggacacccccgcgctcgcccgggctccgctgggcgctgctgccgctgctgctgttgctacgccagggccaggttgtgtgcacaggtgcggcgcccagtcctgtatttgacgttgaagctgtcaccagccctaccagtgtggtattaacgtggaaacacaatgattcagccacttcagaatataagataaatgagggaaacacgctgcggtatactgttaaaaaccagacatcatttaacatcacaggcttaagcccagctacttcctacaaattctccatcaccttgggaacagtcaatgagacctcaggaaagcccacatacaaaaatatcacaacagagccctggccagtgtcggatctccaagtcgcctacatcggtgtgacacaggctcttctcgcctggagcaatgcaaacggcaccgcttcttaccggatgcagattgtagagctgaccacaaattcatcaggtggtatttcagatctgaagccggggacccataagagcttggctgtccaaggatcgaatgagacccagcatgacttatgggtcacagaaggcgtctcggacccaccctctgcccgggatccctccctcacagaggtcttgctcactgagctaaagcctgatactcagtacaaagtcaccatttattctcaagcagcagatggcacagaaggacagcctgggaacaaagtgtttaaaacaaatcccatccaagtttctgacatccgagctgtgaacatcagtgactcgaacatgaccctgacctggaaaagcaacaataatgagtcccatgcttcatttacctacaaaatatacgtggctgggggttccgactccatcaacgaaactgtcaatgagactcaggccgtcatccgtggactcagctccagtaccttgtacaacatcacagtgcttcctttcctgggtcagaccgcgggtataccaggcttcctccaagtgtacacttccccccgtcctgtttctgacttccgagtgacaaatgtcagcctgagggaaatcggtttggcttggagaagcaatgactcggagtcattcgagattttcatcacgcaggagggaagtgagaaacgttggaatgcttcgacgggagacctgagctatatcgttgtaaatttaaagcctgggaccagttaccaatttgaaatattcccacgaggaccaaatgggactgaagggccatcccagacagttgttggtagaactgactgcagtgctgtgactgacatccgcgtggtcagcgttagcaccactgaaatacagctggagtggcagaatacagacagtgcttctggatacacttaccatttagttctagagtctgagaatggctccatcaagaccaacagttctcagaaatggatcacagtggggggccttaccccaggcaccttatacaatgttacaatctttccagaagtggaccagatggagggcaactccagctctattacccagtacacaaggcccagcaatgtgtcctacattgaagtaaacaccaacaccaccgtgggagccatccagtggaagaacctggacgcagcctctgcttcctactcctactccgttcttatcttgaaggctggagatggcagcaatgtaaccagcagggtcagagacatcccttctgtcaccatccctgggctaatccccggcgtctcttccgaagtgaagatctttacaaagattaggaatacagaggttgggaatgaggtacctggtcagaagctgttctgtatggaacctgcacaagtggattccttacactgtgaagtggtcccgaaggagccagcgctggttctgaagtgggcctgcccccctggcatgaactcaggctttgaactgggggtcaggagtgatgcttgggacaatatgacacacttggagaactgcactttggacaatgacacagagtgcaggacagaagtcacctatttgaatttttctacctcgtacaacatcagcattgccaccttgtcatgtggaaagatggcattgcccacccagagcacctgcaccactggcatcacagacccacctcctccagatggatcccctaatattacatcggtcagtcacaattcagtaaaggtgaagttcagtggttttgaagccagccatggacctatcaaagcctatgctgtccttctcaccacaggggaagctggccaaccctccacagatgttttgaagtacacatatgaagatttcaaaaagggagcctccgatacttatgtgacatacctcataaggatagaagagaagggacagtctcagggcttgtctgaagcattgaactatgagattgatgtggggaaccaatccactaccctcggctactacaacgggaggctagagcctctgggctcctaccgggcttgcgttgctggctttaccaatattacctacaaccttcagaacgatggccttattaatggggatgagagctatgtatctttcagtccgtattcagaggccgtgtccttgcctcaagatccaggtgtcatctgcggagcggtgttcggatgtatctttggtgccctggccattgtggctgtgggaggcttcatcttctggagaaagaaaaggaaagatgccaagaataatgaagtgtccttttctcaaattaaacctaaaaaatccaagttaatccgagtggagaattttgaggcctactttaagaaacagcaagccgactccaactgtgggtttgcagaggaatatgaggatctgaagctgattggaataagtttacctaaatatgcagcggaaatagctgaaaacagggggaagaaccgctacaacaacgtcctgccctatgatatttctcgagtcaaactttcagtccagacccattcgacagacgactacatcaatgccaactatatgcctggctaccattccaagaaagatttcattgccacacaaggacctttacccaacactttgaaagatttttggcgtatggtttgggagaaaaacgtatatgccattgttatgctgaccaaatgtgtggagcagggaaggaccaaatgtgaagagtactggccttccaagcaggctcaggactacggggacataactgtggcaatgacatcagaagttgttcttccggaatggaccatcagagattttgtggtgaaaaatatgcagagtagtgagagtcatcctctgcggcagttccatttcacctcctggcctgaccatggtgttcctgacaccaccgacctgctcatcaactttcggtacctggtccgggattacatgaagcagatcccccctgagtcaccaatcctggtgcattgcagtgctggggttggaaggacgggcactttcattgctattgatcgcctgatctatcagatagagaacgagaacaccgtggatgtgtatggcattgtctacgatcttcggatgcacaggcctctgatggtacagacagaggaccagtatgttttcctcaatcagtgtgtgttggatattatcagagcccagaaagactcaaaagttgacctcatctatcagaacacaaccgcaatgacaatctatgaaaacctcgagcgagtgagcatgtttggaaaggctaatggttacatcgcctagtgccaaagcaaacccttctgaagctaaccagaccgtcacacccacagtgaaggagaacgccctgacggggacatgttttatgtgtctcatttctcaattctttgttctgttctgttagaactacttggagggcatggggctgtgcatgtaaaacatggcagataggcagttggggctctcaggggctgtgggcaggtggtgtgtaaatcagctgcattcctgattaccaatgggatgggttcactttctgtttttctttccttgagaattgtggaaaaccaggaaaagtgagctatgtttttgttttccaagcaggtttgttctcctctctttctattcctcccccttctctctctccctcccctgcctctctctgttttttttttttaagacttttatatgattcacatgctaaagccaggattatgttgggttggatatattttaagtatcagaggtctatttttacctactgtatctcggaatctagccgatggaaaagcatggctgtgggtgtttctcatgcagggaggggcacccggtgcctcttctgcgtgggtttctatttgaacatgtgccttttctgaattatgcttccacgggcaaaactcagtagatatctctatttttatattgaatctcagaattggaatatacagaatatttaaacagtagcttagtatcagaggttcgcctcctcagtaacattcctgtcccatcccattagaggaggctttctctgcggcccgcccttgacacacatttgtgctccatgttccttttccttcctttctttttttttttttctcccctcccagttttcgccaaagaacttctgttggtgagattttcatcccattggttgggtgagaatagaagtttaaaatatcaccatgagactctctgtgggtgggagtggggaggaacagaggaatcaacataccatggtgtgtgcaccccttttcatcccgcctcctatgttctctcatttctgtctttggtaagaaggatttttttaaaggtgcaatatgtgacgtgggggttcacgatgctctgggttcttagtaatgttttactcaggttgtcatttatgtgtgtatgtgtgtgtgtgtgtgtgtgtgtgtgtgtgtgtgtgtatgtatgtatgtgtgttttatagcgtggtgatctgtgaacatttcagtgtgaactcagattaacccctttccccccaaagtccactggctttagtctctctgcagtgacacacatcagtatctactgtatatataaatactaaactcttacacaccagtcattcctatgaattgaggtgtacaaagtttgatttgtatcaaagatgtaattattatttttaaaacctctttcactacactgctgctgtaattactttgaatttttaaagaaaaatgtagatttttttgttttgtttttggcttcaggtgtgtattcaccaagagtttcagaatttatgtggttttattttattggtacataatctgcaaacttctcaaggcattaacatgacaagatttgtatctcttatttttatcttgtggctcaggttatatttgaagaagtttcaagtttattttctagtaggcagtgaagtatacataaggtcaatgaaggaatcaccaggtccctggagtcctgtggctgttttccacagttacacatcctgctgtatgtttgggaccatgggtattgtgttgaagatgagggcaggggtcttctccgaggacctggccactgtgagccctggggtaacctgtcctctgatgttctgcactctgttcctcgaatcaccggcactttgatgcctgactggacctgatcatgagtgtttgtagggttagagagcctgaaccatgatgtactgaaagccctcgctgtggttcagcactgtagtgtgcgattcttgctgtgacccagcgctgctgtagcactgttgctgccgtggctcggcgctggggcacaacccctttccctttgcggtactgaaaggattgaaagaagaggtcatggcgctgtcatgctctgggggagaattttacttctcacatggaaactgcactttttgctaaatcctttgcattttgatgttcacttagtgtccagccctttaggatgggctggccagggtaggttggccccctggactcaacttcactggggaacttgacgctgcagttgtgaatcagggaagccatgctgtctccaggtatggggtgaagctgaacatgggctcattatttccaaatgccctgtctggtctgctcactaggatgtgctgactttgggcactatacagaggggcctgttctggcagtagttctgtgcaccgcctatacacctcagtctgtcaggaacggttgtagtgggtggtaaggggtccaatcccacgcagcgcctatgccctgcctctgctgccatggaaagtgcgaggaaaggacccgcccctaaaacagccctgtagaagattagagtccctcaaaaaaaaaaaaaaaactcgag

SEQ ID NO: 49 - protein, 1216 aa, Rattus norvegicus
MKPAARETRTPPRSPGLRWALLPLLLLLRQGQVVCTGAAPSPVFDVEAVTSPTSVVLTWKHNDSATSEYKINEGNTLRYTVKNQTSFNITGLSPATSYKFSITLGTVNETSGKPTYKNITTEPWPVSDLQVAYIGVTQALLAWSNANGTASYRMQIVELTTNSSGGISDLKPGTHKSLAVQGSNETQHDLWVTEGVSDPPSARDPSLTEVLLTELKPDTQYKVTIYSQAADGTEGQPGNKVFKTNPIQVSDIRAVNISDSNMTLTWKSNNNESHASFTYKIYVAGGSDSINETVNETQAVIRGLSSSTLYNITVLPFLGQTAGIPGFLQVYTSPRPVSDFRVTNVSLREIGLAWRSNDSESFEIFITQEGSEKRWNASTGDLSYIVVNLKPGTSYQFEIFPRGPNGTEGPSQTVVGRTDCSAVTDIRVVSVSTTEIQLEWQNTDSASGYTYHLVLESENGSIKTNSSQKWITVGGLTPGTLYNVTIFPEVDQMEGNSSSITQYTRPSNVSYIEVNTNTTVGAIQWKNLDAASASYSYSVLILKAGDGSNVTSRVRDIPSVTIPGLIPGVSSEVKIFTKIRNTEVGNEVPGQKLFCMEPAQVDSLHCEVVPKEPALVLKWACPPGMNSGFELGVRSDAWDNMTHLENCTLDNDTECRTEVTYLNFSTSYNISIATLSCGKMALPTQSTCTTGITDPPPPDGSPNITSVSHNSVKVKFSGFEASHGPIKAYAVLLTTGEAGQPSTDVLKYTYEDFKKGASDTYVTYLIRIEEKGQSQGLSEALNYEIDVGNQSTTLGYYNGRLEPLGSYRACVAGFTNITYNLQNDGLINGDESYVSFSPYSEAVSLPQDPGVICGAVFGCIFGALAIVAVGGFIFWRKKRKDAKNNEVSFSQIKPKKSKLIRVENFEAYFKKQQADSNCGFAEEYEDLKLIGISLPKYAAEIAENRGKNRYNNVLPYDISRVKLSVQTHSTDDYINANYMPGYHSKKDFIATQGPLPNTLKDFWRMVWEKNVYAIVMLTKCVEQGRTKCEEYWPSKQAQDYGDITVAMTSEVVLPEWTIRDFVVKNMQSSESHPLRQFHFTSWPDHGVPDTTDLLINFRYLVRDYMKQIPPESPILVHCSAGVGRTGTFIAIDRLIYQIENENTVDVYGIVYDLRMHRPLMVQTEDQYVFLNQCVLDIIRAQKDSKVDLIYQNTTAMTIYENLERVSMFGKANGYIA

SEQ ID NO: 50 - DNA, 1785 bp, Homo sapiens
atggcagcggagtcaggggaactaatcggggcttgtgagttcatgaaagatcggttatattttgctactttaaggaatagaccaaaaagcacagtaaatacccactatttctccatcgatgaggagctggtctatgaaaatttctatgcagattttggaccgctgaacttggcaatggtgtacagatattgctgcaaactaaacaagaaactaaaatcatacagtttgtcaagaaagaaaatagtgcactacacctgttttgaccaacggaaaagagcaaatgcagcatttttgataggtgcctatgcagtaatctatttaaagaagacaccagaagaagcctacagagcactcctgtctggctcaaaccccccctatcttccattcagggatgcttcctttggaaattgcacttacaatctcaccattctcgactgtttgcagggaatcagaaagggattacaacatggattttttgactttgagacatttgatgtggatgaatatgaacattatgagcgagttgaaaatggtgacttcaactggattgttccaggaaaatttttagcatttagtggaccacatcctaaaagcaaaattgagaatggttatcctcttcacgcccctgaagcctactttccttatttcaaaaagcataatgtgactgcagttgtgaggctaaacaaaaagatttatgaggcaaagcgcttcacagacgctggcttcgagcactatgacctcttcttcatagatggcagcacacccagtgacaacatcgtgcgaaggttcctgaacatctgtgagaacaccgaaggggccatcgccgttcactgcaaagctggtcttggaagaacagggacattgatagcctgttatgtaatgaaacactacaggtttacacatgctgaaataattgcttggattagaatatgccggccaggctctattataggaccccagcagcacttcctggaagaaaaacaagcatcgttgtgggtccaaggagacattttccgatccaaactgaaaaatcgaccatccagtgaaggaagtattaataaaattctttctggcctagatgatatgtctattggtggaaatctttcaaaaacacaaaacatggaacgatttggagaggataacttagaagatgatgatgtggaaatgaaaaatggtataacccagggagacaaactacgtgccttaaaaagtcagagacagccacgtacctcaccatcctgtgcatttaggtcagatgatacaaaaggacatccaagagcagtgtcccagcctttcagattaagttcatccctgcaaggatctgcagttactttgaagacatcaaaaatggcactgtccccttcagcaacggccaagaggatcaacagaacttctttgtcttcgggtgccactgtaagaagcttttccataaactcccggctagccagttctctagggaacttgaatgctgcaacagatgatccagagaacaaaaagacctcctcatcctctaaggcaggcttcacagccagcccgtttaccaacctcttgaatggcagctcccagccaactaccagaaattaccctgagctcaacaataatcagtacaacagaagcagcaacagcaacgggggcaacctgaacagccccccaggcccccacagcgccaagacagaggagcacaccaccatcctccgaccctcctacaccgggctttcttcttcttcagcgagattcctgagccgttctatcccttcccttcagtctgaatatgttcattactaa

SEQ ID NO: 51 - protein, 594 aa, Homo sapiens
MAAESGELIGACEFMKDRLYFATLRNRPKSTVNTHYFSIDEELVYENFYADFGPLNLAMVYRYCCKLNKKLKSYSLSRKKIVHYTCFDQRKRANAAFLIGAYAVIYLKKTPEEAYRALLSGSNPPYLPFRDASFGNCTYNLTILDCLQGIRKGLQHGFFDFETFDVDEYEHYERVENGDFNWIVPGKFLAFSGPHPKSKIENGYPLHAPEAYFPYFKKHNVTAVVRLNKKIYEAKRFTDAGFEHYDLFFIDGSTPSDNIVRRFLNICENTEGAIAVHCKAGLGRTGTLIACYVMKHYRFTHAEIIAWIRICRPGSIIGPQQHFLEEKQASLWVQGDIFRSKLKNRPSSEGSINKILSGLDDMSIGGNLSKTQNMERFGEDNLEDDDVEMKNGITQGDKLRALKSQRQPRTSPSCAFRSDDTKGHPRAVSQPFRLSSSLQGSAVTLKTSKMALSPSATAKRINRTSLSSGATVRSFSINSRLASSLGNLNAATDDPENKKTSSSSKAGFTASPFTNLLNGSSQPTTRNYPELNNNQYNRSSNSNGGNLNSPPGPHSAKTEEHTTILRPSYTGLSSSSARFLSRSIPSLQSEYVHY

SEQ ID NO: 52 - DNA, 2438 bp, Homo sapiens
cgaagaggatccggagcagctgctgccagcccgcgggcactgaagtcctcccggctgccgctcgagtagccacgggcgcgatcgggaccagaagtctcctcctccatgatcactttggaagccgggggaagactttgccctgccctgagagctggtctgcgtttcccaggcgcggcggcggcggagcagcagctgcagcagccgagtccaaataggagcggccacagccaggggcgtgtgcgccccgcgcggagcgagctcgggttcccctcggaatgtccccggggcgcccggcgcgctgaccccgaagccgcctccgccttcggcgcctgctgcctccctcggccaggcttgttgttcgggactgtgagcttcctggctcctgggcagtggggaagcccccgggggcgagtgacctcagctggccacgacccagccctcccccgtgcgtatctcgcttaagatggcagcggagtcaggggaactaatcggggcttgtgagttcatgaaagatcggttatattttgctactttaaggaatagaccaaaaagcacagtaaatacccactatttctccatcgatgaggagctggtctatgaaaatttctatgcagattttggaccgctgaacttggcaatggtgtacagatattgctgcaaactaaacaagaaactaaaatcatacagtttgtcaagaaagaaaatagtgcactacacctgttttgaccaacggaaaagagcaaatgcagcatttttgataggtgcctatgcagtaatctatttaaagaagacaccagaagaagcctacagagcactcctgtctggctcaaaccccccctatcttccattcagggatgcttcctttggaaattgcacttacaatctcaccattctcgactgtttgcagggaatcagaaagggattacaacatggattttttgactttgagacatttgatgtggatgaatatgaacattatgagcgagttgaaaatggtgacttcaactggattgttccaggaaaatttttagcatttagtggaccacatcctaaaagcaaaattgagaatggttatcctcttcacgcccctgaagcctactttccttatttcaaaaagcataatgtgactgcagttgtgaggctaaacaaaaagatttatgaggcaaagcgcttcacagacgctggcttcgagcactatgacctcttcttcatagatggcagcacacccagtgacaacatcgtgcgaaggttcctgaacatctgtgagaacaccgaaggggccatcgccgttcactgcaaagctggtcttggaagaacagggacattgatagcctgttatgtaatgaaacactacaggtttacacatgctgaaataattgcttggattagaatatgccggccaggctctattataggaccccagcagcacttcctggaagaaaaacaagcatcgttgtgggtccaaggagacattttccgatccaaactgaaaaatcgaccatccagtgaaggaagtattaataaaattctttctggcctagatgatatgtctattggtggaaatctttcaaaaacacaaaacatggaacgatttggagaggataacttagaagatgatgatgtggaaatgaaaaatggtataacccagggagacaaactacgtgccttaaaaagtcagagacagccacgtacctcaccatcctgtgcatttaggtcagatgatacaaaaggacatccaagagcagtgtcccagcctttcagattaagttcatccctgcaaggatctgcagttactttgaagacatcaaaaatggcactgtccccttcagcaacggccaagaggatcaacagaacttctttgtcttcgggtgccactgtaagaagcttttccataaactcccggctagccagttctctagggaacttgaatgctgcaacagatgatccagagaacaaaaagacctcctcatcctctaaggcaggcttcacagccagcccgtttaccaacctcttgaatggcagctcccagccaactaccagaaattaccctgagctcaacaataatcagtacaacagaagcagcaacagcaacgggggcaacctgaacagccccccaggcccccacagcgccaagacagaggagcacaccaccatcctccgaccctcctacaccgggctttcttcttcttcagcgagattcctgagccgttctatccctgtaagtgcgcagacaccacctcctggtcctcagaaccctgaatgcaacttctgtgccttgccttcccagccgaggctgccaccaaagaaatttaatagtgccaaggaagccttctgagcgatgccttccctctgtgctgtgaaactgtctatgcactacattctgctagctcctcttcaagtaaacgccaagtcacaaaaaaaaaaaaaaaaaaaaaaaa

SEQ ID NO: 53 - protein, 623 aa, Homo sapiens
MAAESGELIGACEFMKDRLYFATLRNRPKSTVNTHYFSIDEELVYENFYADFGPLNLAMVYRYCCKLNKKLKSYSLSRKKIVHYTCFDQRKRANAAFLIGAYAVIYLKKTPEEAYRALLSGSNPPYLPFRDASFGNCTYNLTILDCLQGIRKGLQHGFFDFETFDVDEYEHYERVENGDFNWIVPGKFLAFSGPHPKSKIENGYPLHAPEAYFPYFKKHNVTAVVRLNKKIYEAKRFTDAGFEHYDLFFIDGSTPSDNIVRRFLNICENTEGAIAVHCKAGLGRTGTLIACYVMKHYRFTHAEIIAWIRICRPGSIIGPQQHFLEEKQASLWVQGDIFRSKLKNRPSSEGSINKILSGLDDMSIGGNLSKTQNMERFGEDNLEDDDVEMKNGITQGDKLRALKSQRQPRTSPSCAFRSDDTKGHPRAVSQPFRLSSSLQGSAVTLKTSKMALSPSATAKRINRTSLSSGATVRSFSINSRLASSLGNLNAATDDPENKKTSSSSKAGFTASPFTNLLNGSSQPTTRNYPELNNNQYNRSSNSNGGNLNSPPGPHSAKTEEHTTILRPSYTGLSSSSARFLSRSIPVSAQTPPPGPQNPECNFCALPSQPRLPPKKFNSAKEAF

SEQ ID NO: 54 - DNA, 1890 bp, Homo sapiens
ccggagcagctgctgccagcccgcgggcactgaagtcctcccggctgccgctcgagtagccacgggcgcgatcgggaccagaagtctcctcctccatgatcactttggaagccgggggaagactttgccctgccctgagagctggtctgcgtttcccaggcgcggcggcggcggagcagcagctgcagcagccgagtccaaataggagcggccacagccaggggcgtgtgcgccccgcgcggagcgagctcgggttcccctcggaatgtccccggggcgcccggcgcgctgaccccgaagccgcctccgccttcggcgcctgctgcctccctcggccaggcttgttgttcgggactgtgagcttcctggctcctgggcagtggggaagcccccgggggcgagtgacctcagctggccacgacccagccctcccccgtgcgtatctcgcttaagatggcagcggagtcaggggaactaatcggggcttgtgagttcatgaaagatcggttatattttgctactttaaggaatagaccaaaaagcacagtaaatacccactatttctccatcgatgaggagctggtctatgaaaatttctatgcagattttggaccgctgaacttggcaatggtgtacagatattgctgcaaactaaacaagaaactaaaatcatacagtttgtcaagaaagaaaatagtgcactacacctgttttgaccaacggaaaagagcaaatgcagcatttttgataggtgcctatgcagtaatctatttaaagaagacaccagaagaagcctacagagcactcctgtctggctcaaaccccccctatcttccattcagggatgcttcctttggaaattgcacttacaatctcaccattctcgactgtttgcagggaatcagaaagggattacaacatggattttttgactttgagacatttgatgtggatgaatatgaacattatgagcgagttgaaaatggtgacttcaactggattgttccaggaaaatttttagcatttagtggaccacatcctaaaagcaaaattgagaatggttatcctcttcacgcccctgaagcctactttccttatttcaaaaagcataatgtgactgcagttgtgaggctaaacaaaaagatttatgaggcaaagcgcttcacagacgctggcttcgagcactatgacctcttcttcatagatggcagcacacccagtgacaacatcgtgcgaaggttcctgaacatctgtgagaacaccgaaggggccatcgccgttcactgcaaagctggtcttggaagaacagggacattgatagcctgttatgtaatgaaacactacaggtttacacatgctgaaataattgcttggattagaatatgccggccaggctctattataggaccccagcagcacttcctggaagaaaaacaagcatcgttgtgggtccaaggagacattttccgatccaaactgaaaaatcgaccatccagtgaaggaagtattaataaaattctttctggcctagatgatatgtctattggtggaaatctttcaaaaacacaaaacatggaacgatttggagaggtaagttttccctaggagattctatcttcttaaaactgatgttctgcatttgtttctcagttggacctatataacatagcagtgtcttttctctggatgccacgagtaccaagtttttagaagtagagccatccgtctatatagcaagaagcagaggaaagaaaccaattgcccttaaaaaaaaaaagctataatttaaggagtaaattataaaggaggctactctggtaaggggtaatatttatagaaaggaaacagaaaagcaaactttctatttgaaaaaaaaaaaaaaaaaaaaaa

SEQ ID NO: 55 - protein, 383 aa, Homo sapiens
MAAESGELIGACEFMKDRLYFATLRNRPKSTVNTHYFSIDEELVYENFYADFGPLNLAMVYRYCCKLNKKLKSYSLSRKKIVHYTCFDQRKRANAAFLIGAYAVIYLKKTPEEAYRALLSGSNPPYLPFRDASFGNCTYNLTILDCLQGIRKGLQHGFFDFETFDVDEYEHYERVENGDFNWIVPGKFLAFSGPHPKSKIENGYPLHAPEAYFPYFKKHNVTAVVRLNKKIYEAKRFTDAGFEHYDLFFIDGSTPSDNIVRRFLNICENTEGAIAVHCKAGLGRTGTLIACYVMKHYRFTHAEIIAWIRICRPGSIIGPQQHFLEEKQASLWVQGDIFRSKLKNRPSSEGSINKILSGLDDMSIGGNLSKTQNMERFGEVSFP

SEQ ID NO: 56 - DNA, 4624 bp, Homo sapiens
cacggaacagccctcctggggtccccacgagccgcgtcctgctgtgccccggcgcctacgcagcagcggccgcggccgcggtgggcacgcacggttaccccgggcagctccggccgccagctgcagccccgtcgcctcggccgcgccagccggctgcgggcacctgggggcgggctgggggcgccggccgcggcaggaggcgctgtagcgagggctgcggcgccggtcctgcggcggccgcgggaggcagcggggcaggcgctgtgggccgggctcctcctccggctcctgcgcgaccgcctcccgccgggctctgccggcgcccgccgtccccgcagcgccgctctgcgcccgccgccccgagcgcccgcgcggggctggcgggagcctcggcgggcgcgcgggcgcgcggggccatggtcgtggccccctgacgggccgcggccgcctccatgaagcggaaaagcgagcggcggtcgagctgggccgccgcgcccccctgctcgcggcgctgctcgtcgacctcgccgggtgtgaagaagatccgcagctccacgcagcaagacccgcgccgccgggacccccaggacgacgtgtacctggacatcaccgatcgcctttgttttgccattctctacagcagaccaaagagtgcatcaaatgtacattatttcagcatagataatgaacttgaatatgagaacttctacgcagattttggaccactcaatctggcaatggtttacagatattgttgcaagatcaataagaaattaaagtccattacaatgttaaggaagaaaattgttcattttactggctctgatcagagaaaacaagcaaatgctgccttccttgttggatgctacatggttatatatttggggagaaccccagaagaagcatatagaatattaatctttggagagacatcctatattcctttcagagatgctgcctatggaagttgcaatttctacattacacttcttgactgttttcatgcagtaaagaaggcaatgcagtatggcttccttaatttcaactcatttaaccttgatgaatatgaacactatgaaaaagcagaaaatggagatttaaattggataataccagaccgatttattgccttctgtggacctcattcaagagccagacttgaaagtggttaccaccaacattctcctgagacttatattcaatattttaagaatcacaatgttactaccattattcgtctgaataaaaggatgtatgatgccaaacgctttacggatgctggcttcgatcaccatgatcttttctttgcggatggcagcacccctactgatgccattgtcaaagaattcctagatatctgtgaaaatgctgagggtgccattgcagtacattgcaaagctggccttggtcgcacgggcactctgatagcctgctacatcatgaagcattacaggatgacagcagccgagaccattgcgtgggtcaggatctgcagacctggctcggtgattgggcctcagcagcagtttttggtgatgaagcaaaccaacctctggctggaaggggactattttcgtcagaagttaaaggggcaggagaatggacaacacagagcagccttctccaaacttctctctggcgttgatgacatttccataaatggggtcgagaatcaagatcagcaagaacccgaaccgtacagtgatgatgacgaaatcaatggagtgacacaaggtgatagacttcgggccttgaaaagcagaagacaatccaaaacaaacgctattcctctcacagtaattcttcaatccagtgttcagagctgtaaaacatctgaacctaacatttctggcagtgcaggcattactaaaagaaccaccagatctgcttcaaggaaaagcagtgttaaaagtctctccatttcaaggactaaaacagtcttgcgttaagtaaaaacctgtgaccagagctgaaggaagactctaggactgaaaactgcaacagaaattagcacaatttgaaaacaaaacaaaattgcaaaagccttagttgctttttccacctaagaagttgatcaatggagaaaatgtccactggagtttgaataatgaactttgagtttgggtgcaagcaaatgactcagagaagggtccagctctcaagctgaatgacaaacatgctgttgtaaatttagtctcaggtgtaaatacccaagccctctggtacccagggagctggctggtctgtggtgcatgtgtgtccctgtgatggcaatcattgtagttgctggccttcagaagaattgaggatctgatggagtttttttatgtatttattttctgttcaccttgtgaccctgtgtcaaaatttataaagatacaaaaggcattactgaaatggtactttctgtaatttgatactatttggcttaatcatcttcacttgactatttgtaatactgttgtaatgttaactctgttaagtacccaagctgcttgtcttccaccaaagagtgctttattaacaagaatctgtgaaaatcacatttaaacactgttgcatgttgtaagaccaggtggtaccttagtaacctaaaacttgcaagagaatattaatggtagctttagaagactcaggaggagaaactgacttcagagttggaagatgttgcaagtcgttcctttttctgtccttcagggactgaagaactgggaggctgcccattgtttggttgccagtcatacaaattaaaatcatatttccttccatgaatggaagaaacacactattggtttttccccttggaaacagcaatcccaaataatgtcggcttacaaaaaaaaaaagttaccacttttttagagtccttccctgtaacattggattttttttttcccttatgagatccacctaaggccattgacgtggcctgcgatctcagtgacaatgatctgcttctggatctcactgttgcctttggttagggaacacaactagtaactctgcagagtgccttctcccgcagccctactggaacacagcagagtctgtgccatgaagcagttacagaaacagaattgatgtgctgccaaaaaaaaaaaaaaaatggggcccgaaataaaagaatatatagtactcacctcagttccttccataagaagtgggtggtttaatgattgttaagccatttttgcctgtgccgggagcatggagggctgagatgtcgacaggcagtgggaaacaaatgccctcctaagccacaaggcgtgcgccagattagtaggcaactccattttaagaagctgcctttttcacaaaactggaagaaataaaagcggttggaataaacaagttaaaagtctttaatgcaaaaagtaattgaaaggcagtgcctccattttggtgtactttcttggaagaaagtataaaattgaccggcatcatgagagacggaagatgccgtgttctcagccaaacaagcaactctttccccgccaggcactgtcgggtggggtcaggccagcttttaaacactggggactggatcacagaaaaacagtggttttctgtccctggaaatgaataggcacaaagacccacttggctgtgggcagactactcttcaataagatttgggtgggaggaggaacattccttttgctattttgagctgagacaatataaatattcaaactgtgccatgcataaagcattgaattctcagggcacctcttcttccccttaccccttttaaggccatcccctccattaataataatccaggtagttgtgaaaatcgtgcttctatctgatcccttcttagtttggcttttcatcccatcagaacaagtaaacgtaggcgccacagctcttgtgagtactgtctccctcacggtgaatgagcctcctggtgtttcgtccaagaaaagaaagggtgtcactggaaccacagccctttttcattttataaactgcctcttcatgttgcctgctcaagtttccacctagaattgctatcactgtggctctttctaaaaatctttctatttaactggttcactgaaattagtcatagaaaacttgtgatttggtgaagaggcattccttgtaataaccaaatgacttgggatggtgtgcatagcaagggcagtgttacacttatgaggactgtctctagcatccaggaagtctctgggtctgagggatggaaagttcttcctgctatgaatgagagtggactcttcccctcacccccaactgaaaccacaaacaaccagaatcttctggaattctgacttagagtcgttgttatagaagaccttgttgctatggaacatgaaactgtgtgtcagatggagagatccccttaacctaagagccttaaatagccctgaaagtacactgggacggtttgcgatggaattaaaattggaagtgaatatttttaggtgctcttgaagctttctggggactcaaaattatcaaaagtcagggacagtccggaggaagagcgtctgcaaaactgggttcctagaagtatagacggacttagctttttgtagaatttggtgaggagcagcgcctcgtgagagcagaatggcctggcgtggccagtgcttcccgg

SEQ ID NO: 57 - protein, 498 aa, Homo sapiens
MKRKSERRSSWAAAPPCSRRCSSTSPGVKKIRSSTQQDPRRRDPQDDVYLDITDRLCFAILYSRPKSASNVHYFSIDNELEYENFYADFGPLNLAMVYRYCCKINKKLKSITMLRKKIVHFTGSDQRKQANAAFLVGCYMVIYLGRTPEEAYRILIFGETSYIPFRDAAYGSCNFYITLLDCFHAVKKAMQYGFLNFNSFNLDEYEHYEKAENGDLNWIIPDRFIAFCGPHSRARLESGYHQHSPETYIQYFKNHNVTTIIRLNKRMYDAKRFTDAGFDHHDLFFADGSTPTDAIVKEFLDICENAEGAIAVHCKAGLGRTGTLIACYIMKHYRMTAAETIAWVRICRPGSVIGPQQQFLVMKQTNLWLEGDYFRQKLKGQENGQHRAAFSKLLSGVDDISINGVENQDQQEPEPYSDDDEINGVTQGDRLRALKSRRQSKTNAIPLTVILQSSVQSCKTSEPNISGSAGITKRTTRSASRKSSVKSLSISRTKTVLR

SEQ ID NO: 58 - DNA, 4960 bp, Homo sapiens
cacggaacagccctcctggggtccccacgagccgcgtcctgctgtgccccggcgcctacgcagcagcggccgcggccgcggtgggcacgcacggttaccccgggcagctccggccgccagctgcagccccgtcgcctcggccgcgccagccggctgcgggcacctgggggcgggctgggggcgccggccgcggcaggaggcgctgtagcgagggctgcggcgccggtcctgcggcggccgcgggaggcagcggggcaggcgctgtgggccgggctcctcctccggctcctgcgcgaccgcctcccgccgggctctgccggcgcccgccgtccccgcagcgccgctctgcgcccgccgccccgagcgcccgcgcggggctggcgggagcctcggcgggcgcgcgggcgcgcggggccatggtcgtggccccctgacgggccgcggccgcctccatgaagcggaaaagcgagcggcggtcgagctgggccgccgcgcccccctgctcgcggcgctgctcgtcgacctcgccgggtgtgaagaagatccgcagctccacgcagcaagacccgcgccgccgggacccccaggacgacgtgtacctggacatcaccgatcgcctttgttttgccattctctacagcagaccaaagagtgcatcaaatgtacattatttcagcatagataatgaacttgaatatgagaacttctacgcagattttggaccactcaatctggcaatggtttacagatattgttgcaagatcaataagaaattaaagtccattacaatgttaaggaagaaaattgttcattttactggctctgatcagagaaaacaagcaaatgctgccttccttgttggatgctacatggttatatatttggggagaaccccagaagaagcatatagaatattaatctttggagagacatcctatattcctttcagagatgctgcctatggaagttgcaatttctacattacacttcttgactgttttcatgcagtaaagaaggcaatgcagtatggcttccttaatttcaactcatttaaccttgatgaatatgaacactatgaaaaagcagaaaatggagatttaaattggataataccagaccgatttattgccttctgtggacctcattcaagagccagacttgaaagtggttaccaccaacattctcctgagacttatattcaatattttaagaatcacaatgttactaccattattcgtctgaataaaaggatgtatgatgccaaacgctttacggatgctggcttcgatcaccatgatcttttctttgcggatggcagcacccctactgatgccattgtcaaagaattcctagatatctgtgaaaatgctgagggtgccattgcagtacattgcaaagctggccttggtcgcacgggcactctgatagcctgctacatcatgaagcattacaggatgacagcagccgagaccattgcgtgggtcaggatctgcagacctggctcggtgattgggcctcagcagcagtttttggtgatgaagcaaaccaacctctggctggaaggggactattttcgtcagaagttaaaggggcaggagaatggacaacacagagcagccttctccaaacttctctctggcgttgatgacatttccataaatggggtcgagaatcaagatcagcaagaacccgaaccgtacagtgatgatgacgaaatcaatggagtgacacaaggtgatagacttcgggccttgaaaagcagaagacaatccaaaacaaacgctattcctctcaccgatggttggctgtcccaggctgtcacctttctagaccggcttctgatctggctcgggatccacaaggactagacctgcggggaaggtctctcctggacacgcccgttgcccactgcaagttctctccaggtgcaattgaagcctctcagcagcggaggccgccatgtggagagagcaggcaggcccactgctgctgagaacagggcaggcacgggcagctcctgttctgcctttcccagcttcggagacgcaggctcagctgctccgaagcacctgccagcaccgcacagtacagtttcagaggacagcagtctccttcccgtgaagctcccatgtgctggaatggcatggacttgctgatcaacagaaggaaatggtctgaagtctgaccagcacaaggaaggaggctggctggctcagaggggcccaccttgcgtggaatgaaaacgccaaaggctcatgagcaacattaggctagaggggtcttgttcaaagcatccaactctgacttcggaggcattcccagccggcagcagtgtgtccagcctgcctcttcccaggctggtctgacatgcagcttaggctttcatcccaagttaggtactgacccctccctcttgggcagcacctccctttttaaaaaaatttttttttcttccaaagacagagtcttgctcttgttgtccaggctggagtgcagtggcgcgatctaggctcactgcaacctccttctcccaggttcaagcgactctcctgcctcagcctcctgagtagctgggattataggcgtctgccaccacgcccggctaatttctgtatttttagtagagacagggtttcaccatgttggccaggctggtctcgaactcctgacctcaagtgatctgcctgccttggcctcccaaagtgctgggattacaggtgtcagccaccgcacccagccaagcacccctatctctagaggatctggccccccagcccagttactgcagggcagctttccccacctggtgacaggctgtgcgcagcagccccaggacctcaccctgagctgagtcttcaggagccgccctggtggcacaactcagacacccctgaggcctagcagtcaactcctgattcagacatgatccagtccagcctgggcttggctataaccagctcaaacttgcttgacctccacttttcaggagacttggggacgacagccctcatcggcgtctttcatggggttaatctgcttgagtctaagtcgccagccagaaacgtggtgcccagggtgccctgcctcaggacatgtccacacccacgtcacaagcacctgaggagtccggccggggcactgtggtccaaaaggtcctgccgcctccgcatctgactgtcccaacggcatgctggtgacacccccctgcccttcgcttctgtcctccctggcttctctggggcacttggggctatgtacaacctggcacgatccagaaagggtgcaaacaaaatgcctacatccaggcacacgaccaagtcagcgagagctagccctggtaagcaaacatagcccattacaggttcagaacgtgcaccgggttccccaaaactgtcttcaaccacatgactcaacagctctatgggataggaactgtcagtgtttttgcaactgcaacattaaaccaagtgctgtgggcttttcaagtattattcacaacactaaaggaaagtttcttcaaagggctctctggctaatcttcaaagccgcagttaggcaaaatgacagtgtgacagcttcaaagccactgactcatgacacagccctgatgttgtaccggctaggttcagatttcagaaatcagggcacttgcatccattgccttttccaggaaagggaagaaaacactcagttgataaaccttagtactcagataataaataagagaccaaaagtaggctatcacccaaagcaaacatccttaactgaccctaacgtgtatggattcaactttgattattcaacaaaatcatgaccgactgctgtggcctggagtaaccaaaggactgttttctctacacaaagtcaggagcgaataccaacctttatttgcacttgggttccagttcaaagccaccttagacagtgtggcaaagtgggaaaaagcacagatcctgggaccaaggttcagattccatctcaagcgagcatatgaactgtgtgacaacaggcagacagtacctctgtgtctatgagaaagcggggagagcaacaccccagcttctagcagctctacagctgcctggacctgcaggccctcctaggcccacttcctccccagcacagtgtgtgttcccgggcgtgtgtggctctgggtccagctctgttcagggtgggactccaggtgaattactgaacctctgaggtgtacccccaaccccaaactttcaccaaaagcaataaagaggaactctagaactggagccaggactaagtgagaaaaactgcttataagtgcttaataaatactagttatttacaacttttgctcaagccgagggcagaggcctttgtacgcagctgccgaactctgactctagttctgcggaagaaaaggatgcggtatttgcttttgccatgatccctttccatttgattggcaggttaaataacatggtttttgaagtcacatacttaatattcttcctaaaaaccacccaaacactagatgtgtgtgtgcacacacacagaaaccacggggtagtttaaatcaccattaaaaatcaacgctttctctgattctgtgtcacagagtggtggccagtggctacaatttttaaatgattggttaagtgaaaaccagaactcaaaatattccaggagagaagataacatttacaagtaaacagtaagtgcaattgtattttaatttcttggtctccgaaaactcagctgtgactgctttccattaacagttccagctctatgtgtttcctctaacgctaaaggcacagcccccgggaatctactgcttcctaagagtctccatggagtctattttacaacctcctttccctccatgcttccgcggaggagtctatactatctctatatacacattttaaacattattcttcatttgaaattccttcaataaaaacacagtcaccattaaaaaaaa

SEQ ID NO: 59 - protein, 471 aa, Homo sapiens
MKRKSERRSSWAAAPPCSRRCSSTSPGVKKIRSSTQQDPRRRDPQDDVYLDITDRLCFAILYSRPKSASNVHYFSIDNELEYENFYADFGPLNLAMVYRYCCKINKKLKSITMLRKKIVHFTGSDQRKQANAAFLVGCYMVIYLGRTPEEAYRILIFGETSYIPFRDAAYGSCNFYITLLDCFHAVKKAMQYGFLNFNSFNLDEYEHYEKAENGDLNWIIPDRFIAFCGPHSRARLESGYHQHSPETYIQYFKNHNVTTIIRLNKRMYDAKRFTDAGFDHHDLFFADGSTPTDAIVKEFLDICENAEGAIAVHCKAGLGRTGTLIACYIMKHYRMTAAETIAWVRICRPGSVIGPQQQFLVMKQTNLWLEGDYFRQKLKGQENGQHRAAFSKLLSGVDDISINGVENQDQQEPEPYSDDDEINGVTQGDRLRALKSRRQSKTNAIPLTDGWLSQAVTFLDRLLIWLGIHKD

SEQ ID NO: 60 - DNA, 2646 bp, Homo sapiens
  misc_feature 2300: n = A,T,C or G
atgaagcggaaaagcgagcggcggtcgagctgggccgccgcgcccccctgctcgcggcgctgctcgtcgacctcgccgggtgtgaagaagatccgcagctccacgcagcaagacccgcgccgccgggacccccaggacgacgtgtacctggacatcaccgatcgcctttgttttgccattctctacagcagaccaaagagtgcatcaaatgtacattatttcagcatagataatgaacttgaatatgagaacttctacgcagattttggaccactcaatctggcaatggtttacagatattgttgcaagatcaataagaaattaaagtccattacaatgttaaggaagaaaattgttcattttactggctctgatcagagaaaacaagcaaatgctgccttccttgttggatgctacatggttatatatttggggagaaccccagaagaagcatatagaatattaatctttggagagacatcctatattcctttcagagatgctgcctatggaagttgcaatttctacattacacttcttgactgttttcatgcagtaaagaaggcaatgcagtatggcttccttaatttcaactcatttaaccttgatgaatatgaacactatgaaaaagcagaaaatggagatttaaattggataataccagaccgatttattgccttctgtggacctcattcaagagccagacttgaaagtggttaccaccaacattctcctgagacttatattcaatattttaagaatcacaatgttactaccattattcgtctgaataaaaggatgtatgatgccaaacgctttacggatgctggcttcgatcaccatgatcttttctttgcggatggcagcacccctactgatgccattgtcaaagaattcctagatatctgtgaaaatgctgagggtgccattgcagtacattgcaaagctggccttggtcgcacgggcactctgatagcctgctacatcatgaagcattacaggatgacagcagccgagaccattgcgtgggtcaggatctgcagacctggctcggtgattgggcctcagcagcagtttttggtgatgaagcaaaccaacctctggctggaaggggactattttcgtcagaagttaaaggggcaggagaatggacaacacagagcagccttctccaaacttctctctggcgttgatgacatttccataaatggggtcgagaatcaagatcagcaagaacccgaaccgtacagtgatgatgacgaaatcaatggagtgacacaaggtgatagacttcgggccttgaaaagcagaagacaatccaaaacaaacgctattcctctcactctctccatttcaaggactaaaacagtcttgcgttaagtaaaaacctgtgaccagagctgaaggaagactctaggactgaaaactgcaacagaaattagcacaatttgaaaacaaaacaaaattgcaaaagccttagttgctttttccacctaagaagttgatcaatggagaaaatgtccactggagtttgaataatgaactttgagtttgggtgcaagcaaatgactcagagaagggtccagctctcaagctgaatgacaaacatgctgttgtaaatttagtctcaggtgtaaatacccaagccctctggtacccagggagctggctggtctgtggtgcatgtgtgtccctgtgatggcaatcattgtagttgctggccttcagaagaattgaggatctgatggaggttttttatgtatttattttctgttcaccttgtgaccctgtgtcaaaatttataaagatacaaaaggcattactgaaatggtactttctgtaatttgatactatttggcttaatcatcttcacttgactatttgtaatactgttgtaatgttaactctgttaagtacccaagctgcttgtcttccaccaaagagtgctttattaacaagaatctgtgaaaatcacatttaaacactgttgcatgttgtaagaccaggtggtaccttagtaacctaaaacttgcaagagaatattaatggtagctttagaagactcaggaggagaaactgacttcagagttggaagatgttgcaagtcgttcctttttctgtccttcagggactgaagaactgggaggctgcccattgtttggttgccagtcatacaaattaaaatcatatttccttccatgaatggaagaaacacactattggtttttccccttggaaacagcaatcccaaataatgtcggcttacaaaaaaaaaagttaccacttttttagagtccttnccctgtaacattggatttttttttcccttatgagatccacctaaggccattgacgtggcctgcgatctcagtgacaatgatctgctttctggatctcactgttgcctttggttagggaacacagagtgcttctcccgcagccctactggaacacagcagagtctgtgccatgaagcagttacagaaacagaattgatgtgctgctaaaaaaaaaaaaaaaaatggggcccgggggggcgtccgccggccctgcgggccgccggtgaaataccactactctgatcgttttttcactgacccggtgaggcgggggggcgagccccgaggggctctcgcttctggcgcg

SEQ ID NO: 61 - protein, 459 aa, Homo sapiens
MKRKSERRSSWAAAPPCSRRCSSTSPGVKKIRSSTQQDPRRRDPQDDVYLDITDRLCFAILYSRPKSASNVHYFSIDNELEYENFYADFGPLNLAMVYRYCCKINKKLKSITMLRKKIVHFTGSDQRKQANAAFLVGCYMVIYLGRTPEEAYRILIFGETSYIPFRDAAYGSCNFYITLLDCFHAVKKAMQYGFLNFNSFNLDEYEHYEKAENGDLNWIIPDRFIAFCGPHSRARLESGYHQHSPETYIQYFKNHNVTTIIRLNKRMYDAKRFTDAGFDHHDLFFADGSTPTDAIVKEFLDICENAEGAIAVHCKAGLGRTGTLIACYIMKHYRMTAAETIAWVRICRPGSVIGPQQQFLVMKQTNLWLEGDYFRQKLKGQENGQHRAAFSKLLSGVDDISINGVENQDQQEPEPYSDDDEINGVTQGDRLRALKSRRQSKTNAIPLTLSISRTKTVLR

SEQ ID NO: 62 - DNA, 2160 bp, Homo sapiens
gaccagaccggcccccccgagactatagccttcactttccctcggtccaccatggagcccttgtgtccactcctgctggtgggttttagcttgccgctcgccagggctctcaggggcaacgagaccactgccgacagcaacgagacaaccacgacctcaggccctccggacccgggcgcctcccagccgctgctggcctggctgctactgccgctgctgctcctcctcctcgtgctccttctcgccgcctacttcttcaggttcaggaagcagaggaaagctgtggtcagcaccagcgacaagaagatgcccaacggaatcttggaggagcaagagcagcaaagggtgatgctgctcagcaggtcaccctcagggcccaagaagtattttcccatccccgtggagcacctggaggaggagatccgtatcagatccgccgacgactgcaagcagtttcgggaggagttcaactcattgccatctggacacatacaaggaacttttgaactggcaaataaagaagaaaacagagaaaaaaacagatatcccaacatccttcccaatgaccattctagggtgattctgagccaactggatggaattccctgttcagactacatcaatgcttcctacatagatggttacaaagagaagaataaattcatagcagctcaaggtcccaaacaggaaacggttaacgacttctggagaatggtctgggagcaaaagtctgcgaccatcgtcatgttaacaaacttgaaagaaaggaaagaggaaaagtgccatcagtactggcccgaccaaggctgctggacctatggaaacatccgggtgtgcgtggaggactgcgtggttttggtcgactacaccatccggaagttctgcatacagccacagctccccgacggctgcaaagcccccaggctggtctcacagctgcacttcaccagctggcccgacttcggagtgccttttacccccattgggatgctgaagttcctcaagaaagtaaagacgctcaaccccgtgcacgctgggcccatcgtggtccactgtagcgcgggcgtgggccggacgggcaccttcattgtgatcgatgccatgatggccatgatgcacgcggagcagaaggtggatgtgtttgaatttgtgtctcgaatccgtaatcagcgccctcagatggttcaaacggatatgcagtacacgttcatctaccaagccttactcgagtactacctctacggggacacagagctggacgtgtcctccctggagaagcacctgcagaccatgcacggcaccaccacccacttcgacaagatcgggctggaggaggagttcaggaaattgacaaatgtccggatcatgaaggagaacatgaggacgggcaacttgccggcaaacatgaagaaggccagggtcatccagatcatcccgtatgacttcaaccgagtgatcctttccatgaaaaggggtcaagaatacacagactacatcaacgcatccttcatagacggctaccgacagaaggactatttcatcgccacccaggggccactggcacacacggttgaggacttctggaggatgatctgggaatggaaatcccacactatcgtgatgctgacggaggtgcaggagagagagcaggataaatgctaccagtattggccaaccgagggctcagttactcatggagaaataacgattgagataaagaatgataccctttcagaagccatcagtatacgagactttctggtcactctcaatcagccccaggcccgccaggaggagcaggtccgagtagtgcgccagtttcacttccacggctggcctgagatcgggattcccgccgagggcaaaggcatgattgacctcatcgcagccgtgcagaagcagcagcagcagacaggcaaccaccccatcaccgtgcactgcagtgccggagctgggcgaacaggtacattcatagccctcagcaacattttggagcgagtaaaagccgagggacttttagatgtatttcaagctgtgaagagtttacgacttcagagaccacatatggtgcaaaccctggaacagtatgaattctgctacaaagtggtacaagattttattgatatattttctgattatgctaatttcaaatgaagattc

SEQ ID NO: 63 - protein, 700 aa, Homo sapiens
MEPLCPLLLVGFSLPLARALRGNETTADSNETTTTSGPPDPGASQPLLAWLLLPLLLLLLVLLLAAYFFRFRKQRKAVVSTSDKKMPNGILEEQEQQRVMLLSRSPSGPKKYFPIPVEHLEEEIRIRSADDCKQFREEFNSLPSGHIQGTFELANKEENREKNRYPNILPNDHSRVILSQLDGIPCSDYINASYIDGYKEKNKFIAAQGPKQETVNDFWRMVWEQKSATIVMLTNLKERKEEKCHQYWPDQGCWTYGNIRVCVEDCVVLVDYTIRKFCIQPQLPDGCKAPRLVSQLHFTSWPDFGVPFTPIGMLKFLKKVKTLNPVHAGPIVVHCSAGVGRTGTFIVIDAMMAMMHAEQKVDVFEFVSRIRNQRPQMVQTDMQYTFIYQALLEYYLYGDTELDVSSLEKHLQTMHGTTTHFDKIGLEEEFRKLTNVRIMKENMRTGNLPANMKKARVIQIIPYDFNRVILSMKRGQEYTDYINASFIDGYRQKDYFIATQGPLAHTVEDFWRMIWEWKSHTIVMLTEVQEREQDKCYQYWPTEGSVTHGEITIEIKNDTLSEAISIRDFLVTLNQPQARQEEQVRVVRQFHFHGWPEIGIPAEGKGMIDLIAAVQKQQQQTGNHPITVHCSAGAGRTGTFIALSNILERVKAEGLLDVFQAVKSLRLQRPHMVQTLEQYEFCYKVVQDFIDIFSDYANFK

SEQ ID NO: 64 - DNA, 2827 bp, Mus musculus
  misc_feature 63, 295, 296: n = A,T,C or G
cacgggtcccagccctgctttaccagttgcgaaaccttaagtgccacactactttgtggtgtncagagctaaggctaaatacgaagggacgttgagttttctctatggctcgtctgagtgccatcaccttttaggatcctccctgatagagtgtcagtcagagtgcaggagagaggcatgttcctgcatcctcccagtgtaaagccaaagctccagagggtgagcgccgcgtgcgctcccagagccaggcaccaggcgtcgccgtcgcccctcgcagctccacggccccgccccnnccgcggagctggagctggagccggagccggagccctagcccagagctggaggcggccggaccgggccagacagatttcctgctctcctgtagggctgagggctgccggctgcgggctacgggctgcgggctacaggctacgggctacggactgcgggctgcgagagaagtcaccggcggcgcaggtacctcactgcctacgcgcgtccccacgaccctccctcgcgccggcggggacagcggacgccccggagcggagcgcgtcgggcgggcgcccgggagatgcggagtcgccgcggcggagcgatcggggctacagcaccggtccctgggagactatagccctcatttccccttggtgcaccatggagcccttctgtccactcctgctggcaagttttagcttgtcgctcgccagagctggccagggcaacgacaccaccccaacagagagcaactggaccagcacaactgcaggccctccggaccctggtgcatcccagccgctgctcacctggctgctgctgcccctgctcctcctcctgttcctgcttgcagcctacttcttcaggttccggaagcagaggaaggccgtggtcagcagcaacgacaagaaaatgcctaacgggatcttagaagagcaagagcagcagagagtgatgctgctgagcagatctccatcaggccccaagaagttcttccccatccccgtggagcacctggaggaggagatccgggtgagatctgcggatgactgcaagcgattccgagaggagttcaattcattgccatctggacacatacaaggaacctttgaactagcaaataaagaagaaaacagagaaaaaaacagataccccaacattctgcccaatgatcattgcagagtgattttgagccaagtggatggaatcccctgctctgactacattaatgcttcctacatcgatggctacaaagaaaagaacaaattcatagcagctcaaggccctaagcaggagacagtgaatgacttctggagaatggtctgggagcaaaggtcagccaccatcgtcatgttgacgaacctgaaggagaggaaggaggagaagtgctaccagtactggccagaccagggctgttggacctacggcaacatccgggtgtgtgtagaggactgcgtggtcctggtggattacacgatccgaaagttctgcatccatccgcaactcccagacagctgcaaagccccgcggctggtctcacagctgcacttcaccagctggcctgacttcggggtgccgtttacccccatcgggatgctcaagttcctgaagaaagtgaagacactcaacccctcacatgctgggcccattgtggttcactgtagcgcgggcgtgggtcggactggcaccttcattgtgatcgatgccatgatggacatgatacactcggagcagaaggttgacgtctttgagtttgtgtctagaatccgcaatcagcgccctcagatggtccagacggatgttcagtatacattcatctaccaagccttactggaatactacctctatggggacacagagctggatgtgtcctccctggagaggcacctgcagacgctccatagcacagccacccattttgacaagatcgggctggaggaagagttcaggaagctgaccaacgtgcgaatcatgaaggagaacatgaggacgggcaacctgcctgccaacatgaagaaggcccgcgtcatccagatcattccatatgacttcaatcgggtcatcctgtccatgaaaagagggcaagagttcacagactatatcaacgcatccttcatagatggctacaggcagaaggactacttcatggccacacaggcgcctctggctcacacagttgaggacttctggaggatggtatgggagtggaagtctcacacaatgctcatgctgacggaggtgcaggagcgggaacaggataaatgctaccagtattggccaacggagggctcggtgactcatggagatataactatagagataaagagcgacaccctgtctgaagcaatcagcgtacgagactttctggttaccttcaaacagcccctggcccgccaggaagagcaggtccgcatggtgagacaattccatttccatggctggcctgaggttggcatccccgctgaaggcaaaggcatgattgacctgattgcagcagtgcagaagcagcagcagcagacgggcaaccaccccatcaccgtgcactgcagcgcgggagcagggcggacaggtacattcatagcactcagtaacattttggaacgagtgaaagccgagggactcctagacgtgtttcaagctgtgaagagcttaagacttcagagaccacacatggtgcagaccctggagcaatatgaattctgctacaaagtggtacaagattttatcgatatattttctgattatgctaatttcaaatgaagattcctgccttaaaatattttttaatttaatggtcagtatattttgtaaaaaaaaaaaaaaaaaa

SEQ ID NO: 65 - protein, 699 aa, Mus musculus
MEPFCPLLLASFSLSLARAGQGNDTTPTESNWTSTTAGPPDPGASQPLLTWLLLPLLLLLFLLAAYFFRFRKQRKAVVSSNDKKMPNGILEEQEQQRVMLLSRSPSGPKKFFPIPVEHLEEEIRVRSADDCKRFREEFNSLPSGHIQGTFELANKEENREKNRYPNILPNDHCRVILSQVDGIPCSDYINASYIDGYKEKNKFIAAQGPKQETVNDFWRMVWEQRSATIVMLTNLKERKEEKCYQYWPDQGCWTYGNIRVCVEDCVVLVDYTIRKFCIHPQLPDSCKAPRLVSQLHFTSWPDFGVPFTPIGMLKFLKKVKTLNPSHAGPIVVHCSAGVGRTGTFIVIDAMMDMIHSEQKVDVFEFVSRIRNQRPQMVQTDVQYTFIYQALLEYYLYGDTELDVSSLERHLQTLHSTATHFDKIGLEEEFRKLTNVRIMKENMRTGNLPANMKKARVIQIIPYDFNRVILSMKRGQEFTDYINASFIDGYRQKDYFMATQAPLAHTVEDFWRMVWEWKSHTMLMLTEVQEREQDKCYQYWPTEGSVTHGDITIEIKSDTLSEAISVRDFLVTFKQPLARQEEQVRMVRQFHFHGWPEVGIPAEGKGMIDLIAAVQKQQQQTGNHPITVHCSAGAGRTGTFIALSNILERVKAEGLLDVFQAVKSLRLQRPHMVQTLEQYEFCYKVVQDFIDIFSDYANFK

SEQ ID NO: 66 - DNA, 2155 bp, Rattus norvegicus
cccaaacagttcagataccgctggtcaggcctgcgctgtacacagctcagccatgagcagcagaaagaacttttccaggctcacctggttcaggaagcagaggaaagccgtggtcaacagcaacgacaagaagatgcctaacgggatcctagaagaacaagagcagcagagagtgatgctactgagcagatctccatcgggtcccaagaagtacttccccatccctgtggagcacctggaggaggagatccgggtgagatctgcggatgactgcaagcggttccgagaggagttcaattcattgccatccggacacatacaaggaacctttgaactagcaaataaagaagaaaacagagaaaaaaacagataccccaacattctgcccaatgatcattgcagagtgattttgagccaattggatggaatcccctgctctgactacattaatgcttcctacatagatggctacaaagaaaagaacaaattcatagcagctcaaggccccaagcaggagacagtgaatgacttctggagaatggtctgggagcaaaggtcagccaccattgtcatgttgacgaacctgaaggaaaggaaggaggagaagtgctaccagtactggccagaccagggctgttggacttatgggaacatccgggtgtgtgtagaggactgcgtggtcctggtggattacaccatccgcaagttctgcatccatccgcaactccccgacagctgcaaagctccacggctggtctcacagctgcacttcaccagctggcctgactttggggtaccatttacccccatcggaatgctgaagttcctgaagaaagtgaagacactcaacccctcacatgctgggcccattgtggtccactgtagcgcgggcgtgggtcggactggcaccttcattgtgatcgatgccatgatggacatgatccactcggaacagaaggttgacgtcttcgagtttgtgtctagaatccgcaatcagcgccctcagatggtccagacagatgttcagtatacattcatctaccaagccttactggaatactacctctatggggacacggagctggatgtgtcctccttggagaggcacctgcagacgctacatggcacagccacccattttgacaagatcgggctggaggaagaattcaggaaactgaccaacgtgcgaatcatgaaggagaacatgaggacgggcaacctgccggccaacatgaagaaggcccgtgtaatccagatcatcccatatgacttcaatcgggtaatcctgtccatgaaaagagggcaagagttcacagactacatcaatgcatccttcatagatggctacaggcagaaggactacttcatggctacacaggggcctctggcgcacacagttgaggacttctggaggatggtctgggagtggaagtcacacacaatcgtcatgctgactgaggtgcaggagcgggagcaggataaatgctaccagtattggccaaccgagggctcagtgactcacggagatataactatagaaataaagagtgacaccctgtctgaagcaatcagcatacgagactttctggttactttcacacagcccctggcccgccaggaagagcaggtccggatggtgagacaattccatttccatgcctggcctgaggttggaatccccactgagggtaaaggcatgattgacctgctctcggcagtgcagaagcagcagcagcagacagacaaccaccccatcaccgtgcactgcagtgcgggagcagggcggacaggtacattcatagcactcagtaacattttggaacgagtgaaagccgagggactcctagatgtgtttcaagctgtgaagagcttaagacttcagagaccacacatggtgcaaaccctggagcaatatgaattctgctacaaagtggtacaagattttatcgatatattttctgattatgctaatttcaaatgaagatccctgccttaaaatattttttaatttaatggtcagtatattttgtaaaaaaaatcatgttaatttatttcctagtggatattaatatccgtcctgattcctttggtatatattttgttatgttctaaaggctacctgctgtaagattattaaatcataaatgtccttttt

SEQ ID NO: 67 - protein, 659 aa, Rattus norvegicus
PNSSDTAGQACAVHSSAMSSRKNFSRLTWFRKQRKAVVNSNDKKMPNGILEEQEQQRVMLLSRSPSGPKKYFPIPVEHLEEEIRVRSADDCKRFREEFNSLPSGHIQGTFELANKEENREKNRYPNILPNDHCRVILSQLDGIPCSDYINASYIDGYKEKNKFIAAQGPKQETVNDFWRMVWEQRSATIVMLTNLKERKEEKCYQYWPDQGCWTYGNIRVCVEDCVVLVDYTIRKFCIHPQLPDSCKAPRLVSQLHFTSWPDFGVPFTPIGMLKFLKKVKTLNPSHAGPIVVHCSAGVGRTGTFIVIDAMMDMIHSEQKVDVFEFVSRIRNQRPQMVQTDVQYTFIYQALLEYYLYGDTELDVSSLERHLQTLHGTATHFDKIGLEEEFRKLTNVRIMKENMRTGNLPANMKKARVIQIIPYDFNRVILSMKRGQEFTDYINASFIDGYRQKDYFMATQGPLAHTVEDFWRMVWEWKSHTIVMLTEVQEREQDKCYQYWPTEGSVTHGDITIEIKSDTLSEAISIRDFLVTFTQPLARQEEQVRMVRQFHFHAWPEVGIPTEGKGMIDLLSAVQKQQQQTDNHPITVHCSAGAGRTGTFIALSNILERVKAEGLLDVFQAVKSLRLQRPHMVQTLEQYEFCYKVVQDFIDIFSDYANFK

SEQ ID NO: 68 - DNA, 2938 bp, Homo sapiens
gccggggggacgcgggaggatggagcaagtggagatcctgaggaaattcatccagagggtccaggccatgaagagtcctgaccacaatggggaggacaacttcgcccgggacttcatgcggttaagaagattgtctaccaaatatagaacagaaaagatatatcccacagccactggagaaaaagaagaaaatgttaaaaagaacagatacaaggacatactgccatttgatcacagccgagttaaattgacattaaagactccttcacaagattcagactatatcaatgcaaattttataaagggcgtctatgggccaaaagcatatgtagcaactcaaggacctttagcaaatacagtaatagatttttggaggatggtatgggagtataatgttgtgatcattgtaatggcctgccgagaatttgagatgggaaggaaaaaatgtgagcgctattggcctttgtatggagaagaccccataacgtttgcaccatttaaaatttcttgtgaggatgaacaagcaagaacagactacttcatcaggacactcttacttgaatttcaaaatgaatctcgtaggctgtatcagtttcattatgtgaactggccagaccatgatgttccttcatcatttgattctattctggacatgataagcttaatgaggaaatatcaagaacatgaagatgttcctatttgtattcattgcagtgcaggctgtggaagaacaggtgccatttgtgccatagattatacgtggaatttactaaaagctgggaaaataccagaggaatttaatgtatttaatttaatacaagaaatgagaacacaaaggcattctgcagtacaaacaaaggagcaatatgaacttgttcatagagctattgcccaactgtttgaaaaacagctacaactatatgaaattcatggagctcagaaaattgctgatggagtgaatgaaattaacactgaaaacatggtcagctccatagagcctgaaaaacaagattctcctcctccaaaaccaccaaggacccgcagttgccttgttgaaggggatgctaaagaagaaatactgcagccaccggaacctcatccagtgccacccatcttgacaccttctcccccttcagcttttccaacagtcactactgtgtggcaggacaatgatagataccatccaaagccagtgttgcatatggtttcatcagaacaacattcagcagacctcaacagaaactatagtaaatcaacagaacttccagggaaaaatgaatcaacaattgaacagatagataaaaaattggaacgaaatttaagttttgagattaagaaggtccctctccaagagggaccaaaaagttttgatgggaacacacttttgaataggggacatgcaattaaaattaaatctgcttcaccttgtatagctgataaaatctctaagccacaggaattaagttcagatctaaatgtcggtgatacttcccagaattcttgtgtggactgcagtgtaacacaatcaaacaaagtttcagttactccaccagaagaatcccagaattcagacacacctccaaggccagaccgcttgcctcttgatgagaaaggacatgtaacgtggtcatttcatggacctgaaaatgccatacccatacctgatttatctgaaggcaattcctcagatatcaactatcaaactaggaaaactgtgagtttaacaccaagtcctacaacacaagttgaaacacctgatcttgtggatcatgataacacttcaccactcttcagaacacccctcagttttactaatccacttcactctgatgactcagactcagatgaaagaaactctgatggtgctgtgacccagaataaaactaatatttcaacagcaagtgccacagtttctgctgccactagtactgaaagcatttctactaggaaagtattgccaatgtccattgctagacataatatagcaggaacaacacattcaggtgctgaaaaagatgttgatgttagtgaagattcacctcctcccctacctgaaagaactcctgaatcgtttgtgttagcaagtgaacataatacacctgtaagatcggaatggagtgaacttcaaagtcaggaacgatctgaacaaaaaaagtctgaaggcttgataacctctgaaaatgagaaatgtgatcatccagcgggaggtattcactatgaaatgtgcatagaatgtccacctactttcagtgacaagagagaacaaatatcagaaaatccaacagaagccacagatattggttttggtaatcgatgtggaaaacccaaaggaccaagagatccaccttcagaatggacatgattcagggagctagaagacactttaagttatactggaaaattcaggtgccactgaaagccagatttatagtattccatctttaatatgtgggactaacagcagtgtagattgttaccttaatattttttgctgggaccatctacctgccttatactacacttaggaaaaagtattacatatggtttattttgaaacttcaagtattattgccttaatgtctcttaaccctgttacacgctgcttgtagacatgttaatatagtaatacctttatgatatattgagtttaaggactaccctttttctgttttatcatgtattcattattttgtatatgtacagggcaagtaggtatataatttgataaagttgcaattgaaatattattaacagaagatgtaagaaatttctgcatggtctaaatctttgtgtactttatttgtaaattatttgccctggagttttagaaaatagtttctgaattttaaacttgctggattcatgcagccagctttgcaggttatcagagatcaaagattgtaataataattttgtaaattgtaagcaaacattctgc

SEQ ID NO: 69 - protein, 780 aa, Homo sapiens
MEQVEILRKFIQRVQAMKSPDHNGEDNFARDFMRLRRLSTKYRTEKIYPTATGEKEENVKKNRYKDILPFDHSRVKLTLKTPSQDSDYINANFIKGVYGPKAYVATQGPLANTVIDFWRMVWEYNVVIIVMACREFEMGRKKCERYWPLYGEDPITFAPFKISCEDEQARTDYFIRTLLLEFQNESRRLYQFHYVNWPDHDVPSSFDSILDMISLMRKYQEHEDVPICIHCSAGCGRTGAICAIDYTWNLLKAGKIPEEFNVFNLIQEMRTQRHSAVQTKEQYELVHRAIAQLFEKQLQLYEIHGAQKIADGVNEINTENMVSSIEPEKQDSPPPKPPRTRSCLVEGDAKEEILQPPEPHPVPPILTPSPPSAFPTVTTVWQDNDRYHPKPVLHMVSSEQHSADLNRNYSKSTELPGKNESTIEQIDKKLERNLSFEIKKVPLQEGPKSFDGNTLLNRGHAIKIKSASPCIADKISKPQELSSDLNVGDTSQNSCVDCSVTQSNKVSVTPPEESQNSDTPPRPDRLPLDEKGHVTWSFHGPENAIPIPDLSEGNSSDINYQTRKTVSLTPSPTTQVETPDLVDHDNTSPLFRTPLSFTNPLHSDDSDSDERNSDGAVTQNKTNISTASATVSAATSTESISTRKVLPMSIARHNIAGTTHSGAEKDVDVSEDSPPPLPERTPESFVLASEHNTPVRSEWSELQSQERSEQKKSEGLITSENEKCDHPAGGIHYEMCIECPPTFSDKREQISENPTEATDIGFGNRCGKPKGPRDPPSEWT

SEQ ID NO: 70 - DNA, 3160 bp, Homo sapiens
agcgaccgcagccggggggacgcgggaggatggagcaagtggagatcctgaggaaattcatccagagggtccaggccatgaagagtcctgaccacaatggggaggacaacttcgcccgggacttcatgcggttaagaagattgtctaccaaatatagaacagaaaagatatatcccacagccactggagaaaaagaagaaaatgttaaaaagaacagatacaaggacatactgccatttgatcacagccgagttaaattgacattaaagactccttcacaagattcagactatatcaatgcaaattttataaagggcgtctatgggccaaaagcatatgtagcaactcaaggacctttagcaaatacagtaatagatttttggaggatgatatgggagtataatgttgtgatcattgtaatggcctgccgagaatttgagatgggaaggaaaaaatgtgagcgctattggcctttgtatggagaagaccccataacgtttgcaccatttaaaatttcttgtgaggatgaacaagcaagaacagactacttcatcaggacactcttacttgaatttcaaaatgaatctcgtaggctgtatcagtttcattatgtgaactggccagaccatgatgttccttcatcatttgattctattctggacatgataagcttaatgaggaaatatcaagaacatgaagatgttcctatttgtattcattgcagtgcaggctgtggaagaacaggtgccatttgtgccatagattatacgtggaatttactaaaagctgggaaaataccagaggaatttaatgtatttaatttaatacaagaaatgagaacacaaaggcattctgcagtacaaacaaaggagcaatatgaacttgttcatagagctattgcccaactgtttgaaaaacagctacaactatatgaaattcatggagctcagaaaattgctgatggagtgaatgaaattaacactgaaaacatgatcagctccatagagcctgaaaaacaagattctcctcctccaaaaccaccaaggacccgcagttgccttgttgaaggggatgctaaagaagaaatactgcagccaccggaacctcatccagtgccacccatcttgacaccttctcccccttcagcttttccaacagtcactactgtgtggcaggacaatgatagataccatccaaagccagtgttgcatatggtttcatcagaacaacattcagcagacctcaacagaaactatagtaaatcaacagaacttccagggaaaaatgaatcaacaattgaacagatagataaaaaattggaacgaaatttaagttttgagattaagaaggtccctctccaagagggaccaaaaagttttgatgggaacacacttttgaataggggacatgcaattaaaattaaatctgcttcaccttgtatagctgataaaatctctaagccacaggaattaagttcagatctaaatgtcggtgatacttcccagaattcttgtgtggactgcagtgtaacacaatcaaacaaagtttcagttactccaccagaagaatcccagaattcagacacacctccaaggccagaccgcttgcctcttgatgagaaaggacatgtaacgtggtcatttcatggacctgaaaatgccatacccatacctgatttatctgaaggcaattcctcagatatcaactatcaaactaggaaaactgtgagtttaacaccaagtcctacaacacaagttgaaacacctgatcttgtggatcatgataacacttcaccactcttcagaacacccctcagttttactaatccacttcactctgatgactcagactcagatgaaagaaactctgatggtgctgtgacccagaataaaactaatatttcaacagcaagtgccacagtttctgctgccactagtactgaaagcatttctactaggaaagtattgccaatgtccattgctagacataatatagcaggaacaacacattcaggtgctgaaaaagatgttgatgttagtgaagattcacctcctcccctacctgaaagaactcctgaatcgtttgtgttagcaagtgaacataatacacctgtaagatcggaatggagtgaacttcaaagtcaggaacgatctgaacaaaaaaagtctgaaggcttgataacctctgaaaatgagaaatgtgatcatccagcgggaggtattcactatgaaatgtgcatagaatgtccacctactttcagtgacaagagagaacaaatatcagaaaatccaacagaagccacagatattggttttggtaatcgatgtggaaaacccaaaggaccaagagatccaccttcagaatggacatgattcagggagctagaagacactttaagttatactggaaaattcaggtgccactgaaagccagatttatagtattccatctttaatatgtgggactaacagcagtgtagattgttaccttaatattttttgctgggaccatctacctgccttatactacacttaggaaaaagtattacatatggtttattttgaaacttcaagtattattgccttaatgtctcttaaccctgttacacgctgcttgtagacatgttaatatagtaatacctttatgatatattgagtttaaggactactctttttctgttttatcatgtatgcattattttgtatatgtacagggcaagtaggtatataatttgataaagttgcaattgaaatattattaacagaagatgtaagaaatttctgcatggtctaaatctttgtgtactttatttgtaaattatttgccctggagttttagaaaatagtttctgaattttaaacttgctggattcatgcagccagctttgcaggttatcagagatcaaagattgtaataataattttgtaaattgtaagcaaaaagttatttttatattatatacagtctaattgttcatcctaattgttcctgttttcatctagtcagagattcagtaagtgccttggaacaatattgaattctcttagcttgtgtgtgtttctttaatatttgaactcaagtgggattagaagactatcaaaatacatgtatgtttcagatatttgacctgtcattaaaaaaaacaaacagttttacagtg

SEQ ID NO: 71 - protein, 780 aa, Homo sapiens
MEQVEILRKFIQRVQAMKSPDHNGEDNFARDFMRLRRLSTKYRTEKIYPTATGEKEENVKKNRYKDILPFDHSRVKLTLKTPSQDSDYINANFIKGVYGPKAYVATQGPLANTVIDFWRMIWEYNVVIIVMACREFEMGRKKCERYWPLYGEDPITFAPFKISCEDEQARTDYFIRTLLLEFQNESRRLYQFHYVNWPDHDVPSSFDSILDMISLMRKYQEHEDVPICIHCSAGCGRTGAICAIDYTWNLLKAGKIPEEFNVFNLIQEMRTQRHSAVQTKEQYELVHRAIAQLFEKQLQLYEIHGAQKIADGVNEINTENMISSIEPEKQDSPPPKPPRTRSCLVEGDAKEEILQPPEPHPVPPILTPSPPSAFPTVTTVWQDNDRYHPKPVLHMVSSEQHSADLNRNYSKSTELPGKNESTIEQIDKKLERNLSFEIKKVPLQEGPKSFDGNTLLNRGHAIKIKSASPCIADKISKPQELSSDLNVGDTSQNSCVDCSVTQSNKVSVTPPEESQNSDTPPRPDRLPLDEKGHVTWSFHGPENAIPIPDLSEGNSSDINYQTRKTVSLTPSPTTQVETPDLVDHDNTSPLFRTPLSFTNPLHSDDSDSDERNSDGAVTQNKTNISTASATVSAATSTESISTRKVLPMSIARHNIAGTTHSGAEKDVDVSEDSPPPLPERTPESFVLASEHNTPVRSEWSELQSQERSEQKKSEGLITSENEKCDHPAGGIHYEMCIECPPTFSDKREQISENPTEATDIGFGNRCGKPKGPRDPPSEWT

SEQ ID NO: 72 - DNA, 236 bp, Homo sapiens
gttaaaaggaacagatacaaggacatactgccatttgatcacagccgagttaaattgacattaaagactccttcacaagattcagactatatcaatgcaaattttataaagggcgtctatgggccaaaagcatatgtagcaactcaaggacctttagcaaatacagtaatagatttttggaggatggtatgggagtataatgttgtgatcattgtaatggcctgccgagaatttga

SEQ ID NO: 73 - protein, 78 aa, Homo sapiens
VKRNRYKDILPFDHSRVKLTLKTPSQDSDYINANFIKGVYGPKAYVATQGPLANTVIDFWRMVWEYNVVIIVMACREF

SEQ ID NO: 74 - DNA, 2676 bp, Mus musculus
gaattcgcggccgctgtccagctctggcggcggcggcttcttctcagtagtgtaggtctctgggggcggcacgagttggacttccagatcttgaactcctttggaggctcctccggcgctttcaccacatgccgctcccagtcgatctgcttgttcgcctcgtagtaggggaaagcgacgctgctctttaagttgcgcggggagcccggccggtgccgccgcagccgcagccgcctgcgcggcggggagggagggcgggccggagcgagctggggaagacggagcgggcgctgcggcgcgcgggcgggcggcgggggggacgcgggaggatggagcaagtggagatcctgaggaggttcatccagagggtccaggccatgaagagtccggatcacaatggggaggacaacttcgcccgggacttcatgcgattgagaagattgtctaccaaatatagaacagaaaagatttatcccacagccactggagaaaaagaagaaaatgttaaaaagaacagatataaggacatactgccatttgatcacagccgagttaagttgactttgaagactccatcccaagattcagattatatcaatgcaaattttattaagggtgtgtatgggccaaaagcatatgtggcaacccaagggcctttccggaatacagtcatagacttctggaggatgatatgggagtataatgttgtgatgatcgtgatggcctgtcgagaatttgagatgggaaggaaaaagtgtgagcgctactggcctttgtatggagaagatcctataacatttgcaccatttaaaatttcttgtgaaaatgaacaagcaagaaccgactacttcatccgaacacttttacttgaatttcaaaatgaatcccgtcggctctatcagtttcattacgtgaactggccagaccatgatgttccttcgtcatttgattctattctggacatgataagcttaatgaggaaataccaagaacatgaagatgtgcctatttgtattcattgcagtgcaggctgtggacgaacaggtgctatttgtgccatagattacacgtggaacttactgaaagcagggaaaattccagaggaatttaatgtatttaatttaatacaagaaatgagaacacagaggcactcggcagtacaaacaaaggagcagtatgaacttgttcatagggctattgctcaactgtttgaaaaacagctacaactgtatgaaattcatggagcacagaagatccgtgatggtaatgaaattaccactggaactatggtcagttccattgatagcgagaagcaagactctcctccgccaaagccaccgcggactcgaagttgccttgtagaaggggatgccaaggaagaaatactacagccaccagaacctcacccggtgccacccatcctgacgccatcacctccttcagccttcccaaccgttaccactgtgtggcaggacagtgacaggtaccacccaaagccagtgctgcacatggcctcaccagagcaacacccagccgacctcaacagaagctatgataaatcagcggaccaatggggaaaaagtgaatcagctattgagcacatagataagaagttagagcgcaatttaagttttgagattaagaaagtccctctccaagaagggcccaaaagttttgatgggaacacactcttgaataggggacatgcgattaaaattaaatctgcttcatcttctgtagttgacagaacctctaaaccacaggagttaagtgcaggtgccctaaaggttgatgatgtatctcagaattcttgcgcggactgtagtgcggctcattcacacagagctgctgagtcgtcagaggagtcccagagcaactcacacacacctccacggccagactgcttgcctctcgataagaaaggacacgtaacgtggtcacttcatggacctgaaaatgccacacctgtacccgactcacctgacggcaaatccccagataatcattctcagactctgaaaaccgtgagttccacacccaactccaccgcagaagaggaagcccacgatcttacagagcaccacaacagctcccctctgttgaaagctcccctcagctttaccaaccctcttcactctgacgactggcactcagacggagggagctctgatggtgctgtgaccaggaacaaaactagcatttcaacagcaagtgccacagtgtctcctgccagtagtgctgagagtgcttgccataggagagtattgccgatgtccattgccagacaggaagtagcaggcacgccgcattcaggtgctgagaaagatgctgatgttagtgaggagtcgcctcctcctttacctgaacgaactcctgagtcttttgtattagcagatatgcctgtaagacctgagtggcatgaacttccaaatcaggagtggtctgaacaaagggaatctgaaggcttgacaacctctggaaatgaaaaacatgatgcagggggcatccacacagaggcttctgcagactctccacctgctttcagtgacaagaaagatcaaataacaaaaagtccagcagaagtcacagatattggttttggtaatcgctgtggaaaacctaaaggaccaagagagccaccttcagaatggacatgatgcagggagtgaaaggaca

SEQ ID NO: 75 - protein, 775 aa, Mus musculus
MEQVEILRRFIQRVQAMKSPDHNGEDNFARDFMRLRRLSTKYRTEKIYPTATGEKEENVKKNRYKDILPFDHSRVKLTLKTPSQDSDYINANFIKGVYGPKAYVATQGPFRNTVIDFWRMIWEYNVVMIVMACREFEMGRKKCERYWPLYGEDPITFAPFKISCENEQARTDYFIRTLLLEFQNESRRLYQFHYVNWPDHDVPSSFDSILDMISLMRKYQEHEDVPICIHCSAGCGRTGAICAIDYTWNLLKAGKIPEEFNVFNLIQEMRTQRHSAVQTKEQYELVHRAIAQLFEKQLQLYEIHGAQKIRDGNEITTGTMVSSIDSEKQDSPPPKPPRTRSCLVEGDAKEEILQPPEPHPVPPILTPSPPSAFPTVTTVWQDSDRYHPKPVLHMASPEQHPADLNRSYDKSADQWGKSESAIEHIDKKLERNLSFEIKKVPLQEGPKSFDGNTLLNRGHAIKIKSASSSVVDRTSKPQELSAGALKVDDVSQNSCADCSAAHSHRAAESSEESQSNSHTPPRPDCLPLDKKGHVTWSLHGPENATPVPDSPDGKSPDNHSQTLKTVSSTPNSTAEEEAHDLTEHHNSSPLLKAPLSFTNPLHSDDWHSDGGSSDGAVTRNKTSISTASATVSPASSAESACHRRVLPMSIARQEVAGTPHSGAEKDADVSEESPPPLPERTPESFVLADMPVRPEWHELPNQEWSEQRESEGLTTSGNEKHDAGGIHTEASADSPPAFSDKKDQITKSPAEVTDIGFGNRCGKPKGPREPPSEWT

SEQ ID NO: 76 - DNA, 1608 bp, Rattus norvegicus
ccggccggtgccgctggcagccgctagccgcctcgctgctggcggtggtagggtagggcggtgctcggagcgagctggggaagacggagcgggcgctgcggcgcgcgggcgggcggcgggggggacgcgggaggatggagcaagtggagattctgaggaggttcatccagagggtccaggccatgaaaagtccggaccacaatggggaggacaacttcgcccgggacttcatgcgattgagaagattgtctaccaaatatagaacagaaaagatttatcccacagccactggagaaaaagaagaaaatgttaaaaagaacagatataaggacatactgccatttgatcacagccgagttaagttgactttgaagacaccatcccaagattcagattatatcaatgcaaattttattaagggtgtatatgggccgagagcatacgtggcaacccaagggcctttggcgaatacagtcatagacttctggaggatgatatgggagtacaatgtggtgatcattgtaatggcctgtcgtgaatttgagatgggaaggaaaaagtgtgagcgctattggcctttgtatggagaagatcctataacatttgcaccatttaaaatttcttgtgaaaatgaacaagcaagaacagactacttcattcgaacacttttacttgaatttcaaaatgaatcccgtcgactctatcagtttcattacgtgaactggccagaccatgatgtcccttcgtcattcgattctatcctggacatgataagtctaatgaggaagtaccaagagcacgaagatgtgcctatttgtatccattgcagtgcaggctgtggacggacaggtgctatttgtgccatcgactacacgtggaacttactgaaagcagggaaaattccagaggaatttaatgtatttaatttaatacaagaaatgagaacacaaaggcactctgcagtacaaacgaaggagcagtatgaactcgtccatagagctattgcccaactgtttgaaaaacagctacaactgtatgaaatccacggagcccagaaaatcactgatggtaatgaaattagcactggaaacatggtcagttccattgatagtgaaaaacaagattctcctcctccaaagccaccacggactcgaagttgtcttgtagagggggatgccaaggaagaaatcctccagccaccagagcctcacccggtgccgcccatcctgacaccgtcgcctccttcagcattcccaacgttacactgtgtggcaagacagtgacaggtaccacccaaagccagtgctgcacatggcttccccagagcagcccccaacggacctcaacagaaactatgataagtcagcggacctaatggggagaagcgaatctgctgttgagcacacagataaaaagttagaacaaaatttaagttttgagattaagaaagtccctctccaagaagggcccaaaagttttgatgggaacacactcttgaataggggacatgcgattaaaattaagtctgcttcatcttctgtagttgacaaaagctctaagccacaggagttaagttcaggtgatctaaaggttcacgatgtgtctcag

SEQ ID NO: 77 - protein, 382 aa, Rattus norvegicus
MEQVEILRRFIQRVQAMKSPDHNGEDNFARDFMRLRRLSTKYRTEKIYPTATGEKEENVKKNRYKDILPFDHSRVKLTLKTPSQDSDYINANFIKGVYGPRAYVATQGPLANTVIDFWRMIWEYNVVIIVMACREFEMGRKKCERYWPLYGEDPITFAPFKISCENEQARTDYFIRTLLLEFQNESRRLYQFHYVNWPDHDVPSSFDSILDMISLMRKYQEHEDVPICIHCSAGCGRTGAICAIDYTWNLLKAGKIPEEFNVFNLIQEMRTQRHSAVQTKEQYELVHRAIAQLFEKQLQLYEIHGAQKITDGNEISTGNMVSSIDSEKQDSPPPKPPRTRSCLVEGDAKEEILQPPEPHPVPPILTPSPPSAFPTLHCVARQ

SEQ ID NO: 78 - DNA, 4315 bp, Homo sapiens
ggaaattgttcctcgtctgataagacaacagtggagaaaggacgcatgctgtttcttagggacacggctggcttccagatatgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagaccttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcttggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggctaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcataggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgagtgtatatgtatgtgtgtatgggtgtgtgtttgtgtgagagacagagaaagagagagaattc

SEQ ID NO: 79 - protein, 1304 aa, Homo sapiens [sequence omitted]

SEQ ID NO: 80 - DNA, 4597 bp, Homo sapiens
cgacattttaactgaactgcgggataaagtgaaatctttccgtgcagctctacgagaggaggaaattgttcctcgtctgataagacaacagtggagaaaggacgcatgctgtttcttagggacacggctgacttccagatatgaccatgtatttgtggcttaaactcttggcatttggctttgcctttctggacacagaagtatttgtgacagggcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagccttaaatcaaggttcataggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatatatatctatcttatatacttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgttaaatcccattcataactttcattaaagcatttactttgaatttc

SEQ ID NO: 81 - protein, 1143 aa, Homo sapiens
MYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 82 - DNA, 5007 bp, Mus musculus
ggagaccctatttcttaggggcacagctgatctccagatatgaccatgggtttgtggctcaaacttctggcctttggatttgcccttctggacacagaagtctttgtcacagggcaaacacctacacccagtgatgaactgagcacaacagagaatgcccttcttctgcctcaaagtgaccccttacctgctcgcaccactgaatccacacccccaagcatctctgaaagaggaaatggctcttcagagaccacatatcatccaggtgtgttatccacgctgctgcctcacctgtccccacagcctgactcgcagacgccctctgccggaggagctgacactcagacattcagcagccaagctgacaatcccacactcacgcctgctcccggcggcgggactgacccaccaggtgtgccaggggagaggactgtaccggggaccattcctgcagacacagcctttcctgttgatacccccagccttgcacgcaacagctctgctgcctcacctacacacacctccaatgtcagcaccacagatatctcttcaggtgccagcctcacaactcttacaccatccactctgggccttgcaagcactgaccctccaagcacaaccatagctaccacaacgaagcaaacatgtgctgccatgtttgggaacattactgtgaattacacctatgaatctagtaatcagacttttaaggcagacctcaaagatgtccaaaatgctaagtgtggaaatgaggattgtgaaaacgtgttaaataatctagaagaatgctcacagataaaaaacatcagtgtgtctaatgactcatgtgctccagctacaactatagatttatatgtaccaccagggactgacaagttttcgctacatgactgcacaccaaaagaaaaggctaatacttcaatttgtttggagtggaaaacagaaaaccttgatttcagaaaatgcaacagtgacaatatttcatatgtactccactgtgagccagaaaataatacaaaatgcattagaagaaatacattcatacctgaaagatgtcagttggacaaccttcgtgcccaaacaaattacacatgtgtagcagaaatcttatatcgcggtgtaaaactcgtcaaaaatgttataaatgtgcagacagatttggggattccagaaacgcctaagcctagttgtggggatccagctgcaagaaaaacgttagtctcttggcctgagcctgcatctaaacctgatcctgcatctaaaccccatggatatgttttatgctataagaacaattcagaaaaatgtaaaagtttgcctaataatgtgaccagttttgaggtggaaagcttgaaaccttataaatactatgaagtgtccctacttgcctatgtcaatgggaagattcaaagaaatgggactgctgagaagtgcaattttcacacaaaagcagatcgtccagacaaggtcactggaatgaaaacctcccggccgacagacaatagtataaatgttacatgtggtcctccttatgaaactaatggccctaaaaccttttacattttggtagtcagaagtggaggttcttttgttacaaaatacaacaagacaaactgtcagttttatgtagataatctctactattcaactgactatgagtttctggtctcttttcacaatggagtgtacgagggagattcagttataagaaatgagtcaacaaattttaatgctaaagcactgattatattcctggtgtttctgattattgtgacatcaatagccttgcttgttgttttgtataaaatctatgatctgcgcaagaaaagatccagcaatttagatgaacaacaggaactcgttgaaagggatgatgaaaagcagctgatggatgtggagccaatccattctgacattttgttggaaacatacaaaaggaagattgctgatgagggcagactgttcctggctgaatttcagagcattccacgggtattcagcaagtttcccatcaaagatgcccgaaagccccacaatcagaataaaaaccgttatgttgacattcttccctatgattataaccgtgtggaactctctgaaataaatggagatgcagggtccacctacataaatgccagctacattgatggcttcaaggaacccaggaaatacattgctgcacaagggccccgggatgagacagttgatgacttctggaggatgatctgggagcaaaaggccacagttattgtcatggtcacacgatgtgaagaaggaaacaggaacaagtgcgcagaatactggccaagcatggaggaaggcactcgagctttcaaagatattgttgtgacaatcaatgaccacaaacgatgtcctgattacatcattcagaagctgaacgttgcacataaaaaagaaaaagcaactggaagagaagtgactcatatccaattcaccagctggccagaccatggggttcctgaagaccctcacctgctcctcaaacttcgacggagagttaatgcttttagcaacttcttcagtggtcccattgtggtgcactgcagtgctggtgttgggcgtacaggcacctacattggaattgatgccatgctggaaggcctggaagcagagggcaaagtggatgtctatggttatgttgtcaagctaaggcgacagaggtgtctgatggtgcaagtggaggcacagtatatcctgattcatcaggctttagtggaatacaatcagtttggagaaacagaagtgaacttgtctgagttacattcatgcctacacaacatgaagaagagagatccacccagtgacccctcccctctggaggctgaataccagagacttccttcatacaggagttggaggacacagcacattggaaatcaagaagaaaataagaagaagaacaggaattctaatgttgttccatatgactttaacagagtgccacttaagcatgaactggagatgagcaaagagagtgagcctgaatcagatgagtcttcagatgatgacagtgactcagaagaaaccagcaaatacattaatgcatcctttgtgatgagttactggaaaccagaaatgatgattgctgctcaggggccactaaaagaaacgatcggtgacttttggcagatgatattccaaagaaaagtcaaagttattgtgatgttgacagagttagtgaatggagaccaggaagtctgtgctcagtactggggcgaaggaaagcagacttatggagacatggaagtggagatgaaagacacaaacagagcctcagcctacactctccgaacttttgagctgagacattccaagaggaaggagcccagaactgtgtaccagtaccagtgtaccacatggaaaggggaagagctgcctgcagaacccaaagacctggtgtctatgattcaggacctcaaacagaagcttcccaaggcttccccagaagggatgaagtatcacaagcatgcatccatcctcgtccactgcagagatggatcccagcagacagggttgttctgtgccttgttcaatctcttggaaagtgcagaaacagaagatgtggttgatgttttccaagtggtaaagtctctacgcaaagcacggcctggggtggtgtgcagctatgagcaataccagttcctctatgacatcatcgccagcatctatcccgcccagaatggacaagtcaagaaaacaaacagccaagacaaaattgaatttcataatgaagtggatggaggcaagcaggatgctaactgtgtccgtccagatggtcctctgaataaagcccaggaagacagcagaggggtgggaaccccggagcctaccaatagtgctgaggaaccagaacatgctgccaatggttctgcgagcccagctccaacccagagttcataggaaaggagtcatgtgggacaacgcagactctcacattagttctttctatttttctagacctaatgaaagaaaatggctgtgcagtggtttatggaatctgtgttcacctttgccactgtataaaaatatttaagtttgtcaaaacattttgtacagttttatgcttattttaaaagtgtatctatgtcattcagcaggaatgtatatgtgagagagggtgtctgtgtgtgtgagagtgtgtttatgtatgagtgactgtgtgtgtgcatgtttgtgcgtgtgtatgacatctaaatgtgattggagaatactttcaagccatttcaaatgctttcgagaaacagtgtgccttttctcctcttgaggaaactatacattttatatctaaactgttaatttgtttgagggattaattttttaaaatcccattgaaagtggattcagttgtaagaataacaatgtgtaccattctggaatgacctcaaggtgtcctccttgtcctgttgatgatcttgtagtttaagatgctctttttggatatagataagcgtatgtaagagtgctgtgggtgtgtacagctgatctgggacgtgaacaaaatcaacatgtgagacttatgttccatatactgtcatttcatcactatctcttaatgcatatttaatcaaacatgaaaatctcaagggagactatttttgtatccacatgggaagtagaacattgcaagtcagttgctgtctacacaatagataaaaattactagttaatgctcttggtcatatcgatatatgctatgaacctaaataattgcccttagccaaatataatgtatgttaaaaacacatagaataaaaacaggggcatgaaaacttgtttgtactgaatatttacataggtaacctcgtacagttagttctgttatggaattcaccatttatgggaaatgtaaaattgactatggccatttcctatgcttaagaccatctttgacttgcattactgtgtatttatcttgaatttccccactgttttgtttactcttactgagatataatattgataaccataataaactttcaactatta

SEQ ID NO: 83 - protein, 1291 aa, Mus musculus
MGLWLKLLAFGFALLDTEVFVTGQTPTPSDELSTTENALLLPQSDPLPARTTESTPPSISERGNGSSETTYHPGVLSTLLPHLSPQPDSQTPSAGGADTQTFSSQADNPTLTPAPGGGTDPPGVPGERTVPGTIPADTAFPVDTPSLARNSSAASPTHTSNVSTTDISSGASLTTLTPSTLGLASTDPPSTTIATTTKQTCAAMFGNITVNYTYESSNQTFKADLKDVQNAKCGNEDCENVLNNLEECSQIKNISVSNDSCAPATTIDLYVPPGTDKFSLHDCTPKEKANTSICLEWKTENLDFRKCNSDNISYVLHCEPENNTKCIRRNTFIPERCQLDNLRAQTNYTCVAEILYRGVKLVKNVINVQTDLGIPETPKPSCGDPAARKTLVSWPEPASKPDPASKPHGYVLCYKNNSEKCKSLPNNVTSFEVESLKPYKYYEVSLLAYVNGKIQRNGTAEKCNFHTKADRPDKVTGMKTSRPTDNSINVTCGPPYETNGPKTFYILVVRSGGSFVTKYNKTNCQFYVDNLYYSTDYEFLVSFHNGVYEGDSVIRNESTNFNAKALIIFLVFLIIVTSIALLVVLYKIYDLRKKRSSNLDEQQELVERDDEKQLMDVEPIHSDILLETYKRKIADEGRLFLAEFQSIPRVFSKFPIKDARKPHNQNKNRYVDILPYDYNRVELSEINGDAGSTYINASYIDGFKEPRKYIAAQGPRDETVDDFWRMIWEQKATVIVMVTRCEEGNRNKCAEYWPSMEEGTRAFKDIVVTINDHKRCPDYIIQKLNVAHKKEKATGREVTHIQFTSWPDHGVPEDPHLLLKLRRRVNAFSNFFSGPIVVHCSAGVGRTGTYIGIDAMLEGLEAEGKVDVYGYVVKLRRQRCLMVQVEAQYILIHQALVEYNQFGETEVNLSELHSCLHNMKKRDPPSDPSPLEAEYQRLPSYRSWRTQHIGNQEENKKKNRNSNVVPYDFNRVPLKHELEMSKESEPESDESSDDDSDSEETSKYINASFVMSYWKPEMMIAAQGPLKETIGDFWQMIFQRKVKVIVMLTELVNGDQEVCAQYWGEGKQTYGDMEVEMKDTNRASAYTLRTFELRHSKRKEPRTVYQYQCTTWKGEELPAEPKDLVSMIQDLKQKLPKASPEGMKYHKHASILVHCRDGSQQTGLFCALFNLLESAETEDVVDVFQVVKSLRKARPGVVCSYEQYQFLYDIIASIYPAQNGQVKKTNSQDKIEFHNEVDGGKQDANCVRPDGPLNKAQEDSRGVGTPEPTNSAEEPEHAANGSASPAPTQSS

SEQ ID NO: 84 - DNA, 3541 bp, Rattus norvegicus
tgggtgggtgtctcaaaaaacaaaaaactcagaagatacaaatggagaaaaaaaatctaaacaccccaaaagaaacccaaggatgacagtagtaagaattttaacgcaaacctagaaggtgacaaaaaacctaagtgtgaatatacggattgtgaaaaagagttaaaaaatctaccagaatgctcacagaaaaacgtcactctctccaatggctcatgtactccagataaaattataaatttagatgtaccaccagggactcacaactttaacttaacaaactgcacaccagacatagaagctaatacctcaatttgtttggagtggaaaataaaaaacaaatttacctgcgacattcaaaagatttcatacaatttccgttgtacaccagagatgaaaacatttgctttggacaaacacggaacactttggttacacaaccttacagtccgaacaaattacacatgtgctgcggaagtcctctacaataacgtaatacttttaaaacaagacagaagggtgcagactgattttgggactccagaaatgcttccccatgttcaatgtaagaattcaactaacagcacaacattagtctcctgggctgagccagcatctaaacaccatggatacattttatgctataaaaagaccccttcagaaaaatgtgaaaatttggctaatgatgtgaacagttttgaggtgaaaaacctgaggccttatacagagtacacagtgtctctatttgcctatgttattgggagggtgcaacgaaatggccctgctaaggattgcaactttcggacaaaagcagctcgtccaggcaaagtcaatggtatgaaaacctcccgggcgtcagacaatagtataaatgtcacgtgtaattctccttatgaaattaatggccctgaagcgcgttacattttggaagtcaaaagtggaggttctttagttaaaactttcaaccaatccacatgtaaatttgttgtagacaatctctattattcaactgactatgagtttctggtctatttttacaatggagagtacctgggagacccagaaataaaacctcaatcaacatcttataattctaaagcactgattatattcctggtgtttctgattattgtgacatcaatagccctgcttgttgttttgtataaaatctatgatctgcgtaagaaaagatccagcaatttagatgaacagcaggaactcgttgaaagggatgaggagaagcagctgataaatgtggacccaattcattctgaccttttgttggaaacatacaaaagaaagattgccgatgagggtagactgttcctggctgaatttcagagcattccacgggtattcagcaagtttcccatcaaagatgcccgaaagtcccaaaaccagaacaaaaaccgttatgtggacattctcccctatgattacaaccgtgtggagctctctgaaataaatggagacgcagggtccacctacataaatgccagctacattgatggcttcaaggaacccaggaaatacattgctgcacaagggccccgggatgagacagttgatgacttctggaagatgatctgggagcagaaggccacagttattgtcatggtcacacgatgtgaagaaggaaacaggaacaagtgtgcagaatattggccatgcatggaggaaggcactcggactttcagagatgttgtcgtgacgatcaatgaccacaaacgatgtcctgattacattatccagaagctgagcattgcccacaaaaaagaaaaagcaactggaagagaagtgactcatattcaattcaccagttggccagaccatggggttcctgaagaccctcacctgctcctgaaacttcgacggagagttaatgcttttagcaacttcttcagtggacccattgtggtgcactgcagtgctggcgttgggcgtacaggcacctacattggaattgatgccatgctcgaaagcctagaagcagagggcaaagtggatgtctatggctatgttgtcaacctaaggcgacagagatgtctgatggtgcaagtggaggcccagtacatcctgatccatcaggccttagtggagtacaatcaatttggggaaacagaagtgaacttgtctgagttacattcatgtctacagaatctgaagaagagagatccacccagtgacccgtctcctttggaggctgagtaccagagacttccttcatacaggagctggaggacacagcacattggaaatcaagaagaaaataaaaagaaaaacaggagttctaacgttgttccatatgactttaacagagtgccacttaagcatgaactagagatgagcaaagagagcgaggctgaatccgacgaatcttcagatgaggacagtgactcggaagaaaccagcaaatacattaatgcgtcatttgtgatgagttactggaaaccagaaatgatgattgctgcccagggtccactaaaagagactattggtgacttttggcagatgatattccaaagaaaagtcaaggttattgtgatgttgacagagttaatgagtggagaccaggaagtctgtgctcaatactggggagaaggaaagcagacttatggagacatggaagtaatgttgaaagacacgaacaaatcctcagcctatattctgcgagcatttgaactgagacattccaagagaaaggagcctagaactgtgtaccagtaccagtgtaccacatggaaaggggaggagctccctgcagaacccaaagatctggtgactctgattcagaacatcaaacagaagcttcccaagagtggctcagaagggatgaagtaccacaagcatgcatcaatcctagtccactgcagggatggatcccagcagacggggttgttctgtgctctgttcaatctcctggaaagtgcagaaacagaagatgtggttgatgtttttcaagtggtaaagtctctacgcaaagcacggccggggatggtgggcagctttgagcaataccagttcctctatgacatcatggccagcatctatcccacccagaatggacaagtgaagaaagcaaacagccaagacaaaattgaatttcataacgaagtggacggagccaagcaggacgcaaactgtgttcagccagctgatcctctgaacaaagcccaggaagacagcaaagaagttggagcttcagagcctgcaagtggttctgaggagccagaacattctgcaaatggtcccatgagcccagctctaaccccgagctcataggaacggattcgagtgggacaacgcaaaatgtcaaattagttgtttctatttttctagacctaatgaaaaaatatggctgtacagtggtccgtggaatctgtgttcacctttgccactgtataaaaatatttaagtttgtcaaaaggttttgtacagttttatgcctattttaaaaatgtatctatatcatttagcaagagtgtgtgtaggagagtatgtctgtgtgtgagagagtgtgtgcttctgttagtgtgtgttt

SEQ ID NO: 85 - protein, 962 aa, Rattus norvegicus
MKTFALDKHGTLWLHNLTVRTNYTCAAEVLYNNVILLKQDRRVQTDFGTPEMLPHVQCKNSTNSTTLVSWAEPASKHHGYILCYKKTPSEKCENLANDVNSFEVKNLRPYTEYTVSLFAYVIGRVQRNGPAKDCNFRTKAARPGKVNGMKTSRASDNSINVTCNSPYEINGPEARYILEVKSGGSLVKTFNQSTCKFVVDNLYYSTDYEFLVYFYNGEYLGDPEIKPQSTSYNSKALIIFLVFLIIVTSIALLVVLYKIYDLRKKRSSNLDEQQELVERDEEKQLINVDPIHSDLLLETYKRKIADEGRLFLAEFQSIPRVFSKFPIKDARKSQNQNKNRYVDILPYDYNRVELSEINGDAGSTYINASYIDGFKEPRKYIAAQGPRDETVDDFWKMIWEQKATVIVMVTRCEEGNRNKCAEYWPCMEEGTRTFRDVVVTINDHKRCPDYIIQKLSIAHKKEKATGREVTHIQFTSWPDHGVPEDPHLLLKLRRRVNAFSNFFSGPIVVHCSAGVGRTGTYIGIDAMLESLEAEGKVDVYGYVVNLRRQRCLMVQVEAQYILIHQALVEYNQFGETEVNLSELHSCLQNLKKRDPPSDPSPLEAEYQRLPSYRSWRTQHIGNQEENKKKNRSSNVVPYDFNRVPLKHELEMSKESEAESDESSDEDSDSEETSKYINASFVMSYWKPEMMIAAQGPLKETIGDFWQMIFQRKVKVIVMLTELMSGDQEVCAQYWGEGKQTYGDMEVMLKDTNKSSAYILRAFELRHSKRKEPRTVYQYQCTTWKGEELPAEPKDLVTLIQNIKQKLPKSGSEGMKYHKHASILVHCRDGSQQTGLFCALFNLLESAETEDVVDVFQVVKSLRKARPGMVGSFEQYQFLYDIMASIYPTQNGQVKKANSQDKIEFHNEVDGAKQDANCVQPADPLNKAQEDSKEVGASEPASGSEEPEHSANGPMSPALTPSS

SEQ ID NO: 86 - DNA, 2090 bp, Homo sapiens
  misc_feature 2090: n = A,T,C or G
cttagtccctgagctctctgcctgcccagactagctgcacctcctcattccctgcgcccccttcctctccggaagcccccaggatggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtgagcggtgctgtcctcaggtggccatgcctcagccctgaccctgtggaaggatttcgcgatggacagactcacaacctgaacctaggagatgtcgtattcttttgtaatttaaatggctgtatcccccccctaacctctccctgaccctgtatatagcccagccaggcccagcagggccacccttctcctcttgtaaataaagccctgggatcactgtgan

SEQ ID NO: 87 - protein, 595 aa, Homo sapiens
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 88 - DNA, 2277 bp, Homo sapiens
caagaagacggggattgaggaggcctcaggcgcctttgtctacctgcggcagccgtactatgccacgagggtgaatgcggctgacattgagaaccgagtgttggaactgaacaagaagcaggagtccgaggaggaagtggctgattactgagcggttcttcctcacctggcttgggccactgtgcacagctgtgccgctggctcagccccgccccctgcggccctccgccgtggcttccccctccctacagagagatgctgtcccgtgggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtagagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtgagcggtgctgtcctcaggtggccatgcctcagccctgaccctgtggaagcatttcgcgatggacagactcacaacctgaacctaggagtgccccattcttttgtaatttaaatggctgcatcccccccacctctccctgaccctgtatatagcccagccaggccccaggcagggccaacccttctcctcttgtaaataaagccctgggatcactgaaaaaaaaaaaaaa

SEQ ID NO: 89 - protein, 597 aa, Homo sapiens
MLSRGWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 90 - DNA, 2145 bp, Homo sapiens
cggcagaactgggaccaccgggggtggtgaggcggcccggcactgggagctgcatctgaggcttagtccctgagctctctgcctgcccagactagctgcacctcctcattccctgcgcccccttcctctccggaagcccccaggatggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtggtgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcaacggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtgagcggtgctgtcctcaggtggccatgcctcagccctgaccctgtggaagcatttcgcgatggacagactcacaacctgaacctaggagtgccccattcttttgtaatttaaatggctgcatcccccccacctctccctgaccctgtatatagcccagccaggccccaggcagggccaacccttctcctcttgtaaataaagccctgggatcact

SEQ ID NO: 91 - protein, 595 aa, Homo sapiens [sequence omitted]

SEQ ID NO: 92 - DNA, 4301 bp, Homo sapiens
  misc_feature 387, 388, 3718, 3799, 4224: n = A,T,C or G
ccgaagggggaagattgcttgagctcaggagtttaaaaccagcctgggcaacatagagagaccccatctctattaaaaaaaaatactgggtatgatggcccaagcatgtggtagtcctagcagtttgggagtctgaggtgggaggatcacttgagcccaagagttcaagaccaccctgggcaacatagggagagacctcatctctactacgactacgactactactactactaataaatagctggatgtagtggcatgcacctgtggtctcagttacttggaaggctgaggcaggaggatcacctgacccaaggaggtcgacgctgcagtgagttggattgtgacactgcacttcagcctgggtgataaagcaagattctgtgtccnnaaaaaaaaaaaaaagagagggaaggaaagaaggaagggaaggaagaaagaaaaagagaaagaaggaaaaaaaggaaagagcgagaaagaagaaagaaaaggaaggaaggaaagaaagaaaagaaaggaaagaaaaagaaaaagtgacacccagtcgaaagaagaaaggaaagaaaaagaaaaagtgacaaccggtcgaaagaaaaaagaaaaagtgacaaccggctgggcatggtggctcaagcctgtaatcccagcactttgggaggccgaggcaggtggatcacgaggtcaggagttcaagaccagcctggccaacatggtgaaaccctgtctcaactaaagatacaaaaaaaaaattaggctggcacagtggtgcgcacctgtgagtcccagctactagggaggctgaggcaggagaattgcttgaacccaggaggcggaggttgcagtgagccgagattgcgtcactgcactccagcctgagtgcagcgggagagactccatctcaaaaaaaaaaaaaaaagaaaagaaaaagtgacaacctgcttacagagtactggcgagtttgtgggtgggtggctccctagccctgctgattcttgcttctcacactcatgtctgcccctgccccagtgcacatcttgtcactgtcggccccaccgatggggttcctactgagtcttctggtccctgatcccgtctgtggtcattttcctgccaggtagcttggccaggcctcccctggtgcagatttcatccttggtttctcagcctggccttgaatgaccctctacagcagggtccccacctctcagaacaactttgctccagccacatggcttgctcacggccaggcactgcccatgtggactctgtgcgtgccacctctttgccctgacccatgttgcctctgggggagcacttcttcctccaccttccatcatgggctgtggcagtgcccatcccatctgcccccgacgctgtctgctgcagtatggttgttgggggaaagggcaccaggctccggcgtctgacagccgtgttttacccaccttcctactcactagcttgtgaccttgggcaattacttaacatctctgagtcttagtttctgtttctaaaattgggtgaataacacctactaagtagggttggcctgaggattaatagtataatgtaaaagctggcagcactgaaaccctgccacttaccagcttttcacatcagtatttgggaaatattgttaagctcatttgtcaggcggggattctgaggctcagagcagttccagaactttctacagattattttgccttgtttgcgcttccagactgcctatcttcttgtatcaccattgatcttgatctgtatggtttttaatttttttttttttgagacggagtttcactctgttgcccaggctggagtgcggtggcatgatctcggctcactgcaacctccacctcctgagaagctgggattacaggcatgtgccaccacacccggctaatctttgtatttttagtagagttggggtttcactatgttggccaggctggtctcgatctcctgacctcgtgatccgccagccttggcctcccaaagtgctgggattacaggcgtgagccactgcgcccggccaacttcacgtttatacacacccatgcaaacagcatccagatagagacaaagagccttccctgtaccctaaaagtttcccagaaattgttcccagttagcatatttatttttataaaggtaatgcatgcccatcatataacattcaaaaaggtatgtagagaaccaagtgtctcccccagccctgtcctccagccacccagtttccctccctaggggaagccaccaatatgtgtttcttatgtatcccctgttgagctgcttttcctcgttttggtttggcggtgttgatgtttgtatttggaattacaggtaggcagcatcatataccttagtgtttagggcctctaagatcaaccagccctgagaaaatcagccatggtgaggaccttgtcccccagccccccaggagataggccccctggtgggagtgctggggcagggcagaggcctagggacaagaattagaaaggacccatgttgacagggctgctcagggtcatgttgtccatccctctgccacagtggcatggacaaactgcatatgttggttagaggagggcacccttctctcttgcaagcattggcaaggtcttaactattagtctcctgctcccatggcagcccctttggacaaggaggctcttaatctctgttctttgaagccctgagggctggtgtataggagttcaaagcactggctttggaaccggactgtctgggtttgaatcctggcactgcagctgactcactgatggactcaggcaatgccttaaactccctgagcctcaggttccttgtctgtaaaatgataaagatagcccctgtttcatagggctgtggtgagaaaccaatcagacaaggcatgtgaacgccattatagcacagcgcccggcatccagcaggactcactcgatgacagttgtcaccgccatcattgttattagcgtgggccagggagggctgcgtaaaagcagctggtggaggagggagagatgccgtgggaccgtctgggttcgcatgcgtgaagtattatctgggcctggagtgtgcaaggcacacatgtgtccttactgcatgtgttgtcacatatgtgcaatgccatgctcctgagcctttgattgcagacgtgtgggaagtgggccccgtccccacccccagtgccaccctgctctgcttctcttcccttgctgtgctctaaaacgagaagtacaagtgagttcccccaaggggtcggccgcgcctcttcctgtccccgccctgccggctgccccaggccagtggagtggcagccccagaactgggaccaccgggggtggtgaggcggcccggcactgggagctgcatctgaggcttagtccctgagctctctgcctgcccagactagctgcacctcctcattccctgcgcccccttcctctccggaagcccccaggatggtgaggtaagggcctgccacccacggtagacaggaggcaagggtgcctggtgcccacgggacccctcctcactgccctgcctgggccgcccaggtggtttcaccgagacctcagtgggctggatgcagagaccctgctcaagggccgaggtgtccacggtagcttcctggctcggcccagtcgcaagaaccagggtgacttctcgctctccgtcaggtaggtgggccccccgcaaccccgggcattttggncactctcttgtgccatccaggccctgaaccactcattcctggttccccgtggcagtgctgactccccgtctgttcccttgnccccaacccccacactccccatccctgtctgtgcccacccatgcccatgtgtgcccccacccaggacctcagccgatccctgccctcctgcctctactcctgcaccgactggcctcaccgcctggtgccctgcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtgagggctccgcacccccgccattcccaagcagggatgagccggctcccaccctgaacagccagggaggcagggagactggcagccggcgctgnctaccctccatcccctcccctccctgcaccagctggggctctcaatgtccctcctccctgctgtcctgggacctggt

SEQ ID NO: 93 - DNA, 8545 bp, Homo sapiens
  misc_feature 1958, 2678, 3190, 4689, 4712, 5166, 5632, 5777, 7758,8183: n = A,T,C or G
actccctcttgcaggtgtccttaagtttgctcgcttggtcaagtcctacgaagcccaggatcctgagatcgccagcctgtcaggcaagctgaaggcgctgtttctgccgcccatgaccctgccaccccatgggcctgctgctggtggcagcgtggccgcctcctgagagttggccctcccttgtgccactgccaggggaggaaaggccttgatgttccagacaataataaatgcgcctgtgacttagccttggtgtcagtctcttgcggacctgacaacccccatctctccttccctgattccctctgcctttccaggccccatccccctgaacagctcctccctatggtcctggctgggcctaaccctgccccagggcctaaccctacctgaggctcctccccttcccccggggcaggttgagaggctggagtgggtccctcagcgccctgggtgggtgggcctgcacagggggtacctccttctctgaggaactgggctgttagggattttccttaggccctttggtttccgcctacggagaggtttcccccattggttgctcttcctcagccagggttacttcctggtctgttcccctacccaataccccgccgctctgtcagcttgagctccaggtggagctccaggtggctcctcctctcccgggggaaggcggccctggaccagcaggcgggcctgctgtactcccgctttggggctgcagggaagctggccgctgtgggcggtctcgggccagccccgccccacctgtccttttcctggagactattagtccagggtttgtccctgcagtgccattggcctggcaggcaggatcgaggaggaagtggctgattactgagcggttcttcctcacctggcttgggccactgtgcacagctgtgccgctggctcagccccgccccctgcggccctccgccgtggcttccccctccctacagagagatgctgtcccgtgggtaagtcccgggcaccatcggggtcccagtctcctgttagttttggagggagggagggctttgttgatgctcactccgacgtgtgtgaacgtgagtgcgatctgccgctgccctgcgcctgtttccggtccctatgaacttccccttcccgcaaggtgtgaggacccccggctcactcatgctcctctgccccctctttaacattttcccctggacaagtgtgtatctgttctctccattgcatttctacttccagcctctgggctcctgcttctgcctcctgcttaggacctgtccccctgggtagctcacaacacctcaaacatagcagtcagaggccacccgcgaaggccctcccacgtccagccaacttctccgcacttcccaacatcagactttggtcccatcttctttgtttcctttcacttccctttcccctgcatcattcattcaacaggtacgtgttgagcatctattatgcaccaggtgctgtttaagatgctggtaatactggagtgaacaagacagacatggtctctgctctcacggagcttacattccagtgggaggttacagaccgaacaaataacccaataaattggatcattgcagattctcagaagtattacgcagaaaatagacagccttggccgggtgtagtggttcacacctgtgatcccagcactgtgggaggctgaggcgagaggattgcttgagcccaggagtttgagaccagcctggccaatatagtgagaccctgtctctacaaaaaataagaaattagctgggtgtggtggcacacgtcctgtggttccagctatggagaggctaaggtgagaggcttgcttgagcctgggaggtcaaggctgcagtcagcgatgattgcaccactgcacaccagcctgggcgacagagtgagaccttgtctcaaaaaaaaaaaaaaaaaagaaaatgaaccagcttcatatgctagcnagtgactgggtgtgcaggtgacattactagctggagggatcagggaggccttcccgaggaggtgacatttgagctgagacccggatgaggaggaagaggagctggccatgtgacgtagtgatcaagagtcaagcatctctgggcagaggagatggtgagcacaaagccctaatgtgggaacaaacaaaaaaaggacagtgtgcccgtggcagaggaccctagtggagcggaggcagggccacagcaggttagaccatgttggagctaggatgttgaaagtgaaaacctgacgagatgaggtggcgcacgtctgtgatcccagcactttgggaggccgaagggggaagattgcttgagctcaggagtttaaaaccagcctgggcaacatagagagaccccatctctattaaaaaaaaatactgggtatgatggcccaagcatgtggtagtcctagcagtttgggaggctgaggtgggaggatcacttgagcccaagagttcaagaccaccctgggcaacatagggagagacctcatctctactacgactacgactactattactactaataaatagctggatgtagtggcatgcacctgtggtctcagttacttggaaggctgaggcaggaggatcacctgagccaaggaggtcgacgctgcagtgagttggattgtgacactgcacttcagcctgggtgataaagcaagattctgtgtcnaaaaaaaaaaaaaaaagagagggaaggaaagaaggaagggaaggaagaaagaaaaagagaaagaaggaaaaaaaggaaagagcgagaaagaagaaagaaaaggaaggaaggaaagaaagaaaagaaaggaaagaaaaagaaaaagtgacacccagtcgaaagaagaaaggaaagaaaaagaaaaagtgacaaccggtcgaaagaaaaaagaaaaagtgacaaccggctgggcatggtggctcaagcctgtaatcccagcactttgggaggccgaggcaggtggatcacgaggtcaggagttcaagaccagcctggccaacatggtgaaaccctgtctcaactaaagatacaaaaaaaaaattaggctggcacagtggtgcgcacctgtgagtcccagctactagggaggctgaggcaggagaattgcttgaacccaggaggcggaggttgcagtgagccgagattgcgtcactgcactccagcctgagtgcagcgggagagactccatctcnaaaaaaaaaaaaaaagaaaagaaaaagtgacaacctgcttacagagtactggcgagtttgtgggtgggtggctccctagccctgctgattcttgcttctcacactcatgtctgcccctgccccagtgcacatcttgtcactgtcggccccaccgatggggttcctactgagtcttctggtccctgatcccgtctgtggtcattttcctgccaggtagcttggccaggcctcccctggtgcagatttcatccttggtttctcagcctggccttgaatgaccctctacagcagggtccccacctctcagaacaactttgctccagccacatggcttgctcacggccaggcactgcccatgtggactctgtgcgtgccacctctttgccctgacccatgttgcctctgggggagcacttcttcctccaccttccatcatgggctgtggcagtgcccatcccatctgcccccgacgctgtctgctgcagtatggttgttgggggaaagggcaccaggctccggcgtctgacagccgtgttttacccaccttcctactcactagcttgtgaccttgggcaattacttaacatctctgagtcttagtttctgtttctaaaattgggtgaataacacctactaagtagggttggcctgaggattaatagtataatgtaaaagctggcagcactgaaaccctgccacttaccagcttttcacatcagtatttgggaaatattgttaagctcatttgtcaggcggggattctgaggctcagagcagttccagaactttctacagattattttgccttgtttgcgcttccagactgcctatcttcttgtatcaccattgatcttgatctgtatggtttttaatttttttttttttgagacggagtttcactctgttgcccaggctggagtgcggtggcatgatctcggctcactgcaacctccacctcctgagaagctgggattacaggctagtagagatggggttccactgtgttgcccagctggtctcgaactcctgacctcaagtgatcctcccacctcggcctcccaaagtgctgggattacaggtgtaagtcactgcgcccagctgtatttttattttttgagacagggtctcactctgtcacccaggccggattacagtggcacaaccatggctcactgcagcctcgaccaccccaggctcaagcgatcctcccatctcagtctcccaagtacctggggctacaggggtgtgctaccacacctggctaaattttgtattttttgtagagacagggtttctccaggtttcctaggctgctctcaaacttggtgtcaagtaatccaccagcctcacccccacaaagtgctgggattacaggcgtgagccactgcgcctggccttgatctctacttttatcttcctgcttccaaggaaatatttttttcttctgaattatcaggcatttactcttgtaattcttagtctccctacgcttgtttccncccataagaaaatggggaaaatnattcctacatcacagggctgtctgaggcctaaatgagatcgtgtatgtgaaagtgatctgcaaaccccacaccatgccaaggtaagggaggtagttttttattttcctgccaaaggatagcagaagctgtacctcttgtctgagttctgtctcttggcttgacacccttcagaggaattcctgcctcttccatgggtcaggaagaggtgcctagttagtttcttctgggtgccgagttaattccttcccaccaagtgggtttaagccctggaaggagtgtcttggggccagggtgcagtgggcgtttggtgctgagcatggacctgaaacttcgtgtgtggtcagatttatgttccatgcgtggggatgtgcaccggaccaggtatgtgtgtaggtggacatggatgatgaggtgtgtgtgctgtgactgtgtgtgcacccttgcctgcgtatnacaagcaggctgtgtgtgtaggaccaggaagctgtacttgtggccaggtatgtggctatggactgacgagcttgtttgttgaacacttgctcagtgccaggcatcgcactggactctgaacactccgagatgagcgagagcgccagcgggtgtcccgcgctgcagccagctttgcatgtgctcttcttgctccttggcgttgggcgtgggggttccgcggatttccggggctctggggtggtgtccaagctgaaggggtagtcaaagctggaccatgcaataaacccaccaggcagattccccagggcaccagtgagagaagaaaaccagaaacaaatagaacaaaaagaaagaaagaaaagtggaaaagccttttttgggggaaaacattgatgtttgatgtttctaaaaatgataatgtagttatcatgggaaaattagacttgttgggcttaaaactttttcntcttttacctgaagcagaacatgcataatgttcataaatattaagcacacaacctggctccattttttttttttttttgaaatagagtcttactgtgttgccaggctggagtgcagtggtgtgatcttggctcgctgcaacctcngcttcctgggttcaagtgattctcctgcttcaacctcccgagtagctgggattacaggcgcccaccaccatgcccagctaatttttttgtatttttagtagagagggggtttcaccatgttggccaggatggtctcgatctcctgacctcgtgatccgccagccttggcctcccaaagtgctgggattacaggcgtgagccactgcgcccggccaacttcacgtttatacacacccatgcaaacagcatccagatagagacaaagagccttccctgtaccctaaaagtttcccagaaattgttcccagttagcatatttatttttataaaggtaatgcatgcccatcatataacattcaaaaaggtatgtagagaaccaagtgtctcccccagccctgtcctccagccacccagtttccctccctaggggaagccaccaatatgtgtttcttatgtatcccctgttgagctgcttttcctcgttttggtttggcggtgttgatgtttgtatttggaattacaggtaggcagcatcatataccttagtgtttagggcctctaagatcaaccagccctgagaaaatcagccatggtgaggaccttgtcccccagcccccaggagataggccccctggtgggagtgctggggcagggcagaggcctagggacaagaattagaaaggacccatgttgacagggctgctcagggtcatgttgtccatccctctgccacagtggcatggacaaactgcatatgttggttagaggagggcacccttctctcttgcaagcattggcaaggtcttaactattagtctcctgctcccatggcagcccctttggacaaggaggctcttaatctctgttctttgaagccctgagggctggtgtataggagttcaaagcactggctttggaaccggactgtctgggtttgaatcctggcactgcagctgactcactgatggactcaggcaatgccttaaactccctgagcctcaggttccttgtctgtaaaatgataaagatagcccctgtttcatagggctgtggtgagaaaccaatcagacaaggcatgtgaacgccattatagcacagcgcccggcatccagcaggactcactcgatgacagttgtcaccgccatcattgttattagcgtgggccagggagggctgcgtaaaagcagctggtggaggagggagagatgccgtgggaccgtctgggttcgcatgcgtgaagtattatctgggcctggagtgtgcaaggcacacatgtgtccttactgcatgtgttgtcacatatgtgcaatgccatgctcctgagcctttgattgcagacgtgtgggaagtgggccccgtccccacccccagtgccaccctgctctgcttctcttcccttgctgtgctctaaaacgagaagtacaagtgagttcccccaaggggtcggccgcgcctcttcctgtccccgccctgccggctgccccaggccagtggagtggcagccccagaactgggaccaccgggggtggtgaggcggcccggcactgggagctgcatctgaggcttagtccctgagctctctgcctgcccagactagctgcacctcctcattccctgcgcccccttcctctccggaagcccccaggatggtgaggtaagggcctgccacccacggtagacaggaggcaagggtgcctggtgcccacgggacccctcctcactgccctgcctgggccgcccaggtggtttcaccgagacctcagtgggctggatgcagagaccctgctcaagggccgaggtgtccacggtagcttcctggctcggcccagtcgcaagaaccagggtgacttctcgctctccgtcaggtaggtgggccccccgcaaccccgggcattttggccactctcttgtgccatccaggccctgaaccactcattcctggttccccgtggcagtgctgactccccgtctgttcccttgnccccaacccccacactccccatccctgtctgtgcccacccatgcccatgtgtgcccccacccaggacctcagccgatccctgccctcctgcctctactcctgcaccgactggcctcaccgcctggtgccctgcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtgagggctccgcacccccgccattcccaagcagggatgagccggctcccaccctgaacagccagggaggcagggagactggcagccggcgctgnctaccctccatcccctcccctccctgcaccggctggggctctcaatgtccctcctccctgctgtcctgggacctggtgtctcagagcctaacctaccaccctttccacctaaccccgaggaagccacagaaagctgcctcgccctactccgggagccctggccgctgcaacccaggtcccactggagacagggaggccactgctggtggccagcatgtcgtgcaggccagctctgttgttagaaagctcttcttcctctggaatcgagcctgccttcctccgtctgtccctcaccccaggacatgttaggacagtgaggagctgacactgggggtgaagatggggatgaattgcttggcaag

SEQ ID NO: 94 - DNA, 2734 bp, Mus musculus
cctccagcacgctgtgctcctgcagcagcagtggcttcttggtgctgccttggccatgacacatatttgacatgccctcccttaacctactcacagactcttgctctgcggcatggaccaaagagaaattctgcagcaactactgaaagaagcccagaaaaagaaactcaacagtgaggagtttgccagtgaatttctgaagctgaaaaggcaatccaccaagtacaaggccgacaaaatctatcctacaactgtggctcagaggcccaagaatatcaagaaaaacagatacaaggatattttgccctatgatcacagcctggtagagctgtctctgttaacttccgatgaggattccagttatatcaatgccagctttattaagggtgtctatggacccaaggcttatattgctacccagggtcctttatctacaactctcctggacttctggaggatgatttgggagtaccgcatcttggtcattgtcatggcatgtatggagtttgaaatgggaaagaaaaaatgtgagcgttattgggccgaaccaggagaaacgcagctgcaatttggccccttttctatatcctgtgaagctgagaaaaagaaatctgattataaaatcaggactctgaaggccaagttcaataatgaaactcgaattatttaccagtttcattataagaattggccagaccatgatgtgccttcatctatagaccctattcttcagctcatctgggatatgcgttgttaccaagaagatgactgtgttcctatatgcattcactgcagtgccggctgcggaaggacaggtgtcatttgtgctgttgattatacatggatgctgctgaaagatgggataattcctaagaacttcagtgtttttaatttgattcaggagatgcgaacacagaggccttcgctagttcaaactcaggaacagtacgaactggtctacagtgctgtgttagagctgtttaagaggcacatggatgttatctctgataatcaccttggaagagagattcaagcacaatgctcaattcctgaacaaagcctcacggtagaagctgactcttgtcctctggatttaccaaaaaacgccatgagggatgtgaaaacgacaaaccagcatagcaaacaaggggctgaagcggagagcactggagggtcttcccttggccttaggacttctacgatgaatgccgaggaagagttggttttgcactcggctaaatcaagcccttcttttaactgtttagagctaaactgcgggtgtaacaacaaggctgtcataaccaggaacgggcaggcaagggcttctccagtcgtgggagagccccttcagaagtatcaaagtctggattttggttccatgttgtttgggtcgtgtcctagtgctctgcccataaacacagcggacaggtatcacaattcaaaggggccggtaaaacggaccaaatcaactccctttgaactgattcagcagagaaaaacaaatgacttggccgtgggagacggtttttcatgcctggaatctcagctgcatgagcattacagtctcagggagctgcaggtgcaaagagtggcccatgtttcttcagaagagctgaattattcactgcctggtgcctgtgatgcgtcgtgtgtgccccggcacagccccggcgctttgagagtgcatctgtacacatctttagcggaagatccttatttttcatcatcccctccgaatagtgctgattcgaagatgtcttttgatctgcctgagaaacaggatggagccacttcccctggcgctctattgccagcctcttctacaacctccttcttttatagcaacccacacgactccctagtgatgaacactctgaccagcttttccccaccgttaaaccaagagacagctgtagaagctccttctcggaggacagatgatgaaatccccccgccactccctgaacggacacccgagtcttttattgtggttgaggaagccggagagccctcaccacgtgttaccgaatccttacctctggtggtaacatttggagcatcaccagaatgcagtgggacatctgaaatgaagagccatgactctgtagggtttacaccaagcaagaatgtgaaactccgaagtcccaaatcagatcgacatcaggatggttctcctccacctcctctcccagaaagaactctagagtccttctttcttgctgatgaggactgtatacaggcccaagccgtgcaaacttcttctactagctatcctgaaaccacagagaactccacatcttctaaacaaacattgaggacccctggaaaaagtttcacaaggagtaagagtttgaagatttttcgaaatatgaaaaaaagtgtttgtaattcttcctcaccaagcaagcctacagaacgtgttcagccaaaaaattccagctcctttctgaattttggtttcggaaatcgtttttcaaaacccaaaggaccaaggaacccgccatcagcttggaatatgtaacgcacctctggatttataagaatatttgttaaaattgtacctgcagataaagctacttgaatactgctataataataatatcagtaatgaggatatattttgtcaatagtcttgaaaaggaaagcaatatgtgacaaacttccagcaaacttgtattttcatattccttatatttcactgggaactgccaataaagtttgtacttcaaaaaaa

SEQ ID NO: 95 - protein, 802 aa, Mus musculus
MDQREILQQLLKEAQKKKLNSEEFASEFLKLKRQSTKYKADKIYPTTVAQRPKNIKKNRYKDILPYDHSLVELSLLTSDEDSSYINASFIKGVYGPKAYIATQGPLSTTLLDFWRMIWEYRILVIVMACMEFEMGKKKCERYWAEPGETQLQFGPFSISCEAEKKKSDYKIRTLKAKFNNETRIIYQFHYKNWPDHDVPSSIDPILQLIWDMRCYQEDDCVPICIHCSAGCGRTGVICAVDYTWMLLKDGIIPKNFSVFNLIQEMRTQRPSLVQTQEQYELVYSAVLELFKRHMDVISDNHLGREIQAQCSIPEQSLTVEADSCPLDLPKNAMRDVKTTNQHSKQGAEAESTGGSSLGLRTSTMNAEEELVLHSAKSSPSFNCLELNCGCNNKAVITRNGQARASPVVGEPLQKYQSLDFGSMLFGSCPSALPINTADRYHNSKGPVKRTKSTPFELIQQRKTNDLAVGDGFSCLESQLHEHYSLRELQVQRVAHVSSEELNYSLPGACDASCVPRHSPGALRVHLYTSLAEDPYFSSSPPNSADSKMSFDLPEKQDGATSPGALLPASSTTSFFYSNPHDSLVMNTLTSFSPPLNQETAVEAPSRRTDDEIPPPLPERTPESFIVVEEAGEPSPRVTESLPLVVTFGASPECSGTSEMKSHDSVGFTPSKNVKLRSPKSDRHQDGSPPPPLPERTLESFFLADEDCIQAQAVQTSSTSYPETTENSTSSKQTLRTPGKSFTRSKSLKIFRNMKKSVCNSSSPSKPTERVQPKNSSSFLNFGFGNRFSKPKGPRNPPSAWNM

SEQ ID NO: 96 - DNA, 2176 bp, Rattus norvegicus
gaattcggcacgagaggggcttggctcaaagtgccattggtttgacaggctggatgaggaggaagtggccgaaaccgaaatattcttcctgaaggtctggatccccgaacagctgtgccactcgattggccccgcccctgtcgccctttgcctgtgacttcccccactcctccagggagatgctgtcccgcgggtggtttcaccgggacctcagtgggcctgatgccgagaccctgctcaagggccggggagtccctgggagcttcctggctcggcccagtcgcaagaaccagggtgacttctccctctcagtcagggtggatgaccaggtgactcatattcggatccagaactcaggggacttctatgacctgtatggaggggagaagtttgcgacgtcgacagagctggtggagtattacactcagcagcagggcatcctgcaggaccgagacggcaccatcatccacctcaagtacccactgaactgctcggaccccaccagcgagaggtggtatcatggtcacatgtctggagggcaggcagagtcactgctgcaggccaagggcgagccctggacatttcttgtgcgtgagagtctcagccaacctggtgattttgtgctctctgtgctcaatgaccagcccaaggctgccccgggttccccgctcagggtcacgcacatcaaggttatgtgtgagggtggacgatacactgtgggtggctcagagacattcgacagcctcacagacctggtggagcacttcaagaagacggggattgaggaggcctcaggtgcctttgtctacctgaggcagccttactatgccactcgggtaaatgcagcagacattgagaaccgggtcttggaactgaacaagaagcaggagtcagaggacacagccaaggccggcttctgggaggagtttgagagtctgcaaaagcaagaggcaaagaacttgcaccagcgtctggaagggcagcggccggagaacaagagcaagaaccgctacaagaacattcttccctttgaccacagccgagtgatcctgcagggacgtgacagtaacatcccagggtctgattacatcaatgccaactacgttaagaaccagctgctaggtccggatgagaactctaagacctacatcgccagtcagggctgtctggacgctaccgtcaatgacttctggcagatggcttggcaggagaacactcgtgtcatcgtcatgactaccagagaggtggagaaaggccggaacaaatgtgtcccatactggcctgaggtgggcactcagcgcgtctatgggctctactctgtgaccaactgtaaagagcatgacacagcagagtacaaacttcgaacattgcagatctccccactggacaatggggacctggttcgggagatatggcactaccagtacctgagctggcctgaccatggggttcccagtgagcctggaggtgtcctcagctttctggatcagatcaaccagcggcaggaaagtttgcctcacgcggggcccatcattgtgcattgcagcgctggcatcggccgcaccggcaccatcatcgtcattgatatgctcatggagagcgtctccaccaaggggctagactgtgacattgacatccagaagaccatccagatggtacgggcacagcgctctggcatggtgcagacagaggcacagtacaagtttatttatgtggccatcgcccagttcatcgaaacaaccaagaagaaactggagatcatacaatcccagaggggccaggagtcggagtatgggaacatcacctaccctccggctttgaggagtgcccacgccaaagcctcccgtacctcgtccaaacacaaggaggaggtgtacgaaaacgtgcatagcaagaacaagaaggaagagaaagtaaagaagcagcgatcggcagacaaggagaagaacaaaggttctctcaagaggaacatcagccttactccgtgcagaggcctccgctgggcagacagagacctgtagtccacaccacccccatcttgttgtaatttaagtgaccgtggtcctctgaacctgtatatggctcagcaagcctcagggagagtcagacccttctcttcttgtaaataaagcccctggacaactgtgtaaaaaaaaaaaaaaaaaaaaaactcgag

SEQ ID NO: 97 - protein, 613 aa, Rattus norvegicus
MLSRGWFHRDLSGPDAETLLKGRGVPGSFLARPSRKNQGDFSLSVRVDDQVTHIRIQNSGDFYDLYGGEKFATSTELVEYYTQQQGILQDRDGTIIHLKYPLNCSDPTSERWYHGHMSGGQAESLLQAKGEPWTFLVRESLSQPGDFVLSVLNDQPKAAPGSPLRVTHIKVMCEGGRYTVGGSETFDSLTDLVEHFKKTGIEEASGAFVYLRQPYYATRVNAADIENRVLELNKKQESEDTAKAGFWEEFESLQKQEAKNLHQRLEGQRPENKSKNRYKNILPFDHSRVILQGRDSNIPGSDYINANYVKNQLLGPDENSKTYIASQGCLDATVNDFWQMAWQENTRVIVMTTREVEKGRNKCVPYWPEVGTQRVYGLYSVTNCKEHDTAEYKLRTLQISPLDNGDLVREIWHYQYLSWPDHGVPSEPGGVLSFLDQINQRQESLPHAGPIIVHCSAGIGRTGTIIVIDMLMESVSTKGLDCDIDIQKTIQMVRAQRSGMVQTEAQYKFIYVAIAQFIETTKKKLEIIQSQRGQESEYGNITYPPALRSAHAKASRTSSKHKEEVYENVHSKNKKEEKVKKQRSADKEKNKGSLKRNISLTPCRGLRWADRDL

SEQ ID NO: 98 - protein, 11 aa, Artificial Sequence
  Unique signature sequence motif found within conserved domain
  VARIANT 1: Xaa = Ile or Val
  VARIANT 4, 7, 8: Xaa = Any Amino Acid
  VARIANT 10: Xaa = Ser or Thr
XHCXAGXXRXG